SEQUENCE LISTING (409 sequences)

SEQ ID NO: 1 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1245 aa, Ruminococcus bromii
MQERKKISHLTHRNSVKKTIRMQLNPVGKTMDYFQAKQILENDEKLKENYQKIKEIADRFYRNLNEDVLSKTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTNDGTKVKGINEYINLYNQQVSKRDKIPNLQILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIRKKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCKDEKYYLAIIDKSNNRILENIDFQDCDESDCYEKIIYKLLPTPNKMLPKVFFAKKHKKLLSPSDEILKIYKNGTFKKGDKFSLDDCHKLIDFYKESFKKYPKWLIYNFKFKKTNGYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKRASTFPYDLIKDKRYTKWQFSLHFPITMNFKDPDKAMINDDVRNLLKSCNNNFIIGIDRGERNLLYVSVINSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYEKIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 4 - protein, 1562 aa, unidentified
  source 1..1562: note = Leptospira ilyithenensis
MKATSIWDNFTRKYSVSKTLRFELRPVGKTEENIVKKEIIDAEWISGKNIPKGTDADRARDYKIVKKLLNQLHILFINQALSSENVKEFEKEDKKSKTFVAWSDLLATHFDNWIQYTRDKSNSTVLKSLEKSKKDLYSKLGKLLNSKANAWKAEFISYHKIKSPDNIKIRLSASNVQILFGNTSDPIQLLKYQIELDNIKFLKDDGSEYTTKELADLLSTFEKFGTYFSGFNQNRANVYDIDGEISTSIAYRLFNQNIEFFFQNIKRWEQFTSSIGHKEAKENLKLVQWDIQSKLKELDMEIVQPRFNLKFEKLLTPQSFIYLLNQEGIDAFNTVLGGIPAEVKAEKKQGVNELINLTRQKLNEDKRKFPSLQIMYKQIMSERKTNFIDQYEDDVEMLKEIQEFSNDWNEKKKRHSASSKEIKESAIAYIQREFHETFDSLEERATVKEDFYLSEKSIQNLSIDIFGGYNTIHNLWYTEVEGMLKSGERPLTRVEKEKLKKQEYISFAQIERLISKHSQQYLDSTPKEANDRSLFKEKWKKTFKNGFKVSEYTNLKLNELISEGETFQKIDQETGKETTIKIPGLFESYENAILVESIKNQSLGTNKKESVPSIKEYLDSCLRLSKFIESFLVNSKDLKEDQSLDGCSDFQNTLTQWLNEEFDVFILYNKVRNHVTKKPGNTDKIKINFDNATLLDGWDVDKEAANFGFLLKKADNYYLGIADSSFNQDLKYFNEGERLDEIEKNRKNLEKEESKNISKIDQEKVKKYKEVIDDLKAISNLNKGRYSKAFYKQSKFTTLIPKCTTQLNEVIEHFKKFDTDYRIENKKFAKPFIITKEVFLLNNTVYDTATKKFTLKIGEDEDTKGLKKFQIGYYRATDDKKGYESALRNWITFCIEFTKSYKSCLNYNYSSLKSVSEYKSLDEFYKDLNGIGYTIDFVDISEEYINKKINEGKLYLFQIYNKDFSEKSKGKENLHTTYWKLLFDSKNLEDVVIKLNGQAEVFFRPASIHEKEKITHFKNQEIQNKNPNAVKKTSKFEYDIIKDNRFTKNKFLFHCPITLNFKADGNPYVNNEVQENIAKNPNVNIIGIDRGEKHLLYFTVINQQGQILDAGSLNSIKSEYKDKNQQSVSFETPYHKILDKKESERKEARESWQEIENIKELKAGYLSHVVHQLSNLIVKYNAIVVLEDLNKGFKRGRFKVEKQVYQKFEKSLIEKLNYLVFKDRKESNEPGHHLNAYQLTNKFLSFERLGKQSGVLFYATASYTSKVDPVTGFMQNIYDPYHKEKTREFYKNFTKIVYNGNYFEFNYDLNSVKPDSEEKRYRTNWTVCSCVIRSEYDSNSKTQKTYNVNDQLVKLFEDAKIKIENGNDLKSTILEQDDKFIRDLHFYFIAIQKMRVVDSKIEKGEDSNDYIQSPVYPFYCSKEIQPNKKGFYELPSNGDSNGAYNIARKGIVILDKIRLRVQIEKLFEDGTKIDWQKLPNLISKVKDKKLLMTVFEEWAELTHQGEVQQGDLLGKKMSKKGEQFAEFIKGLNVTKEDWEIYTQNEKVVQKQIKTWKLFSNST

SEQ ID NO: 5 - protein, 1262 aa, Fibrobacter succinogenes
MNLNTYFSQFTGLYPVSKTLRFELKPMGKTLEKIKETGIIENDKKRHNDYFDAKKIIDKYHKYFIDAALSKFPCIDWNPLKEAIERSLDRSDASKKKLEKTQTEFRKKIAKALTTHGHYKELTASTPKDLFLKVFPDHFGKQPAIDTFDGFSSYFTGFQENRQNIYSDEAISTAIPYRLVHDNFPKFLSNIEVYNILKDNAPSVLSDAENELKDFLNGKPLANIFELNAYNDVLTQSGIDFFNQVIGGFSGEGGEKKTRGINEFSNLYRQQHPEFAQKRLATKMIPLYKQILSDRETKSFILESYSTDSQVQESVKEFFESQILNCDIAGRKVNVLKELSSLIKRITEFDLGSIYVNQEELSSISLELFKSWNTINAILFKNAENRIGSAEKAANKKKIDAWMKSNEFSIATLNLAIAESDSEEISRVKIESYWNNFEAKVQSILCGDNRRNLDEFISATFNENNALREDSKVIEKLKAFLDALIEIMHSIKPLISDAENRDLSFYNELMPLYDQLSLVVPLYNKIRNYATQKLTESEKFKLNFDNPTLADGWDQNKEEANTAILLLKNGLYYLGIMNAKNKPKIKDFKTSESEDCYDKMVYKLLPGPNKMLPKVFFSEKGLATFKPPKDILDGYNAGKHKKGDLFDIGFCHQLIDFFKESIAKHPDWKKFDFKFSDTSSYEDISGFYKEVTDQGYKITFSKIPTPQIDEWVNEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLDGELSDEAKRYLDVAVVKDVKHEIVKDRRYTQDKFEFHVPLTLNFKADSKNEYMNERVRHFLKDNPDVNIIGIDRGERHLLYMTLINQKGEILKQKSFNIVESVNYQAKLVQREKERDTARRSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKEFLSHFEDITYDGKNDRFLFSFDYKKFKCFQTDYIKKWTVYSQGKRIVYDKESKSAKAISPVEIIKAALAKQNIALTDQLDVLSAINSVEASRETASFFGDICYAFEKTLQMRNSIPNTDEDYLVSPVMNKKGEFYDSRSCGDSLPKNADANGAYHIALKGLYLIKNVFDAGGKDLKISHEDWFKFAQSRNR

SEQ ID NO: 6 - protein, 1275 aa, Clostridium sp.
MGKNQNFQEFIGVSPLQKTLRNELIPTETTKKNIAQLDLLTEDEVRAQNREKLKEMMDDYYRDVIDSTLRGELLIDWSYLFSCMRNHLSENSKESKRELERTQDSVRSQIHDKFAERADFKDMFGASIITKLLPTYIKQNSKYSERYDESVKIMKLYGKFTTSLTDYFETRKNIFSKEKISSAVGYRIVEENAEIFLQNQNAYDRICKIAGLDLHGLDNEITAYVDGKTLKEVCSDEGFAKVITQGGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 7 - protein, 1463 aa, unidentified
  source 1..1463: note = Saccharobesus litoralis
MSDRLDVLTNQYPLSKTLRFELKPVGATADWIRKHNVIRYHNGKLVGKDAIRFQNYKYLKKMLDEMHRLFLQQALVLEPNSNQAQELTALLRAIENNYCNNNDLLAGDYPSLSTDKTIKISNGLSKLTTDLFDKKFEDWAYQYKEDMPNFWRQDIAELEQKLQVSANAKDQKFYKGIIKKLKNKIQKSELKAETHKGLYSPTESLQLLEWLVRRGDIKLTYLEIGKENEKLNELVPLVELKDIHRNFNNFATYLSGFSKNRENVYSTKFDRRSGYKATSVIARTFEQNLMFCLGNIAKWHKVTEFINQANNYELLQEHGIDWNKQIAALEHKLDVCLAEFFALNNFSQTLAQQGIEKYNQVLAGIAEIAGQPKTQGLNELINLARQKLSAKRSQLPTLQLLYKQILSKGDKPFIDDFKSDQELIAELNEFVSSQIHGEHGAIKLINHELESFINEARAAQQQIYVPKDKLTELSLLLTGSWQAINQWRYKLFDQKQLDKQQKQYSFSLAQVERWLATEVEQQNFYQTEKERQQHKDTQPANVTTSSDGHSILTAFEQQVQTLLTNICVAAEKYRQLSDNLTAIDKQRESESSKGFEQIAVIKTLLDACNELNHFLARFTVNKKDKLPEDRAEFWYEKLQAYIDAFPIYELYNKVRNYLSKKPFSTEKVKINFDNSHFLSGWTADYERHSALLFKFNENYLLGVVNENLSSEEEEKLKLVGGEEHAKRFIYDFQKIDNSNPPRVFIRSKGSSFAPAVEKYQLPIGDIIDIYDQGKFKTEHKKKNEAEFKDSLVRLIDYFKLGFSRHDSYKHYPFKWKASHQYSDIAEFYAHTASFCYTLKEENINFNVLRELSSAGKVYLFEIYNKDFSKNKRGQGRDNLHTSYWKLLFSAENLKDVVLKLNGQAEIFYRPASLAETKAYTHKKGEVLKHKAYSKVWEALDSPIGTRLSWDDALKIPSITEKTNHNNQRVVQYNGQEIGRKAEFAIIKNRRYSVDKFLFHCPITLNFKANGQDNINARVNQFLANNKKINIIGIDRGEKHLLYISVINQQGEVLHQESFNTITNSYQTANGEKRQVVTDYHQKLDMSEDKRDKARKSWSTIENIKELKAGYLSHVVHRLAQLIIEFNAIVALEDLNHGFKRGRFKIEKQVYQKFEKALIDKLSYLAFKDRTSCLETGHYLNAFQLTSKFKGFNNLGKQSGILFYVNADYTSTTDPLTGYIKNVYKTYSSVKDSTEFWQRFNSIRYIASENRFEFSYDLADLKQKSLESKTKQTPLAKTQWTVSSHVTRSYYNQQTKQHELFEVTARIQQLLSKAEISYQHQNDLIPALASCQSKALHKELIWLFNSILTMRVTDSSKPSATSENDFILSPVAPYFDSRNLNKQLPENGDANGAYNIARKGIMLLERIGDFVPEGNKKYPDLLIRNNDWQNFVQRPEMVNKQKKKLVKLKTEYSNGSLFNDLAFK

SEQ ID NO: 8 - protein, 1334 aa, Succinivibrio dextrinosolvens
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 9 - protein, 1310 aa, unidentified
  source 1..1310: note = Sedimentisphaera cyanobacteriorum
MKEFTNQYSLTKTLRFELRPVGETAEKIEDFKSGGLKQTVEKDRERTEAYKQLKEVIDSYHRDFIEQAFARQQTLSEEDFKQTYQLYKEAQKEKDGETLTKQYEHLRKKIAAMFSKATKEWAVMGENNELIGKNKESKLYQWLEKNYRAGRIEKEEFDHNAGLIEYFEKFSTYFVGFDKNRANMYSKEAKATAISFRTINENMVKHFDNCQRLEKIKSKYPDLAEELKDFEEFFKPSYFINCMNQSGIDYYNISAIGGKDEKDQKANMKINLFTQKNHLKGSDKPPFFAKLYKQILSDREKSVVIDEFEKDSELTEALKNVFSKDGLINEEFFTKLKSALENFMLPEYQGQLYIRNAFLTKISANIWGSGSWGIIKDAVTQAAENNFTRKSDKEKYAKKDFYSIAELQQAIDEYIPTLENGVQNASLIEYFRKMNYKPRGSEEDAGLIEEINNNLRQAGIVLNQAELGSGKQREENIEKIKNLLDSVLNLERFLKPLYLEKEKMRPKAANLNKDFCESFDPLYEKLKTFFKLYNKVRNYATKKPYSKDKFKINFDTATLLYGWSLDKETANLSVIFRKREKFYLGIINRYNSQIFNYKIAGSESEKGLERKRSLQQKVLAEEGEDYFEKMVYHLLLGASKTIPKCSTQLKEVKAHFQKSSEDYIIQSKSFAKSLTLTKEIFDLNNLRYNTETGEISSELSDTYPKKFQKGYLTQTGDVSGYKTALHKWIDFCKEFLRCYRNTEIFTFHFKDTKEYESLDEFLKEVDSSGYEISFDKIKASYINEKVNAGELYLFEIYNKDFSEYSKGKPNLHTIYWKSLFETQNLLDKTAKLNGKAEIFFRPRSIKHNDKIIHRAGETLKNKNPLNEKPSSRFDYDITKDRRFTKDKFFLHCPITLNFKQDKPVRFNEQVNLYLKDNPDVNIIGIDRGERHLLYYTLINQNGEILQQGSLNRIGEEESRPTDYHRLLDEREKQRQQARETWKAVEGIKDLKAGYLSRVVHKLAGLMVQNNAIVVLEDLNKGFKRGRFAVEKQVYQNFEKALIQKLNYLVFKEVNSKDAPGHYLKAYQLTAPFISFEKLGTQSGFLFYVRAWNTSKIDPATGFTDQIKPKYKNQKQAKDFMSSFDSVRYNRKENYFEFEADFEKLAQKPKGRTRWTICSYGQERYSYSPKERKFVKHNVTQNLAELFNSEGISFDSGQCFKDEILKVEDASFFKSIIFNLRLLLKLRHTCKNAEIERDFIISPVKGNNSSFFDSRIAEQENITSIPQNADANGAYNIALKGLMNLHNISKDGKAKLIKDEDWIEFVQKRKF

SEQ ID NO: 10 - protein, 1278 aa, Ruminococcus sp.
MSININKFSDECRKIDFFTDLYNIQKTLRFSLIPIGATADNFEFKGRLSKEKDLLDSAKRIKEYISKYLADESDICLSQPVKLKHLDEYYELYITKDRDEQKFKSVEEKLRKELADLLKEILKRLNKKILSDYLPEYLEDDEKALEDIANLSSFSTYFNSYYDNCKNMYTDKEQSTAIPYRCINDNLPKFIDNMKAYEKALEELKPSDLEELRNNFKGVYDTTVDDMFTLDYFNCVLSQSGIDSYNAIIGNDKVKGINEYINLHNQTAEQGHKVPNLKRLYKQIGSQKKTISFLPSKFESDNELLKAVYDFYNTGDAEKNFTALKDTITEFEKIFDNLSEYNLDGVFVRNDISLTNLSQSMFNDWSVFRNLWNDQYDKVNNPEKAKDIDKYNDKRHKVYKKSESFSINQLQELIATTLEEDINSKKITDYFSCDFHRVTTEVENKYQLVKDLLSSDYPKNKNLKTSEEDVALIKDFLDSVKSLESFVKILTGTGKESGKDELFYGSFTKWFDQLRYIDKLYDKVRNYITEKPYSLDKIKLSFDNPQFLGGWQHSKETDYSAQLFMKDGLYYLGVMDKETKREFKTQYNTPENDSDTMVKIEYNQIPNPGRVIQNLMLVDGKIVKKNGRKNADGVNAVLEELKNQYLPENINRIRKTESYKTTSNNFNKDDLKAYLEYYIARTKEYYCKYNFVFKSADEYGSFNEFVDDVNNQAYQITKVKVSEKQLLSLVEQGKLYLFKIYNKDFSEYSKGKKNLHTMYFQMLFDDRNLENLVYKLQGGAEMFYRPASIKKDSEFKHDANVEIIKRTCEDKVNDKDNPTDDEKAKYYSKFDYDIVKNKRFTKDQFSLHLTLAMNCNQPDHYWLNNDVRELLKKSNKNHIIGIDRGERNLIYVTIINSDGVIVDQINFNIIENSYNGKKYKTDYQKKLNQREEDRQKARKTWKTIETIKELKDGYISQVVHQICKLIVQYDAIVVMENLNGGFKRGRTKVEKQVYQKFETMLINKLNYYVDKGTDYKECGGLLKAYQLTNKFETFERIGKQSGIIFYVDPYLTSKIDPVTGFANLLYPKYETIPKTHNFISNIDDIRYNQSEDYFEFDIDYDKFPQGSYNYRKKWTICSYGNRIKYYKDSRNKTASVVVDITEKFKETFTNAGIDFVNDNIKEKLLLVNSKELLKSFMDTLKLTVQLRNSEINSDVDYIISPIKDRNGNFYYSENYKKSNNEVPSQPQDGDANGAYNIARKGLMIINKLKKADDVTNNELLKISKKEWLEFAQKGDLGE

SEQ ID NO: 11 - protein, 1264 aa, Prevotella brevis
MKQFTNLYQLSKTLRFELKPIGKTLEHINANGFIDNDAHRAESYKKVKKLIDDYHKDYIENVLNNFKLNGEYLQAYFDLYSQDTKDKQFKDIQDKLRKSIASALKGDDRYKTIDKKELIRQDMKTFLKKDTDKALLDEFYEFTTYFTGYHENRKNMYSDEAKSTAIAYRLIHDNLPKFIDNIAVFKKIANTSVADNFSTIYKNFEEYLNVNSIDEIFSLDYYNIVLTQTQIEVYNSIIGGRTLEDDTKIQGINEFVNLYNQQLANKKDRLPKLKPLFKQILSDRVQLSWLQEEFNTGADVLNAVKEYCTSYFDNVEESVKVLLTGISDYDLSKIYITNDLALTDVSQRMFGEWSIIPNAIEQRLRSDNPKKTNEKEEKYSDRISKLKKLPKSYSLGYINECISELNGIDIADYYATLGAINTESKQEPSIPTSIQVHYNALKPILDTDYPREKNLSQDKLTVMQLKDLLDDFKALQHFIKPLLGNGDEAEKDEKFYGELMQLWEVIDSITPLYNKVRNYCTRKPFSTEKIKVNFENAQLLDGWDENKESTNASIILRKNGMYYLGIMKKEYRNILTKPMPSDGDCYDKVVYKFFKDITTMVPKCTTQMKSVKEHFSNSNDDYTLFEKDKFIAPVVITKEIFDLNNVLYNGVKKFQIGYLNNTGDSFGYNHAVEIWKSFCLKFLKAYKSTSIYDFSSIEKNIGCYNDLNSFYGAVNLLLYNLTYRKVSVDYIHQLVDEDKMYLFMIYNKDFSTYSKGTPNMHTLYWKMLFDESNLNDVVYKLNGQAEVFYRKKSITYQHPTHPANKPIDNKNVNNPKKQSNFEYDLIKDKRYTVDKFMFHVPITLNFKGMGNGDINMQVREYIKTTDDLHFIGIDRGERHLLYICVINGKGEIVEQYSLNEIVNNYKGTEYKTDYHTLLSERDKKRKEERSSWQTIEGIKELKSGYLSQVIHKITQLMIKYNAIVLLEDLNMGFKRGRQKVESSVYQQFEKALIDKLNYLVDKNKDANEIGGLLHAYQLTNDPKLPNKNSKQSGFLFYVPAWNTSKIDPVTGFVNLLDTRYENVAKAQAFFKKFDSIRYNKEYDRFEFKFDYSNFTAKAEDTRTQWTLCTYGTRIETFRNAEKNSNWDSREIDLTTEWKTLFTQHNIPLNANLKEAILLQANKNFYTDILHLMKLTLQMRNSVTGTDIDYMVSPVANECGEFFDSRKVKEGLPVNADANGAYNIARKGLWLAQQIKNANDLSDVKLAITNKEWLQFAQKKQYLKD

SEQ ID NO: 12 - protein, 1315 aa, Helcococcus ovis
MSFERLTNIASISKTLRFRLKPVGKTLENLEKLGKLDKDFERNNFYPILKNIADDYYRQYIRNRLTDLNLDWIKLYYAHELLNSTDKESKKNLTTIQSEYRKILLNILSGELDKNGEKFSKDIVKKNKELYGKLFKKEFILEILPKFVETTNIYNKEYFNGINLYNKFTTRLSNFWEARKNIFTDKDIATGIPFRVVNENFVYFYKNIQVFNKNIKYLEDKLDNLEKNLKSEGIMSIDKSIKDFFNPNGFNYVITQKGIDTYQAIRGGFTKENGEKVQGINEILNLTQQKLRRNPYTKNIKLGVLTKLRKQILEYSESTSFLIDQIEDDNDLVDRINKFNVSFFESTEVSPSIFVQLENLYNSLRTANSEDIYIDARNTQKFSQMLFGQWDVIRRGYSLKITEGTKEEKKKYKKYIELDETSKAKGYLTLMEIQELVSSVEGYEEIDVFNVLLEKFKINIIERLKVETPIYGSPMKLEAIKEYLEKHLEEYHKWKLLLINNDELDLDEAFYPLLNEVISDYNIIQLYNLTRNYLTRKYSDKEKIKINFDFPTLADGWSESKISDNRSIILRKDGNYYLGILEDNKLLDNNITNFLENCYEIMKYNLFPDAAKMIPKCSISKKEVKNHFENGEDKSIYLSNQFVGRLEISKELYELQNNLVDGKKKYQIDYLRNTDDKVGYRNALNQWITFCKKFLNKYQGTQDFDYSKLKEAKYYDKLDQFYADVDSYGYSLDFDTINEDLVNKAVEDGKLLLFQIYNKDFSPESKGKKNLHTLYWLSMFSDENLKARKLKLNGQAEIFYRKKLEKKPIIHKEGSILLNKIDKDGNTIPENIYHECYRYLNKKIGRKDLSDKAITLFNKDVLNYKEARFDIIKDRRYSESQFFFHVPITFNWDLKSNQNVNSIVQNMIKDREIKHIIGIDRGERHLLYYSVIDLEGNIVEQGSLNTLNQNRFDNSIVEVDYQDKLRTREEDRDRARKNWTNINKIKELKDGYLSHVVHKLSKLIIDYEAIVILENLNQGFKRGRFKVERQVYQKFELALMNKLSALSFKETYDEGKNLEPSGILNPIQACYPVDSYQDLQGQNGIVFYLPAAYTSVIDPVTGFTNLFRLNSINTTKYEEFIKGFKNIYFDNEDLDFKFIFDYKNFEKFNFVSFKNKKSKKWIVSTRGERISYNSKKKEYFYVKPTEILKNKLIELGINFEDKDKDIISLIDKINDSKKIKLLKVVFDAFKYSVQLRNHDNIQDYIISPVADENGNYYNSNDVAIKNLKLPDNGDANGAFNIARKGLLLIERISNSDDSKVDLKIKNEDWIDFIIS

SEQ ID NO: 13 - protein, 1089 aa, Fibrobacter sp.
MQAIHQFCGQKNGYSRSITLRNRLIPIGKTEENIQKFLESDKNRADKYPGAKQLIDNLHRDFIAEVLSTHSFDWQPLADSIEKFQKTKDARDKKNLQTQQTNLRKQIAKAFSSSEKGKKLFSKELFTELLPEYIKGKVDEKANEEIVKEFDRFTTYFTGFYDNRKNMYSDEEQATAISFRLVNENFPKFLTNAKLFQEIKGKYPEIINDALKSLKNEKIDSYFEVNGFNACLTQQGIDAYNQVLGGTAAEAGQEKSKGLNECINLYKQQHSDVKIGKMSMLYKQILSDRDGSFIDAFEKDEDVFKAVQSYHEILISQLSEIEKLFVDAEYDLDKIIVPVKKLTEYSQVCTGRWNVVEESIRQNFIAKHGEPKKKKDEDALDKELKKDQSLLELKNILASAPSMEGINIVDYLNNDNLVKQTFSSVELEVKNLEEGFVTLIQKISYKDGSDLKQKDDDVEHIKIYLDCALNLYHYLELVDYRGEAEKDGDFYSTYEKVIERLSGILFLYNKVRNYVTKKIDTEKKFKLNFDSPTLANGWDANKESANNAIILRKNGKYYLGIFNPNDKPKIDNEATCDASDCYEKMVYKLLPGPNKMLPKVFFSKKGLETFNPPKEILEGYTKEQYKKGDTFDIIFCHKLIDWFKDAINQHPDWKKFNFKFSKTESYADISEFYREITEQGYKISFTKIAESEIQNLVDCGKLFLFQIYNKDYAENSCGSKNLHTLYWENLFSEENLKNTVLKLNGEAELFFRPQVIKEDKIIAHKKDSYLVNRIGKDGKRIPESFYQEIYKKANGIIDKISDEAKEFEKNAVVKKATHDIVKDRRFTQNVYQFHCPITMNFKAAELTGKKFNERVQELLAKDPTVKVIGIDRGERHLLYLSLINQKGEIELQKTLNLVELNRNGQTVQVDYQQKLTLKEKERDNARKNWKTINNIKEIKEGYLSAVVHEIAKMMVEHNAIVVMEELNYGFKRGRFPVERQVYQKFELALIEKLNFLVFKNKNVSEAGGVLNAFQLTQKPDSLTDFGKQNGWIFYIPAAYTSKIDPKTGFIDFFKLSKVATKNLTNMDAKKSFFKGSSSTCVGGFATLFC

SEQ ID NO: 14 - protein, 1259 aa, Clostridium sp.
MNYKTGLEDFIGKESLSKTLRNALIPTESTKIHMEEMGVIRDDELRAEKQQELKEIMDDYYRAFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYLVDFYDRVLTQPGIEYYNGICGKINEHMNQFCQKNRINKNDFRMKKLHKQILCKKSSYYEIPFRFESDQEVYDALNEFIKTMKEKEIICRCVHLGQKCDDYDLGKIYISSNKYEQISNALYGSWDTIRKCIKEEYMDALPGKGEKKEEKAEAAAKKEEYRSIADIDKIISLYGSEMDRTISAKKCITEICDMAGQISTDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPVVHKKGSVLVNRSYTQTVGDKEIRVSIPEEYYTEIYNYLNHIGRGKLSTEAQRYLEERKIKSFTATKDIVKNYRYCCDHYFLHLPITINFKAKSDIAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTMLASTKWKVYTNGTRLKRIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 15 - protein, 1296 aa, unidentified
  source 1..1296: note = Brumimicrobium aurantiacum
MKNQINLFTNKFQLSKTLRFELKPQGKTLEHINSKGFIKNDEKRADSYKKMKATIDAFHRDFIDLAMSNVKLTNLIDFEEIYNASNADKKDEKYKTKLSKIQEILRKEIAKGFKGEEVKDIFSKIDKKDLITKLLEEWIIENKIEDIHFDPEFKNFTTYFSGFHQNRKNMYTDQEQSTAIAYRLIHENLPRFIDNINIFQKINKVPDLEENLKKLYQEIEEYLGINAINEAFELEYFNETLSQKGIDIYNLILGGRTAEEGKQKIQGLNEYINLYNQKQDKKNRVPKLKVLYKQILSDRTRTSFLPDTFEDDEESSASQKVLDSINNFYLENLIDYLPNDKNSTINVLENLKLLLAELINFELDKVYIKNDTSITNISMKIFKNYSVIREALNYFYENKIDPNFAHNFNNANTDKKREKLEKEKAKITKQTYLSISFIEEAIHLYINENSNGNQYKNTYKPNCIANYFKDFFIAENKEGSNKEFDFISKIKARYNTIKGVLNTPFPDNKRLHQEKNNIDNIKHFLDSIMEYLHFAKPLVLSGSFAFEKDEQFYTNFDELYNQLELIIPLYNKVRNYATQKPYSTEKFKLNFENSTLLNGWDVNKEEANTSILFIKNGFYYLGIMDKNHNKIFRNTPKSTNTDIYKKVNYKLLPGASKMLPKVFFGKKNLDYYKPSKDILRIRNHGTHTKGGKPQSGFDKLDFNLNDCHKLIDFFKDSIQKHPDWSKFKFKFSDTQIYESIDQFYRELEPQAYSITYTNIDSSFIEEQINEGKLYLFQIYNKDFSKFSNGKPNLHTLYWKALFDEQNLKDVTYKLNGEAEIFYRKKSIQHDRQIIHKRNQPIINKNPNNEKKESIFKYNIIKDKRYTIDKFQFHVPITLNFKAKGTDYINYDVLDYLKENPDVKIIGLDRGERHLIYLTLIDQKGKILEQISLNEIVNKKHNITTSYHNLLETKEIERDKARKNWGTVETIKELKEGYISQVVHKISKMMIEHNAIVVMEDLNMGFKRGRFKVEKQVYQKLEKMLIDKLNYLVLKDRQPNEPAGIYNALQLTNKFESFQKLGKQSGFLFYVPAWNTSKIDPTTGFVNLFHVKYESVRKSQEFFNKFNSIKYNPKEAIFEFDFDYNEFTTRAEGTKTNWTVCTYGDRIKTFRNPEKLNQWDNKEINITTAFEDFFGRHNITYGNGSDIKSQLISREEKDFFSELIHLFRLTLQMRNSKTNSEIDYLISPVKNENGFFYDSRHADKNLPKDADANGAYHIAKKGLQWIKEIQSFEGNEWKKLKLDKTNKGWLKFVQENQ

SEQ ID NO: 16 - protein, 1206 aa, unidentified
  source 1..1206: note = Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 17 - protein, 1298 aa, Thiomicrospira sp.
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 18 - protein, 973 aa, unidentified
  source 1..973: note = Sneathia vaginalis
MTEEDTKSFVDEILLTPESVIKTIDNFIDSIIMNDIEGLKEEFLKISLENFEGIYISNKKLNEISNRKFGDYNSINMMIKQSMNEKGILSKKEINELIPDLENINKPKVKSFNLSFIFENLTKEHKELIIDYIRENICNVIENVKITIEKYRNIDNKIEFKNNAEKVSKIKEMLESINELCKLIKEFNTDEIEKNNEFYNILNKNFEIFESSYKVLNKVRNFVTKKEVIENKMKLNFSNYQLGNGWHKNKEKDCSIILFRKRNNERWIYYLGILKHGTKIKENDYLSSVDTGFYKMDYYAQNSLSKMIPKCSITVKNVKNAPEDESVILNDSKKFNEPLEITPEIRKLYGNNEHIKGDKFKKESLVKWIDFCKEFLLKYKSFEKAKKEILKLKESNLYENLEEFYSDAEEKAYFLEFINIDEDKIKKLVKEKNLYLFQIYNKDFSAYSTGNKNLHTMYFEELFTDENLKKPVFKLNGNTEVFYRIASSKPKIVHNKGEKLVNKTYLDDGIIKTIPDSVYEEISEKVKNNEDYSKLLEENNIKNLEIKVATHEIVKDKRYFENKFLFYLPITLNKKVSNKNTNKNINKNVIDEIKDCNEYNVIGIDRGERNLISLCIINQNGEIILQKEMNIIQSSDKYNVDYNEKLEIKSKERDNAKKNWSEIGKIKDLKSGYLSAVVHEIVKLAIEYNAVIILEDLNNGFKNSRKKVDKQIYQKFERALIEKLQFLIFKNYDKNEKGGLRNAFQLTPELKNITKVASQQGIIIYTNPAYTSKIDPTTGYANIIKKSNNNEESIVKAIDKISYDKEKDMFYFDINLSNSSFNLTVKNVLKKEWRIYTNGERIIYKDRKYITLNITQEMKDILSKCGIDYLNIDNLKQDILKNKLHKKVYYIFELANKMRNENKDVDYIISPVLNKDGKFFMTQEINELTPKDADLNGAYNIALKGKLMIDNLNKKEKFVFLSNEDWLNFIQGR

SEQ ID NO: 19 - protein, 1250 aa, Bacillus sp.
MNNNMFKDFINKYSVPKTLKFELIPIGNTSENIKKYKIIETDRELEKGYEKVKLLIDEYHRSFISRVLNNIEFGESLLKYEEFYSHNTDLKREKFEIHKKEMRKKISKAFKDAGAAELFKNTLITKLLPGLYEGKDEVLNVLNLFKKFTTYFKNFHENRKNIYSSEEKSTAISYRIINENLPIFIHNIKTYERICSLIDFDTALEDSFLNQIKKELNCQFFSEFFNIHTFNRVLSQEGINSYNLLLGGKSEEDGTKIKGVNELLNLFCQETKEKLPKFKFLKKQILSDMDSKSFVLDAFNSDSDVLEAISSYHEYLMENIEGSEITLKEFIGQMKNENLDTIYIKDKQSLKSISQKVFGSWSTITEAIYSFEYDEKNGGKGSTNSVKYNEKKKNEFNKYYEQMAKSNTKLKKIYSLSYINKCIKYFGKSEDICDYFIGMGQYGVKEEVPGENLIEAITSNYSAIKFNFIDKILSENELLIEKVKRYLDSIKELQMFLKPLNKEGDKNPLFYGEFDRFYGALESVTPLYNMVRNYVTKKPYSKDKLKLNFSNAQLLNGWDKDKESDYLSLLFKKGSKYYLGVLNNKIPKVGKCFDCQFEVDSEDYYEKMEYKQLSNVVANIPRIAFSDSNKSLFSPSNEILKIQERGSYLKSSIDFDIKDLHQMIDFFKNGLKKKYSEYDFNFSNTSSYSNISDFYQEVIKATYKVKFRKVPSKFIDDLVDVGKLFLFQLHNKDYSIKSKGKKNLHTIYWESLFSEANLKNPVHKLNGEAEIFFRKSSIQRHITHPKGQLIESKREKGKFNKFSYDLIKDKRYTEDKFFLHVPITLNRSANDKGTNFNTEVCNVLSKFPEPHIIGVDRGERHLIYLSVIDSRGKIIHQESLNTITNSYIDGSGKEQVTEINYHSKLDKSEEERSQSRKNWKKIENIKELKQGYLSHVVHRITSLMFKYNGIVILEDLNFGFKRGRFHVEKQVYQKFEKALIDKLNLIVSKNVNENELGGIRKPYQLTSKFTSFKELGKQTGFLFYVNPNYTSKLDPTTGFSNQLLIKYESINKTKEFLEKFDEIKFNKEENYFEFHVDFSKFTQKKVGKTKWVICTKGDRISNFNRKQVYLTNELKELFNKYEIDYKNDIQEQFRQLELSKAFYESFLGYLRLTLQMRNNDPNKKDENGNEIDYIISPVKNDNNRFFDSRDVKNEHGLPVNGDANGAYNIALKGLMLLNEIKEATKEGRRPNLAIKNEDWFKFVQNKEYNG

SEQ ID NO: 20 - protein, 1323 aa, Treponema porcinum
MKISEEFCGQGNGYSISKTLRFELKPKGKTLENIEKEKLLESDFKKSQDYKDVKIILDNYHKYFIDDVLQKVNLDWTKLAASITDYNKNKEDDSSVIKEQDFLRKEIVKIISKDKRFACLTASTPKDLFNSILLEWFEKSTEFSLNKKAVETFKRFSSYFKGFQENRKNMYKDEPIPTAVPYRIVNENFPKFLQNAESFKEIQKKCPEVIELVEKELSAYLGNDKLSDIFCVKNFNRYLCQTGAENQRGIDYYNQIIGGIVQKENDVKLRGINEFLNLFWQQHVDFAKDNRRIKFVPLYKQILSDRSSLSFKIQTLESDEELKEAVLSFAKKLDSKNKDGKNIFDLVMELTENINQYDLSQIYINQKDMNAVSKTLTGDWAYLQKRMNIFAEETLTKSEQKRWKKELDDDTSKSKGIFSFEELNKVLEYSSENCSAVSIKIQEYFETTKRWYFEKQTGIFTKGEEIIEPSISGLCGQIKSNFDEVNKVFGNVSSENTLRENPEEVEKIKNYLDSVQNLLHRIKPLKVNGIGDTSFYSEYDEIYSVLYEVISLYNKTRNYISKKSGIPEKFKLNFDNPTLADGWDQNKEQANTSVILIKDDEYFLGIMNANNKPKFLENYEGNTEKCYQKMIYKLLPGPNKMLPKVFFSTKGIETFNPPKEILNGYNAGKHKKGDSFDLDFCHSLIDWFKDAINRHEDWKKFDFKFSETSSYKDISEFYREISEQGYKLTFTAIPESVVEKMVTDGNLFLFQIYNKDFAKGASGKPNMHTLYWKQLFSKENLSDTILKLNGEAEIFYREPGIKEPIVHKTGSKLVNKVTKDGVSVPAEIYNEIYKVQNGMQTELSETAQVFVKEHEVSVKTASHDITKDKHFTEAKFLFHVPITINFKAQGNSLTMNERVRKFLKNNPEVNIIGLDRGERHLIYFSLINQKGEILKQFTFNEVERKQNDRIIKVDYHEKLDNREKERDAARKNWTAIGKIAELKEGYLSAVIHELTKMMIQYNAVIVMEDLNFGFKRGRFHVEKQVYQKFERMLIDKLNYLVFKDKGFTEPGGVLNGYQLAGQFESFQKLGKQSGFLFYVPAGYTSKIDPKSGFADLFNLRDLTNVRRKREFFSKFDSIKYDSETMSFSFAFDYKNFDGKGKTEMAKTKWTVFSKDKRIVYFPKNKSYSDVFPTDELKQTFEQAEIKIHDDENLLDVIMEIGADLKPDEKPNQNVASFWDSLLRNFKLILQMRNSNAQTGEDYIISPVKADDGTFFDSRNQLSLGKEAKLPIDADANGAYHIALKGLELLRRFNETDEIKLKKADMKISNADWFKFVQEKQYLN

SEQ ID NO: 21 - protein, 1259 aa, Synergistes jonesii
MANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 22 - protein, 1197 aa, unidentified
  source 1..1197: note = Sulfurimonas crateris
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 23 - protein, 1284 aa, unidentified
  source 1..1284: note = Limihaloglobus sulfuriphilus
MKDFTHQYSLSKTLRFELKPVGETAERIEDFKNQGLKSIVEEDRQRAEDYKKMKRILDDYHKEFIEEVLNDDIFTANEMESAFEVYRKYMASKNDDKLKKEITEIFTDLRKKIAKAFENKSKEYCLYKGDFSKLINEKKTGKDKGPGKLWYWLKAKADAGVNEFGDGQTFEQAEEALAKFNNFSTYFTGFNQNRDNIYTDAEQQTAISYRVINENMTRYFDNCIRYSSIENKYPELVKQLEPLSGKFAPGNYKDYLSQTAIDIYNEAVGHKSDDINAKGINQFINEYRQRNSIKGRELPIMSVLYKQILSDINKDLIIDKFENAGELLDAVKTLHRELTDKKILLKIKQTLNEFLTEDNSEDIYIKSGTDLTAVSNAIWGEWSVIPKALEMYAENITDMNAKAREKWLKREAYHLKTVQEAIEAYLKDNEEFETRNISEYFTNFKSGENDLIQVVQSAYAKMESIFGIEDFHKDRRPVTESGEPGEGFRQVELVREYLDSLINVEHFIKPLHMFRSGKPIELEDCNSNFYDPLNEAYKELDVVFGIYNKVRNYVTQKPYSKDKFKINFQNSTLLDGWDVNKESANSSVLLLKNGKYYLGVMKQGASNILNYRPEPSDSKNKINAKKQLSEIALAGATDDYYEKMIYKLLPDPAKMLPKVFFSAKNIEFYNPSQEIIYIRENGLFKKDAGDKESLKKWIGFMKTSLLKHPEWGSYFNFEFEPAEDYQDISIFYKQVAEQGYSVTFDKIKTSYIEEKVASGELYLFEIYNKDFSPHSKGRPNLHTMYWKSLFEKENLQNLVTKLNGEAEVFFRQHSIKRNEKVVHRANRPIQNKNPLTEKKQSIFEYDLVKDRRFTKDKFFLHCPITLNFKEAGPGRFNDKVNKYIAGNPDIRIIGIDRGERHLLYYSLIDQSGRIVEQGTLNQITSTLNSGGREIPKTTDYRGLLDTKEKERDKARKSWSMIENIKELKSGYLSHIVHKLAKLMVKNNAVVVLEDLNFGFKRGRFKVEKQVYQKFEKALIEKLNYLVFKDARPAEPGHYLNAYQLTAPLESFKKLGKQSGFIYYVPAWNTSKIDPVTGFVNQFYIEKNSMQYLKNFFGKFDSIRFNPDKNYFEFGFDYKNFHNKAAKSKWTICTHGDKRSWYNRKQRKLEIHNVTENLASLLSGKGINFADGGSIKDKILSVDDASFFKSLAFNFKLTAQLRHTFEDNGEEIDCIISPVAAADGTFFCSETAKKLNMELPHDADANGAYNIARKGLMVLRQIRESGKPKPISNADWLDFAQQNED

SEQ ID NO: 24 - protein, 1217 aa, Pseudobutyrivibrio xylanivorans
MIIGRDFNMYYQNLTKMYPISKTLRNELIPVGKTLENIRKNGILEADIQRKADYEHVKKLMDNYHKQLINEALQGVHLSDLSDAYDLYFNLSKEKNSVDAFSKCQDKLRKEIVSFLKNHENFPKIGNKEIIKLIQSLNDNDADNNALDSFSNFYTYFSSYNEVRKNLYSDEEKSSTVAYRLINENLPKSLDNIKAYAIAKKAGVRAEGLSEEEQDCLFIIETFERTLTQDGIDNYNADIGKLNTAINLYNQQNKKQEGFRKVPQMKCLYKQILSDREEAFIDEFSDDEDLITNIESFAENMNVFLNSEIITDFKNALVESDGSLVYIKNDVSKTLFSNIVFGSWNAIDEKLSDEYDLANSKKKKDEKYYEKRQKELKKNKSYDLETIIGLFDDSIDVIGKYIEKLESDITAIAEAKNDFDEIVLRKHDKNKSLRKNTNAVEAIKSYLDTVKDFERDIKLINGSGQEVEKNLVVYAEQENILAEIKNVDSLYNMSRNYLTQKPFSTEKFKLNFENPTLLNGWDRNKEKDYLGILFEKEGMYYLGIINNNHRKIFENEKLCTGKESCFNKIVYKQISNAAKYLSSKQINPQNPPKEIAEILLKRKADSSSLSRKETELFIDYLKDDFLVNYPMIINSDGENFFNFHFKQAKDYGSLQEFFKEVEHQAYSLKTRPIDDSYIYRMIDEGKLYLFQIHNKDFSPYSKGNLNLHTIYLQMLFDQRNLNNVVYKLNGEAEVFYRPASINDEEVIIHKAGEEIKNKNSKRAVDKPTSKFGYDIIKDRRYSKDKFMLHIPVTMNFGVDETRRFNDVVNDALRNDEKVRVIGIDRGERNLLYVVVVDTDGTILEQISLNSIINNEYSIETDYHKLLDEKEGDRDRARKNWTTIENIKELKEGYLSQVVNVIAKLVLKYNAIICLEDLNFGFKRGRQKVEKQVYQKFEKMLIDKLNYLVIDKSRKQEKPEEFGGALNALQLTSKFTSFKDMGKQTGIIYYVPAYLTSKIDPTTGFANLFYVKYENVEKAKEFFSRFDSISYNNESGYFEFAFDYKKFTDRACGARSQWTVCTYGERIIKYRNADKNNSFDDKTIVLSEEFKELFSIYGISYEDGAELKNKIMSVDEADFFRCLTGLLQKTLQMRNSSNDGTRDYIISPIMNDRGEFFNSEACDASKPKDADANGAFNIARKGLWVLEQIRNTPSGDKLNLAMSNAEWLEYAQRNQIAS

SEQ ID NO: 25 - protein, 1235 aa, Anaerovibrio sp.
MVAFIDEFVGQYPVSKTLRFEARPVPETKKWLESDQCSVLFNDQKRNEYYGVLKELLDDYYRAYIEDALTSFTLDKALLENAYDLYCNRDTNAFSSCCEKLRKDLVKAFGNLKDYLLGSDQLKDLVKLKAKVDAPAGKGKKKIEVDSRLINWLNNNAKYSAEDREKYIKAIESFEGFVTYLTNYKQARENMFSSEDKSTAIAFRVIDQNMVTYFGNIRIYEKIKAKYPELYSALKGFEKFFSPTAYSEILSQSKIDEYNYQCIGRPIDDADFKGVNSLINEYRQKNGIKARELPVMSMLYKQILSDRDNSFMSEVINRNEEAIECAKNGYKVSYALFNELLQLYKKIFTEDNYGNIYVKTQPLTELSQALFGDWSILRNALDNGKYDKDIINLAELEKYFSEYCKVLDADDAAKIQDKFNLKDYFIQKNALDATLPDLDKITQYKPHLDAMLQAIRKYKLFSMYNGRKKMDVPENGIDFSNEFNAIYDKLSEFSILYDRIRNFATKKPYSDEKMKLSFNMPTMLAGWDYNNETANGCFLFIKDGKYFLGVADSKSKNIFDFKKNPHLLDKYSSKDIYYKVKYKQVSGSAKMLPKVVFAGSNEKIFGHLISKRILEIREKKLYTAAAGDRKAVAEWIDFMKSAIAIHPEWNEYFKFKFKNTAEYDNANKFYEDIDKQTYSLEKVEIPTEYIDEMVSQHKLYLFQLYTKDFSDKKKKKGTDNLHTMYWHGVFSDENLKAVTEGTQPIIKLNGEAEMFMRNPSIEFQVTHEHNKPIANKNPLNTKKESVFNYDLIKDKRYTERKFYFHCPITLNFRADKPIKYNEKINRFVENNPDVCIIGIDRGERHLLYYTVINQTGDILEQGSLNKISGSYTNDKGEKVNKETDYHDLLDRKEKGKHVAQQAWETIENIKELKAGYLSQVVYKLTQLMLQYNAVIVLENLNVGFKRGRTKVEKQVYQKFEKAMIDKLNYLVFKDRGYEMNGSYAKGLQLTDKFESFDKIGKQTGCIYYVIPSYTSHIDPKTGFVNLLNAKLRYENITKAQDTIRKFDSISYNAKADYFEFAFDYRSFGVDMARNEWVVCTCGDLRWEYSAKTRETKAYSVTDRLKELFKAHGIDYVGGENLVSHITEVADKHFLSTLLFYLRLVLKMRYTVSGTENENDFILSPVEYAPGKFFDSREATSTEPMNADANGAYHIALKGLMTIRGIEDGKLHNYGKGGENAAWFKFMQNQEYKNNG

SEQ ID NO: 26 - protein, 1302 aa, Acinetobacter sp.
MNYQLLFQKFVHLYPISKTLRFELIPQGATQKFITEKQVLLQDEVRARKYPEMKQAIDGYHKDFIQRALGNIDSQSFEQALQTFQELFLRSQAERSTEAYKKEFETTQTKLRELIVNSFEKGEFKQEYKSLFDKNLITNLLKPWVEKQSQTGDNNYTYHNDFNKFTTYFLGFHDNRKNIYSKEPHKTALAYRLIHENLPKFLENNKILRKIQNDHPALWEQLQALHHTMPQLFNGWDLSQLLQVSFFSNTLTQTGIDQYNTIIGGISEGENRQKIQGINELINLYNQKQDKKNRVAKLKQLYKQILSDRSTLSFLPQQFADDAELYHAINMFYLDHLHYQSMVNGHSYTLLERVQLLINELANYDLSKVYLAPNQLSAVSHQMFGDFGYISRALSYYYMQVIQPDYELLLASAKTTAKIEAIEKLKTAFLDAPHSLVVIQAAIDKYLQLQPSSKPHTQLTDFIISLLKQYETVADDQSIKIINIFSDIEGKYSCIKGLVNTESTSESKREILQNEKLATDIKAFMDAINNVIKLLKPFALNEKLAASVEKDARFYSDFEEIYQALLVFVPLYNKVRNYITQKPYSTEKFKLNFNKPTLLSGWDANKEADNLSILLRKNGNYYLAIMDTAKGANKAFEPKALNQLKVDDTTDCYEKMVYKLLPGPNKMFPKVFFSESRKAQFNPPQHIIESYNKKEHISSEAHFDLKKCHALIDWFKQCIELHEDWKHFNFKFSPTSQYSNISDFYKEVSEQNYKVHFQDIPADYIEQLVAEGKLYLFQIYNKDFSPHAKGKENLHTMYFKALFSEENLKQPVFKLSGEAEMFYRPASLQLENTTIHKAGEAMVAKNPLTPDATRTLAYDIIKDRRFTTDKYLLHIPISLNFHAQESMSIKKHNDLVRQMIKHNHQDLHIIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFKTFDSIIFNAKKDYFEFKVNLNQFAGLKAQAARAEWTICSYGPERHVYQKKNAQQGETVIVNVTEELKALFAKNNIEVAEGVELKEMICAQTQVDFFKRLIWLLQVLLALRYSSSKDKLDYILSPVANVLGEFFDSRHASTHLPQDSDANGAYHIALKGLWVIEQLKTAANTEKVNLAISNDEWLRFAQEKLYLT

SEQ ID NO: 27 - protein, 1262 aa, unidentified
  source 1..1262: note = Bacteroidetes oral taxon 274 strain F0058
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 28 - protein, 1265 aa, Eubacterium rectale
  source 1..1265: note = strain 2789STDY5834884
MNNGTNNFQNFIGISSLQKTLRNALTPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDMKDSLKKMSLEKIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPEKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDVVLKLNGEAEIFFRKSSIKNPIIHKKGSILVNRTYEAEEKDQFGNIQIVRKTIPENIYQELYKYFNDKSDKELSDEAAKLKNAVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYLTS

SEQ ID NO: 29 - protein, 1477 aa, unidentified
  source 1..1477: note = Candidatus Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 30 - protein, 1224 aa, unidentified
  source 1..1224: note = Lachnospiraceae bacterium
MGLYDGFVNRYSVSKTLRFELIPQGRTREYIETNGILSDDEERAKDYKTIKRLIDEYHKDYISRCLKNVNISCLEEYYHLYNSSNRDKRHEELDALSDQMRGEIASFLTGNDEYKEQKSRDIIINERIINFASTDEELAAVKRFRKFTSYFTGFFTNRENMYSAEKKSTAIAHRIIDVNLPKYVDNIKAFNTAIEAGVFDIAEFESNFKAITDEHEVSDLLDITKYSRFIRNEDIIIYNTLLGGISMKDEKIQGLNELINLHNQKHPGKKVPLLKVLYKQILGDSQTHSFVDDQFEDDQQVINAVKAVTDTFSETLLGSLKIIINNIGHYDLDRIYIKAGQDITTLSKRALNDWHIITECLESEYDDKFPKNKKSDTYEEMRNRYVKSFKSFSIGRLNSLVTTYTEQACFLENYLGSFGGDTDKNCLTDFTNSLMEVEHLLNSEYPVTNRLITDYESVRILKRLLDSEMEVIHFLKPLLGNGNESDKDLVFYGEFEAEYEKLLPVIKVYNRVRNYLTRKPFSTEKIKLNFNSPTLLCGWSQSKEKEYMGVILRKDGQYYLGIMTPSNKKIFSEAPKPDEDCYEKMVLRYIPHPYQMLPKVFFSKSNIAFFNPSDEILRIKKQESFKKGKSFNRDDCHKFIDFYKDSINRHEEWRKFNFKFSDTDSYEDISRFYKEVENQAFSMSFTKIPTVYIDSLVDEGKLYLFKLHNKDFSEHSKGKPNLHTVYWNALFSEYNLQNTVYQLNGSAEIFFRKASIPENERVIHKKNVPITRKVAELNGKKEVSVFPYDIIKNRRYTVDKFQFHVPLKMNFKADEKKRINDDVIEAIRSNKGIHVIGIDRGERNLLYLSLINEEGRIIEQRSLNIIDSGEGHTQNYRDLLDSREKDREKARENWQEIQEIKDLKTGYLSQAIHTITKWMKEYNAIIVLEDLNDRFTNGRKKVEKQVYQKFEKMLIDKLNYYVDKDEEFDRMGGTHRALQLTEKFESFQKLGRQTGFIFYVPAWNTSKLDPTTGFVDLLYPKYKSVDATKDFIKKFDFIRFNSEKNYFEFGLHYSNFTERAIGCRDEWILCSYGNRIVNFRNAAKNNSWDYKEIDITKQLLDLFEKNGIDVKQENLIDSICEMKDKPFFKSLIANIKLILQIRNSASGTDIDYMISPAMNDRGEFFDTRKGLQQLPLDADANGAYNIAKKGLWIVDQIRNTTGNNVKMAMSNREWMHFAQESRLA

SEQ ID NO: 31 - protein, 1257 aa, Prevotella bryantii
MQINNLKIIYMKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 32 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 33 - protein, 1261 aa, Moraxella bovoculi
MLFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 34 - protein, 1251 aa, Moraxella lacunata
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGSNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 35 - protein, 1208 aa, Butyrivibrio sp.
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLLAS

SEQ ID NO: 36 - protein, 1312 aa, Helcococcus kunzii
MFEKLSNIVSISKTIRFKLIPVGKTLENIEKLGKLEKDFERSDFYPILKNISDDYYRQYIKEKLSDLNLDWQKLYDAHELLDSSKKESQKNLEMIQAQYRKVLFNILSGELDKSGEKNSKDLIKNNKALYGKLFKKQFILEVLPDFVNNNDSYSEEDLEGLNLYSKFTTRLKNFWETRKNVFTDKDIVTAIPFRAVNENFGFYYDNIKIFNKNIEYLENKIPNLENELKEADILDDNRSVKDYFTPNGFNYVITQDGIDVYQAIRGGFTKENGEKVQGINEILNLTQQQLRRKPETKNVKLGVLTKLRKQILEYSESTSFLIDQIEDDNDLVDRINKFNVSFFESTEVSPSLFEQIERLYNALKSIKKEEVYIDARNTQKFSQMLFGQWDVIRRGYTVKITEGSKEEKKKYKEYLELDETSKAKRYLNIREIEELVNLVEGFEEVDVFSVLLEKFKMNNIERSEFEAPIYGSPIKLEAIKEYLEKHLEEYHKWKLLLIGNDDLDTDETFYPLLNEVISDYYIIPLYNLTRNYLTRKHSDKDKIKVNFDFPTLADGWSESKISDNRSIILRKGGYYYLGILIDNKLLINKKNKSKKIYEILIYNQIPEFSKSIPNYPFTKKVKEHFKNNVSDFQLIDGYVSPLIITKEIYDIKKEKKYKKDFYKDNNTNKNYLYTIYKWIEFCKQFLYKYKGPNKESYKEMYDFSTLKDTSLYVNLNDFYADVNSCAYRVLFNKIDENTIDNAVEDGKLLLFQIYNKDFSPESKGKKNLHTLYWLSMFSEENLRTRKLKLNGQAEIFYRKKLEKKPIIHKEGSILLNKIDKEGNTIPENIYHECYRYLNKKIGREDLSDEAIALFNKDVLKYKEARFDIIKDRRYSESQFFFHVPITFNWDIKTNKNVNQIVQGMIKDGEIKHIIGIDRGERHLLYYSVIDLEGNIVEQGSLNTLEQNRFDNSTVKVDYQNKLRTREEDRDRARKNWTNINKIKELKDGYLSHVVHKLSRLIIKYEAIVIMENLNQGFKRGRFKVERQVYQKFELALMNKLSALSFKEKYDERKNLEPSGILNPIQACYPVDAYQELQGQNGIVFYLPAAYTSVIDPVTGFTNLFRLKSINSSKYEEFIKKFKNIYFDNEEEDFKFIFNYKDFAKANLVILNNIKSKDWKISTRGERISYNSKKKEYFYVQPTEFLINKLKELNIDYENIDIIPLIDNLEEKAKRKILKALFDTFKYSVQLRNYDFENDYIISPTADDNGNYYNSNEIDIDKTNLPNNGDANGAFNIARKGLLLKDRIVNSNESKVDLKIKNEDWINFIISAS

SEQ ID NO: 37 - protein, 1308 aa, Lachnospira pectinoschiza
  source 1..1308: note = strain 2789STDY5834836
MIMNNVTGDFSEFVAISKVQKTLRNELRPTPLTMKHIKQKGIITEDEYKTQQSLELKRIADGYYRDYITHKLNDTNNLDFRNLFEAIEEKYKKNDKDNRDKLDLVEKSKRGEIAKLLSADDNFKSMFEAKLITQLLPVYVEQNYIGEDKEKALETIALFKGFTTYFTDYFNIRKNMFKENGGASSICYRIVNVNASIFYDNLKTFMCIKEKAETEIALIEEELTELLDSWRLEHIFSEDYYNELLAQKGIDYYNQICGDVNKHMNLYCQQNKLKANVFKMTKLQKQIMGISEKAFEIPPMYQNDEEVYAAFNGFISRLEEVKLIDRLGNVLQNSNIYDTAKIYINARCYTNVSSYVYGGWGVIESAIERYWYNTIAGKGQSKAKKIEKAKKDNKFMSVKELDSIVSDYEPDYFNASNMDDDNSGRAFSGHGVLGYFNKMSKLLANMSLHTITYDSGDSLIENKETALNIKKDLDDIMSIYHWLQTFIIDEVVEKDNAFYAELEDIYYELENVVTLYDRIRNYVTRKPYSTQKFKLNFASPTLASGWSRSKEFDNNAIILLRNNKYYIAIFNVNNKPDKQIIKGSEEQRLSTDYKKMVYNLLPGPNKMLPWVFIKSNTGKRDYNPSSYILEGYEKNRHIKSSGNFDINYCHDLIDYYKACINKHPEWKNYGFKFKETTQYNDIGQFYKDVEKQGYSISWAYISEADINRLDEEGKIYLFEIYNKDLSSHSTGKDNLHTMYLKNIFSEDNLKNICIELNGNAELFYRKSSMKRNITHKKDTVLVNKTYINEAGVRVSLTDEDYIKVYNYYNNDYVIDVEKDKKLVEILERIGHRKNPIDIIKDKRYTEDKYFLHFPITINYGVDDENINAKMIEYIAKHNNMNVIGIDRGERNLIYISVINNKGNIIEQKSFNLVNNYDYKNKLKNMEKTRDNARKNWQEIGKIKDVKNGYLSGVISKIARMVVDYNAIIVMEDLNRGFKRGRFKVERQVYQKFENMLISKLNYLVFKEKKADENGGILKGYQLTYLPKSALQIGKQCGCIFYVPAAYTSKIDPATGFINIFDFKKYSGSAINAKVKDKKEFLMSMNSIRYVNEGSAEYEKIGHRQLFAFSFDYNNFKTYNVSIPVNEWTTYTYGERIKKLYKDGRWSGSEVLNLTEDLIELMEQYGIEYKDGHDIREDISHMDEMRNADFICNLFEKFKYTVQLRNSKSEAEGDDYDRLVSPVLNSHNGFFDSSDYKENEKSDDIIDDKQIMPKDADANGAYCIALKGLYEINKIKENWSDDKKLKESELYIGVTEWLDYIQNRRFEAS

SEQ ID NO: 38 - protein, 1227 aa, unidentified
  source 1..1227: note = Candidatus Methanomethylophilus alvus Mx1201
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 39 - protein, 1352 aa, unidentified
  source 1..1352: note = Parcubacteria group bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 40 - protein, 1215 aa, Pseudobutyrivibrio ruminis
MIIGRDFNMYYQNLTKMYPISKTLRNELIPVGKTLENIRKNGILEADIQRKADYEHVKKLMDNYHKQLINEALQGVHLSDLSDAYDLYFNLSKEKNSVDAFSKCQDKLRKEIVSLLKNHENFPKIGNKEIIKLLQSLYDNDTDYKALDSFSNFYTYFSSYNEVRKNLYSDEEKSSTVAYRLINENLPKFLDNIKAYAIAKKAGVRAEGLSEEDQDCLFIIETFERTLTQDGIDNYNAAIGKLNTAINLFNQQNKKQEGFRKVPQMKCLYKQILSDREEAFIDEFSDDEDLITNIESFAENMNVFLNSEIITDFKIALVESDGSLVYIKNDVSKTSFSNIVFGSWNAIDEKLSDEYDLANSKKKKDEKYYEKRQKELKKNKSYDLETIIGLFDDNSDVIGKYIEKLESDITAIAEAKNDFDEIVLRKHDKNKSLRKNTNAVEAIKSYLDTVKDFERDIKLINGSGQEVEKNLVVYAEQENILAEIKNVDSLYNMSRNYLTQKPFSTEKFKLNFNRATLLNGWDKNKETDNLGILFEKDGMYYLGIMNTKANKIFVNIPKATSNDVYHKVNYKLLPGPNKMLPKVFFAQSNLDYYKPSEELLAKYKAGTHKKGDNFSLEDCHALIDFFKASIEKHPDWSSFGFEFSETCTYEDLSGFYREVEKQGYKITYTDVDADYITSLVERDELYLFQIYNKDFSPYSKGNLNLHTIYLQMLFDQRNLNNVVYKLNGEAEVFYRPASINDEEVIIHKAGEEIKNKNSKRAVDKPTSKFGYDIIKDRRYSKDKFMLHIPVTMNFGVDETRRFNDVVNDALRNDEKVRVIGIDRGERNLLYVVVVDTDGTILEQISLNSIINNEYSIETDYHKLLDEKEGDRDRARKNWTTIENIKELKEGYLSQVVNVIAKLVLKYNAIICLEDLNFGFKRGRQKVEKQVYQKFEKMLIDKLNYLVIDKSRKQDKPEEFGGALNALQLTSKFTSFKDMGKQTGIIYYVPAYLTSKIDPTTGFANLFYVKYENVEKAKEFFSRFDSISYNNESGYFEFAFDYKKFTDRACGARSQWTVCTYGERIIKFRNTEKNNSFDDKTIVLSEEFKELFSIYGISYEDGAELKNKIMSVDEADFFRSLTRLFQQTMQMRNSSNDVTRDYIISPIMNDRGEFFNSEACDASKPKDADANGAFNIARKGLWVLEQIRNTPSGDKLNLAMSNAEWLEYAQRNQIAS

SEQ ID NO: 41 - protein, 1301 aa, Francisella tularensis
  source 1..1301: note = subsp. novicida strain U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNG

SEQ ID NO: 42 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 43 - protein, 1307 aa, Succiniclasticum ruminis
MGNFGEFTHKYQVSKTLRFELIPQGKTLENVAKYGIVDDDKRRSENYKKLKPVIDRIYKYFIDESLKNVSIDWQPLYEAIIAYRKEQTTANVVRLKEEQEACRKAIAAWFEGKVPDKGSKDLKEFNKTQSKLFKELFGKELFTESVTQLLPGLSLTEEEKELLASFNKFTSYFKGFYVNRKNVFSADDISTSIPHRLVQENFPKFMDNCEAYRRIVEEYPELKAKLEGTAQATGIFIGFKLDNIFKVSFYNHLLQQSQIDLYNQFLCGIAGEEGTMRVQGLNVTLNLAMKQDKVLGQKLKSMPHRFIPLYKQILSDRTTLSFIPEAFQNDEEVLLTVEEYRKSLEAERTTGAVSDIFNSLQAADLRHVYVNPAKLTAFSQMLFEDWSLCRESLRNWKLRSYGKAATKKVREEIESWLKESAISLDELQAALADGTLSVIINQKVQSVITTLEQELAKPLPKKLKTAEEKESLKSLLDSVQEACHSLEMFAVGENMDTDPCFYVPLREAMEAIQPIIPLYNKVRNFATQKPYSIEKFKLNFSNPILASGWDENRERQTCAILFRKGEKYYLGIYNAKVKPDFSIIKAVKGGNCFEKVVYRQFPDFSKMMPKCTTQLKEVQQHFASSSEDYVLYNKKFIKPLTITKEIYDLNNVLFDGKKKFQIDYLRKTKDEDGYYHALHTWINFAKEFVASYESTSIYDTSTVLSTEQYVKLNDFYGDLDNLFYRIKFESVSEETISEFVDEGKLFLFQIYNKDFAEGATGAPNLHTIYWKAVFDPENMKNVVVKLNGQAELFYRPKSAMDIVRHKVGEKLVNRRLKDGTSLTEELHEELYLYANGKLKKKLSEAAAAVLPQAVIYDVHHEIVKDRRFTEDKFFFHVPLTLNYKCDKNAVQFNASVQEYLKENPDTYIIGIDRGERNLIYAVVIDPQGNIVEQKSFNVINGFDYHNKLEQREKERNKARQDWTTVGKIKELKQGYLSLVVHEITSMMVKYNAIVVLENLNVGFKRIRSGIAEKAVYQQFEKMLINKLNYLMFKDVEGAKPGSVLNAYQLTDRFESFASMRNQTGFLFYIPAAFTSKIDPATGFVDPFCWSAIKTLDDKKTFISGFDTLKYDNVTGNFILHFEMKKNKDFQKKLEGFMPEWDIVVEANKDRRDAEGKTFISGKRIEFVRENNGHGHYEDYLPCKKLVEILRQYDILFEDGKDVLPLIMKNGDSKLIHEVFKVIRLSLQMRNSNAESGEDFISSPVENNEGICFDSRLGVETLPKDADANGAYHIALKGLLLLEKIRHDERKLGISNSEWLNHIQSLRG

SEQ ID NO: 44 - protein, 1230 aa, unidentified
  source 1..1230: note = Lachnospiraceae bacterium
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 45 - protein, 1238 aa, unidentified
  source 1..1238: note = Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 46 - protein, 1272 aa, Clostridium sp.
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKCLEKIQEKKRKEIYQYLSDDAVFSEMFKEKMISGILPDFIRCNEEYSEEEKEEKLKTVALFHRFTSSFNDFFLNRKNVFTKEAIATAIGYRVVHENAEIFLENMVAFQNIQKSAESQISIIERKNEHYFMEWKLSHIFTADYYMMLMTQKAIEHYNEMCGVVNQHMKEYCQKEKKNWNLYRMKRLHKQILSNASTSFKIPEKYENDAEVYESVNSFLQNVMEKTVMERIAVLKNNTDNFDLSKIYITAPYYEKISNYLCGSWNTIADCLTHYYEQQIAGKGARKDQKVKAAVKADKWKSLSEIEQLLKEYARAEEVKRKPEEYIAEIENIVSLKEVHLLEYHPEVNLIENEKYATEIKDVLDNYMELFHWMKWFYIEEAVEKEVNFYGELDDLYEEIRDIVPLYNKVRNYVTQKPYSDTKIKLNFGTPTLANGWSKSKEYDYNAILLQKDGKYYMGIFNPVQKPEKEIIEGHSHPLEGNEYKKMVYYYLPSANKMLPKVLLSKKGMEIYQPSEYIINGYKERRHIKSEEKFDLQFCHDLIDYFKSGIERNPDWKVFGFHFSDTDTYQDISGFYREVEDQGYKIDWTYIKEADIDRLNEEGKLYLFQIYNKDFSEKSTGRENLHTMYLKNLFSEENIREQVLKLNGEAEIFFRKSSVKKPIIHKKGTMLVNRTYMEEMHGESVKKNIPEKEYQEIYNYMNHRWKGELSAEAKEYLKKAVCHETKKDIVKDYRYSVDKFFIHLPITINYRASGKEALNSVAQRYIAHQNDMHVIGIDRGERNLIYVSVINMQGEIIEQKSFNVVNKYNYKEKLKEREQNRDEARKNWKEIGQIKDLKEGYLSGVIHEIAKMMIKYHAIVAMEDLNYGFKRGRFKVERQVYQKFENMLIQKLNYLVFKDRSADEDGGVLRGYQLAYIPDSVKKLGRQCGMIFYVPAAFTSKIDPATGFVDIFNHKAYTTDQAKREFILSFDEICYDVERQLFRFTFDYANFATHNVTLARNNWTIYTNGTRTQKEFVNRRVRDKKEVFDPTEKMLKLLELEGVEYQSGANLLPKLEKISDPHLFHELQRIVRFTVQLRNSKNEENDVDYDHVISPVLNEEGKFFDSSKYENKEEKKESLLPVDADANGAYCIALKGLYIMQAIQKNWSEEKALSPDVLRLNNNDWFDYIQNKRYR

SEQ ID NO: 47 - protein, 1230 aa, unidentified; identical to SEQ ID NO: 44
  source 1..1230: note = Lachnospiraceae bacterium COE1

SEQ ID NO: 48 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELRNLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 49 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELYNLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 50 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 51 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIWTILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 52 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLAEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 53 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLKEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 54 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMYRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 55 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVVPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 56 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVDPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 57 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKIWTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 58 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKIRTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 59 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKIHTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 60 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGADRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 61 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGYDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 62 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFLNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 63 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFINSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 64 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFVNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 65 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFMNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 66 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFFNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 67 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFRNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 68 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFANSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 69 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFHNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 70 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFNNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 71 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFQNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 72 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFSNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 73 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFTNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 74 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFYNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 75 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFWNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 76 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKESRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 77 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKVSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 78 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKLSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 79 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNQRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 80 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNKRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 81 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNWRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 82 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSREKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 83 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRMKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 84 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRKKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 85 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRGKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 86 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYDKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 87 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYEKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 88 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 89 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYMKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 90 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFEWFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 91 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFETFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 92 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFENFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 93 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 94 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESGKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 95 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFFSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 96 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFERFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 97 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNQRVKVEKQVYQKFEKMLIDKLNYMVDKKSNPCATGGALKGYQITNKFESFKSGSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 98 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNQRVKVEKQVYQKFEKMLIDKLNYMVDKKSNPCATGGALKGYQITNKFESFKSFSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 99 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNQRVKVEKQVYQKFEKMLIDKLNYMVDKKSNPCATGGALKGYQITNKFESFKSLSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 100 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNQRVKVEKQVYQKFEKMLIDKLNYMVDKKSNPCATGGALKGYQITNKFESFKSSSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 101 - protein, 1228 aa, synthetic construct; identical to SEQ ID NO: 95

SEQ ID NO: 102 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFFNSRVKVEKQVYQKFEKMLIDKLNYMVDKKSNPCATGGALKGYQITNKFESLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 103 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFFNSRVKVEKQVYQKFEKMLIDKLNYMVDKKSNPCATGGALKGYQITNKFESFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 104 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFRNSRVKVEKQVYDKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 105 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFRNSRVKVEKQVYQKFEKMLIDKLNYMVDKKSNPCATGGALKGYQITNKFESLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 106 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFRNSRVKVEKQVYQKFEKMLIDKLNYMVDKKSNPCATGGALKGYQITNKFESFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 107 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFYNSRVKVEKQVYQKFEKMLIDKLNYMVDKKSNPCATGGALKGYQITNKFESLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 108 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFYNSRVKVEKQVYQKFEKMLIDKLNYMVDKKSNPCATGGALKGYQITNKFESFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 109 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKLSRVKVEKQVYMKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 110 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRGKVEKQVYDKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 111 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRGKVEKQVYQKFEKMLIDKLNYMVDKKSNPCATGGALKGYQITNKFEWFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 112 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRGKVEKQVYQKFEKMLIDKLNYMVDKKSNPCATGGALKGYQITNKFESFKSGSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 113 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRGKVEKQVYQKFEKMLIDKLNYMVDKKSNPCATGGALKGYQITNKFESFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 114 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYDKFEKMLIDKLNYMVDKKSNPCATGGALKGYQITNKFEWFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 115 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYDKFEKMLIDKLNYMVDKKSNPCATGGALKGYQITNKFESLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 116 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRXKVEKQVYDKFEKMLIDKLNYMVDKKSNPCATGGALKGYQITNKFESFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 117 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFEWLKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 118 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFEWFKSMSFQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 119 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESGKSGSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 120 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 121 - protein, 208 aa, synthetic construct
STDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 122 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 123 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 124 - protein, 198 aa, synthetic construct
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 125 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 126 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 127 - protein, 177 aa, Escherichia coli
RRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 128 - protein, 159 aa, Staphylococcus aureus
GSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 129 - protein, 159 aa, Bacillus subtilis
TQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLS

SEQ ID NO: 130 - protein, 182 aa, unidentified
  source 1..182: note = Salmonella typhimurium
PPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 131 - protein, 163 aa, Shewanella putrefaciens
DEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 132 - protein, 172 aa, Haemophilus influenzae
DAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 133 - protein, 159 aa, Caulobacter crescentus
RTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRRRKAKI

SEQ ID NO: 134 - protein, 178 aa, Geobacter sulfurreducens
SSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 135 - protein, 806 aa, unidentified
  source 1..806: note = Streptomyces cyanogenus
MSHLSERPEKPVVGVSMPHESAVQHVTGAALYTDDLVQRTKDVLHAYPVQVMKARGRVTALRTGAALAVPGVVRVLTGADVPGVNDAGMKHDEPLFPDEVMFHGHAVAWVLGETLEAARIGAAAVEVDLEELPSVITLQDAIAADSYHGARPVMTHGDVDAGFADSAHVFTGEFQFSGQEHFYLETHAALAQVDENGQVFIQSSTQHPSETQEIVSHVLGVPAHEVTVQCLRMGGGFGGKEMQPHGFAAIAALGAKLTGRPVRFRLNRTQDLTMSGKRHGFHATWKIGFDTEGRIQALDATLTADGGWSLDLSEPVLARALCHIDNTYWIPNARVAGRIARTNTVSNTAFRGFGGPQGMLVIEDILGRCAPRLGVDAKELRERNFYRPGQGQTTPYGQPVTQPERIAAVWQQVQDNGHIADREREIAAFNAAHPHTKRALAVTGVKFGISFNLTAFNQGGALVLIYKDGSVLINHGGTEMGQGLHTKMLQVAATTLGIPLHKVRLAPTRTDKVPNTSATAASSGADLNGGAVKNACEQLRERLLRVAASQLGTNASDVRIVEGVARSLGSDQELAWDDLVRTAYFQRVQLSAAGYYRTEGLHWDAKSFRGSPFKYFAIGAAATEVEVDGFTGAYRIRRVDIVHDVGDSLSPLIDIGQVEGGFVQGAGWLTLEDLRWDTGDGPNRGRLLTQAASTYKLPSFSEMPEEFNVTLLENATEEGAVFGSKAVGEPPLMLAFSVREALRQAAAAFGPRGTAVELASPATPEAVYWAIESARQGGTAGDGRTHGAAASDAVAVRTGVEALSGA

SEQ ID NO: 136 - protein, 1347 aa, Ceratitis capitata
MTTNGNSFIVPVEKESPLIFFVNGKKVIDPTPDPECTLLTYLREKLRLCGTKLGCGEGGCGACTVMLSRVDRATNSVKHLAVNACLMPVCAMHGCAVTTIEGIGSTRTRLHPVQERLAKAHGSQCGFCTPGIVMSMYALLRSMPLPSMKDLEVAFQGNLCRCTGYRPILEGYKTFTKEFSCGMGEKCCKLQSNGNDVEKNGDDKLFERSAFLPFDPSQEPIFPPELHLNSQFDAENLLFKGPRSTWYRPVELSDLLKLKSENPHGKIIVGNTEVGVEMKFKQFLYTVHINPIKVPELNEMQELEDSILFGSAVTLMDIEEYLRERIAKLPEHETRFFRCAVKMLHYFAGKQIRNVASLGGNIMTGSPISDMNPILTAACAKLKVCSLVEGRIETREVCMGPGFFTGYRKNTIQPHEVLVAIHFPKSKKDQHFVAFKQARRRDDDIAIVNAAVNVTFESNTNIVRQIYMAFGGMAPTTVMVPKTSQIMAKQKWNRVLVERVSESLCAELPLAPTAPGGMIAYRRSLVVSLFFKAYLAISQELVKSNVIEEDAIPEREQSGAAIFHTPILKSAQLFERVCVEQSTCDPIGRPKVHASAFKQATGEAIYCDDIPRHENELYLALVLSTKAHAKIVSVDESDALKQAGVHAFFSSKDITEYENKVGSVFHDEEVFASERVYCQGQVIGAIVADSQVLAQRAARLVHIKYEELTPVIITIEQAIKHKSYFPNYPQYIVQGDVATAFEEADHVYENSCRMGGQEHFYLETNACVATPRDSDEIELFCSTQNPTEVQKLVAHVLSVPCHRVVCRSKRLGGGFGGKESRSIILALPVALASYRLRRPVRCMLDRDEDMMTTGTRHPFLFKYKVGFTKEGLITACDIECYNNAGCSMDLSFSVLDRAMNHFENCYRIPNVKVAGWVCRTNLPSNTAFRGFGGPQGMFAAEHIVRDVARIVGKDYLDIMQMNFYKTGDYTHYNQKLENFPIEKCFTDCLNQSEFHKKRLAIEEFNKKNRWRKRGIALVPTKYGIAFGAMHLNQAGALINIYGDGSVLLSHGGVEIGQGLHTKMIQCCARALGIPTELIHIAETATDKVPNTSPTAASVGSDINGMAVLDACEKLNQRLKPIREANPKATWQECISKAYFDRISLSASGFYKMPDVGDDPKTNPNARTYNYFTNGVGVSVVEIDCLTGDHQVLSTDIVMDIGSSLNPAIDIGQIEGAFMQGYGLFVLEELIYSPQGALYSRGPGMYKLPGFADIPGEFNVSLLTGAPNPRAVYSSKAVGEPPLFIGSTVFFAIKQAIAAARAERGLSITFELDAPATAARIRMACQDEFTDLIEQPSPGTYTPWNVVP

SEQ ID NO: 137 - protein, 1347 aa, unidentified; identical to SEQ ID NO: 136
  source 1..1347: note = Neurospora crassa

SEQ ID NO: 138 - protein, 1273 aa, unidentified
  source 1..1273: note = Myxococcus hansupus
MSNMFEFRLNGATVRVDGVSPNTTLLDFLRNRGLTGTKQGCAEGDCGACTVALVDRDAQGNRCLRAFNACIALVPMVAGRELVTVEGVGSSEKPHPVQQAMVKHYGSQCGFCTPGFIVSMAEGYSRKDVCTPSSVADQLCGNLCRCTGYRPIRDAMMEALAERDADASPATAIPSAPLGGPAEPLSALHYEATGQTFLRPTSWKELLDLRARHPEAHLVAGATELGVDITKKARRFPFLISTEGVESLREVRREKDCWYVGGAASLVALEEALGDALPEVTKMLNVFASRQIRQRATLAGNLVTASPIGDMAPVLLALDARLVLGSVRGERTVALSEFFLAYRKTALQADEVVRHIVIPHPAVPERGQRLSDSFKVSKRRELDISIVAAGFRVELDAHGVVSLARLGYGGVAATPVRAVRAEAALTGQPWTRETVDQVLPVLAEEITPISDQRGSAEYRRGLVAGLFEKFFAGTYSPVLDAAPGFEKGDAQVPADAGRALRHESAMGHVTGSARYVDDFAQRQPMFEVWPVCAPHAHARIFKRDPTAARKVPGVVRVFMAEDIPGTNDTGPIRHDEPFFADREVFFHGQIVAFVVGESVEACRAGARAVEVEYEPFPAIFTVEDAMAQGSYHTEPHVIRRGDVDAAFASSPHRFSGTMAIGGQEHFYFETQAAFAERGDDGDITVVSSTQHPSEVQAIISHVFHFPRSRVVVKSPRMGGGFGGKETQGNSPAAFVAFASWHTGRPTRWMMDRDVDMVVTGKRHPFHAAYEVGFDDEGKFFAFRVQFVSNGGWSFDFSESETDRAFFHEDNAYYVPAFTYTGRVAKTHFVSNTAFRGFGGPQGMLVTEEVLAHVARSVGVPADVVRERNLYRGTGETNTTHYGQELEDERIHRVWEELKRTSDFEQRRAEVDAFNARSPFIKRGLAITPMKFGISFTATFLNQAGALVHLYRDGSVMVSHGGTEMGQGFHTKVQGVAMREFGVEASAVRIAKTATDKVPNTSATAASSGSDENGAAVRFACITFRERFAPVAVRFFADRHGRTVAPEAFFFSEGKVGFRGEPEVSLPFANVVEAAYLARVGLSATGYYQTPGIGYDKAKGRGRPFLYFAYGASVCEVEVDGHTGVKRVLRVDLLEDVGDSLNPGVDRGQIEGGFVQGLGWLTGEELRWDANGRLLTHSASTYAVPAFSDAPIDFRVRLLERAHQHNTIHGSKAVGEPPLMLAMSAREALRDAVGAFGQAGGGVALASPATHEALFLAIQKRLSRGAREDGREAA

SEQ ID NO: 139 - protein, 732 aa, Enterobacter cloacae
MKFDKPATTNPIDTLRVVGQPHTRIDGPRKTTGSAHYAYEWHDIAPNAAYGHVVGAPIAKGRITAIDTKAAEAAPGVLAVITADNAGPLGKGEKNTATLLGGPEIEHYHQAVALVVAETFEQARAAAALVKVTCKRAQGAYDLAAEKASVTEPPEDTPDKNVGDVATAFASAAVKLDAIYTTPDQSHMAMEPHASMAVWEGDNVTVWTSNQMIDWCRTDLALTLKIPPENVRIVSPYIGGGFGGKLFLRSDALLAALGARAVKRPVKVMLPRPTIPNNTTHRPATLQHIRIGTDTEGKIVAIAHDSWSGNLPGGTPETAVQQTELLYAGANRHTGLRLATLDLPEGNAMRAPGEAPGLMALEIAIDEIADKAGVDPVAFRILNDTQVDPANPERRFSRRQLVECLQTGAERFGWQKRHAQPGQVRDGRWLVGMGMAAGFRNNLVATSGARVHLNADGSVAVETDMTDIGTGSYTIIAQTAAEMLGLPLEKVDVRLGDSRFPVSAGSGGQWGANTSTAGVYAACVKLREAIARQLGFDPATAEFADETISAQGRSAPLAEAAKSGVLTAEDSIEFGDLDKEYQQSTFAGHFVEVGVDSATGEVRVRRMLAVCAAGRILNPITARSQVIGAMTMGLGAALMEELAVDTRLGYFVNHDMAAYEVPVHADIPEQEVIFLEDTDPISSPMKAKGVGELGLCGVSAAIANAIYNATGVRVRDYPITLDKLIDALPDAV

SEQ ID NO: 140 - protein, 623 aa, unidentified
  source 1..623: note = Sciurus snoursei
MSHDPVPHLPPAAPLPHPLGAPSVRREGREKVTGAARYAAEHTPPGCAYAWPVPATVARGRITELDTAAALALPGVIAVLTHENAPRLASTGDPTLAVLQEDRVPHRGWYVALAVADTLEAARDAAEAVHVGYATEPHDVRITADHPRLYVPEEVFGGPGARERGDFDAAFAAAPATVDVAYTVPPLHNHPMEPHAATAQWTDGHLTVHDSSQGATRVCEDLAALFKLGTDEITVVSEHVGGGFGAKGTPRPQVVLAAMAARHTGRPVKLALPRRQLPGVVGHRAPTLHRVRIGAGHDGVITALAHEIVTHTSTVTEFVEQAAIPARMMYTSPHSRTVHRLAALDVPTPSWMRAPGEAPGMYALESALDELAVVLDIDPVELRIRNDPATEPDTGRPFSSRHLVECLRAGAERFGWLPRDPRPAVRRRGDLLLGTGVAAATYPVQISETEAEAHAAADGGYRIRVNATDIGTGARTVLTQIAAAVLGAPEDRVRVDIGSSDLPPAVLAGGSTGTASWGWAVHKACTSLLARLRAHHGPLPAEGIMAELSEWAPMALRAWRIISGLGLPTKYGSTPVALVMRAATEPVAGSGPSVEGPVSSGLVAMKRAPFSMSRMALVSASKL

SEQ ID NO: 141 - protein, 723 aa, Streptomyces albulus
MTPPPTTRTRAMSHPPEEAPFPPGPPPHPLGDPLVRREGREKVTGTARYAAEHTPDGCAYAWPVPATVVRGRITELDTGAALALPGVIAVLTHENAPRLAPTGDPTLALLQEDRVPHRGWYVALAVADTLEAARDAAEAVHVSYATEPHDVTLTADHPRLYVPAEVFGGPGARERGDFDTAFAAAPATVDVTYTVPPLHNHPMEPHAATALWTHGHLTVHDSSQGATRVREDLAALFKLGQDQITVHSEHVGGGFGSKGTPRPQVVLAAMAARHTGRPVKLALPRRHLPAVVGHRAPTLHRVRLGAGPDGVITALAHEIVTHTSTVAEFVEQAAMPARIMYTSPHSRTVHRLAALDVPTPSWMRAPGEAPGMYALESAVDELAVVLDLDPIDLRIRNEPGTEPDTGRPFSSRHLVDCLRAGAARFGWSSRDPRPAVRRQGDLLLGTGVAAATYPVQISATDAEAHAAADGTFRVRVNATDIGTGARTVLAQIAAAALGAPADRVRVEIGSSDLPPAVLAGGSTGTASWGWAVHKACTVLLARLREHRGPLPAEGVTVTEDTRRETEQPSPYSRHAFGAVFAEVQVDTRTGEVRARRLLGQYAAGHILNPRTARSQFVGGMVMGLGMALTEDSALDPVYGDFTARDLAAYHVPACADVPAIEAHWLDEEDPHLNPMGSKGIGEIGIVGTPAAIGNAVWHATGVRLRDLPLTPDRILTARTVPLT

SEQ ID NO: 142 - protein, 710 aa, Streptomyces himastatinicus
MTRVDGLDKVTGAATYAYEFPTPDVGYVWPVQATIARGRVTEVDGAPALARPGVLAVLDSGNAPRLNTEAQAGPDLFVLQSPEVAYHGQIVAAVVATSLEAAREGAAAVRVSYEQEPHDVVLRFDDERAQVAETVTDGSPGFVEHGDAEGALAAAPVRTEAMYTTPVEHTSPMEPHATIAAWDEDRLTLYNADQGPFMSSQLLAAVFGLDQGAVEVVAEYIGGGFGSKGIPRSPAVLAALAAKHLGRPVKIALTRQQMFQLIPYRAPTIQRIRLGAERDGRLTAIDHEVVQQRSAMAEFADQTGSSTRVMYAAPNIRTTVKTAPLDVLTPAWFRAPGHTPGMFALESAMDELATELEIDPVELRIRNDTGVDPDSGKPFSSRGLVACLREGAARFDWALRDPKPGIRREGRWLVGTGVASAHHPDYVFPSSATARAEADGTFTVRVGAVDIGTGGRTALTQLAADALGIPVERLRLEIGRASLGPAPFAGGSLGTASWGWAVDKACRALLAELDTYGGAVPDGGLEVRADTTEDVELRASFSRHSFGAHFAQVRVDTDTGEIRVDRMLGVFAAGRIVNPKTARSQFVGAMTMGLSMALLEIGEVDPVFGDFANHDFAGYHVAANADVPKLEALWLDEQDDNPNPVRGKGIGELGIVGAAAAVTNAFHHATGQRVRDLPIRVERSREALRAARAEAQKRGPGAAEQGKPVG

SEQ ID NO: 143 - protein, 806 aa, unidentified
  source 1..806: note = Streptomyces lividans
MSHLSERPEKPVVGVSMPHESAVQHVTGAALYTDDLVQRTKDVLHAYPVQVMKARGRVTALRTGAALAVPGVVRVLTGADVPGVNDAGMKHDEPLFPDEVMFHGHAVAWVLGETLEAARIGAAAVEVDLEELPSVITLQDAIAADSYHGARPVMTHGDVDAGFADSAHVFTGEFQFSGQEHFYLETHAALAQVDENGQVFIQSSTQHPSETQEIVSHVFGVPAHEVTVQCFRMGGGFGGKEMQPHGFAAIAAFGAKFTGRPVRFRFNRTQDFTMSGKRHGFHATWKIGFDTEGRIQAFDATFTADGGWSFDFSEPVFARAFCHIDNTYWIPNARVAGRIARTNTVSNTAFRGFGGPQGMFVIEDIFGRCAPRFGVDAKEFRERNFYRPGQGQTTPYGQPVTQPERIAAVWQQVQDNGHIADREREIAAFNAAHPHTKRAFAVTGVKFGISFNFTAFNQGGAFVFIYKDGSVFINHGGTEMGQGFHTKMFQVAATTFGIPFHKVRFAPTRTDKVPNTSATAASSGADENGGAVKNACEQFRERFFRVAASQFGTNASDVRIVEGVARSFGSDQEFAWDDFVRTAYFQRVQFSAAGYYRTEGFHWDAKSFRGSPFKYFAIGAAATEVEVDGFTGAYRIRRVDIVHDVGDSFSPFIDIGQVEGGFVQGAGWFTFEDFRWDTGDGPNRGRFFTQAASTYKFPSFSEMPEEFNVTFFENATEEGAVFGSKAVGEPPFMFAFSVREAFRQAAAAFGPRGTAVEFASPATPEAVYWAIESARQGGTAGDGRTHGAAASDAVAVRTGVEAESGA

SEQ ID NO: 144 - protein, 494 aa, synthetic construct
MLASGMLLVALLVCLTVMVLMSVWQQRKSKGKLPPGPTPLPFIGNYLQLNTEQMYNSLMKISERYGPVFTIHLGPRRVVVLCGHDAVREALVDQAEEFSGRGEQATFDWVFKGYGVVFSNGERAKQLRRFSIATLRDFGVGKRGIEERIQEEAGFLIDALRGTGGANIDPTFFLSRTVSNVISSIVFGDRFDYKDKEFLSLLRMMLGIFQFTSTSTGQLYEMFSSVMKHLPGPQQQAFQLLQGLEDFIAKKVEHNQRTLDPNSPRDFIDSFLIRMQEEEKNPNTEFYLKNLVMTTLNLFIGGTETVSTTLRYGFLLLMKHPEVEAKVHEEIDRVIGKNRQPKFEDRAKMPYMEAVIHEIQRFGDVIPMSLARRVKKDTKFRDFFLPKGTEVFPMLGSVLRDPSFFSNPQDFNPQHFLNEKGQFKKSDAFVPFSIGKRNCFGEGLARMELFLFFTTVMQNFRFKSSQSPKDIDVSPKHVGFATIPRNYTMSFFPR

SEQ ID NO: 145 - protein, 494 aa, synthetic construct
MLASGMLLVALLVCLTVMVLMSVWQQRKSKGKLPPGPTPLPFIGNYLQLNTEQMYNSLMKISERYGPVFTIHLGPRRVVVLCGHDAVREALVDQAEEFSGRGEQATFDWVFKGYGVVFSNGERAKQLRRFSIATLRDFGVGKRGIEERIQEEAGFLIDALRGTGGANIDPTFFLSRTVSNVISSIVFGDRFDYKDKEFLSLLRMMLGIFQFTSTSTGQLYEMFSSVMKHLPGPQQQAFQLLQGLEDFIAKKVEHNQRTLDPNSPRDFIDSFLIRMQEEEKNPNTEFYLKNLVMTTLNLFIGGTETVSTTLRYGFLLLMKHPEVEAKVHEEIDRVIGKNRQPKFEDRAKMPYMEAVIHEIQRFGDVIPMSLARRVKKDTKFRDFFLPKGTEVFPMLGSVLRDPSFFSNPQDFNPQHFLNEKGQFKKSDAFVPFSIGKRNCFGEGLARMELFLFFTTVMQNFRLKSSQSPKDIDVSPKHVGFATIPRNYTMSFLPR

SEQ ID NO: 146 - protein, 503 aa, synthetic construct
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 147 - protein, 2136 aa, synthetic construct
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 148 - protein, 721 aa, synthetic construct
MGSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 149 - protein, 2002 aa, synthetic construct
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGFEEVQNINRRNSPYSQTMKSSACKIQVSCSNNTHFVSENKEQTTHPEFFAGNKTQNFHHMQYFPNNVIPKQDFFHRCFQEQEQKSQQASVFQGYKNRNQDMSGQQAAQFAQQRYFIHNHANVFPVPDQGGSHTQTPPQKDTQKHAAFRWHFFQKQEQQQTQQPQTESCHSQMHRPIKVEPGCKPHACMHTAPPENKTWKKVTKQENPPASCDNVQQKSIIETMEQHFKQFHAKSFFDHKAFTFKSQKQVKVEMSGPVTVFTRQTTAAEFDSHTPAFEQQTTSSEKTPTKRTAASVFNNFIESPSKFFDTPIKNFFDTPVKTQYDFPSCRCVEQIIEKDEGPFYTHFGAGPNVAAIREIMEERFGQKGKAIRIERVIYTGKEGKSSQGCPIAKWVVRRSSSEEKFFCFVRERAGHTCEAAVIVIFIFVWEGIPFSFADKFYSEFTETFRKYGTFTNRRCAFNEERTCACQGFDPETCGASFSFGCSWSMYYNGCKFARSKIPRKFKFFGDDPKEEEKFESHFQNFSTFMAPTYKKFAPDAYNNQIEYEHRAPECRFGFKEGRPFSGVTACFDFCAHAHRDFHNMQNGSTFVCTFTREDNREFGGKPEDEQFHVFPFYKVSDVDEFGSVEAQEEKKRSGAIQVFSSFRRKVRMFAEPVKTCRQRKFEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 150 - protein, 1659 aa, synthetic construct
MDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAWESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 151 - protein, 216 aa, Escherichia coli
MLDLFADAEPWQEPLAAGAVILRRFAFNAAEQLIRDINDVASQSPFRQMVTPGGYTMSVAMTNCGHLGWTTHRQGYLYSPIDPQTNKPWPAMPQSFHNLCQRAATAAGYPDFQPDACLINRYAPGAKLSLHQDKDEPDLRAPIVSVSLGLPAIFQFGGLKRNDPLKRLLLEHGDVVVWGGESRLFYHGIQPLKAGFHPLTIDCRYNLTFRQAGKKE

SEQ ID NO: 152 - protein, 170 aa, Homo sapiens
MEEKRRRARVQGAWAAPVKSQAIAQPATTAKSHLHQKPGQTWKNKEHHLSDREFVFKEPQQVVRRAPEPRVIEEGVYEISLSPTGVSRVCLYPGFVDVKEADWILEQLCQDVPWKQRTGIREDSILQLTFKKSAPVSGTATAPQSCWYERPSPPHIPGPAILTRTRLWAP

SEQ ID NO: 153 - protein, 525 aa, Escherichia coli
MTENIHKHRILILDFGSQYTQLVARRVRELGVYCELWAWDVTEAQIRDFNPSGIILSGGPESTTEENSPRAPQYVFEAGVPVFGVCYGMQTMAMQLGGHVEASNEREFGYAQVEVVNDSALVRGIEDALTADGKPLLDVWMSHGDKVTAIPSDFITVASTESCPFAIMANEEKRFYGVQFHPEVTHTRQGMRMLERFVRDICQCEALWTPAKIIDDAVARIREQVGDDKVILGLSGGVDSSVTAMLLHRAIGKNLTCVFVDNGLLRLNEAEQVLDMFGDHFGLNIVHVPAEDRFLSALAGENDPEAKRKIIGRVFVEVFDEEALKLEDVKWLAQGTIYPDVIESAASATGKAHVIKSHHNVGGLPKEMKMGLVEPLKELFKDEVRKIGLELGLPYDMLYRHPFPGPGLGVRVLGEVKKEYCDLLRRADAIFIEELRKADLYDKVSQAFTVFLPVRSVGVMGDGRKYDWVVSLRAVETIDFMTAHWAHLPYDFLGRVSNRIINEVNGISRVVYDISGKPPATIEWE

SEQ ID NO: 154 - protein, 349 aa, unidentified
  source 1..349: note = Staphylococcus sciuri
MNFNNKTKYGKIQEFLRSNNEPDYRIKQITNAIFKQRISRFEDMKVLPKLLREDLINNFGETVLNIKLLAEQNSEQVTKVLFEVSKNERVETVNMKYKAGWESFCISSQCGCNFGCKFCATGDIGLKKNLTVDEITDQVLYFHLLGHQIDSISFMGMGEALANRQVFDALDSFTDPNLFALSPRRLSISTIGIIPSIKKITQEYPQVNLTFSLHSPYSEERSKLMPINDRYPIDEVMNILDEHIRLTSRKVYIAYIMLPGVNDSLEHANEVVSLLKSRYKSGKLYHVNLIRYNPTISAPEMYGEANEGQVEAFYKVLKSAGIHVTIRSQFGIDIDAACGQLYGNYQNSQ

SEQ ID NO: 155 - protein, 392 aa, Aquifex aeolicus
MEIVQEGIAKIIVPEIPKTVSSDMPVFYNPRMRVNRDLAVLGLEYLCKKLGRPVKVADPLSASGIRAIRFLLETSCVEKAYANDISSKAIEIMKENFKLNNIPEDRYEIHGMEANFFLRKEWGFGFDYVDLDPFGTPVPFIESVALSMKRGGILSLTATDTAPLSGTYPKTCMRRYMARPLRNEFKHEVGIRILIKKVIELAAQYDIAMIPIFAYSHLHYFKLFFVKERGVEKVDKLIEQFGYIQYCFNCMNREVVTDLYKFKEKCPHCGSKFHIGGPLWIGKLWDEEFTNFLYEEAQKREEIEKETKRILKLIKEESQLQTVGFYVLSKLAEKVKLPAQPPIRIAVKFFNGVRTHFVGDGFRTNLSFEEVMKKMEELKEKQKEFLEKKKQG

SEQ ID NO: 156 - protein, 570 aa, Saccharomyces cerevisiae
MEGFFRIPLKRANLHGMLKAAISKIKANFTAYGAPRINIEDFNIVKEGKAEILFPKKETVFYNPIQQFNRDLSVTCIKAWDNLYGEECGQKRNNKKSKKKRCAETNDDSSKRQKMGNGSPKEAVGNSNRNEPYINILEALSATGLRAIRYAHEIPHVREVIANDLLPEAVESIKRNVEYNSVENIVKPNLDDANVLMYRNKATNNKFHVIDLDPYGTVTPFVDAAIQSIEEGGLMLVTCTDLSVLAGNGYPEKCFALYGGANMVSHESTHESALRLVLNLLKQTAAKYKKTVEPLLSLSIDFYVRVFVKVKTSPIEVKNVMSSTMTTYHCSRCGSYHNQPLGRISQREGRNNKTFTKYSVAQGPPVDTKCKFCEGTYHLAGPMYAGPLHNKEFIEEVLRINKEEHRDQDDTYGTRKRIEGMLSLAKNELSDSPFYFSPNHIASVIKLQVPPLKKVVAGLGSLGFECSLTHAQPSSLKTNAPWDAIWYVMQKCDDEKKDLSKMNPNTTGYKILSAMPGWLSGTVKSEYDSKLSFAPNEQSGNIEKLRKLKIVRYQENPTKNWGPKARPNTS

SEQ ID NO: 157 - protein, 659 aa, Homo sapiens
MQGSSLWLSLTFRSARVLSRARFFEWQSPGLPNTAAMENGTGPYGEERPREVQETTVTEGAAKIAFPSANEVFYNPVQEFNRDLTCAVITEFARIQLGAKGIQIKVPGEKDTQKVVVDLSEQEEEKVELKESENLASGDQPRTAAVGEICEEGLHVLEGLAASGLRSIRFALEVPGLRSVVANDASTRAVDLIRRNVQLNDVAHLVQPSQADARMLMYQHQRVSERFDVIDLDPYGSPATFLDAAVQAVSEGGLLCVTCTDMAVLAGNSGETCYSKYGAMALKSRACHEMALRIVLHSLDLRANCYQRFVVPLLSISADFYVRVFVRVFTGQAKVKASASKQALVFQCVGCGAFHLQRLGKASGVPSGRAKFSAACGPPVTPECEHCGQRHQLGGPMWAEPIHDLDFVGRVLEAVSANPGRFHTSERIRGVLSVITEELPDVPLYYTLDQLSSTIHCNTPSLLQLRSALLHADFRVSLSHACKNAVKTDAPASALWDIMRCWERECPVKRERLSETSPAFRILSVEPRLQANFTIREDANPSSRQRGLKRFQANPEANWGPRPRARPGGKAADEAMEERRRLLQNKRKEPPEDVAQRAARLKTFPCKRFKEGTCQRGDQCCYSHSPPTPRVSADAAPDCPETSNQTPPGPGAAAGPGID

SEQ ID NO: 158 - protein, 269 aa, Escherichia coli
MSFSCPLCHQPLSREKNSYICPQRHQFDMAKEGYVNLLPVQHKRSRDPGDSAEMMQARRAFLDAGHYQPLRDAIVAQLRERLDDKATAVLDIGCGEGYYTHAFADALPEITTFGLDVSKVAIKAAAKRYPQVTFCVASSHRLPFSDTSMDAIIRIYAPCKAEELARVVKPGGWVITATPGPRHLMELKGLIYNEVHLHAPHAEQLEGFTLQQSAELCYPMRLRGDEAVALLQMTPFAWRAKPEVWQTLAAKEVFDCQTDFNIHLWQRSY

SEQ ID NO: 159 - protein, 255 aa, Escherichia coli
MWIGIISLFPEMFRAITDYGVTGRAVKNGLLSIQSWSPRDFTHDRHRTVDDRPYGGGPGMLMMVQPLRDAIHAAKAAAGEGAKVIYLSPQGRKLDQAGVSELATNQKLILVCGRYEGIDERVIQTEIDEEWSIGDYVLSGGELPAMTLIDSVSRFIPGVLGHEASATEDSFAEGLLDCPHYTRPEVLEGMEVPPVLLSGNHAEIRRWRLKQSLGRTWLRRPELLENLALTEEQARLLAEFKTEHAQQQHKHDGMA

SEQ ID NO: 160 - protein, 339 aa, Homo sapiens
MSSEMLPAFIETSNVDKKQGINEDQEESQKPRLGEGCEPISKRQMKKLIKQKQWEEQRELRKQKRKEKRKRKKLERQCQMEPNSDGHDRKRVRRDVVHSTLRLIIDCSFDHLMVLKDIKKLHKQIQRCYAENRRALHPVQFYLTSHGGQLKKNMDENDKGWVNWKDIHIKPEHYSELIKKEDLIYLTSDSPNILKELDESKAYVIGGLVDHNHHKGLTYKQASDYGINHAQLPLGNFVKMNSRKVLAVNHVFEIILEYLETRDWQEAFFTILPQRKGAVPTDRACESASHDNQSVRMEEGGSDSDSSEEEYSRNELDSPHEEKQDKENHTESTVNSLPH

SEQ ID NO: 161 - protein, 336 aa, Methanocaldococcus jannaschii
MPLCLKINKKHGEQTRRILIENNLLNKDYKITSEGNYLYLPIKDVDEDILKSILNIEFELVDKELEEKKIIKKPSFREIISKKYRKEIDEGLISLSYDVVGDLVILQISDEVDEKIRKEIGELAYKLIPCKGVFRRKSEVKGEFRVRELEHLAGENRTLTIHKENGYRLWVDIAKVYFSPRLGGERARIMKKVSLNDVVVDMFAGVGPFSIACKNAKKIYAIDINPHAIELLKKNIKLNKLEHKIIPILSDVREVDVKGNRVIMNLPKFAHKFIDKALDIVEEGGVIHYYTIGKDFDKAIKLFEKKCDCEVLEKRIVKSYAPREYILALDFKINKK

SEQ ID NO: 162 - protein, 330 aa, unidentified
  source 1..330: note = Pyrococcus abyssi
MTLAVKVPLKEGEIVRRRLIELGALDNTYKIKREGNFLLIPVKFPVKGFEVVEAELEQVSRRPNSYREIVNVPQELRRFLPTSFDIIGNIAIIEIPEELKGYAKEIGRAIVEVHKNVKAVYMKGSKIEGEYRTRELIHIAGENITETIHRENGIRLKLDVAKVYFSPRLATERMRVFKMAQEGEVVFDMFAGVGPFSILLAKKAELVFACDINPWAIKYLEENIKLNKVNNVVPILGDSREIEVKADRIIMNLPKYAHEFLEHAISCINDGGVIHYYGFGPEGDPYGWHLERIRELANKFGVKVEVLGKRVIRNYAPRQYNIAIDFRVSF

SEQ ID NO: 163 - protein, 1610 aa, synthetic construct
MGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 164 - protein, 1617 aa, synthetic construct
MPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 165 - protein, 1626 aa, synthetic construct
MKRPAATKKAGQAKKKKGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 166 - protein, 1630 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 167 - protein, 1723 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 168 - protein, 1641 aa, synthetic construct
MGSKRTADGSEFESPKKKRKVGSGTNLSDIIEKETGKQLVIQESILMLPEEVEEVIGNKPESDILVHTAYDESTDENVMLLTSDAPEYKPWALVIQDSNGENKIKMLGSSGGSGGSGGSGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSPKKKRKV

SEQ ID NO: 169 - protein, 1638 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSTNLSDIIEKETGKQLVIQESILMLPEEVEEVIGNKPESDILVHTAYDESTDENVMLLTSDAPEYKPWALVIQDSNGENKIKMLGSSGGSGGSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSPKKKRKV

SEQ ID NO: 170 - protein, 1776 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 171 - protein, 1748 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKVSGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQ

SEQ ID NO: 172 - protein, 1777 aa, synthetic construct
MGSKRTADGSEFESPKKKRKVGSGAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSGSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 173 - protein, 1612 aa, synthetic construct
MGSKRTADGSEFESPKKKRKVGSGSTDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAVSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 174 - protein, 1566 aa, synthetic construct
MGSKRTADGSEFESPKKKRKVGSGSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKLSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 175 - protein, 1632 aa, synthetic construct
MGSKRTADGSEFESPKKKRKVGSGSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 176 - protein, 1602 aa, synthetic construct
MGSKRTADGSEFESPKKKRKVGSGEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 177 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 178 - protein, 1615 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKVEFGSG

SEQ ID NO: 179 - protein, 1582 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKVSGGSMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQ

SEQ ID NO: 180 - protein, 1612 aa, synthetic construct
MKRTADGSEFESPKKKRKVGSGAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 181 - protein, 1543 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSPKKKRKV

SEQ ID NO: 182 - protein, 1775 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESESGGSPKKKRKV

SEQ ID NO: 183 - protein, 1806 aa, synthetic construct
MGSKRTADGSEFESPKKKRKVGSGANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESESGGSSGGSSGSETPGTSESATPESSGGSSGGSGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSPKKKRKV

SEQ ID NO: 184 - protein, 1859 aa, synthetic construct
MGSKRTADGSEFESPKKKRKVGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKELSGGSPKKKRKV

SEQ ID NO: 185 - protein, 1890 aa, synthetic construct
MGSKRTADGSEFESPKKKRKVGSGIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKELSGGSSGGSSGSETPGTSESATPESSGGSSGGSGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKKRPAATKKAGQAKKKKGSSGGSGGSGGSPKKKRKV

SEQ ID NO: 186 - DNA, 20 bp, synthetic construct
  source 1..20: mol_type = other DNA
agcctcacccctctagccct

SEQ ID NO: 187 - DNA, 20 bp, synthetic construct
  source 1..20: mol_type = other DNA
ttctcccctctgctggatac

SEQ ID NO: 188 - DNA, 23 bp, synthetic construct
  source 1..23: mol_type = other DNA
tgctgcaagtaagcatgcatttg

SEQ ID NO: 189 - DNA, 23 bp, synthetic construct
  source 1..23: mol_type = other DNA
tgctgcaagtaagcatgcatttg

SEQ ID NO: 190 - DNA, 23 bp, synthetic construct
  source 1..23: mol_type = other DNA
gtttaaacacaccgggttaataa

SEQ ID NO: 191 - DNA, 20 bp, synthetic construct
  source 1..20: mol_type = other DNA
ctgatggtccatgtctgtta

SEQ ID NO: 192 - DNA, 20 bp, synthetic construct
  source 1..20: mol_type = other DNA
agcctcacccctctagccct

SEQ ID NO: 193 - protein, 7 aa, synthetic construct
PKKKRKV

SEQ ID NO: 194 - protein, 16 aa, synthetic construct
KRPAATKKAGQAKKKK

SEQ ID NO: 195 - protein, 18 aa, synthetic construct
KRTADGSEFESPKKKRKV

SEQ ID NO: 196 - protein, 20 aa, synthetic construct
  REGION 3..20: note = Amino acids 3-20 may be absent
GSGSGSGSGSGSGSGSGSGS

SEQ ID NO: 197 - protein, 30 aa, synthetic construct
  REGION 4..30: note = Amino acids 4-30 may be absent
GGSGGSGGSGGSGGSGGSGGSGGSGGSGGS

SEQ ID NO: 198 - protein, 30 aa, synthetic construct
  REGION 4..30: note = Amino acids 4-30 may be absent
GSSGSSGSSGSSGSSGSSGSSGSSGSSGSS

SEQ ID NO: 199 - protein, 40 aa, synthetic construct
  REGION 5..40: note = Amino acids 5-40 may be absent
GGSSGGSSGGSSGGSSGGSSGGSSGGSSGGSSGGSSGGSS

SEQ ID NO: 200 - protein, 50 aa, synthetic construct
  REGION 6..50: note = Amino acids 6-50 may be absent
SGGGSSGGGSSGGGSSGGGSSGGGSSGGGSSGGGSSGGGSSGGGSSGGGS

SEQ ID NO: 201 - protein, 40 aa, synthetic construct
  REGION 5..40: note = Amino acids 5-40 may be absent
SGGSSGGSSGGSSGGSSGGSSGGSSGGSSGGSSGGSSGGS

SEQ ID NO: 202 - protein, 14 aa, synthetic construct
GSSGGSGGSGGSGS

SEQ ID NO: 203 - protein, 16 aa, synthetic construct
SGSETPGTSESATPES

SEQ ID NO: 204 - protein, 32 aa, synthetic construct
SGGSSGGSSGSETPGTSESATPESSGGSSGGS

SEQ ID NO: 205 - protein, 10 aa, synthetic construct
SGGSGGSGGS

SEQ ID NO: 206 - protein, 7 aa, synthetic construct
GGGGGGS

SEQ ID NO: 207 - protein, 12 aa, synthetic construct
GSSGGSGGSGGS

SEQ ID NO: 208 - protein, 4 aa, synthetic construct
SGGS

SEQ ID NO: 209 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 210 - protein, 297 aa, synthetic construct
VTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 211 - protein, 228 aa, synthetic construct
ANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE

SEQ ID NO: 212 - protein, 312 aa, synthetic construct
IGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 213 - protein, 114 aa, synthetic construct
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQ

SEQ ID NO: 214 - DNA, 40 bp, synthetic construct
  source 1..40: mol_type = other DNA
gtttcagcctcacccctctagccctacatctctctttctt

SEQ ID NO: 215 - DNA, 40 bp, synthetic construct; identical to SEQ ID NO: 214
  source 1..40: mol_type = other DNA

SEQ ID NO: 216 - DNA, 40 bp, synthetic construct
  source 1..40: mol_type = other DNA
gtttcagcctcatttttttagccctacatctctctttctt

SEQ ID NO: 217 - DNA, 40 bp, synthetic construct
  source 1..40: mol_type = other DNA
gtttcagccttatttttttagccctacatctctctttctt

SEQ ID NO: 218 - DNA, 40 bp, synthetic construct
  source 1..40: mol_type = other DNA
gtttcagcctcacccctttagccctacatctctctttctt

SEQ ID NO: 219 - DNA, 40 bp, synthetic construct
  source 1..40: mol_type = other DNA
gtttcagcctcacctttttagccctacatctctctttctt

SEQ ID NO: 220 - DNA, 40 bp, synthetic construct
  source 1..40: mol_type = other DNA
gtttcagcctcatccctttagccctacatctctctttctt

SEQ ID NO: 221 - DNA, 40 bp, synthetic construct
  source 1..40: mol_type = other DNA
gtttcagccttatttttttagctctacatctctctttctt

SEQ ID NO: 222 - DNA, 40 bp, synthetic construct
  source 1..40: mol_type = other DNA
gtttcagcctcatttctttagccctacatctctctttctt

SEQ ID NO: 223 - DNA, 40 bp, synthetic construct
  source 1..40: mol_type = other DNA
gtttcagcctcacccttttagccctacatctctctttctt

SEQ ID NO: 224 - DNA, 40 bp, synthetic construct
  source 1..40: mol_type = other DNA
gtttcagcctcacttttttagccctacatctctctttctt

SEQ ID NO: 225 - DNA, 40 bp, synthetic construct
  source 1..40: mol_type = other DNA
gtttcagcctcacctctctagccctacatctctctttctt

SEQ ID NO: 226 - DNA, 40 bp, synthetic construct
  source 1..40: mol_type = other DNA
gtttcagcctcacttctttagccctacatctctctttctt

SEQ ID NO: 227 - DNA, 40 bp, synthetic construct
  source 1..40: mol_type = other DNA
gtttcagcctcactcctttagccctacatctctctttctt

SEQ ID NO: 228 - DNA, 40 bp, synthetic construct
  source 1..40: mol_type = other DNA
gtttcagcctcatttttttagctctacatctctctttctt

SEQ ID NO: 229 - DNA, 39 bp, synthetic construct
  source 1..39: mol_type = other DNA
gtttcagcctcaccctttagccctacatctctctttctt

SEQ ID NO: 230 - DNA, 40 bp, synthetic construct
  source 1..40: mol_type = other DNA
gtttcagcctcacccctgtagccctacatctctctttctt

SEQ ID NO: 231 - DNA, 40 bp, synthetic construct
  source 1..40: mol_type = other DNA
gttttagcctcatttttttagccctacatctctctttctt

SEQ ID NO: 232 - DNA, 40 bp, synthetic construct
  source 1..40: mol_type = other DNA
gtttcagcctcatttttttagccctacatctctttttctt

SEQ ID NO: 233 - DNA, 40 bp, synthetic construct
  source 1..40: mol_type = other DNA
gtttcagcctcatctttttagccctacatctctctttctt

SEQ ID NO: 234 - DNA, 40 bp, synthetic construct
  source 1..40: mol_type = other DNA
gtttcagccttatctttttagccctacatctctctttctt

SEQ ID NO: 235 - DNA, 40 bp, synthetic construct
  source 1..40: mol_type = other DNA
gtttcggcctcacccctctagccctacatctctctttctt

SEQ ID NO: 236 - DNA, 40 bp, synthetic construct
  source 1..40: mol_type = other DNA
gtttcagcctcattcttttagccctacatctctctttctt

SEQ ID NO: 237 - DNA, 40 bp, synthetic construct
  source 1..40: mol_type = other DNA
gtttcagcctcatttttctagccctacatctctctttctt

SEQ ID NO: 238 - DNA, 40 bp, synthetic construct
  source 1..40: mol_type = other DNA
gtttcagcctcacccctctagccccacatctctctttctt

SEQ ID NO: 239 - DNA, 40 bp, synthetic construct
  source 1..40: mol_type = other DNA
gttgcagcctcacccctctagccctacatctctctttctt

SEQ ID NO: 240 - DNA, 40 bp, synthetic construct; identical to SEQ ID NO: 214
  source 1..40: mol_type = other DNA

SEQ ID NO: 241 - DNA, 40 bp, synthetic construct; identical to SEQ ID NO: 218
  source 1..40: mol_type = other DNA

SEQ ID NO: 242 - DNA, 40 bp, synthetic construct; identical to SEQ ID NO: 214
  source 1..40: mol_type = other DNA

SEQ ID NO: 243 - DNA, 40 bp, synthetic construct
  source 1..40: mol_type = other DNA
gtttcagcctcacccctttagtcctacatctctctttctt

SEQ ID NO: 244 - DNA, 22 bp, synthetic construct
  source 1..22: mol_type = other DNA
gtttcagcctcacccctttctt

SEQ ID NO: 245 - DNA, 40 bp, synthetic construct; identical to SEQ ID NO: 224
  source 1..40: mol_type = other DNA

SEQ ID NO: 246 - DNA, 39 bp, synthetic construct
  source 1..39: mol_type = other DNA
gtttcagcctcacccctttagcctacatctctctttctt

SEQ ID NO: 247 - DNA, 31 bp, synthetic construct
  source 1..31: mol_type = other DNA
gtttcagcctcagcctacatctctctttctt

SEQ ID NO: 248 - DNA, 37 bp, synthetic construct
  source 1..37: mol_type = other DNA
gtttcagcctcacccctctagcccatctctctttctt

SEQ ID NO: 249 - DNA, 39 bp, synthetic construct
  source 1..39: mol_type = other DNA
gtttcagcctcatttttttagcctacatctctctttctt

SEQ ID NO: 250 - DNA, 40 bp, synthetic construct; identical to SEQ ID NO: 220
  source 1..40: mol_type = other DNA

SEQ ID NO: 251 - DNA, 40 bp, synthetic construct; identical to SEQ ID NO: 216
  source 1..40: mol_type = other DNA

SEQ ID NO: 252 - DNA, 36 bp, synthetic construct
  source 1..36: mol_type = other DNA
gtttcagcctcacccctctagccatctctctttctt

SEQ ID NO: 253 - DNA, 40 bp, synthetic construct
  source 1..40: mol_type = other DNA
gtttcagcctcacccctctagccctaacttaacatctctc

SEQ ID NO: 254 - DNA, 26 bp, synthetic construct
  source 1..26: mol_type = other DNA
gtttcagcctcacccctctctttctt

SEQ ID NO: 255 - DNA, 32 bp, synthetic construct
  source 1..32: mol_type = other DNA
gtttcagcctcacccctttagctctctttctt

SEQ ID NO: 256 - DNA, 39 bp, synthetic construct
  source 1..39: mol_type = other DNA
gtttcagcctcatttttttagcctatatctctctttctt

SEQ ID NO: 257 - DNA, 32 bp, synthetic construct
  source 1..32: mol_type = other DNA
gtttcagcctcacccctacatctctctttctt

SEQ ID NO: 258 - DNA, 40 bp, synthetic construct
  source 1..40: mol_type = other DNA
gtttcagcctcacccctctagtcctacatctctctttctt

SEQ ID NO: 259 - DNA, 11 bp, synthetic construct
  source 1..11: mol_type = other DNA
ctctgctggat

SEQ ID NO: 260 - DNA, 40 bp, synthetic construct
  source 1..40: mol_type = other DNA
gtttcagcctcactcctttagtcctacatctctctttctt

SEQ ID NO: 261 - DNA, 39 bp, synthetic construct
  source 1..39: mol_type = other DNA
gtttcagcctcacccctctagcccacatctctctttctt

SEQ ID NO: 262 - DNA, 40 bp, synthetic construct
  source 1..40: mol_type = other DNA
gtttcagcctcacctctttagccctacatctctctttctt

SEQ ID NO: 263 - DNA, 40 bp, synthetic construct; identical to SEQ ID NO: 226
  source 1..40: mol_type = other DNA

SEQ ID NO: 264 - DNA, 40 bp, synthetic construct
  source 1..40: mol_type = other DNA
ctttcttctcccctctgctggatacctctgggactcccca

SEQ ID NO: 265 - DNA, 40 bp, synthetic construct; identical to SEQ ID NO: 264
  source 1..40: mol_type = other DNA

SEQ ID NO: 266 - DNA, 40 bp, synthetic construct
  source 1..40: mol_type = other DNA
ctttcttctcccttttgctggatacctctgggactcccca

SEQ ID NO: 267 - DNA, 40 bp, synthetic construct
  source 1..40: mol_type = other DNA
ctttcttctcccctttgctggatacctctgggactcccca

SEQ ID NO: 268 - DNA, 40 bp, synthetic construct
  source 1..40: mol_type = other DNA
ctttcttctaccctgtgctggatacctctgggactcccca

SEQ ID NO: 269 - DNA, 40 bp, synthetic construct
  source 1..40: mol_type = other DNA
ctttcttctcccttttgttggatacctctgggactcccca

SEQ ID NO: 270 - DNA, 40 bp, synthetic construct
  source 1..40: mol_type = other DNA
ctttcttctcccctatgctggatacctctgggactcccca

SEQ ID NO: 271 - DNA, 40 bp, synthetic construct
  source 1..40: mol_type = other DNA
ctttcttctcctttttgctggatacctctgggactcccca

SEQ ID NO: 272 - DNA, 40 bp, synthetic construct
  source 1..40: mol_type = other DNA
ctttcttctcccttttgctggatatctctgggactcccca

SEQ ID NO: 273 - DNA, 40 bp, synthetic construct
  source 1..40: mol_type = other DNA
ctttcttctcccttctgctggatacctctgggactcccca

SEQ ID NO: 274 - DNA, 40 bp, synthetic construct
  source 1..40: mol_type = other DNA
ctttcttctcccttctgttggatacctctgggactcccca

SEQ ID NO: 275 - DNA, 40 bp, synthetic construct
  source 1..40: mol_type = other DNA
atttcttctcccctctgctggatacctctgggactcccca

SEQ ID NO: 276 - DNA, 40 bp, synthetic construct
  source 1..40: mol_type = other DNA
ctttcttctcccctctgcgggatacctctgggactcccca

SEQ ID NO: 277 - DNA, 40 bp, synthetic construct
  source 1..40: mol_type = other DNA
ctttcttctcccctctgccggatacctctgggactcccca

SEQ ID NO: 278 - DNA, 40 bp, synthetic construct
  source 1..40: mol_type = other DNA
ctttcttctcctttttgctggatacctcagggactcccca

SEQ ID NO: 279 - DNA, 40 bp, synthetic construct
  source 1..40: mol_type = other DNA
ctttcttcccccctctgctggatacctctgggactcccca

SEQ ID NO: 280 - DNA, 40 bp, synthetic construct
  source 1..40: mol_type = other DNA
ctttcttctaccctctgctggatacctctgggactcccca

SEQ ID NO: 281 - DNA, 39 bp, synthetic construct
  source 1..39: mol_type = other DNA
ctttcttctcccctctgctggatacctctgggactccca

SEQ ID NO: 282 - DNA, 40 bp, synthetic construct
  source 1..40: mol_type = other DNA
ctttcttctcccctttgttggatacctctgggactcccca

SEQ ID NO: 283 - DNA, 40 bp, synthetic construct
  source 1..40: mol_type = other DNA
ctttcttctcccctgtgctggatacctctgggactcccca

SEQ ID NO: 284 - DNA, 40 bp, synthetic construct
  source 1..40: mol_type = other DNA
ctttcttctcccctttgccggatacctctgggactcccca

SEQ ID NO: 285 - DNA, 40 bp, synthetic construct
  source 1..40: mol_type = other DNA
ttttcttctcccttttgctggatacctctgggactcccca

SEQ ID NO: 286 - DNA, 40 bp, synthetic construct
  source 1..40: mol_type = other DNA
ctttcttctcccctttgctggatatctctgggactcccca

SEQ ID NO: 287 - DNA, 40 bp, synthetic construct
  source 1..40: mol_type = other DNA
ctttcttctcccttctgctggatatctctgggactcccca

SEQ ID NO: 288 - DNA, 40 bp, synthetic construct
  source 1..40: mol_type = other DNA
ctttcttttcccttttgctggatacctctgggactcccca

SEQ ID NO: 289 - DNA, 39 bp, synthetic construct
  source 1..39: mol_type = other DNA
ctttcttctccctttgttggatacctctgggactcccca

SEQ ID NO: 290 - DNA, 40 bp, synthetic construct; identical to SEQ ID NO: 264
  source 1..40: mol_type = other DNA

SEQ ID NO: 291 - DNA, 40 bp, synthetic construct; identical to SEQ ID NO: 267
  source 1..40: mol_type = other DNA

SEQ ID NO: 292 - DNA, 40 bp, synthetic construct; identical to SEQ ID NO: 266
  source 1..40: mol_type = other DNA

SEQ ID NO: 293 - DNA, 40 bp, synthetic construct; identical to SEQ ID NO: 264
  source 1..40: mol_type = other DNA

SEQ ID NO: 294 - DNA, 30 bp, synthetic construct
  source 1..30: mol_type = other DNA
ctttcttctcccctttgctgggactcccca

SEQ ID NO: 295 - DNA, 29 bp, synthetic construct
  source 1..29: mol_type = other DNA
ctttcttctcccctttgctggactcccca

SEQ ID NO: 296 - DNA, 40 bp, synthetic construct
  source 1..40: mol_type = other DNA
ctttcttctcccctttgctggatacctttgggactcccca

SEQ ID NO: 297 - DNA, 14 bp, synthetic construct
  source 1..14: mol_type = other DNA
ctttcttctcccca

SEQ ID NO: 298 - DNA, 32 bp, synthetic construct
  source 1..32: mol_type = other DNA
ctttcttctccccttacctctgggactcccca

SEQ ID NO: 299 - DNA, 40 bp, synthetic construct
  source 1..40: mol_type = other DNA
ctttcttctcctttttgctggatacctacctctgggactc

SEQ ID NO: 300 - DNA, 40 bp, synthetic construct; identical to SEQ ID NO: 271
  source 1..40: mol_type = other DNA

SEQ ID NO: 301 - DNA, 29 bp, synthetic construct
  source 1..29: mol_type = other DNA
ctttcttctcccttttgctggactcccca

SEQ ID NO: 302 - DNA, 30 bp, synthetic construct
  source 1..30: mol_type = other DNA
ctttcttctcccttttgctgggactcccca

SEQ ID NO: 303 - DNA, 30 bp, synthetic construct
  source 1..30: mol_type = other DNA
ctttcttctcccttttgttgggactcccca

SEQ ID NO: 304 - DNA, 18 bp, synthetic construct
  source 1..18: mol_type = other DNA
ctttcttctcccctttgc

SEQ ID NO: 305 - DNA, 21 bp, synthetic construct
  source 1..21: mol_type = other DNA
ctttctttacctcactcccca

SEQ ID NO: 306 - DNA, 29 bp, synthetic construct
  source 1..29: mol_type = other DNA
ctttcttctcccttttttgggactcccca

SEQ ID NO: 307 - DNA, 40 bp, synthetic construct; identical to SEQ ID NO: 273
  source 1..40: mol_type = other DNA

SEQ ID NO: 308 - DNA, 33 bp, synthetic construct
  source 1..33: mol_type = other DNA
ctttcttctcccctctgcctctgggactcccca

SEQ ID NO: 309 - DNA, 28 bp, synthetic construct
  source 1..28: mol_type = other DNA
ctttcttctctacctctgggactcccca

SEQ ID NO: 310 - DNA, 40 bp, synthetic construct
  source 1..40: mol_type = other DNA
ctttctttttctttgtgttggatacccctgggactcccca

SEQ ID NO: 311 - DNA, 15 bp, synthetic construct
  source 1..15: mol_type = other DNA
ctttcttctccccca

SEQ ID NO: 312 - DNA, 19 bp, synthetic construct
  source 1..19: mol_type = other DNA
ctttcttctcccttttgca

SEQ ID NO: 313 - DNA, 38 bp, synthetic construct
  source 1..38: mol_type = other DNA
ctttcttctcccttttgctggacctctgggactcccca

SEQ ID NO: 314 - DNA, 40 bp, synthetic construct
  source 1..40: mol_type = other DNA
ttttcttcttccttttgctggacttaacaactaaaactcc

SEQ ID NO: 315 - DNA, 40 bp, synthetic construct
  source 1..40: mol_type = other DNA
ctttcttctcccttttgctggatacctcctcggactcccc

SEQ ID NO: 316 - DNA, 40 bp, synthetic construct
  source 1..40: mol_type = other DNA
atttctgctgcaagtaagcatgcatttgtaggcttgatgc

SEQ ID NO: 317 - DNA, 40 bp, synthetic construct; identical to SEQ ID NO: 316
  source 1..40: mol_type = other DNA

SEQ ID NO: 318 - DNA, 40 bp, synthetic construct
  source 1..40: mol_type = other DNA
atttctgctgcgggtgggcgtgcatttgtaggcttgatgc

SEQ ID NO: 319 - DNA, 40 bp, synthetic construct
  source 1..40: mol_type = other DNA
atttctgctgcaagtgagcatgcatttgtaggcttgatgc

SEQ ID NO: 320 - DNA, 40 bp, synthetic construct
  source 1..40: mol_type = other DNA
atttctgctgcgggtgagcatgcatttgtaggcttgatgc

SEQ ID NO: 321 - DNA, 40 bp, synthetic construct
  source 1..40: mol_type = other DNA
atttctgctgcaagtgagcgtgcatttgtaggcttgatgc

SEQ ID NO: 322 - DNA, 40 bp, synthetic construct
  source 1..40: mol_type = other DNA
atttctgctgcaggtgggcgtgcatttgtaggcttgatgc

SEQ ID NO: 323 - DNA, 40 bp, synthetic construct
  source 1..40: mol_type = other DNA
atttctgctgcaagtgggcatgcatttgtaggcttgatgc

SEQ ID NO: 324 - DNA, 40 bp, synthetic construct
  source 1..40: mol_type = other DNA
atttctgctgcgggtgggcgtgcgtttgtaggcttgatgc

SEQ ID NO: 325 - DNA, 40 bp, synthetic construct
  source 1..40: mol_type = other DNA
atttctgctgcaggtgggcgtgcgtttgtaggcttgatgc

SEQ ID NO: 326 - DNA, 40 bp, synthetic construct
  source 1..40: mol_type = other DNA
atttctgctgcaggtgggcatgcatttgtaggcttgatgc

SEQ ID NO: 327 - DNA, 40 bp, synthetic construct
  source 1..40: mol_type = other DNA
atttctgctgcaagtgggcgtgcatttgtaggcttgatgc

SEQ ID NO: 328 - DNA, 40 bp, synthetic construct
  source 1..40: mol_type = other DNA
atttctgctgcgagtgggcgtgcgtttgtaggcttgatgc

SEQ ID NO: 329 - DNA, 40 bp, synthetic construct
  source 1..40: mol_type = other DNA
atttctgctgcaggtgagcatgcatttgtaggcttgatgc

SEQ ID NO: 330 - DNA, 40 bp, synthetic construct
  source 1..40: mol_type = other DNA
atttctgctgccagtgagcatgcatttgtaggcttgatgc

SEQ ID NO: 331 - DNA, 40 bp, synthetic construct
  source 1..40: mol_type = other DNA
atttctgctgcgggtgggcatgcatttgtaggcttgatgc

SEQ ID NO: 332 - DNA, 40 bp, synthetic construct
  source 1..40: mol_type = other DNA
atttctgctgcgagtgggcgtgcatttgtaggcttgatgc

SEQ ID NO: 333 - DNA, 40 bp, synthetic construct
  source 1..40: mol_type = other DNA
atttctgctgcgggtgggcatgcgtttgtaggcttgatgc

SEQ ID NO: 334 - DNA, 40 bp, synthetic construct
  source 1..40: mol_type = other DNA
atttctgctgcgggtgagcgtgcgtttgtaggcttgatgc

SEQ ID NO: 335 - DNA, 40 bp, synthetic construct
  source 1..40: mol_type = other DNA
atttctgctgcaagtaagcatgcatttgtgggcttgatgc

SEQ ID NO: 336 - DNA, 40 bp, synthetic construct
  source 1..40: mol_type = other DNA
atttctgctgcaagtaagcatgtatttgtaggcttgatgc

SEQ ID NO: 337 - DNA, 40 bp, synthetic construct
  source 1..40: mol_type = other DNA
atttctgctgcaagtaggcatgcatttgtaggcttgatgc

SEQ ID NO: 338 - DNA, 40 bp, synthetic construct
  source 1..40: mol_type = other DNA
atttctgctgcaggtaagcatgcatttgtaggcttgatgc

SEQ ID NO: 339 - DNA, 40 bp, synthetic construct
  source 1..40: mol_type = other DNA
atttctgctgcaagtaagcgtgcatttgtaggcttgatgc

SEQ ID NO: 340 - DNA, 40 bp, synthetic construct
  source 1..40: mol_type = other DNA
atttctgctgcaagtaagcatgcatttgtaggctcgatgc

SEQ ID NO: 341 - DNA, 40 bp, synthetic construct
  source 1..40: mol_type = other DNA
atttctgttgcaagtaagcatgcatttgtaggcttgatgc

SEQ ID NO: 342 - DNA, 40 bp, synthetic construct; identical to SEQ ID NO: 316
  source 1..40: mol_type = other DNA

SEQ ID NO: 343 - DNA, 40 bp, synthetic construct; identical to SEQ ID NO: 316
  source 1..40: mol_type = other DNA

SEQ ID NO: 344 - DNA, 40 bp, synthetic construct; identical to SEQ ID NO: 319
  source 1..40: mol_type = other DNA

SEQ ID NO: 345 - DNA, 40 bp, synthetic construct; identical to SEQ ID NO: 321
  source 1..40: mol_type = other DNA

SEQ ID NO: 346 - DNA, 40 bp, synthetic construct; identical to SEQ ID NO: 339
  source 1..40: mol_type = other DNA

SEQ ID NO: 347 - DNA, 40 bp, synthetic construct
  source 1..40: mol_type = other DNA
atttctgctgcaagtgagcatgcatttgcatttgtaggct

SEQ ID NO: 348 - DNA, 26 bp, synthetic construct
  source 1..26: mol_type = other DNA
atttctgctgcaagtaagcatgatgc

SEQ ID NO: 349 - DNA, 36 bp, synthetic construct
  source 1..36: mol_type = other DNA
atttctgctgcaagtaagcatttgtaggcttgatgc

SEQ ID NO: 350 - DNA, 38 bp, synthetic construct
  source 1..38: mol_type = other DNA
atttctgctgcaagtgagcatgcatttaggcttgatgc

SEQ ID NO: 351 - DNA, 40 bp, synthetic construct; identical to SEQ ID NO: 329
  source 1..40: mol_type = other DNA

SEQ ID NO: 352 - DNA, 30 bp, synthetic construct
  source 1..30: mol_type = other DNA
atttctgctgcaagtaagcatgcttgatgc

SEQ ID NO: 353 - DNA, 26 bp, synthetic construct
  source 1..26: mol_type = other DNA
atttctgctgcaagtaggcttgatgc

SEQ ID NO: 354 - DNA, 26 bp, synthetic construct
  source 1..26: mol_type = other DNA
atttctgctgcaagtaagcttgatgc

SEQ ID NO: 355 - DNA, 26 bp, synthetic construct
  source 1..26: mol_type = other DNA
atttctgctgcaagtgagcttgatgc

SEQ ID NO: 356 - DNA, 40 bp, synthetic construct; identical to SEQ ID NO: 323
  source 1..40: mol_type = other DNA

SEQ ID NO: 357 - DNA, 36 bp, synthetic construct
  source 1..36: mol_type = other DNA
atttctgctgcaagtaagcatgtgtaggcttgatgc

SEQ ID NO: 358 - DNA, 26 bp, synthetic construct
  source 1..26: mol_type = other DNA
atttctgctgcaagtgagcgtgatgc

SEQ ID NO: 359 - DNA, 40 bp, synthetic construct
  source 1..40: mol_type = other DNA
atttctgctgcaagtaagcatgcatttcatttgtaggctt

SEQ ID NO: 360 - DNA, 34 bp, synthetic construct
  source 1..34: mol_type = other DNA
atttctgctgcaagtaagcatgtaggcttgatgc

SEQ ID NO: 361 - DNA, 40 bp, synthetic construct
  source 1..40: mol_type = other DNA
atttctgctgcaagtaagcatgcatttgcatttgtaggct

SEQ ID NO: 362 - DNA, 27 bp, synthetic construct
  source 1..27: mol_type = other DNA
atttctgctgctttgtaggcttgatgc

SEQ ID NO: 363 - DNA, 39 bp, synthetic construct
  source 1..39: mol_type = other DNA
atttctgctgcaagtaagcatgctttgtaggcttgatgc

SEQ ID NO: 364 - DNA, 30 bp, synthetic construct
  source 1..30: mol_type = other DNA
atttctgctgcaagtgagcgtgcttgatgc

SEQ ID NO: 365 - DNA, 40 bp, synthetic construct
  source 1..40: mol_type = other DNA
atttctgctgcaggtgagcatgcatttggtaggcttgatg

SEQ ID NO: 366 - DNA, 27 bp, synthetic construct
  source 1..27: mol_type = other DNA
tttggtttaaacacaccgggttaataa

SEQ ID NO: 367 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 368 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDLGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 369 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDKGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 370 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 371 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFENSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 372 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 373 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEGQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 374 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 375 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNGGFGGSRGKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 376 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEGQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 377 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 378 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 379 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 380 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 381 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKKVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 382 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 383 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVFKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 384 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 385 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 386 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKKVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 387 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVFKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 388 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 389 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDLGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 390 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDKGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 391 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 392 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFENSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 393 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 394 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEGQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 395 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 396 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNGGFGGSRGKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 397 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEGQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 398 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 399 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 400 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 401 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 402 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKKVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 403 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 404 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVFKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 405 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGEGNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVYKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 406 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSGVKVEKQVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 407 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDMGDRNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKKVTQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 408 - protein, 1464 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIATILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGSRVKVEKQVFKKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKSGGSKRTADGSEFEPKKKRKV

SEQ ID NO: 409 - DNA, 27 bp, synthetic construct
  source 1..27: mol_type = other DNA
tttctgctgcaagtaagcatgcatttg

(19) United States
(12) Patent Application Publication
(10) Pub. No.: US 2025/0388886 A1
(43) Pub. Date: Dec. 25, 2025

(54) Cas12a Endonuclease Variants and Methods of Use

(71) Applicant: ENSOMA ApS, Frederiksberg (DK)

(72) Inventors: Stefano STELLA, Frederiksberg (DK); Nikolai WULFF, Frederiksberg (DK); Faizaan MOHAMMAD, Frederiksberg (DK)

(73) Assignee: ENSOMA ApS, Frederiksberg (DK)

(21) Appl. No.: 18/726,872

(22) PCT Filed: Jan. 6, 2023
(86) PCT No.: PCT/IB2023/000043

§ 371 Date: Jul. 15, 2024

Related U.S. Application Data
(60) Provisional application No. 63/297,182, filed on Jan. 6, 2022.
(60) Provisional application No. 63/297,189, filed on Jan. 6, 2022.

(51) Int. Cl. C12N 9/22; C12N 9/78; C12N 15/11; C12N 15/90

(52) U.S. Cl. CPC C12N 9/226; C12N 9/78; C12N 15/11; C12N 15/907; C12Y 305/04004; C12Y 305/04005; C07K 2319/00; C12N 2310/20

(57) ABSTRACT

The present disclosure provides endonuclease variants having improved properties, such as hyperactivity and/or low indiscriminate single strand DNase activity, relative to the corresponding wild-type endonucleases.

### RELATED APPLICATIONS

This application is a national stage application under 35 U.S.C. § 371 of International Patent Application No. PCT/IB2023/000043, filed Jan. 6, 2023, which claims the benefit under 35 U.S.C. § 119(e) of U.S. provisional application No. 63/297,182, filed Jan. 6, 2022, and U.S. provisional application No. 63/297,189, filed Jan. 6, 2022. The disclosures of the aforementioned priority applications are incorporated herein by reference in their entirety.

### REFERENCE TO AN ELECTRONIC SEQUENCE LISTING

The contents of the electronic sequence listing (125665.US004.xml; Size: 634,614 bytes; and Date of Creation: Jul. 30, 2024) is herein incorporated by reference in its entirety.

### BACKGROUND

Prokaryotes have developed an adaptive immune system called Clustered regularly interspaced short palindromic repeats (CRISPR) that associate with Cas proteins to constitute an adaptive immune system that can combat attacks by foreign mobile genetic elements such as plasmids and phages. The CRISPR-Cas systems are classified into two classes (Classes 1 and 2) that are subdivided into six types (types I through VI). Class 1 (types I, III and IV) systems use multiple Cas proteins in their CRISPR ribonucleoprotein effector nucleases and Class 2 systems (types II, V and VI) use a single Cas protein. Class 2 type V is further classified into 4 subtypes (V-A, V-B, V-C, V-U). At present, V-C and V-U remain widely uncharacterized and no structural information on these systems is available. V-A encodes the protein Cas12a (also known as Cpf1) and recently several high-resolution structures of Cas12a have provided an insight into its working mechanism.

Class 2 type V CRISPR-Cas12a is an RNA-guided endonuclease that has been harnessed as a genome editing tool. Broader use of these enzymes for gene and epigenetic editing requires improvement of certain properties.

### SUMMARY

The present disclosure provides, in some aspects, variant Cas12a endonucleases with improved properties, such as hyperactivity and low indiscriminate single strand DNA degradation activity. Broad use of wild-type Cas12a has been limited, in part, due to its lower editing efficiency, relative to Cas9, and its indiscriminate single strand DNA degradation activity. The lid region, which is involved in the checkpoints for accurate target recognition, is responsible for this indiscriminate ssDNA degradation activity displayed by all wild-type Cas12a orthologs. Surprisingly, the data described herein demonstrate that certain modifications to the lid region of Cas12a can impact not only indiscriminate single strand deoxyribonuclease (ssDNase) activity but also targeted cleavage activity-both double strand and single strand cleavage activity.

Engineered variant endonucleases, in some embodiments, exhibit more efficient cleavage activity, relative to their wild-type reference Cas12a endonuclease. In other embodiments, engineered variant endonucleases of the present disclosure exhibit low to no indiscriminate single strand DNase activity. Also provided herein, in some embodiments, are variant Cas12a endonucleases that exhibit a preference for cleavage of one strand over the other strand of a double strand DNA.

From structural studies of the LbCas12a ND2006 endonuclease, Applicants have identified a particular domain, referred to herein as the “LID-hub domain,” that is involved in a subset of catalytic events. For example, certain substitutions made at positions K932, N933, and V936 increase cleavage efficiency (“hyperactivity”) and certain substitutions made at positions K932, N933, V936, Q944, F983, and M986 reduce indiscriminate ssDNase activity. Certain amino acid substitutions within the vicinity of the LID and LID-hub domains also impact activity (e.g., V938 or Q941).

Further still, Applicants have also unexpectedly shown that modifications to the LID stabilizing charge network (defined by its three-dimensional structure to include at least positions E835, R836, R935, and/or K940, with reference to amino acid position numbering of LbCas12a ND2006) shift Cas12a cleavage preferences (e.g., from double strand DNA cleavage activity).

In some embodiments, variant Cas12a endonucleases comprise amino acid mutations at one or more amino acid positions within the lid region. For example, in some embodiments, a variant Cas12a endonuclease comprises one or more mutations at an amino acid position corresponding to positions 925 to 937 of Lachnospiraceae bacterium ND2006 (e.g., SEQ ID NO: 1). In some embodiments, a variant Cas12a endonuclease comprises one or more mutations at an amino acid position corresponding to positions 936 to 948 of Lachnospiraceae bacterium COE1 (e.g., SEQ ID NO: 47).

As described herein, the term “variant Cas12a endonuclease(s)” is interchangeable with the term “Cas12a variant.”

Some aspects relate to an engineered variant Cas12a endonuclease comprising a polypeptide sequence comprising a mutation at an amino acid position corresponding to position E95, E125, N256, R747, H759, N813, K932, N933, S934, V936, S982, or K984 with reference to amino acid position numbering of LbCas12a ND2006. In some embodiments, as any one or more of the foregoing variant Cas12a endonucleases exhibits hyperactivity.

Other aspects relate to an engineered variant Cas12a endonuclease comprising a polypeptide sequence comprising a mutation at an amino acid position corresponding to position E95R, E95Y, E125A, E125W, N256A, R747Y, H759V, H759D, N813R, N813H, K932L, N933E, N933V, S934Q, V936E, V936M, V936K, S982N, or K984R with reference to amino acid position numbering of LbCas12a ND2006. In some embodiments, as any one or more of the foregoing variant Cas12a endonucleases exhibits hyperactivity.

Some aspects relate to an engineered variant Cas12a endonuclease comprising a polypeptide sequence comprising a mutation at an amino acid position corresponding to position N256, 1831, K932, N933, S934, V936, Q944, S982, F983, K984, M986, or T988 with reference to amino acid position numbering of LbCas12a ND2006. In some embodiments, as any one or more of the foregoing variant Cas12a endonucleases exhibits hypoactivity.

Other aspects relate to an engineered variant Cas12a endonuclease comprising a polypeptide sequence comprising a mutation at an amino acid position corresponding to position N256K, I831A, I831Y, K932A, K932F, K932H, K932M, K932N, K932Q, K932R, K932S, K932T, K932W, K932Y, N933L, S934W, V936G, Q944D, Q944E, Q944K, Q944M, S982T, S982W, F983G, F983L, K984F, M986G, M986L, M986S, or T988F with reference to amino acid position numbering of LbCas12a ND2006. In some embodiments, as any one or more of the foregoing variant Cas12a endonucleases exhibits hypoactivity.

Yet other aspects relate to an engineered variant Cas12a endonuclease comprising a polypeptide sequence comprising mutations at an amino acid positions corresponding to positions: K932F and F983L; K932F and T988F; K932R and Q944D; K932R and F983L; K932R and T988F; K932Y and F983L; K932Y and T988F; N933L and Q944M; V936G and Q944D; V936G and S982W; V936G and M986G; V936G and T988F; Q944D and S982W; Q944D and F983L; Q944D and T988F; S982W and F983L; S982W and T988F; or F983G and M986G with reference to amino acid position numbering of LbCas12a ND2006. In some embodiments, as any one or more of the foregoing variant Cas12a endonucleases exhibits hypoactivity.

Some aspects relate to an engineered variant Cas12a endonuclease comprising a polypeptide sequence comprising a mutation at an amino acid position corresponding to position N813, 1831, K932, N933, S934, V936, Q944, S982, F983, K984, M986, or T988 with reference to amino acid position numbering of LbCas12a ND2006. In some embodiments, as any one or more of the foregoing variant Cas12a endonucleases exhibits low (or no) ssDNase activity, such as low (or no) indiscriminate ssDNase activity.

Other aspects relate to an engineered variant Cas12a endonuclease comprising a polypeptide sequence comprising a mutation at an amino acid position corresponding to position N813H, N813R, N813W, I831A, I831Y, K932A, K932F, K932H, K932M, K932N, K932Q, K932R, K932S, K932T, K932W, K932Y, N933E, N933L, S934K, S934Q, V936E, V936G, Q944D, Q944E, Q944K, S982W, F983G, F983L, K984F, M986F, M986G, or T988F with reference to amino acid position numbering of LbCas12a ND2006. In some embodiments, as any one or more of the foregoing variant Cas12a endonucleases exhibits low (or no) ssDNase activity, such as low (or no) indiscriminate ssDNase activity.

Yet other aspects relate to an engineered variant Cas12a endonuclease comprising a polypeptide sequence comprising mutations at an amino acid positions corresponding to positions: N933L and Q944M; or F983G and M986G with reference to amino acid position numbering of LbCas12a ND2006. In some embodiments, as any one or more of the foregoing variant Cas12a endonucleases exhibits low (or no) ssDNase activity, such as low (or no) indiscriminate ssDNase activity.

In some embodiments, an engineered variant Cas12a endonuclease is fused to an effector protein.

In some embodiments, an engineered variant Cas12a endonuclease provided herein comprises an amino acid sequence having at least 85%, at least 90%, or least 95%, but less than 100% identity with the amino acid sequence of a wild-type Cas12a endonuclease selected from Acidaminococcus sp., Lachnospiraceae sp., and Francisella sp.

In some embodiments, an engineered variant Cas12a endonuclease further comprises no more than 1, 2, 3, 4, 5, 6, 7, 8, 9, or 10 additional amino acid substitutions relative to a wild-type reference Cas12a endonuclease. In some embodiments, a variant Cas12a endonuclease further comprises no more than 5 additional amino acid substitutions relative to a wild-type reference Cas12a endonuclease.

The present disclosure also provides an engineered variant Cas12a endonuclease comprising a polypeptide sequence comprising the amino acid sequence of any one of SEQ ID NOs: 48-119 and 367-387, or an ortholog thereof.

Also provided herein are polynucleotides encoding an engineered variant Cas12a endonuclease of the present disclosure.

Further provided herein are cells comprising (a) an engineered variant Cas12a endonuclease of the present disclosure or a polynucleotide endonuclease of the present disclosure and (b) a guide RNA or a polynucleotide encoding a guide RNA.

Some aspects herein relate to a method comprising introducing into a cell (a) an engineered variant Cas12a endonuclease of the present disclosure or a polynucleotide of the present disclosure and optionally (b) a guide RNA or a polynucleotide encoding a guide RNA.

The present disclosure also provides uses of an engineered variant Cas12a endonuclease of the present disclosure for cleaving a nucleic acid.

In some embodiments, a method for introducing a double strand break in a target nucleic acid comprises introducing into a cell comprising a target nucleic acid (a) an engineered variant Cas12a endonuclease of the present disclosure and (b) a guide RNA and incubating the cell to produce a double strand break in the target nucleic acid.

In other embodiments, a method for introducing a double strand break in a target nucleic acid comprises introducing into a cell comprising a target nucleic acid (a) an engineered variant Cas12a endonuclease of the present disclosure and (b) a guide RNA and incubating the cell to produce a double strand break in the target nucleic acid.

In some embodiments, the off-target single strand nucleic acid cleavage in the cell is reduced relative to off-target single strand nucleic acid cleavage in a control cell comprising a wild-type Cas12a endonuclease and a guide RNA.

In some embodiments, a method for introducing a single strand break in a target nucleic acid, comprises introducing into a cell comprising a target nucleic acid (a) an engineered variant Cas12a endonuclease of the present disclosure (b) a guide RNA, and incubating the cell to produce a single strand break in the target nucleic acid.

Some aspects relate to an engineered polypeptide comprising the amino acid sequence of any one of SEQ ID NOs: 48-119 and 367-387 or a variant thereof having at least 80%, at least 85%, at least 90%, at least 95%, at least 96%, at least 97%, at least 98%, or at least 99% identity to the amino acid sequence of any one of SEQ ID NOs: 48-119 and 367-387, optionally wherein the engineered polypeptide is an endonuclease that exhibits hyperactive, hypoactivity, and/or low ssDNase activity (e.g., low indiscriminate ssDNase activity) relative to a naturally-occurring Cas12a endonuclease (e.g., SEQ ID NO: 1).

Some aspects relate to a fusion protein comprising an engineered variant Cas12a endonuclease of any one of the preceding aspects or embodiments and a base editing enzyme.

Some aspects relate to an engineered polypeptide comprising the amino acid sequence of any one of SEQ ID NOs: 163-185 and 388-408 or a variant thereof having at least 80%, at least 85%, at least 90%, at least 95%, at least 96%, at least 97%, at least 98%, or at least 99% identity to the amino acid sequence of any one of SEQ ID NOs: 163-185 and 388-408.

Further aspects relate to fusion protein comprising an engineered variant Cas12a endonuclease an amino acid sequence having at least 80%, at least 85%, at least 90%, at least 95%, at least 96%, at least 97%, at least 98%, at least 99%, or 100% identity to the amino acid sequence of any one of SEQ ID NOs: 367-387.

In some embodiments, the base editing enzyme is capable of converting a purine into a different purine or a pyrimidine into a different pyrimidine.

In some embodiments, the base editing enzyme comprises a deaminase, a guanine oxidase, or a guanine methyltransferase.

In some embodiments, the deaminase is a cytidine deaminase or an adenosine deaminase.

In some embodiments, the deaminase comprises a rAPOBEC1 polypeptide, an evoAPOBEC1 polypeptide, a hAPOBEC3A polypeptide, an evoCDA polypeptide, an evoFERNY polypeptide, or a TadA polypeptide.

In some embodiments, fusion protein further comprises a uracil glycosylase inhibitor (UGI).

In some embodiments, fusion protein further comprises one or more nuclear localization signal (NLS), optionally selected from an SV40 NLS, a nucleoprotein (NP) NLS, and a bipartite (BP) NLS.

In some embodiments, fusion protein further comprises a uracil DNA glycosylase (UNG), optionally a human UNG (hUNG) or an Escherichia coli UNG (eUNG).

In some embodiments, fusion protein further comprises a N-methyl purine glycosylase (MPG), optionally wherein the MPG is positioned at or near the N-terminal or C-terminal ends of the fusion protein.

In some embodiments, fusion protein further comprises one or more linker.

In some embodiments, the linker comprises the sequence of SGSETPGTSESATPES (SEQ ID NO: 203).

In some embodiments, the linker comprises the sequence of SGGSSGGSSGSETPGTSESATPESSGGSSGGS (SEQ ID NO: 204)

In some embodiments, fusion protein further comprises a DNA binding domain (DBD).

In some embodiments, the DBD is a Rad51 DBD.

Some aspects relate to a polynucleotide encoding the fusion protein of any one of the preceding aspects or embodiments.

Other aspects relate to a cell comprising: a target nucleic acid comprising a target strand and a non-target strand; a guide RNA (gRNA) or a nucleic acid encoding a gRNA that binds to the target strand; and the fusion protein of any one of the preceding aspects or embodiments or the polynucleotide of any one of the preceding aspects or embodiments.

In some embodiments, the cell is a human cell.

In some embodiments, the human cell is from a human subject, wherein the human subject has a disease, disorder or condition associated with the target nucleic acid.

In some embodiments, the cell is a plant cell.

Some aspects relate to a method comprising introducing into a cell (a) the fusion protein of any one of the preceding aspects or embodiments or the polynucleotide of any one of the preceding aspects or embodiments and optionally (b) a guide RNA or a polynucleotide encoding a guide RNA.

Some aspects relate to a method of gene editing comprising (i) contacting a target nucleic acid sequence with the fusion protein of any one of the preceding aspects or embodiments and a guide RNA, wherein the target nucleic acid comprises a target nucleobase; and (ii) modifying the target nucleobase.

In some embodiments, the target nucleic acid is a target double-stranded DNA nucleic acid.

In some embodiments, the guide RNA directs the fusion protein to bind to a specific segment of the target nucleic acid and in proximity to the target nucleobase.

In some embodiments, the fusion protein cleaves the target nucleic acid.

In some embodiments, the fusion protein comprises a cytidine deaminase and the target nucleobase is a cytidine. In some embodiments, the fusion protein comprises a guanine oxidase and the target nucleobase is a guanosine. In some embodiments, the fusion protein comprises a guanine methyltransferase and the target nucleobase is a guanosine.

In some embodiments, the method is performed in a cell, optionally a human cell or a plant cell.

In some embodiments, the method is performed in vitro or ex vivo. In other embodiments, the method is performed in vivo.

In some embodiments, the target nucleic acid is a gene comprising a nucleobase mutation relative to a wild-type gene.

In some embodiments, the gene comprising a nucleobase mutation is associated with a disease or disorder.

### BRIEF DESCRIPTION OF THE DRAWINGS

FIGS. 1A-1X show an alignment of various wild-type Cas12a endonuclease amino acid sequences using the Clustal Omega online multiple sequence alignment program (top to bottom SEQ ID NO: 29, 39, 12, 36, 8, 2, 43, 18, 37, 6, 46, 14, 28, 38, 45, 20, 13, 5, 21, 7, 42, 24, 40, 16, 35, 19, 41, 3, 10, 30, 1, 44, 22, 11, 27, 31, 4, 25, 26, 34, 32-33, 9, 17, and 23.

FIGS. 2A-2D show experimental data for various examples of the hyperactive Cas12a endonuclease variants of the present disclosure.

FIGS. 3A-3E show experimental data for various examples of the hypoactive Cas12a endonuclease variants of the present disclosure.

FIGS. 4A-4C show experimental data for various examples of the hypoactive Cas12a endonuclease variants of the present disclosure.

FIG. 5 shows experimental data for various examples of the Cas12a endonuclease variants of the present disclosure having low indiscriminate ssDNase activity.

FIGS. 6A-6C provide graphs of data comparing percent (%) of total reads having a C-to-T nucleotide edit at genomic positions corresponding to positions C8, C9, C10, C11, and C13 of the guide RNA (gRNA) using the LbBEv2 base editor in U2OS cells.

FIGS. 7A-7D provide graphs of data comparing percent (%) of total reads having a C-to-T nucleotide edit at genomic positions corresponding to positions C8 and C10 of the gRNA using the LbBEv2, LbBEv3, LbBEv4, or LbBEv5 base editor in U20S cells.

FIGS. 8A-8D provide graphs of data comparing percent (%) of total reads having a C-to-T nucleotide edit at genomic positions corresponding to positions C9, C10, and C15 of the gRNA using the LbBEv2, LbBEv3, LbBEv4, or LbBEv5 base editor in U2OS cells.

FIGS. 9A-9F provide data showing increased efficiency and specificity of base editing with LbBEv5 C-to-T base editor containing TBN04 (LbCas12a) as compared to LbBEv5 base editor containing inactive LbCas12a in U2OS cells (top to bottom SEQ ID NOs: 214-263).

FIGS. 10A-10F provide data showing increased efficiency and specificity of base editing with the LbBEv5 C-to-T base editor (top to bottom SEQ ID NOs: 264-315).

FIGS. 11A-11F provide data showing increased efficiency and specificity of base editing with the LbABE8e A-to-G base editor (top to bottom SEQ ID NO: 316-365).

FIGS. 12A-12C provide data showing increased efficiency and specificity of base editing with A-to-G base editors comprising mutant Cas12a.

FIGS. 13A-13B provide data showing the ability of base editors comprising a N-methyl purine glycosylase (MPG) to perform A-to-C base editing (SEQ ID NO: 409 and 366).

### DETAILED DESCRIPTION

### I. Cas12a Endonuclease

Provided herein are variants of the Class II type V CRISPR-Cas12a endonuclease. An “endonuclease” is an enzyme capable of cleaving the phosphodiester bond within a polynucleotide chain. Some endonucleases are specific (i.e., they recognize a given nucleotide sequence which directs the site of cleavage), while some are non-specific. The present disclosure provides specific variant Cas12a endonucleases. An endonuclease may cleave both strands of a double strand polynucleotide, or an endonuclease may demonstrate a preference for cleave cleaving one strand over the other strand of a double strand polynucleotide.

The recently discovered clustered regularly interspaced short palindromic repeats (CRISPR)-Cpf1 system, now reclassified as Cas12a, is a DNA-editing platform analogous to the widely used CRISPR-Cas9 system. The Cas12a system exhibits several distinct features over the CRISPR-Cas9 system, such as increased specificity and a smaller gene size to encode the nuclease and the matching CRISPR guide RNA (crRNA), which could mitigate off-target and delivery problems, respectively, described for the Cas9 system. However, the Cas12a system exhibits reduced gene editing efficiency compared to Cas9. Many of the variant Cas12a endonucleases provided herein exhibit increased gene editing efficiency compared to the wild-type Cas12a systems characterized to date.

RNA sequencing of small RNA molecules extracted from Francisella novicida U112 culture containing Cas12a-based CRISPR loci showed that mature crRNAs for Cas12a are 42-44 nucleotides (nt) in length, with the first 19/20 nt corresponding to the repeat sequence and the remaining 23-25 nt to the spacer sequence. Cas12a processes its own pre-crRNA into mature crRNAs, without the requirement of a tracrRNA, making it a unique effector protein with both endoribonuclease and endonuclease activities. After the pre-crRNA has been transcribed during the expression stage, Cas12a cuts it 4 nt upstream of the hairpin structures formed by the CRISPR repeats, producing intermediate crRNA molecules that undergo further processing in vivo into mature crRNAs.

Type V (Cas12a) CRISPR-Cas systems possesses a characteristic Ruv-C like nuclease domain, which has been shown to be related to IS605 family transposon encoded TnpB proteins. Crystallographic and cryo-EM data reveal that Cas12a adopts a bilobed structure formed by the REC and Nuc lobes. The REC lobe is comprised of REC1 and REC2 domains, and the Nuc lobe is comprised of the RuvC, the PAM-interacting (PI) and the WED domains, and additionally, the bridge helix (BH). The RuvC endonuclease domain of this effector protein is made up of three discontinuous parts (RuvC I-III). The RNase site for processing its own crRNA is situated in the WED-III subdomain, and the DNase site is located in the interface between the RuvC and the Nuc domains. These structural studies have also shown that the only the 5′ repeat region of the crRNA is involved in the assembly of the binary complex. The 19/20 nt repeat region forms a pseudoknot structure through intramolecular base pairing. The crRNA is stabilized through interactions with the WED, RuvC and REC2 domains of the endonuclease, as well as two hydrated Mg2+ ions. This binary interference complex is then responsible for recognizing and degrading foreign DNA. See Paul, B. & Montoya, G. et al. Biomedical Journal 2020; 43 (1): 8-17.

Protospacer adjacent motif (PAM) recognition is a critical initial step in identifying a prospective DNA molecule for degradation because the PAM allows the CRISPR-Cas systems to distinguish their own genomic DNA from invading nucleic acids. Cas12a employs a multistep quality control mechanism to ensure the accurate and precise recognition of target spacer sequences. The WED II-III, REC1 and PAM-interacting domains are responsible for PAM recognition and for initiating the hybridization of the DNA target with the crRNA. After recognition of the dsDNA by WED and REC1 domains, the conserved loop-lysine helix-loop (LKL) region in the PI domain, containing three conserved lysines (K667, K671, K677 in FnCas12a), inserts the helix into the PAM duplex with assistance from two conserved prolines in the LKL region. Structural studies show the helix is inserted at an angle of 45° with respect to the dsDNA longitudinal axis, promoting the unwinding of the helical dsDNA. The critical positioning of the three conserved lysines on the dsDNA initiates the uncoupling of the Watson-Crick interaction between the base pairs of the dsDNA after the PAM. The target dsDNA unzipping allows the hybridization of the crRNA with the strand containing the PAM, the target strand, while the uncoupled DNA strand, non-target strand (NTS), is conducted towards the DNase site by the PAM-interacting domain. Cas12a has been shown to efficiently target spacer sequences following 5′T-rich PAM sequence. The PAM for LbCas12a and AsCas12a has a sequence of 5′-TTTN-3′ and for FnCas12a a sequence of 5′-TTN-3′ and is situated upstream of the 5′end of the non-target strand [26,31,34]. It has also been shown that in addition to the canonical 5′-TTTN-3′ PAM, Cas12a also exhibits relaxed PAM recognition for suboptimal C-containing PAM sequences by forming altered interactions with the targeted DNA duplex. See Paul, B. & Montoya, G. et al.

Exemplary, non-limiting, wild-type Cas12a protein sequences are provided in Table 1.

TABLE 1
Non-limiting Examples of Wild-type Cas12a Sequences

| | | SEQ ID |
|---|---|---|
| Name | Sequence | NO: |
| LbCas12a-ND2006 | MSKLEKFTNCYSLSKTLRFKAIPVGKTQENIDNKRLLVEDEKRAEDYKGVKKLLDRYYLSFIND | 1 |
| Lachnospiraceae | VLHSIKLKNLNNYISLFRKKTRTEKENKELENLEINLRKEIAKAFKGNEGYKSLFKKDIIETIL | |
| bacterium ND2006 | PEFLDDKDEIALVNSFNGFTTAFTGFFDNRENMFSEEAKSTSIAFRCINENLTRYISNMDIFEK | |
| | VDAIFDKHEVQEIKEKILNSDYDVEDFFEGEFFNFVLTQEGIDVYNAIIGGFVTESGEKIKGLN | |
| | EYINLYNQKTKQKLPKFKPLYKQVLSDRESLSFYGEGYTSDEEVLEVFRNTLNKNSEIFSSIKK | |
| | LEKLFKNFDEYSSAGIFVKNGPAISTISKDIFGEWNVIRDKWNAEYDDIHLKKKAVVTEKYEDD | |
| | RRKSFKKIGSFSLEQLQEYADADLSVVEKLKEIIIQKVDEIYKVYGSSEKLFDADFVLEKSLKK | |
| | NDAVVAIMKDLLDSVKSFENYIKAFFGEGKETNRDESFYGDFVLAYDILLKVDHIYDAIRNYVT | |
| | QKPYSKDKFKLYFQNPQFMGGWDKDKETDYRATILRYGSKYYLAIMDKKYAKCLQKIDKDDVNG | |
| | NYEKINYKLLPGPNKMLPKVFFSKKWMAYYNPSEDIQKIYKNGTFKKGDMFNLNDCHKLIDFFK | |
| | DSISRYPKWSNAYDFNFSETEKYKDIAGFYREVEEQGYKVSFESASKKEVDKLVEEGKLYMFQI | |
| | YNKDFSDKSHGTPNLHTMYFKLLFDENNHGQIRLSGGAELFMRRASLKKEELVVHPANSPIANK | |
| | NPDNPKKTTTLSYDVYKDKRFSEDQYELHIPIAINKCPKNIFKINTEVRVLLKHDDNPYVIGID | |
| | RGERNLLYIVVVDGKGNIVEQYSLNEIINNENGIRIKTDYHSLLDKKEKERFEARQNWTSIENI | |
| | KELKAGYISQVVHKICELVEKYDAVIALEDLNSGFKNSRVKVEKQVYQKFEKMLIDKLNYMVDK | |
| | KSNPCATGGALKGYQITNKFESFKSMSTQNGFIFYIPAWLISKIDPSTGFVNLLKTKYTSIADS | |
| | KKFISSFDRIMYVPEEDLFEFALDYKNFSRTDADYIKKWKLYSYGNRIRIFRNPKKNNVEDWEE | |
| | VCLTSAYKELFNKYGINYQQGDIRALLCEQSDKAFYSSFMALMSLMLQMRNSITGRTDVDFLIS | |
| | PVKNSDGIFYDSRNYEAQENAILPKNADANGAYNIARKVLWAIGQFKKAEDEKLDKVKIAISNK | |
| | EWLEYAQTSVKH | |
| AsCas12a | MTQFEGFTNLYQVSKTLRFELIPQGKTLKHIQEQGFIEEDKARNDHYKELKPIIDRIYKTYADQ | 2 |
| Acidaminococcus | CLQLVQLDWENLSAAIDSYRKEKTEETRNALIEEQATYRNAIHDYFIGRTDNLTDAINKRHAEI | |
| sp. BV3L6 | YKGLFKAELFNGKVLKQLGTVTTTEHENALLRSFDKFTTYFSGFYENRKNVFSAEDISTAIPHR | |
| | IVQDNFPKFKENCHIFTRLITAVPSLREHFENVKKAIGIFVSTSIEEVFSFPFYNQLLTQTQID | |
| | LYNQLLGGISREAGTEKIKGLNEVLNLAIQKNDETAHIIASLPHRFIPLFKQILSDRNTLSFIL | |
| | EEFKSDEEVIQSFCKYKTLLRNENVLETAEALFNELNSIDLTHIFISHKKLETISSALCDHWDT | |
| | LRNALYERRISELTGKITKSAKEKVQRSLKHEDINLQEIISAAGKELSEAFKQKTSEILSHAHA | |
| | ALDQPLPTTLKKQEEKEILKSQLDSLLGLYHLLDWFAVDESNEVDPEFSARLTGIKLEMEPSLS | |
| | FYNKARNYATKKPYSVEKFKLNFQMPTLASGWDVNKEKNNGAILFVKNGLYYLGIMPKQKGRYK | |
| | ALSFEPTEKTSEGFDKMYYDYFPDAAKMIPKCSTQLKAVTAHFQTHTTPILLSNNFIEPLEITK | |
| | EIYDLNNPEKEPKKFQTAYAKKTGDQKGYREALCKWIDFTRDFLSKYTKTTSIDLSSLRPSSQY | |
| | KDLGEYYAELNPLLYHISFQRIAEKEIMDAVETGKLYLFQIYNKDFAKGHHGKPNLHTLYWTGL | |
| | FSPENLAKTSIKLNGQAELFYRPKSRMKRMAHRLGEKMLNKKLKDQKTPIPDTLYQELYDYVNH | |
| | RLSHDLSDEARALLPNVITKEVSHEIIKDRRFTSDKFFFHVPITLNYQAANSPSKENQRVNAYL | |
| | KEHPETPIIGIDRGERNLIYITVIDSTGKILEQRSLNTIQQFDYQKKLDNREKERVAARQAWSV | |
| | VGTIKDLKQGYLSQVIHEIVDLMIHYQAVVVLENLNFGFKSKRTGIAEKAVYQQFEKMLIDKLN | |
| | CLVLKDYPAEKVGGVLNPYQLTDQFTSFAKMGTQSGFLFYVPAPYTSKIDPLTGFVDPFVWKTI | |
| | KNHESRKHFLEGFDFLHYDVKTGDFILHFKMNRNLSFQRGLPGFMPAWDIVFEKNETQFDAKGT | |
| | PFIAGKRIVPVIENHRFTGRYRDLYPANELIALLEEKGIVERDGSNILPKLLENDDSHAIDTMV | |
| | ALIRSVLQMRNSNAATGEDYINSPVRDLNGVCFDSRFQNPEWPMDADANGAYHIALKGQLLLNH | |
| | LKESKDLKLQNGISNQDWLAYIQELRN | |
| RbCas12a | MQERKKISHLTHRNSVKKTIRMQLNPVGKTMDYFQAKQILENDEKLKENYQKIKEIADRFYRNL | 3 |
| Ruminococcus | NEDVLSKTGLDKLKDYAEIYYHCNTDADRKRLNKCASELRKEIVKNFKNRDEYNKLFDKRMIEI | |
| bromii | VLPKHLKNEDEKEVVASFKNFTTYFTGFFTNRKNMYSDGEESTAIAYRCINENLPKHLDNVKAF | |
| | EKAISKLSKNAIDDLDATYSGLCGTNLYDVFTVDYFNFLLPQSGITEYNKIIGGYTTNDGTKVK | |
| | GINEYINLYNQQVSKRDKIPNLQILYKQILSESEKVSFIPPKFEDDNELLSAVSEFYANDETFD | |
| | GMPLKKAIDETKLLFGNLDNSSLNGIYIQNDRSVTNLSNSMFGSWSVIEDLWNKNYDSVNSNSR | |
| | IKDIQKREDKRKKAYKAEKKLSLSFLQVLISNSENDEIRKKSIVDYYKTSLMQLTDNLSDKYNE | |
| | AAPLLNENYSNEKGLKNDDKSISLIKNFLDAIKEIEKFIKPLSETNITGEKNDLFYSQFTPLLD | |
| | NISRIDILYDKVRNYVTQKPFSTDKIKLNFGNYQLLNGWDKDKEREYGAVLLCKDEKYYLAIID | |
| | KSNNRILENIDFQDCDESDCYEKIIYKLLPTPNKMLPKVFFAKKHKKLLSPSDEILKIYKNGTF | |
| | KKGDKFSLDDCHKLIDFYKESFKKYPKWLIYNFKFKKINGYNDIREFYNDVALQGYNISKMKIP | |
| | TSFIDKLVDEGKIYLFQLYNKDFSPHSKGTPNLHTLYFKMLFDERNLEDVVYRLNGEAEMFYRP | |
| | ASIKYDKPTHPKNTPIKNKNTLNDKRASTFPYDLIKDKRYTKWQFSLHFPITMNFKDPDKAMIN | |
| | DDVRNLLKSCNNNFIIGIDRGERNLLYVSVINSNGAIIYQHSLNIIGNKFKGKTYETNYREKLA | |
| | TREKDRTEQRRNWKAIESIKELKEGYISQAVHVICQLVVKYDAIIVMEKLTDGFKRGRTKFEKQ | |
| | VYQKFEKMLIDKLNYYVDKKLDPDEEGGLLHAYQLTNKLESFDKLGTQSGFIFYVRPDFTSKID | |
| | PVTGFVNLLYPRYEKIDKAKDMISRFDDIRYNAGEDFFEFDIDYDKFPKTASDYRKKWTICTNG | |
| | ERIEAFRNPANNNEWSYRTIILAEKFKELFDNNSINYRDSDDLKAEILSQTKGKFFEDFFKLLR | |
| | LTLQMRNSNPETGEDRILSPVKDKNGNFYDSSKYDEKSKLPCDADANGAYNIARKGLWIVEQFK | |
| | KADNVSTVEPVIHNDKWLKFVQENDMANN | |
| LiCas12a | MKATSIWDNFTRKYSVSKTLRFELRPVGKTEENIVKKEIIDAEWISGKNIPKGTDADRARDYKI | 4 |
| Leptospira | VKKLLNQLHILFINQALSSENVKEFEKEDKKSKTFVAWSDLLATHEDNWIQYTRDKSNSTVLKS | |
| ilyithenensis | LEKSKKDLYSKLGKLLNSKANAWKAEFISYHKIKSPDNIKIRLSASNVQILFGNTSDPIQLLKY | |
| | QIELDNIKFLKDDGSEYTTKELADLLSTFEKFGTYFSGFNQNRANVYDIDGEISTSIAYRLENQ | |
| | NIEFFFQNIKRWEQFTSSIGHKEAKENLKLVQWDIQSKLKELDMEIVQPRENLKFEKLLTPQSF | |
| | IYLLNQEGIDAFNTVLGGIPAEVKAEKKQGVNELINLTRQKLNEDKRKFPSLQIMYKQIMSERK | |
| | TNFIDQYEDDVEMLKEIQEFSNDWNEKKKRHSASSKEIKESAIAYIQREFHETFDSLEERATVK | |
| | EDFYLSEKSIQNLSIDIFGGYNTIHNLWYTEVEGMLKSGERPLTRVEKEKLKKQEYISFAQIER | |
| | LISKHSQQYLDSTPKEANDRSLFKEKWKKTFKNGFKVSEYTNLKLNELISEGETFQKIDQETGK | |
| | ETTIKIPGLFESYENAILVESIKNQSLGTNKKESVPSIKEYLDSCLRLSKFIESFLVNSKDLKE | |
| | DQSLDGCSDFQNTLTQWLNEEFDVFILYNKVRNHVTKKPGNTDKIKINFDNATLLDGWDVDKEA | |
| | ANFGFLLKKADNYYLGIADSSFNQDLKYFNEGERLDEIEKNRKNLEKEESKNISKIDQEKVKKY | |
| | KEVIDDLKAISNLNKGRYSKAFYKQSKFTTLIPKCTTQLNEVIEHFKKFDTDYRIENKKFAKPF | |
| | IITKEVFLLNNTVYDTATKKFTLKIGEDEDTKGLKKFQIGYYRATDDKKGYESALRNWITFCIE | |
| | FTKSYKSCLNYNYSSLKSVSEYKSLDEFYKDLNGIGYTIDFVDISEEYINKKINEGKLYLFQIY | |
| | NKDFSEKSKGKENLHTTYWKLLFDSKNLEDVVIKLNGQAEVFFRPASIHEKEKITHFKNQEIQN | |
| | KNPNAVKKTSKFEYDIIKDNRFTKNKFLFHCPITLNFKADGNPYVNNEVQENIAKNPNVNIIGI | |
| | DRGEKHLLYFTVINQQGQILDAGSLNSIKSEYKDKNQQSVSFETPYHKILDKKESERKEARESW | |
| | QEIENIKELKAGYLSHVVHQLSNLIVKYNAIVVLEDLNKGFKRGRFKVEKQVYQKFEKSLIEKL | |
| | NYLVFKDRKESNEPGHHLNAYQLTNKFLSFERLGKQSGVLFYATASYTSKVDPVTGFMQNIYDP | |
| | YHKEKTREFYKNFTKIVYNGNYFEFNYDLNSVKPDSEEKRYRTNWTVCSCVIRSEYDSNSKTQK | |
| | TYNVNDQLVKLFEDAKIKIENGNDLKSTILEQDDKFIRDLHFYFIAIQKMRVVDSKIEKGEDSN | |
| | DYIQSPVYPFYCSKEIQPNKKGFYELPSNGDSNGAYNIARKGIVILDKIRLRVQIEKLFEDGTK | |
| | IDWQKLPNLISKVKDKKLLMTVFEEWAELTHQGEVQQGDLLGKKMSKKGEQFAEFIKGLNVTKE | |
| | DWEIYTQNEKVVQKQIKTWKLFSNST | |
| FsCas12a-S85 | MNLNTYFSQFTGLYPVSKTLRFELKPMGKTLEKIKETGIIENDKKRHNDYFDAKKIIDKYHKYF | 5 |
| Fibrobacter | IDAALSKFPCIDWNPLKEAIERSLDRSDASKKKLEKTQTEFRKKIAKALTTHGHYKELTASTPK | |
| succinogenes subsp. | DLFLKVFPDHFGKQPAIDTFDGFSSYFTGFQENRQNIYSDEAISTAIPYRLVHDNFPKFLSNIE | |
| succinogenes S85 | VYNILKDNAPSVLSDAENELKDFLNGKPLANIFELNAYNDVLTQSGIDFFNQVIGGFSGEGGEK | |
| | KTRGINEFSNLYRQQHPEFAQKRLATKMIPLYKQILSDRETKSFILESYSTDSQVQESVKEFFE | |
| | SQILNCDIAGRKVNVLKELSSLIKRITEFDLGSIYVNQEELSSISLELFKSWNTINAILFKNAE | |
| | NRIGSAEKAANKKKIDAWMKSNEFSIATLNLAIAESDSEEISRVKIESYWNNFEAKVQSILCGD | |
| | NRRNLDEFISATFNENNALREDSKVIEKLKAFLDALIEIMHSIKPLISDAENRDLSFYNELMPL | |
| | YDQLSLVVPLYNKIRNYATQKLTESEKFKLNFDNPTLADGWDQNKEEANTAILLLKNGLYYLGI | |
| | MNAKNKPKIKDFKTSESEDCYDKMVYKLLPGPNKMLPKVFFSEKGLATFKPPKDILDGYNAGKH | |
| | KKGDLFDIGFCHQLIDFFKESIAKHPDWKKFDFKFSDTSSYEDISGFYKEVTDQGYKITFSKIP | |
| | TPQIDEWVNEGKLFLFQIYNKDFAPGAKGSPNLHTLYWKSVFSPENLKDVVVKLNGEAELFYRP | |
| | SSVKKPYSHKVGEKLVNRIGKDGLPLPESVFGELFRYFNGKLDGELSDEAKRYLDVAVVKDVKH | |
| | EIVKDRRYTQDKFEFHVPLTLNFKADSKNEYMNERVRHFLKDNPDVNIIGIDRGERHLLYMTLI | |
| | NQKGEILKQKSFNIVESVNYQAKLVQREKERDTARRSWSSVGKIKDLKEGELSQVIHEITTTMI | |
| | ENNAIVVLEDLNFGFKRGRFCVERQVYQKFEKMLIDKLNYLVFKNKPEGDVGGVLKGYQLAEKF | |
| | DSFQKLGKQSGFLFYIPAAYTSKIDPTTGFANLFNMTELTSAEKKKEFLSHFEDITYDGKNDRF | |
| | LFSFDYKKFKCFQTDYIKKWTVYSQGKRIVYDKESKSAKAISPVEIIKAALAKQNIALTDQLDV | |
| | LSAINSVEASRETASFFGDICYAFEKTLQMRNSIPNTDEDYLVSPVMNKKGEFYDSRSCGDSLP | |
| | KNADANGAYHIALKGLYLIKNVFDAGGKDLKISHEDWFKFAQSRNR | |
| CsCas12a-AM42-36 | MGKNQNFQEFIGVSPLQKTLRNELIPTETTKKNIAQLDLLTEDEVRAQNREKLKEMMDDYYRDV | 6 |
| Clostridium sp. | IDSTLRGELLIDWSYLFSCMRNHLSENSKESKRELERTQDSVRSQIHDKFAERADFKDMFGASI | |
| AM42-36 | ITKLLPTYIKQNSKYSERYDESVKIMKLYGKFTTSLTDYFETRKNIFSKEKISSAVGYRIVEEN | |
| | AEIFLQNQNAYDRICKIAGLDLHGLDNEITAYVDGKTLKEVCSDEGFAKVITQGGIDRYNEAIG | |
| | AVNQYMNLLCQKNKALKPGQFKMKRLHKQILCKGTTSFDIPKKFENDKQVYDAVNSFTEIVTKN | |
| | NDLKRLLNITQNANDYDMNKIYVVADAYSMISQFISKKWNLIEECLLDYYSDNLPGKGNAKENK | |
| | VKKAVKEETYRSVSQLNEVIEKYYVEKTGQSVWKVESYISSLAEMIKLELCHEIDNDEKHNLIE | |
| | DDEKISEIKELLDMYMDVFHIIKVFRVNEVLNFDETFYSEMDEIYQDMQEIVPLYNHVRNYVTQ | |
| | KPYKQEKYRLYFHTPTLANGWSKSKEYDNNAIILVREDKYYLGILNAKKKPSKEIMAGKEDCSE | |
| | HAYAKMNYYLLPGANKMLPKVFLSKKGIQDYHPSSYIVEGYNEKKHIKGSKNFDIRFCRDLIDY | |
| | FKECIKKHPDWNKFNFEFSATETYEDISVFYREVEKQGYRVEWTYINSEDIQKLEEDGQLFLFQ | |
| | IYNKDFAVGSTGKPNLHTLYLKNLFSEENLRDIVLKLNGEAEIFFRKSSVQKPVIHKCGSILVN | |
| | RTYEITESGTTRVQSIPESEYMELYRYFNSEKQIELSDEAKKYLDKVQCNKAKTDIVKDYRYTM | |
| | DKFFIHLPITINFKVDKGNNVNAIAQQYIAEQEDLHVIGIDRGERNLIYVSVIDMYGRILEQKS | |
| | FNLVEQVSSQGTKRYYDYKEKLQNREEERDKARKSWKTIGKIKELKEGYLSSVIHEIAQMVVKY | |
| | NAIIAMEDLNYGFKRGRFKVERQVYQKFETMLISKLNYLADKSQAVDEPGGILRGYQMTYVPDN | |
| | IKNVGRQCGIIFYVPAAYTSKIDPTTGFINAFKRDVVSTNDAKENFLMKFDSIQYDIEKGLEKF | |
| | SFDYKNFATHKLTLAKTKWDVYTNGTRIQNMKVEGHWLSMEVELTTKMKELLDDSHIPYEEGQN | |
| | ILDDLREMKDITTIVNGILEIFWLTVQLRNSRIDNPDYDRIISPVLNNDGEFFDSDEYNSYIDA | |
| | QKAPLPIDADANGAFCIALKGMYTANQIKENWVEGEKLPADCLKIEHASWLAFMQGERG | |
| SlCas12a | MSDRLDVLTNQYPLSKTLRFELKPVGATADWIRKHNVIRYHNGKLVGKDAIRFQNYKYLKKMLD | 7 |
| Saccharobesus | EMHRLFLQQALVLEPNSNQAQELTALLRAIENNYCNNNDLLAGDYPSLSTDKTIKISNGLSKLT | |
| litoralis | TDLFDKKFEDWAYQYKEDMPNFWRQDIAELEQKLQVSANAKDQKFYKGIIKKLKNKIQKSELKA | |
| | ETHKGLYSPTESLQLLEWLVRRGDIKLTYLEIGKENEKLNELVPLVELKDIHRNENNFATYLSG | |
| | FSKNRENVYSTKFDRRSGYKATSVIARTFEQNLMFCLGNIAKWHKVTEFINQANNYELLQEHGI | |
| | DWNKQIAALEHKLDVCLAEFFALNNFSQTLAQQGIEKYNQVLAGIAEIAGQPKTQGLNELINLA | |
| | RQKLSAKRSQLPTLQLLYKQILSKGDKPFIDDFKSDQELIAELNEFVSSQIHGEHGAIKLINHE | |
| | LESFINEARAAQQQIYVPKDKLTELSLLLTGSWQAINQWRYKLFDQKQLDKQQKQYSFSLAQVE | |
| | RWLATEVEQQNFYQTEKERQQHKDTQPANVTTSSDGHSILTAFEQQVQTLLINICVAAEKYRQL | |
| | SDNLTAIDKQRESESSKGFEQIAVIKTLLDACNELNHFLARFTVNKKDKLPEDRAEFWYEKLQA | |
| | YIDAFPIYELYNKVRNYLSKKPFSTEKVKINFDNSHFLSGWTADYERHSALLFKFNENYLLGVV | |
| | NENLSSEEEEKLKLVGGEEHAKRFIYDFQKIDNSNPPRVFIRSKGSSFAPAVEKYQLPIGDIID | |
| | IYDQGKFKTEHKKKNEAEFKDSLVRLIDYFKLGFSRHDSYKHYPFKWKASHQYSDIAEFYAHTA | |
| | SFCYTLKEENINFNVLRELSSAGKVYLFEIYNKDFSKNKRGQGRDNLHTSYWKLLFSAENLKDV | |
| | VLKLNGQAEIFYRPASLAETKAYTHKKGEVLKHKAYSKVWEALDSPIGTRLSWDDALKIPSITE | |
| | KTNHNNQRVVQYNGQEIGRKAEFAIIKNRRYSVDKFLFHCPITLNFKANGQDNINARVNQFLAN | |
| | NKKINIIGIDRGEKHLLYISVINQQGEVLHQESENTITNSYQTANGEKRQVVTDYHQKLDMSED | |
| | KRDKARKSWSTIENIKELKAGYLSHVVHRLAQLIIEFNAIVALEDLNHGFKRGRFKIEKQVYQK | |
| | FEKALIDKLSYLAFKDRTSCLETGHYLNAFQLTSKFKGFNNLGKQSGILFYVNADYTSTTDPLT | |
| | GYIKNVYKTYSSVKDSTEFWQRFNSIRYIASENRFEFSYDLADLKQKSLESKTKQTPLAKTQWT | |
| | VSSHVTRSYYNQQTKQHELFEVTARIQQLLSKAEISYQHQNDLIPALASCQSKALHKELIWLEN | |
| | SILTMRVTDSSKPSATSENDFILSPVAPYFDSRNLNKQLPENGDANGAYNIARKGIMLLERIGD | |
| | FVPEGNKKYPDLLIRNNDWQNFVQRPEMVNKQKKKLVKLKTEYSNGSLENDLAFK | |
| SdCas12a | MSSLTKFTNKYSKQLTIKNELIPVGKTLENIKENGLIDGDEQLNENYQKAKIIVDDFLRDFINK | 8 |
| Succinivibrio | ALNNTQIGNWRELADALNKEDEDNIEKLQDKIRGIIVSKFETFDLFSSYSIKKDEKIIDDDNDV | |
| dextrinosolvens | EEEELDLGKKTSSFKYIFKKNLFKLVLPSYLKTTNQDKLKIISSFDNFSTYFRGFFENRKNIFT | |
| | KKPISTSIAYRIVHDNFPKFLDNIRCFNVWQTECPQLIVKADNYLKSKNVIAKDKSLANYFTVG | |
| | AYDYFLSQNGIDFYNNIIGGLPAFAGHEKIQGLNEFINQECQKDSELKSKLKNRHAFKMAVLFK | |
| | QILSDREKSFVIDEFESDAQVIDAVKNFYAEQCKDNNVIFNLLNLIKNIAFLSDDELDGIFIEG | |
| | KYLSSVSQKLYSDWSKLRNDIEDSANSKQGNKELAKKIKTNKGDVEKAISKYEFSLSELNSIVH | |
| | DNTKFSDLLSCTLHKVASEKLVKVNEGDWPKHLKNNEEKQKIKEPLDALLEIYNTLLIFNCKSF | |
| | NKNGNFYVDYDRCINELSSVVYLYNKTRNYCTKKPYNTDKFKLNFNSPQLGEGFSKSKENDCLT | |
| | LLFKKDDNYYVGIIRKGAKINFDDTQAIADNTDNCIFKMNYFLLKDAKKFIPKCSIQLKEVKAH | |
| | FKKSEDDYILSDKEKFASPLVIKKSTFLLATAHVKGKKGNIKKFQKEYSKENPTEYRNSLNEWI | |
| | AFCKEFLKTYKAATIFDITTLKKAEEYADIVEFYKDVDNLCYKLEFCPIKTSFIENLIDNGDLY | |
| | LFRINNKDFSSKSTGTKNLHTLYLQAIFDERNLNNPTIMLNGGAELFYRKESIEQKNRITHKAG | |
| | SILVNKVCKDGTSLDDKIRNEIYQYENKFIDTLSDEAKKVLPNVIKKEATHDITKDKRFTSDKF | |
| | FFHCPLTINYKEGDTKQFNNEVLSFLRGNPDINIIGIDRGERNLIYVTVINQKGEILDSVSENT | |
| | VTNKSSKIEQTVDYEEKLAVREKERIEAKRSWDSISKIATLKEGYLSAIVHEICLLMIKHNAIV | |
| | VLENLNAGFKRIRGGLSEKSVYQKFEKMLINKLNYFVSKKESDWNKPSGLINGLQLSDQFESFE | |
| | KLGIQSGFIFYVPAAYTSKIDPTTGFANVLNLSKVRNVDAIKSFFSNFNEISYSKKEALFKFSF | |
| | DLDSLSKKGFSSFVKFSKSKWNVYTFGERIIKPKNKQGYREDKRINLTFEMKKLLNEYKVSFDL | |
| | ENNLIPNLTSANLKDTFWKELFFIFKTTLQLRNSVINGKEDVLISPVKNAKGEFFVSGTHNKTL | |
| | PQDCDANGAYHIALKGLMILERNNLVREEKDTKKIMAISNVDWFEYVQKRRGVL | |
| ScCas12a | MKEFTNQYSLTKTLRFELRPVGETAEKIEDFKSGGLKQTVEKDRERTEAYKQLKEVIDSYHRDF | 9 |
| Sedimentisphaera | IEQAFARQQTLSEEDFKQTYQLYKEAQKEKDGETLTKQYEHLRKKIAAMFSKATKEWAVMGENN | |
| cyanobacteriorum | ELIGKNKESKLYQWLEKNYRAGRIEKEEFDHNAGLIEYFEKFSTYFVGFDKNRANMYSKEAKAT | |
| | AISFRTINENMVKHFDNCQRLEKIKSKYPDLAEELKDFEEFFKPSYFINCMNQSGIDYYNISAI | |
| | GGKDEKDQKANMKINLFTQKNHLKGSDKPPFFAKLYKQILSDREKSVVIDEFEKDSELTEALKN | |
| | VFSKDGLINEEFFTKLKSALENFMLPEYQGQLYIRNAFLTKISANIWGSGSWGIIKDAVTQAAE | |
| | NNFTRKSDKEKYAKKDFYSIAELQQAIDEYIPTLENGVQNASLIEYFRKMNYKPRGSEEDAGLI | |
| | EEINNNLRQAGIVLNQAELGSGKQREENIEKIKNLLDSVLNLERFLKPLYLEKEKMRPKAANLN | |
| | KDFCESFDPLYEKLKTFFKLYNKVRNYATKKPYSKDKFKINFDTATLLYGWSLDKETANLSVIF | |
| | RKREKFYLGIINRYNSQIFNYKIAGSESEKGLERKRSLQQKVLAEEGEDYFEKMVYHLLLGASK | |
| | TIPKCSTQLKEVKAHFQKSSEDYIIQSKSFAKSLTLTKEIFDLNNLRYNTETGEISSELSDTYP | |
| | KKFQKGYLTQTGDVSGYKTALHKWIDFCKEFLRCYRNTEIFTFHFKDTKEYESLDEFLKEVDSS | |
| | GYEISFDKIKASYINEKVNAGELYLFEIYNKDFSEYSKGKPNLHTIYWKSLFETQNLLDKTAKL | |
| | NGKAEIFFRPRSIKHNDKIIHRAGETLKNKNPLNEKPSSRFDYDITKDRRFTKDKFFLHCPITL | |
| | NFKQDKPVRFNEQVNLYLKDNPDVNIIGIDRGERHLLYYTLINQNGEILQQGSLNRIGEEESRP | |
| | TDYHRLLDEREKQRQQARETWKAVEGIKDLKAGYLSRVVHKLAGLMVQNNAIVVLEDLNKGFKR | |
| | GRFAVEKQVYQNFEKALIQKLNYLVFKEVNSKDAPGHYLKAYQLTAPFISFEKLGTQSGFLFYV | |
| | RAWNTSKIDPATGFTDQIKPKYKNQKQAKDFMSSFDSVRYNRKENYFEFEADFEKLAQKPKGRT | |
| | RWTICSYGQERYSYSPKERKFVKHNVTQNLAELFNSEGISFDSGQCFKDEILKVEDASFFKSII | |
| | FNLRLLLKLRHTCKNAEIERDFIISPVKGNNSSFFDSRIAEQENITSIPQNADANGAYNIALKG | |
| | LMNLHNISKDGKAKLIKDEDWIEFVQKRKF | |
| RsCas12a | MSININKFSDECRKIDFFTDLYNIQKTLRFSLIPIGATADNFEFKGRLSKEKDLLDSAKRIKEY | 10 |
| Ruminococcus sp. | ISKYLADESDICLSQPVKLKHLDEYYELYITKDRDEQKFKSVEEKLRKELADLLKEILKRLNKK | |
| JE7A12 | ILSDYLPEYLEDDEKALEDIANLSSFSTYFNSYYDNCKNMYTDKEQSTAIPYRCINDNLPKFID | |
| | NMKAYEKALEELKPSDLEELRNNFKGVYDTTVDDMFTLDYFNCVLSQSGIDSYNAIIGNDKVKG | |
| | INEYINLHNQTAEQGHKVPNLKRLYKQIGSQKKTISFLPSKFESDNELLKAVYDFYNTGDAEKN | |
| | FTALKDTITEFEKIFDNLSEYNLDGVFVRNDISLTNLSQSMFNDWSVFRNLWNDQYDKVNNPEK | |
| | AKDIDKYNDKRHKVYKKSESFSINQLQELIATTLEEDINSKKITDYFSCDFHRVTTEVENKYQL | |
| | VKDLLSSDYPKNKNLKTSEEDVALIKDFLDSVKSLESFVKILTGTGKESGKDELFYGSFTKWFD | |
| | QLRYIDKLYDKVRNYITEKPYSLDKIKLSFDNPQFLGGWQHSKETDYSAQLFMKDGLYYLGVMD | |
| | KETKREFKTQYNTPENDSDTMVKIEYNQIPNPGRVIQNLMLVDGKIVKKNGRKNADGVNAVLEE | |
| | LKNQYLPENINRIRKTESYKTTSNNFNKDDLKAYLEYYIARTKEYYCKYNFVFKSADEYGSFNE | |
| | FVDDVNNQAYQITKVKVSEKQLLSLVEQGKLYLFKIYNKDFSEYSKGKKNLHTMYFQMLFDDRN | |
| | LENLVYKLQGGAEMFYRPASIKKDSEFKHDANVEIIKRTCEDKVNDKDNPTDDEKAKYYSKFDY | |
| | DIVKNKRFTKDQFSLHLTLAMNCNQPDHYWLNNDVRELLKKSNKNHIIGIDRGERNLIYVTIIN | |
| | SDGVIVDQINFNIIENSYNGKKYKTDYQKKLNQREEDRQKARKTWKTIETIKELKDGYISQVVH | |
| | QICKLIVQYDAIVVMENLNGGFKRGRTKVEKQVYQKFETMLINKLNYYVDKGTDYKECGGLLKA | |
| | YQLTNKFETFERIGKQSGIIFYVDPYLTSKIDPVTGFANLLYPKYETIPKTHNFISNIDDIRYN | |
| | QSEDYFEFDIDYDKFPQGSYNYRKKWTICSYGNRIKYYKDSRNKTASVVVDITEKFKETFTNAG | |
| | IDFVNDNIKEKLLLVNSKELLKSFMDTLKLTVQLRNSEINSDVDYIISPIKDRNGNFYYSENYK | |
| | KSNNEVPSQPQDGDANGAYNIARKGLMIINKLKKADDVTNNELLKISKKEWLEFAQKGDLGE | |
| PbCas12a | MKQFTNLYQLSKTLRFELKPIGKTLEHINANGFIDNDAHRAESYKKVKKLIDDYHKDYIENVLN | 11 |
| Prevotella brevis | NFKLNGEYLQAYFDLYSQDTKDKQFKDIQDKLRKSIASALKGDDRYKTIDKKELIRQDMKTFLK | |
| | KDTDKALLDEFYEFTTYFTGYHENRKNMYSDEAKSTAIAYRLIHDNLPKFIDNIAVFKKIANTS | |
| | VADNFSTIYKNFEEYLNVNSIDEIFSLDYYNIVLTQTQIEVYNSIIGGRTLEDDTKIQGINEFV | |
| | NLYNQQLANKKDRLPKLKPLFKQILSDRVQLSWLQEEFNTGADVLNAVKEYCTSYFDNVEESVK | |
| | VLLTGISDYDLSKIYITNDLALTDVSQRMFGEWSIIPNAIEQRLRSDNPKKTNEKEEKYSDRIS | |
| | KLKKLPKSYSLGYINECISELNGIDIADYYATLGAINTESKQEPSIPTSIQVHYNALKPILDTD | |
| | YPREKNLSQDKLTVMQLKDLLDDFKALQHFIKPLLGNGDEAEKDEKFYGELMQLWEVIDSITPL | |
| | YNKVRNYCTRKPFSTEKIKVNFENAQLLDGWDENKESTNASIILRKNGMYYLGIMKKEYRNILT | |
| | KPMPSDGDCYDKVVYKFFKDITTMVPKCTTQMKSVKEHFSNSNDDYTLFEKDKFIAPVVITKEI | |
| | FDLNNVLYNGVKKFQIGYLNNTGDSFGYNHAVEIWKSFCLKFLKAYKSTSIYDFSSIEKNIGCY | |
| | NDLNSFYGAVNLLLYNLTYRKVSVDYIHQLVDEDKMYLFMIYNKDFSTYSKGTPNMHTLYWKML | |
| | FDESNLNDVVYKLNGQAEVFYRKKSITYQHPTHPANKPIDNKNVNNPKKQSNFEYDLIKDKRYT | |
| | VDKFMFHVPITLNFKGMGNGDINMQVREYIKTTDDLHFIGIDRGERHLLYICVINGKGEIVEQY | |
| | SLNEIVNNYKGTEYKTDYHTLLSERDKKRKEERSSWQTIEGIKELKSGYLSQVIHKITQLMIKY | |
| | NAIVLLEDLNMGFKRGRQKVESSVYQQFEKALIDKLNYLVDKNKDANEIGGLLHAYQLTNDPKL | |
| | PNKNSKQSGFLFYVPAWNTSKIDPVTGFVNLLDTRYENVAKAQAFFKKFDSIRYNKEYDRFEFK | |
| | FDYSNFTAKAEDTRTQWTLCTYGTRIETFRNAEKNSNWDSREIDLTTEWKTLFTQHNIPLNANL | |
| | KEAILLQANKNFYTDILHLMKLTLQMRNSVTGTDIDYMVSPVANECGEFFDSRKVKEGLPVNAD | |
| | ANGAYNIARKGLWLAQQIKNANDLSDVKLAITNKEWLQFAQKKQYLKD | |
| HoCas12a | MSFERLTNIASISKTLRFRLKPVGKTLENLEKLGKLDKDFERNNFYPILKNIADDYYRQYIRNR | 12 |
| Helcococcus ovis | LTDLNLDWIKLYYAHELLNSTDKESKKNLTTIQSEYRKILLNILSGELDKNGEKFSKDIVKKNK | |
| | ELYGKLFKKEFILEILPKFVETTNIYNKEYFNGINLYNKFTTRLSNFWEARKNIFTDKDIATGI | |
| | PFRVVNENFVYFYKNIQVENKNIKYLEDKLDNLEKNLKSEGIMSIDKSIKDFFNPNGFNYVITQ | |
| | KGIDTYQAIRGGFTKENGEKVQGINEILNLTQQKLRRNPYTKNIKLGVLTKLRKQILEYSESTS | |
| | FLIDQIEDDNDLVDRINKFNVSFFESTEVSPSIFVQLENLYNSLRTANSEDIYIDARNTQKFSQ | |
| | MLFGQWDVIRRGYSLKITEGTKEEKKKYKKYIELDETSKAKGYLTLMEIQELVSSVEGYEEIDV | |
| | FNVLLEKFKINIIERLKVETPIYGSPMKLEAIKEYLEKHLEEYHKWKLLLINNDELDLDEAFYP | |
| | LLNEVISDYNIIQLYNLTRNYLTRKYSDKEKIKINFDFPTLADGWSESKISDNRSIILRKDGNY | |
| | YLGILEDNKLLDNNITNFLENCYEIMKYNLFPDAAKMIPKCSISKKEVKNHFENGEDKSIYLSN | |
| | QFVGRLEISKELYELQNNLVDGKKKYQIDYLRNTDDKVGYRNALNQWITFCKKELNKYQGTQDE | |
| | DYSKLKEAKYYDKLDQFYADVDSYGYSLDFDTINEDLVNKAVEDGKLLLFQIYNKDFSPESKGK | |
| | KNLHTLYWLSMFSDENLKARKLKLNGQAEIFYRKKLEKKPIIHKEGSILLNKIDKDGNTIPENI | |
| | YHECYRYLNKKIGRKDLSDKAITLFNKDVLNYKEARFDIIKDRRYSESQFFFHVPITFNWDLKS | |
| | NQNVNSIVQNMIKDREIKHIIGIDRGERHLLYYSVIDLEGNIVEQGSLNTLNQNRFDNSIVEVD | |
| | YQDKLRTREEDRDRARKNWTNINKIKELKDGYLSHVVHKLSKLIIDYEAIVILENLNQGFKRGR | |
| | FKVERQVYQKFELALMNKLSALSFKETYDEGKNLEPSGILNPIQACYPVDSYQDLQGQNGIVFY | |
| | LPAAYTSVIDPVTGFTNLFRLNSINTTKYEEFIKGFKNIYFDNEDLDFKFIFDYKNFEKFNFVS | |
| | FKNKKSKKWIVSTRGERISYNSKKKEYFYVKPTEILKNKLIELGINFEDKDKDIISLIDKINDS | |
| | KKIKLLKVVFDAFKYSVQLRNHDNIQDYIISPVADENGNYYNSNDVAIKNLKLPDNGDANGAFN | |
| | IARKGLLLIERISNSDDSKVDLKIKNEDWIDFIIS | |
| FsCas12a-UWH8 | MQAIHQFCGQKNGYSRSITLRNRLIPIGKTEENIQKFLESDKNRADKYPGAKQLIDNLHRDFIA | 13 |
| Fibrobacter sp. | EVLSTHSFDWQPLADSIEKFQKTKDARDKKNLQTQQTNLRKQIAKAFSSSEKGKKLFSKELFTE | |
| UWH8 | LLPEYIKGKVDEKANEEIVKEFDRFTTYFTGFYDNRKNMYSDEEQATAISFRLVNENFPKFLTN | |
| | AKLFQEIKGKYPEIINDALKSLKNEKIDSYFEVNGFNACLTQQGIDAYNQVLGGTAAEAGQEKS | |
| | KGLNECINLYKQQHSDVKIGKMSMLYKQILSDRDGSFIDAFEKDEDVFKAVQSYHEILISQLSE | |
| | IEKLFVDAEYDLDKIIVPVKKLTEYSQVCTGRWNVVEESIRQNFIAKHGEPKKKKDEDALDKEL | |
| | KKDQSLLELKNILASAPSMEGINIVDYLNNDNLVKQTFSSVELEVKNLEEGFVTLIQKISYKDG | |
| | SDLKQKDDDVEHIKIYLDCALNLYHYLELVDYRGEAEKDGDFYSTYEKVIERLSGILFLYNKVR | |
| | NYVTKKIDTEKKFKLNFDSPTLANGWDANKESANNAIILRKNGKYYLGIFNPNDKPKIDNEATC | |
| | DASDCYEKMVYKLLPGPNKMLPKVFFSKKGLETFNPPKEILEGYTKEQYKKGDTFDIIFCHKLI | |
| | DWFKDAINQHPDWKKFNFKFSKTESYADISEFYREITEQGYKISFTKIAESEIQNLVDCGKLFL | |
| | FQIYNKDYAENSCGSKNLHTLYWENLFSEENLKNTVLKLNGEAELFFRPQVIKEDKIIAHKKDS | |
| | YLVNRIGKDGKRIPESFYQEIYKKANGIIDKISDEAKEFEKNAVVKKATHDIVKDRRFTQNVYQ | |
| | FHCPITMNFKAAELTGKKFNERVQELLAKDPTVKVIGIDRGERHLLYLSLINQKGEIELQKTLN | |
| | LVELNRNGQTVQVDYQQKLTLKEKERDNARKNWKTINNIKEIKEGYLSAVVHEIAKMMVEHNAI | |
| | VVMEELNYGFKRGRFPVERQVYQKFELALIEKLNFLVFKNKNVSEAGGVLNAFQLTQKPDSLTD | |
| | FGKQNGWIFYIPAAYTSKIDPKTGFIDFFKLSKVATKNLTNMDAKKSFFKGSSSTCVGGFATLF | |
| | C | |
| CsCas12a-AF34- | MNYKTGLEDFIGKESLSKTLRNALIPTESTKIHMEEMGVIRDDELRAEKQQELKEIMDDYYRAF | 14 |
| 10BH Clostridium | IEEKLGQIQGIQWNSLFQKMEETMEDISVRKDLDKIQNEKRKEICCYFTSDKRFKDLFNAKLIT | |
| sp. AF34-10BH | DILPNFIKDNKEYTEEEKAEKEQTRVLFQRFATAFTNYFNQRRNNFSEDNISTAISFRIVNENS | |
| | EIHLQNMRAFQRIEQQYPEEVCGMEEEYKDMLQEWQMKHIYLVDFYDRVLTQPGIEYYNGICGK | |
| | INEHMNQFCQKNRINKNDFRMKKLHKQILCKKSSYYEIPFRFESDQEVYDALNEFIKTMKEKEI | |
| | ICRCVHLGQKCDDYDLGKIYISSNKYEQISNALYGSWDTIRKCIKEEYMDALPGKGEKKEEKAE | |
| | AAAKKEEYRSIADIDKIISLYGSEMDRTISAKKCITEICDMAGQISTDPLVCNSDIKLLQNKEK | |
| | TTEIKTILDSFLHVYQWGQTFIVSDIIEKDSYFYSELEDVLEDFEGITTLYNHVRSYVTQKPYS | |
| | TVKFKLHFGSPTLANGWSQSKEYDNNAILLMRDQKFYLGIFNVRNKPDKQIIKGHEKEEKGDYK | |
| | KMIYNLLPGPSKMLPKVFITSRSGQETYKPSKHILDGYNEKRHIKSSPKFDLGYCWDLIDYYKE | |
| | CIHKHPDWKNYDFHFSDTKDYEDISGFYREVEMQGYQIKWTYISADEIQKLDEKGQIFLFQIYN | |
| | KDFSVHSTGKDNLHTMYLKNLFSEENLKDIVLKLNGEAELFFRKASIKTPVVHKKGSVLVNRSY | |
| | TQTVGDKEIRVSIPEEYYTEIYNYLNHIGRGKLSTEAQRYLEERKIKSFTATKDIVKNYRYCCD | |
| | HYFLHLPITINFKAKSDIAVNERTLAYIAKKEDIHIIGIDRGERNLLYISVVDVHGNIREQRSF | |
| | NIVNGYDYQQKLKDREKSRDAARKNWEEIEKIKELKEGYLSMVIHYIAQLVVKYNAVVAMEDLN | |
| | YGFKTGRFKVERQVYQKFETMLIEKLHYLVFKDREVCEEGGVLRGYQLTYIPESLKKVGKQCGF | |
| | IFYVPAGYTSKIDPTTGFVNLFSFKNLTNRESRQDFVGKFDEIRYDRDKKMFEFSFDYNNYIKK | |
| | GTMLASTKWKVYTNGTRLKRIVVNGKYTSQSMEVELTDAMEKMLQRAGIEYHDGKDLKGQIVEK | |
| | GIEAEIIDIFRLTVQMRNSRSESEDREYDRLISPVLNDKGEFFDTATADKTLPQDADANGAYCI | |
| | ALKGLYEVKQIKENWKENEQFPRNKLVQDNKTWFDFMQKKRYL | |
| BaCas12a | MKNQINLFTNKFQLSKTLRFELKPQGKTLEHINSKGFIKNDEKRADSYKKMKATIDAFHRDFID | 15 |
| Brumimicrobium | LAMSNVKLTNLIDFEEIYNASNADKKDEKYKTKLSKIQEILRKEIAKGFKGEEVKDIFSKIDKK | |
| aurantiacum | DLITKLLEEWIIENKIEDIHFDPEFKNFTTYFSGFHQNRKNMYTDQEQSTAIAYRLIHENLPRF | |
| | IDNINIFQKINKVPDLEENLKKLYQEIEEYLGINAINEAFELEYFNETLSQKGIDIYNLILGGR | |
| | TAEEGKQKIQGLNEYINLYNQKQDKKNRVPKLKVLYKQILSDRTRTSFLPDTFEDDEESSASQK | |
| | VLDSINNFYLENLIDYLPNDKNSTINVLENLKLLLAELINFELDKVYIKNDTSITNISMKIFKN | |
| | YSVIREALNYFYENKIDPNFAHNENNANTDKKREKLEKEKAKITKQTYLSISFIEEAIHLYINE | |
| | NSNGNQYKNTYKPNCIANYFKDFFIAENKEGSNKEFDFISKIKARYNTIKGVLNTPFPDNKRLH | |
| | QEKNNIDNIKHFLDSIMEYLHFAKPLVLSGSFAFEKDEQFYTNFDELYNQLELIIPLYNKVRNY | |
| | ATQKPYSTEKFKLNFENSTLLNGWDVNKEEANTSILFIKNGFYYLGIMDKNHNKIFRNTPKSTN | |
| | TDIYKKVNYKLLPGASKMLPKVFFGKKNLDYYKPSKDILRIRNHGTHTKGGKPQSGFDKLDENL | |
| | NDCHKLIDFFKDSIQKHPDWSKFKFKFSDTQIYESIDQFYRELEPQAYSITYTNIDSSFIEEQI | |
| | NEGKLYLFQIYNKDFSKFSNGKPNLHTLYWKALFDEQNLKDVTYKLNGEAEIFYRKKSIQHDRQ | |
| | IIHKRNQPIINKNPNNEKKESIFKYNIIKDKRYTIDKFQFHVPITLNFKAKGTDYINYDVLDYL | |
| | KENPDVKIIGLDRGERHLIYLTLIDQKGKILEQISLNEIVNKKHNITTSYHNLLETKEIERDKA | |
| | RKNWGTVETIKELKEGYISQVVHKISKMMIEHNAIVVMEDLNMGFKRGRFKVEKQVYQKLEKML | |
| | IDKLNYLVLKDRQPNEPAGIYNALQLTNKFESFQKLGKQSGFLFYVPAWNTSKIDPTTGFVNLF | |
| | HVKYESVRKSQEFFNKFNSIKYNPKEAIFEFDFDYNEFTTRAEGTKTNWTVCTYGDRIKTFRNP | |
| | EKLNQWDNKEINITTAFEDFFGRHNITYGNGSDIKSQLISREEKDFFSELIHLFRLTLQMRNSK | |
| | TNSEIDYLISPVKNENGFFYDSRHADKNLPKDADANGAYHIAKKGLQWIKEIQSFEGNEWKKLK | |
| | LDKTNKGWLKFVQENQ | |
| LbCas12a-MA2020 | MYYESLTKQYPVSKTIRNELIPIGKTLDNIRQNNILESDVKRKQNYEHVKGILDEYHKQLINEA | 16 |
| Lachnospiraceae | LDNCTLPSLKIAAEIYLKNQKEVSDREDENKTQDLLRKEVVEKLKAHENFTKIGKKDILDLLEK | |
| bacterium MA2020 | LPSISEDDYNALESFRNFYTYFTSYNKVRENLYSDKEKSSTVAYRLINENFPKFLDNVKSYRFV | |
| | KTAGILADGLGEEEQDSLFIVETENKTLTQDGIDTYNSQVGKINSSINLYNQKNQKANGFRKIP | |
| | KMKMLYKQILSDREESFIDEFQSDEVLIDNVESYGSVLIESLKSSKVSAFFDALRESKGKNVYV | |
| | KNDLAKTAMSNIVFENWRTFDDLLNQEYDLANENKKKDDKYFEKRQKELKKNKSYSLEHLCNLS | |
| | EDSCNLIENYIHQISDDIENIIINNETFLRIVINEHDRSRKLAKNRKAVKAIKDFLDSIKVLER | |
| | ELKLINSSGQELEKDLIVYSAHEELLVELKQVDSLYNMTRNYLTKKPFSTEKVKLNFNRSTLLN | |
| | GWDRNKETDNLGVLLLKDGKYYLGIMNTSANKAFVNPPVAKTEKVFKKVDYKLLPVPNQMLPKV | |
| | FFAKSNIDFYNPSSEIYSNYKKGTHKKGNMFSLEDCHNLIDFFKESISKHEDWSKFGFKFSDTA | |
| | SYNDISEFYREVEKQGYKLTYTDIDETYINDLIERNELYLFQIYNKDFSMYSKGKLNLHTLYFM | |
| | MLFDQRNIDDVVYKLNGEAEVFYRPASISEDELIIHKAGEEIKNKNPNRARTKETSTFSYDIVK | |
| | DKRYSKDKFTLHIPITMNFGVDEVKRFNDAVNSAIRIDENVNVIGIDRGERNLLYVVVIDSKGN | |
| | ILEQISLNSIINKEYDIETDYHALLDEREGGRDKARKDWNTVENIRDLKAGYLSQVVNVVAKLV | |
| | LKYNAIICLEDLNFGFKRGRQKVEKQVYQKFEKMLIDKLNYLVIDKSREQTSPKELGGALNALQ | |
| | LTSKFKSFKELGKQSGVIYYVPAYLTSKIDPTTGFANLFYMKCENVEKSKRFFDGEDFIRFNAL | |
| | ENVFEFGFDYRSFTQRACGINSKWTVCINGERIIKYRNPDKNNMFDEKVVVVTDEMKNIFEQYK | |
| | IPYEDGRNVKDMIISNEEAEFYRRLYRLLQQTLQMRNSTSDGTRDYIISPVKNKREAYENSELS | |
| | DGSVPKDADANGAYNIARKGLWVLEQIRQKSEGEKINLAMTNAEWLEYAQTHLL | |
| TsCas12a | MTKTFDSEFFNLYSLQKTVRFELKPVGETASFVEDFKNEGLKRVVSEDERRAVDYQKVKEIIDD | 17 |
| Thiomicrospira sp. | YHRDFIEESLNYFPEQVSKDALEQAFHLYQKLKAAKVEEREKALKEWEALQKKLREKVVKCFSD | |
| XS5 | SNKARFSRIDKKELIKEDLINWLVAQNREDDIPTVETFNNFTTYFTGFHENRKNIYSKDDHATA | |
| | ISFRLIHENLPKFFDNVISFNKLKEGFPELKFDKVKEDLEVDYDLKHAFEIEYFVNFVTQAGID | |
| | QYNYLLGGKTLEDGTKKQGMNEQINLFKQQQTRDKARQIPKLIPLFKQILSERTESQSFIPKQF | |
| | ESDQELFDSLQKLHNNCQDKFTVLQQAILGLAEADLKKVFIKTSDLNALSNTIFGNYSVFSDAL | |
| | NLYKESLKTKKAQEAFEKLPAHSIHDLIQYLEQFNSSLDAEKQQSTDTVLNYFIKTDELYSRFI | |
| | KSTSEAFTQVQPLFELEALSSKRRPPESEDEGAKGQEGFEQIKRIKAYLDTLMEAVHFAKPLYL | |
| | VKGRKMIEGLDKDQSFYEAFEMAYQELESLIIPIYNKARSYLSRKPFKADKFKINFDNNTLLSG | |
| | WDANKETANASILFKKDGLYYLGIMPKGKTFLFDYFVSSEDSEKLKQRRQKTAEEALAQDGESY | |
| | FEKIRYKLLPGASKMLPKVFFSNKNIGFYNPSDDILRIRNTASHTKNGTPQKGHSKVEFNLNDC | |
| | HKMIDFFKSSIQKHPEWGSFGFTFSDTSDFEDMSAFYREVENQGYVISFDKIKETYIQSQVEQG | |
| | NLYLFQIYNKDFSPYSKGKPNLHTLYWKALFEEANLNNVVAKLNGEAEIFFRRHSIKASDKVVH | |
| | PANQAIDNKNPHTEKTQSTFEYDLVKDKRYTQDKFFFHVPISLNFKAQGVSKFNDKVNGFLKGN | |
| | PDVNIIGIDRGERHLLYFTVVNQKGEILVQESLNTLMSDKGHVNDYQQKLDKKEQERDAARKSW | |
| | TTVENIKELKEGYLSHVVHKLAHLIIKYNAIVCLEDLNFGFKRGRFKVEKQVYQKFEKALIDKL | |
| | NYLVFKEKELGEVGHYLTAYQLTAPFESFKKLGKQSGILFYVPADYTSKIDPTTGFVNFLDLRY | |
| | QSVEKAKQLLSDFNAIRFNSVQNYFEFEIDYKKLTPKRKVGTQSKWVICTYGDVRYQNRRNQKG | |
| | HWETEEVNVTEKLKALFASDSKTTTVIDYANDDNLIDVILEQDKASFFKELLWLLKLTMTLRHS | |
| | KIKSEDDFILSPVKNEQGEFYDSRKAGEVWPKDADANGAYHIALKGLWNLQQINQWEKGKTLNL | |
| | AIKNQDWFSFIQEKPYQE | |
| SvCas12a Sneathia | MTEEDTKSFVDEILLTPESVIKTIDNFIDSIIMNDIEGLKEEFLKISLENFEGIYISNKKLNEI | 18 |
| vaginalis | SNRKFGDYNSINMMIKQSMNEKGILSKKEINELIPDLENINKPKVKSFNLSFIFENLTKEHKEL | |
| | IIDYIRENICNVIENVKITIEKYRNIDNKIEFKNNAEKVSKIKEMLESINELCKLIKEFNTDEI | |
| | EKNNEFYNILNKNFEIFESSYKVLNKVRNFVTKKEVIENKMKLNFSNYQLGNGWHKNKEKDCSI | |
| | ILFRKRNNERWIYYLGILKHGTKIKENDYLSSVDTGFYKMDYYAQNSLSKMIPKCSITVKNVKN | |
| | APEDESVILNDSKKENEPLEITPEIRKLYGNNEHIKGDKFKKESLVKWIDFCKEFLLKYKSFEK | |
| | AKKEILKLKESNLYENLEEFYSDAEEKAYFLEFINIDEDKIKKLVKEKNLYLFQIYNKDFSAYS | |
| | TGNKNLHTMYFEELFTDENLKKPVFKLNGNTEVFYRIASSKPKIVHNKGEKLVNKTYLDDGIIK | |
| | TIPDSVYEEISEKVKNNEDYSKLLEENNIKNLEIKVATHEIVKDKRYFENKFLFYLPITLNKKV | |
| | SNKNTNKNINKNVIDEIKDCNEYNVIGIDRGERNLISLCIINQNGEIILQKEMNIIQSSDKYNV | |
| | DYNEKLEIKSKERDNAKKNWSEIGKIKDLKSGYLSAVVHEIVKLAIEYNAVIILEDLNNGFKNS | |
| | RKKVDKQIYQKFERALIEKLQFLIFKNYDKNEKGGLRNAFQLTPELKNITKVASQQGIIIYTNP | |
| | AYTSKIDPTTGYANIIKKSNNNEESIVKAIDKISYDKEKDMFYFDINLSNSSFNLTVKNVLKKE | |
| | WRIYTNGERIIYKDRKYITLNITQEMKDILSKCGIDYLNIDNLKQDILKNKLHKKVYYIFELAN | |
| | KMRNENKDVDYIISPVLNKDGKFFMTQEINELTPKDADLNGAYNIALKGKLMIDNLNKKEKFVF | |
| | LSNEDWLNFIQGR | |
| BsCas12a-OAE603 | MNNNMFKDFINKYSVPKTLKFELIPIGNTSENIKKYKIIETDRELEKGYEKVKLLIDEYHRSFI | 19 |
| Bacillus sp. | SRVLNNIEFGESLLKYEEFYSHNTDLKREKFEIHKKEMRKKISKAFKDAGAAELFKNTLITKLL | |
| OAE603 | PGLYEGKDEVLNVLNLFKKFTTYFKNFHENRKNIYSSEEKSTAISYRIINENLPIFIHNIKTYE | |
| | RICSLIDFDTALEDSFLNQIKKELNCQFFSEFFNIHTFNRVLSQEGINSYNLLLGGKSEEDGTK | |
| | IKGVNELLNLFCQETKEKLPKFKFLKKQILSDMDSKSFVLDAFNSDSDVLEAISSYHEYLMENI | |
| | EGSEITLKEFIGQMKNENLDTIYIKDKQSLKSISQKVFGSWSTITEAIYSFEYDEKNGGKGSTN | |
| | SVKYNEKKKNEFNKYYEQMAKSNTKLKKIYSLSYINKCIKYFGKSEDICDYFIGMGQYGVKEEV | |
| | PGENLIEAITSNYSAIKFNFIDKILSENELLIEKVKRYLDSIKELQMFLKPLNKEGDKNPLFYG | |
| | EFDRFYGALESVTPLYNMVRNYVTKKPYSKDKLKLNFSNAQLLNGWDKDKESDYLSLLFKKGSK | |
| | YYLGVLNNKIPKVGKCFDCQFEVDSEDYYEKMEYKQLSNVVANIPRIAFSDSNKSLFSPSNEIL | |
| | KIQERGSYLKSSIDFDIKDLHQMIDFFKNGLKKKYSEYDFNFSNTSSYSNISDFYQEVIKATYK | |
| | VKFRKVPSKFIDDLVDVGKLFLFQLHNKDYSIKSKGKKNLHTIYWESLFSEANLKNPVHKLNGE | |
| | AEIFFRKSSIQRHITHPKGQLIESKREKGKFNKFSYDLIKDKRYTEDKFFLHVPITLNRSANDK | |
| | GTNFNTEVCNVLSKFPEPHIIGVDRGERHLIYLSVIDSRGKIIHQESINTITNSYIDGSGKEQV | |
| | TEINYHSKLDKSEEERSQSRKNWKKIENIKELKQGYLSHVVHRITSLMFKYNGIVILEDLNFGF | |
| | KRGRFHVEKQVYQKFEKALIDKLNLIVSKNVNENELGGIRKPYQLTSKFTSFKELGKQTGFLFY | |
| | VNPNYTSKLDPTTGFSNQLLIKYESINKTKEFLEKFDEIKENKEENYFEFHVDFSKFTQKKVGK | |
| | TKWVICTKGDRISNFNRKQVYLTNELKELFNKYEIDYKNDIQEQFRQLELSKAFYESFLGYLRL | |
| | TLQMRNNDPNKKDENGNEIDYIISPVKNDNNRFFDSRDVKNEHGLPVNGDANGAYNIALKGLML | |
| | LNEIKEATKEGRRPNLAIKNEDWFKFVQNKEYNG | |
| TpCas12a | MKISEEFCGQGNGYSISKTLRFELKPKGKTLENIEKEKLLESDFKKSQDYKDVKIILDNYHKYF | 20 |
| Treponema | IDDVLQKVNLDWTKLAASITDYNKNKEDDSSVIKEQDFLRKEIVKIISKDKRFACLTASTPKDL | |
| porcinum | FNSILLEWFEKSTEFSLNKKAVETFKRFSSYFKGFQENRKNMYKDEPIPTAVPYRIVNENFPKF | |
| | LQNAESFKEIQKKCPEVIELVEKELSAYLGNDKLSDIFCVKNFNRYLCQTGAENQRGIDYYNQI | |
| | IGGIVQKENDVKLRGINEFLNLFWQQHVDFAKDNRRIKFVPLYKQILSDRSSLSFKIQTLESDE | |
| | ELKEAVLSFAKKLDSKNKDGKNIFDLVMELTENINQYDLSQIYINQKDMNAVSKILTGDWAYLQ | |
| | KRMNIFAEETLTKSEQKRWKKELDDDTSKSKGIFSFEELNKVLEYSSENCSAVSIKIQEYFETT | |
| | KRWYFEKQTGIFTKGEEIIEPSISGLCGQIKSNFDEVNKVFGNVSSENTLRENPEEVEKIKNYL | |
| | DSVQNLLHRIKPLKVNGIGDTSFYSEYDEIYSVLYEVISLYNKTRNYISKKSGIPEKFKLNFDN | |
| | PTLADGWDQNKEQANTSVILIKDDEYFLGIMNANNKPKFLENYEGNTEKCYQKMIYKLLPGPNK | |
| | MLPKVFFSTKGIETFNPPKEILNGYNAGKHKKGDSFDLDFCHSLIDWFKDAINRHEDWKKFDFK | |
| | FSETSSYKDISEFYREISEQGYKLTFTAIPESVVEKMVTDGNLFLFQIYNKDFAKGASGKPNMH | |
| | TLYWKQLFSKENLSDTILKLNGEAEIFYREPGIKEPIVHKTGSKLVNKVTKDGVSVPAEIYNEI | |
| | YKVQNGMQTELSETAQVFVKEHEVSVKTASHDITKDKHFTEAKFLFHVPITINFKAQGNSLTMN | |
| | ERVRKFLKNNPEVNIIGLDRGERHLIYFSLINQKGEILKQFTFNEVERKQNDRIIKVDYHEKLD | |
| | NREKERDAARKNWTAIGKIAELKEGYLSAVIHELTKMMIQYNAVIVMEDLNFGFKRGRFHVEKQ | |
| | VYQKFERMLIDKLNYLVFKDKGFTEPGGVLNGYQLAGQFESFQKLGKQSGFLFYVPAGYTSKID | |
| | PKSGFADLFNLRDLTNVRRKREFFSKFDSIKYDSETMSFSFAFDYKNFDGKGKTEMAKTKWTVF | |
| | SKDKRIVYFPKNKSYSDVFPTDELKQTFEQAEIKIHDDENLLDVIMEIGADLKPDEKPNQNVAS | |
| | FWDSLLRNFKLILQMRNSNAQTGEDYIISPVKADDGTFFDSRNQLSLGKEAKLPIDADANGAYH | |
| | IALKGLELLRRFNETDEIKLKKADMKISNADWFKFVQEKQYLN | |
| SjCas12a | MANSLKDFTNIYQLSKTLRFELKPIGKTEEHINRKLIIMHDEKRGEDYKSVTKLIDDYHRKFIH | 21 |
| Synergistes jonesii | ETLDPAHFDWNPLAEALIQSGSKNNKALPAEQKEMREKIISMFTSQAVYKKLFKKELFSELLPE | |
| | MIKSELVSDLEKQAQLDAVKSFDKFSTYFTGFHENRKNIYSKKDTSTSIAFRIVHQNFPKFLAN | |
| | VRAYTLIKERAPEVIDKAQKELSGILGGKTLDDIFSIESFNNVLTQDKIDYYNQIIGGVSGKAG | |
| | DKKLRGVNEFSNLYRQQHPEVASLRIKMVPLYKQILSDRTTLSFVPEALKDDEQAINAVDGLRS | |
| | ELERNDIFNRIKRLFGKNNLYSLDKIWIKNSSISAFSNELFKNWSFIEDALKEFKENEFNGARS | |
| | AGKKAEKWLKSKYFSFADIDAAVKSYSEQVSADISSAPSASYFAKFTNLIETAAENGRKFSYFA | |
| | AESKAFRGDDGKTEIIKAYLDSLNDILHCLKPFETEDISDIDTEFYSAFAEIYDSVKDVIPVYN | |
| | AVRNYTTQKPFSTEKFKLNFENPALAKGWDKNKEQNNTAIILMKDGKYYLGVIDKNNKLRADDL | |
| | ADDGSAYGYMKMNYKFIPTPHMELPKVFLPKRAPKRYNPSREILLIKENKTFIKDKNFNRTDCH | |
| | KLIDFFKDSINKHKDWRTFGFDFSDTDSYEDISDFYMEVQDQGYKLTFTRLSAEKIDKWVEEGR | |
| | LFLFQIYNKDFADGAQGSPNLHTLYWKAIFSEENLKDVVLKLNGEAELFFRRKSIDKPAVHAKG | |
| | SMKVNRRDIDGNPIDEGTYVEICGYANGKRDMASLNAGARGLIESGLVRITEVKHELVKDKRYT | |
| | IDKYFFHVPFTINFKAQGQGNINSDVNLFLRNNKDVNIIGIDRGERNLVYVSLIDRDGHIKLQK | |
| | DFNIIGGMDYHAKLNQKEKERDTARKSWKTIGTIKELKEGYLSQVVHEIVRLAVDNNAVIVMED | |
| | LNIGFKRGRFKVEKQVYQKFEKMLIDKLNYLVFKDAGYDAPCGILKGLQLTEKFESFTKLGKQC | |
| | GIIFYIPAGYTSKIDPTTGFVNLFNINDVSSKEKQKDFIGKLDSIRFDAKRDMFTFEFDYDKFR | |
| | TYQTSYRKKWAVWINGKRIVREKDKDGKFRMNDRLLTEDMKNILNKYALAYKAGEDILPDVISR | |
| | DKSLASEIFYVFKNTLQMRNSKRDTGEDFIISPVLNAKGRFFDSRKTDAALPIDADANGAYHIA | |
| | LKGSLVLDAIDEKLKEDGRIDYKDMAVSNPKWFEFMQTRKFDF | |
| Sc2Cas12a | MLSNFTNQYQLSKTLRFELKPVGDTLKHIEKSGLIAQDEIRSQEYQEVKTIIDKYHKAFIDEAL | 22 |
| Sulfurimonas | QNVVLSNLEEYEALFFERNRDEKAFEKLQAVLRKEIVAHFKQHPQYKTLFKKELIKADLKNWQE | |
| crateris | LSDAEKELVSHFDNFTTYFTGFHENRANMYTDEAKHSSIAYRIIHENLPIFLINKKLFETIKQK | |
| | APHLAQETQDALLEYLSGAIVEDMFELSYFNHLLSQTHIDLYNQMIGGVKQDSLKIQGLNEKIN | |
| | LYRQANGLSKRELPNLKPLHKQILSDRETLSWLPESFESDEELMQGVQAYFESEVLAFECCDGK | |
| | VNLLEKLPELLHQTQDYDFSKVYFKNDLALTAASQAIFKDYRIIKEALWEVNKPKKSKDLVADE | |
| | EKFFNKKNSYFSIEQIDGALNSAQLSANMMHYFQSESTKVIEQIQLTYNDWKRNSSNKELLKAF | |
| | LDALLSYQRLLKPLNAPNDLEKDVAFYAYFDAYFTSLCGVVKLYDKVRNFMTKKPYSLEKFKLN | |
| | FENSTLLDGWDVNKESDNTAILFRKEGLYYLGIMNKKYNKVFRNISSSQDEGYQKIDYKLLPGA | |
| | NKMLPKVFFSDKNKEYFKPNAKLLERYKAGEHKKGDNFDLDFCHELIDFFKTSIEKHQDWKHFA | |
| | YQFSPTESYEDLSGFYREVEQQGYKISYKNIAASFIDTLVAEGKLYFFQIYNKDFSPYSKGTPN | |
| | MHTLYWRALFDEKNLADVIYKLNGQAEIFFRKKSIEYSQEKLQKGHHHEMLKDKFAYPIIKDRR | |
| | FAFDKFQFHVPITLNFKAEGNENITPKTFEYIRSNPDNIKVIGIDRGERHLLYLSLIDAEGKIV | |
| | EQFTLNQIINSYNGKDHVIDYHAKLDAKEKDRDKARKEWGTVENIKELKEGYLSHVIHKIATLI | |
| | IEHGAVVAMEDLNFGFKRGRFKVEKQVYQKFEKALIDKLNYLVDKKKEPHKLGGLLNALQLTSK | |
| | FQSFEKMGKQNGFLFYVPAWNTSKIDPVTGFVNLFDTRYASVEKSKAFFTKFQSICYNEAKDYF | |
| | ELVFDYNDFTEKAKETRSEWTLCTYGERIVSFRNAEKNHQWDSKTIHLTTEFKNLFGELHGNDV | |
| | KEYILEQNSVEFFKSLIYLLKITLQMRNSITGTDIDYLVSPVADEAGNFYDSRKADTSLPKDAD | |
| | ANGAYNIARKGLMLMHRIQNAEDLKKVNLAISNRDWLRNAQGLDK | |
| LsCas12a | MKDFTHQYSLSKTLRFELKPVGETAERIEDFKNQGLKSIVEEDRQRAEDYKKMKRILDDYHKEF | 23 |
| Limihaloglobus | IEEVLNDDIFTANEMESAFEVYRKYMASKNDDKLKKEITEIFTDLRKKIAKAFENKSKEYCLYK | |
| sulfuriphilus | GDFSKLINEKKTGKDKGPGKLWYWLKAKADAGVNEFGDGQTFEQAEEALAKFNNFSTYFTGENQ | |
| | NRDNIYTDAEQQTAISYRVINENMTRYFDNCIRYSSIENKYPELVKQLEPLSGKFAPGNYKDYL | |
| | SQTAIDIYNEAVGHKSDDINAKGINQFINEYRQRNSIKGRELPIMSVLYKQILSDINKDLIIDK | |
| | FENAGELLDAVKTLHRELTDKKILLKIKQTLNEFLTEDNSEDIYIKSGTDLTAVSNAIWGEWSV | |
| | IPKALEMYAENITDMNAKAREKWLKREAYHLKTVQEAIEAYLKDNEEFETRNISEYFTNFKSGE | |
| | NDLIQVVQSAYAKMESIFGIEDFHKDRRPVTESGEPGEGFRQVELVREYLDSLINVEHFIKPLH | |
| | MFRSGKPIELEDCNSNFYDPLNEAYKELDVVFGIYNKVRNYVTQKPYSKDKFKINFQNSTLLDG | |
| | WDVNKESANSSVLLLKNGKYYLGVMKQGASNILNYRPEPSDSKNKINAKKQLSEIALAGATDDY | |
| | YEKMIYKLLPDPAKMLPKVFFSAKNIEFYNPSQEIIYIRENGLFKKDAGDKESLKKWIGFMKTS | |
| | LLKHPEWGSYFNFEFEPAEDYQDISIFYKQVAEQGYSVTFDKIKTSYIEEKVASGELYLFEIYN | |
| | KDFSPHSKGRPNLHTMYWKSLFEKENLQNLVTKLNGEAEVFFRQHSIKRNEKVVHRANRPIQNK | |
| | NPLTEKKQSIFEYDLVKDRRFTKDKFFLHCPITLNFKEAGPGRENDKVNKYIAGNPDIRIIGID | |
| | RGERHLLYYSLIDQSGRIVEQGTLNQITSTLNSGGREIPKTTDYRGLLDTKEKERDKARKSWSM | |
| | IENIKELKSGYLSHIVHKLAKLMVKNNAVVVLEDLNFGFKRGRFKVEKQVYQKFEKALIEKLNY | |
| | LVFKDARPAEPGHYLNAYQLTAPLESFKKLGKQSGFIYYVPAWNTSKIDPVTGFVNQFYIEKNS | |
| | MQYLKNFFGKFDSIRFNPDKNYFEFGFDYKNFHNKAAKSKWTICTHGDKRSWYNRKQRKLEIHN | |
| | VTENLASLLSGKGINFADGGSIKDKILSVDDASFFKSLAFNFKLTAQLRHTFEDNGEEIDCIIS | |
| | PVAAADGTFFCSETAKKLNMELPHDADANGAYNIARKGLMVLRQIRESGKPKPISNADWLDFAQ | |
| | QNED | |
| PxCas12a | MIIGRDFNMYYQNLTKMYPISKTLRNELIPVGKTLENIRKNGILEADIQRKADYEHVKKLMDNY | 24 |
| Pseudobutyrivibrio | HKQLINEALQGVHLSDLSDAYDLYFNLSKEKNSVDAFSKCQDKLRKEIVSFLKNHENFPKIGNK | |
| xylanivorans strain | EIIKLIQSLNDNDADNNALDSFSNFYTYFSSYNEVRKNLYSDEEKSSTVAYRLINENLPKSLDN | |
| DSM 14809 | IKAYAIAKKAGVRAEGLSEEEQDCLFIIETFERTLTQDGIDNYNADIGKLNTAINLYNQQNKKQ | |
| | EGFRKVPQMKCLYKQILSDREEAFIDEFSDDEDLITNIESFAENMNVELNSEIITDFKNALVES | |
| | DGSLVYIKNDVSKTLFSNIVFGSWNAIDEKLSDEYDLANSKKKKDEKYYEKRQKELKKNKSYDL | |
| | ETIIGLFDDSIDVIGKYIEKLESDITAIAEAKNDFDEIVLRKHDKNKSLRKNTNAVEAIKSYLD | |
| | TVKDFERDIKLINGSGQEVEKNLVVYAEQENILAEIKNVDSLYNMSRNYLTQKPFSTEKFKLNF | |
| | ENPTLLNGWDRNKEKDYLGILFEKEGMYYLGIINNNHRKIFENEKLCTGKESCENKIVYKQISN | |
| | AAKYLSSKQINPQNPPKEIAEILLKRKADSSSLSRKETELFIDYLKDDFLVNYPMIINSDGENF | |
| | FNFHFKQAKDYGSLQEFFKEVEHQAYSLKTRPIDDSYIYRMIDEGKLYLFQIHNKDFSPYSKGN | |
| | LNLHTIYLQMLFDQRNLNNVVYKLNGEAEVFYRPASINDEEVIIHKAGEEIKNKNSKRAVDKPT | |
| | SKFGYDIIKDRRYSKDKFMLHIPVTMNFGVDETRRFNDVVNDALRNDEKVRVIGIDRGERNLLY | |
| | VVVVDTDGTILEQISLNSIINNEYSIETDYHKLLDEKEGDRDRARKNWTTIENIKELKEGYLSQ | |
| | VVNVIAKLVLKYNAIICLEDLNFGFKRGRQKVEKQVYQKFEKMLIDKLNYLVIDKSRKQEKPEE | |
| | FGGALNALQLTSKFTSFKDMGKQTGIIYYVPAYLTSKIDPTTGFANLFYVKYENVEKAKEFFSR | |
| | FDSISYNNESGYFEFAFDYKKFTDRACGARSQWTVCTYGERIIKYRNADKNNSFDDKTIVLSEE | |
| | FKELFSIYGISYEDGAELKNKIMSVDEADFFRCLTGLLQKTLQMRNSSNDGTRDYIISPIMNDR | |
| | GEFFNSEACDASKPKDADANGAFNIARKGLWVLEQIRNTPSGDKLNLAMSNAEWLEYAQRNQIA | |
| | S | |
| AsCas12a-RM50 | MVAFIDEFVGQYPVSKTLRFEARPVPETKKWLESDQCSVLENDQKRNEYYGVLKELLDDYYRAY | 25 |
| Anaerovibrio sp. | IEDALTSFTLDKALLENAYDLYCNRDTNAFSSCCEKLRKDLVKAFGNLKDYLLGSDQLKDLVKL | |
| RM50 | KAKVDAPAGKGKKKIEVDSRLINWLNNNAKYSAEDREKYIKAIESFEGFVTYLTNYKQARENMF | |
| | SSEDKSTAIAFRVIDQNMVTYFGNIRIYEKIKAKYPELYSALKGFEKFFSPTAYSEILSQSKID | |
| | EYNYQCIGRPIDDADFKGVNSLINEYRQKNGIKARELPVMSMLYKQILSDRDNSFMSEVINRNE | |
| | EAIECAKNGYKVSYALFNELLQLYKKIFTEDNYGNIYVKTQPLTELSQALFGDWSILRNALDNG | |
| | KYDKDIINLAELEKYFSEYCKVLDADDAAKIQDKFNLKDYFIQKNALDATLPDLDKITQYKPHL | |
| | DAMLQAIRKYKLFSMYNGRKKMDVPENGIDFSNEFNAIYDKLSEFSILYDRIRNFATKKPYSDE | |
| | KMKLSFNMPTMLAGWDYNNETANGCFLFIKDGKYFLGVADSKSKNIFDFKKNPHLLDKYSSKDI | |
| | YYKVKYKQVSGSAKMLPKVVFAGSNEKIFGHLISKRILEIREKKLYTAAAGDRKAVAEWIDEMK | |
| | SAIAIHPEWNEYFKFKFKNTAEYDNANKFYEDIDKQTYSLEKVEIPTEYIDEMVSQHKLYLFQL | |
| | YTKDFSDKKKKKGTDNLHTMYWHGVFSDENLKAVTEGTQPIIKLNGEAEMFMRNPSIEFQVTHE | |
| | HNKPIANKNPLNTKKESVFNYDLIKDKRYTERKFYFHCPITLNFRADKPIKYNEKINRFVENNP | |
| | DVCIIGIDRGERHLLYYTVINQTGDILEQGSLNKISGSYTNDKGEKVNKETDYHDLLDRKEKGK | |
| | HVAQQAWETIENIKELKAGYLSQVVYKLTQLMLQYNAVIVLENLNVGFKRGRTKVEKQVYQKFE | |
| | KAMIDKLNYLVFKDRGYEMNGSYAKGLQLTDKFESFDKIGKQTGCIYYVIPSYTSHIDPKTGFV | |
| | NLLNAKLRYENITKAQDTIRKFDSISYNAKADYFEFAFDYRSFGVDMARNEWVVCTCGDLRWEY | |
| | SAKTRETKAYSVTDRLKELFKAHGIDYVGGENLVSHITEVADKHELSTLLFYLRLVLKMRYTVS | |
| | GTENENDFILSPVEYAPGKFFDSREATSTEPMNADANGAYHIALKGLMTIRGIEDGKLHNYGKG | |
| | GENAAWFKFMQNQEYKNNG | |
| AsCas12a-YH12106 | MNYQLLFQKFVHLYPISKTLRFELIPQGATQKFITEKQVLLQDEVRARKYPEMKQAIDGYHKDF | 26 |
| Acinetobacter sp. | IQRALGNIDSQSFEQALQTFQELFLRSQAERSTEAYKKEFETTQTKLRELIVNSFEKGEFKQEY | |
| YH12106 | KSLFDKNLITNLLKPWVEKQSQTGDNNYTYHNDENKFTTYFLGFHDNRKNIYSKEPHKTALAYR | |
| | LIHENLPKFLENNKILRKIQNDHPALWEQLQALHHTMPQLFNGWDLSQLLQVSFFSNTLTQTGI | |
| | DQYNTIIGGISEGENRQKIQGINELINLYNQKQDKKNRVAKLKQLYKQILSDRSTLSFLPQQFA | |
| | DDAELYHAINMFYLDHLHYQSMVNGHSYTLLERVQLLINELANYDLSKVYLAPNQLSAVSHQMF | |
| | GDFGYISRALSYYYMQVIQPDYELLLASAKTTAKIEAIEKLKTAFLDAPHSLVVIQAAIDKYLQ | |
| | LQPSSKPHTQLTDFIISLLKQYETVADDQSIKIINIFSDIEGKYSCIKGLVNTESTSESKREIL | |
| | QNEKLATDIKAFMDAINNVIKLLKPFALNEKLAASVEKDARFYSDFEEIYQALLVFVPLYNKVR | |
| | NYITQKPYSTEKFKLNFNKPTLLSGWDANKEADNLSILLRKNGNYYLAIMDTAKGANKAFEPKA | |
| | LNQLKVDDTTDCYEKMVYKLLPGPNKMFPKVFFSESRKAQFNPPQHIIESYNKKEHISSEAHFD | |
| | LKKCHALIDWFKQCIELHEDWKHENFKFSPTSQYSNISDFYKEVSEQNYKVHFQDIPADYIEQL | |
| | VAEGKLYLFQIYNKDFSPHAKGKENLHTMYFKALFSEENLKQPVFKLSGEAEMFYRPASLQLEN | |
| | TTIHKAGEAMVAKNPLTPDATRTLAYDIIKDRRFTTDKYLLHIPISLNFHAQESMSIKKHNDLV | |
| | RQMIKHNHQDLHIIGIDRGEKHLLYVSVIDLKGNIVYQESLNSIKSEAQNFETPYHQLLQHREE | |
| | GRAQARTAWGKIENIKELKDGYLSQVVHRIQQLILKYNAIVMLEDLNFGFKRGRFKIEKQIYQK | |
| | FEKALIHKLNYVVDKSTQADELGGVRKAYQLTAPFESFEKLGKQSGVLFYVPAWNTSKIDPVTG | |
| | FVDLLKPKYENLDKAQAFFKTFDSIIFNAKKDYFEFKVNLNQFAGLKAQAARAEWTICSYGPER | |
| | HVYQKKNAQQGETVIVNVTEELKALFAKNNIEVAEGVELKEMICAQTQVDFFKRLIWLLQVLLA | |
| | LRYSSSKDKLDYILSPVANVLGEFFDSRHASTHLPQDSDANGAYHIALKGLWVIEQLKTAANTE | |
| | KVNLAISNDEWLRFAQEKLYLT | |
| BoCas12a | MRKFNEFVGLYPISKTLRFELKPIGKTLEHIQRNKLLEHDAVRADDYVKVKKIIDKYHKCLIDE | 27 |
| Bacteroidetes oral | ALSGFTFDTEADGRSNNSLSEYYLYYNLKKRNEQEQKTFKTIQNNLRKQIVNKLTQSEKYKRID | |
| taxon 274 strain | KKELITTDLPDFLTNESEKELVEKFKNFTTYFTEFHKNRKNMYSKEEKSTAIAFRLINENLPKF | |
| F0058 | VDNIAAFEKVVSSPLAEKINALYEDFKEYLNVEEISRVFRLDYYDELLTQKQIDLYNAIVGGRT | |
| | EEDNKIQIKGLNQYINEYNQQQTDRSNRLPKLKPLYKQILSDRESVSWLPPKFDSDKNLLIKIK | |
| | ECYDALSEKEKVFDKLESILKSLSTYDLSKIYISNDSQLSYISQKMFGRWDIISKAIREDCAKR | |
| | NPQKSRESLEKFAERIDKKLKTIDSISIGDVDECLAQLGETYVKRVEDYFVAMGESEIDDEQTD | |
| | TTSFKKNIEGAYESVKELLNNADNITDNNLMQDKGNVEKIKTLLDAIKDLQRFIKPLLGKGDEA | |
| | DKDGVFYGEFTSLWTKLDQVTPLYNMVRNYLTSKPYSTKKIKLNFENSTLMDGWDLNKEPDNTT | |
| | VIFCKDGLYYLGIMGKKYNRVFVDREDLPHDGECYDKMEYKLLPGANKMLPKVFFSETGIQRFL | |
| | PSEELLGKYERGTHKKGAGFDLGDCRALIDFFKKSIERHDDWKKFDFKFSDTSTYQDISEFYRE | |
| | VEQQGYKMSFRKVSVDYIKSLVEEGKLYLFQIYNKDFSAHSKGTPNMHTLYWKMLFDEENLKDV | |
| | VYKLNGEAEVFFRKSSITVQSPTHPANSPIKNKNKDNQKKESKFEYDLIKDRRYTVDKFLFHVP | |
| | ITMNFKSVGGSNINQLVKRHIRSATDLHIIGIDRGERHLLYLTVIDSRGNIKEQFSLNEIVNEY | |
| | NGNTYRTDYHELLDTREGERTEARRNWQTIQNIRELKEGYLSQVIHKISELAIKYNAVIVLEDL | |
| | NFGFMRSRQKVEKQVYQKFEKMLIDKLNYLVDKKKPVAETGGLLRAYQLTGEFESFKTLGKQSG | |
| | ILFYVPAWNTSKIDPVTGFVNLFDTHYENIEKAKVFFDKFKSIRYNSDKDWFEFVVDDYTRESP | |
| | KAEGTRRDWTICTQGKRIQICRNHQRNNEWEGQEIDLTKAFKEHFEAYGVDISKDLREQINTQN | |
| | KKEFFEELLRLLRLTLQMRNSMPSSDIDYLISPVANDTGCFFDSRKQAELKENAVLPMNADANG | |
| | AYNIARKGLLAIRKMKQEENDSAKISLAISNKEWLKFAQTKPYLED | |
| ErCas12a | MNNGTNNFQNFIGISSLQKTLRNALTPTETTQQFIVKNGIIKEDELRGENRQILKDIMDDYYRG | 28 |
| Eubacterium rectale | FISETLSSIDDIDWTSLFEKMEIQLKNGDNKDTLIKEQAEKRKAIYKKFADDDRFKNMFSAKLI | |
| strain | SDILPEFVIHNNNYSASEKEEKTQVIKLESRFATSFKDYFKNRANCESADDISSSSCHRIVNDN | |
| 2789STDY5834884 | AEIFFSNALVYRRIVKNLSNDDINKISGDMKDSLKKMSLEKIYSYEKYGEFITQEGISFYNDIC | |
| | GKVNSFMNLYCQKNKENKNLYKLRKLHKQILCIADTSYEVPYKFESDEEVYQSVNGELDNISSK | |
| | HIVERLRKIGDNYNGYNLDKIYIVSKFYESVSQKTYRDWETINTALEIHYNNILPGNGKSKADK | |
| | VKKAVKNDLQKSITEINELVSNYKLCPDDNIKAETYIHEISHILNNFEAQELKYNPEIHLVESE | |
| | LKASELKNVLDVIMNAFHWCSVFMTEELVDKDNNFYAELEEIYDEIYPVISLYNLVRNYVTQKP | |
| | YSTKKIKLNFGIPTLADGWSKSKEYSNNAIILMRDNLYYLGIFNAKNKPEKKIIEGNTSENKGD | |
| | YKKMIYNLLPGPNKMIPKVFLSSKTGVETYKPSAYILEGYKQNKHLKSSKDFDITFCRDLIDYF | |
| | KNCIAIHPEWKNFGFDFSDTSTYEDISGFYREVELQGYKIDWTYISEKDIDLLQEKGQLYLFQI | |
| | YNKDFSKKSTGNDNLHTMYLKNLFSEENLKDVVLKLNGEAEIFFRKSSIKNPIIHKKGSILVNR | |
| | TYEAEEKDQFGNIQIVRKTIPENIYQELYKYFNDKSDKELSDEAAKLKNAVGHHEAATNIVKDY | |
| | RYTYDKYFLHMPITINFKANKTSFINDRILQYIAKEKDLHVIGIDRGERNLIYVSVIDTCGNIV | |
| | EQKSFNIVNGYDYQIKLKQQEGARQIARKEWKEIGKIKEIKEGYLSLVIHEISKMVIKYNAIIA | |
| | MEDLSYGFKKGRFKVERQVYQKFETMLINKLNYLVFKDISITENGGLLKGYQLTYIPEKLKNVG | |
| | HQCGCIFYVPAAYTSKIDPTTGFVNIFKFKDLTVDAKREFIKKFDSIRYDSDKNLFCFTFDYNN | |
| | FITQNTVMSKSSWSVYTYGVRIKRRFVNGRFSNESDTIDITKDMEKTLEMTDINWRDGHDLRQD | |
| | IIDYEIVQHIFEIFKLTVQMRNSLSELEDRNYDRLISPVLNENNIFYDSAKAGDALPKDADANG | |
| | AYCIALKGLYEIKQITENWKEDGKFSRDKLKISNKDWFDFIQNKRYLTS | |
| CPbCas12a | MSNFFKNFTNLYELSKTLRFELKPVGDTLTNMKDHLEYDEKLQTFLKDQNIDDAYQALKPQFDE | 29 |
| Candidatus | IHEEFITDSLESKKAKEIDFSEYLDLFQEKKELNDSEKKLRNKIGETFNKAGEKWKKEKYPQYE | |
| Peregrinibacteria | WKKGSKIANGADILSCQDMLQFIKYKNPEDEKIKNYIDDTLKGFFTYFGGFNQNRANYYETKKE | |
| bacterium | ASTAVATRIVHENLPKFCDNVIQFKHIIKRKKDGTVEKTERKTEYLNAYQYLKNNNKITQIKDA | |
| GW2011 GWA2 33 | ETEKMIESTPIAEKIFDVYYFSSCLSQKQIEEYNRIIGHYNLLINLYNQAKRSEGKHLSANEKK | |
| 10 | YKDLPKFKTLYKQIGCGKKKDLFYTIKCDTEEEANKSRNEGKESHSVEEIINKAQEAINKYFKS | |
| | NNDCENINTVPDFINYILTKENYEGVYWSKAAMNTISDKYFANYHDLQDRLKEAKVFQKADKKS | |
| | EDDIKIPEAIELSGLFGVLDSLADWQTTLFKSSILSNEDKLKIITDSQTPSEALLKMIFNDIEK | |
| | NMESFLKETNDIITLKKYKGNKEGTEKIKQWFDYTLAINRMLKYFLVKENKIKGNSLDTNISEA | |
| | LKTLIYSDDAEWFKWYDALRNYLTQKPQDEAKENKLKLNFDNPSLAGGWDVNKECSNFCVILKD | |
| | KNEKKYLAIMKKGENTLFQKEWTEGRGKNLTKKSNPLFEINNCEILSKMEYDFWADVSKMIPKC | |
| | STQLKAVVNHFKQSDNEFIFPIGYKVTSGEKFREECKISKQDFELNNKVFNKNELSVTAMRYDL | |
| | SSTQEKQYIKAFQKEYWELLFKQEKRDTKLINNEIFNEWINFCNKKYSELLSWERKYKDALTNW | |
| | INFCKYFLSKYPKTTLFNYSFKESENYNSLDEFYRDVDICSYKLNINTTINKSILDRLVEEGKL | |
| | YLFEIKNQDSNDGKSIGHKNNLHTIYWNAIFENFDNRPKLNGEAEIFYRKAISKDKLGIVKGKK | |
| | TKNGTEIIKNYRFSKEKFILHVPITLNFCSNNEYVNDIVNTKFYNFSNLHFLGIDRGEKHLAYY | |
| | SLVNKNGEIVDQGTLNLPFTDKDGNQRSIKKEKYFYNKQEDKWEAKEVDCWNYNDLLDAMASNR | |
| | DMARKNWQRIGTIKEAKNGYVSLVIRKIADLAVNNERPAFIVLEDLNTGFKRSRQKIDKSVYQK | |
| | FELALAKKLNFLVDKNAKRDEIGSPTKALQLTPPVNNYGDIENKKQAGIMLYTRANYTSQTDPA | |
| | TGWRKTIYLKAGPEETTYKKDGKIKNKSVKDQIIETFTDIGFDGKDYYFEYDKGEFVDEKTGEI | |
| | KPKKWRLYSGENGKSLDRFRGEREKDKYEWKIDKIDIVKILDDLFVNEDKNISLLKQLKEGVEL | |
| | TRNNEHGTGESLRFAINLIQQIRNTGNNERDNDFILSPVRDENGKHFDSREYWDKETKGEKISM | |
| | PSSGDANGAFNIARKGIIMNAHILANSDSKDLSLFVSDEEWDLHLNNKTEWKKQLNIFSSRKAM | |
| | AKRKK | |
| LbCas12a-MC2017 | MGLYDGFVNRYSVSKTLRFELIPQGRTREYIETNGILSDDEERAKDYKTIKRLIDEYHKDYISR | 30 |
| Lachnospiraceae | CLKNVNISCLEEYYHLYNSSNRDKRHEELDALSDQMRGEIASFLTGNDEYKEQKSRDIIINERI | |
| bacterium MC2017 | INFASTDEELAAVKRFRKFTSYFTGFFTNRENMYSAEKKSTAIAHRIIDVNLPKYVDNIKAFNT | |
| | AIEAGVFDIAEFESNFKAITDEHEVSDLLDITKYSRFIRNEDIIIYNTLLGGISMKDEKIQGLN | |
| | ELINLHNQKHPGKKVPLLKVLYKQILGDSQTHSFVDDQFEDDQQVINAVKAVTDTFSETLLGSL | |
| | KIIINNIGHYDLDRIYIKAGQDITTLSKRALNDWHIITECLESEYDDKFPKNKKSDTYEEMRNR | |
| | YVKSFKSFSIGRLNSLVTTYTEQACFLENYLGSFGGDTDKNCLTDFTNSLMEVEHLLNSEYPVT | |
| | NRLITDYESVRILKRLLDSEMEVIHFLKPLLGNGNESDKDLVFYGEFEAEYEKLLPVIKVYNRV | |
| | RNYLTRKPFSTEKIKLNFNSPTLLCGWSQSKEKEYMGVILRKDGQYYLGIMTPSNKKIFSEAPK | |
| | PDEDCYEKMVLRYIPHPYQMLPKVFFSKSNIAFFNPSDEILRIKKQESFKKGKSFNRDDCHKFI | |
| | DFYKDSINRHEEWRKFNFKFSDTDSYEDISRFYKEVENQAFSMSFTKIPTVYIDSLVDEGKLYL | |
| | FKLHNKDFSEHSKGKPNLHTVYWNALFSEYNLQNTVYQLNGSAEIFFRKASIPENERVIHKKNV | |
| | PITRKVAELNGKKEVSVFPYDIIKNRRYTVDKFQFHVPLKMNFKADEKKRINDDVIEAIRSNKG | |
| | IHVIGIDRGERNLLYLSLINEEGRIIEQRSLNIIDSGEGHTQNYRDLLDSREKDREKARENWQE | |
| | IQEIKDLKTGYLSQAIHTITKWMKEYNAIIVLEDLNDRFTNGRKKVEKQVYQKFEKMLIDKLNY | |
| | YVDKDEEFDRMGGTHRALQLTEKFESFQKLGRQTGFIFYVPAWNTSKLDPTTGFVDLLYPKYKS | |
| | VDATKDFIKKFDFIRFNSEKNYFEFGLHYSNFTERAIGCRDEWILCSYGNRIVNFRNAAKNNSW | |
| | DYKEIDITKQLLDLFEKNGIDVKQENLIDSICEMKDKPFFKSLIANIKLILQIRNSASGTDIDY | |
| | MISPAMNDRGEFFDTRKGLQQLPLDADANGAYNIAKKGLWIVDQIRNTTGNNVKMAMSNREWMH | |
| | FAQESRLA | |
| Pb2Cas12a | MQINNLKIIYMKFTDFTGLYSLSKTLRFELKPIGKTLENIKKAGLLEQDQHRADSYKKVKKIID | 31 |
| Prevotella bryantii | EYHKAFIEKSLSNFELKYQSEDKLDSLEEYLMYYSMKRIEKTEKDKFAKIQDNLRKQIADHLKG | |
| B14 | DESYKTIFSKDLIRKNLPDFVKSDEERTLIKEFKDFTTYFKGFYENRENMYSAEDKSTAISHRI | |
| | IHENLPKFVDNINAFSKIILIPELREKLNQIYQDFEEYLNVESIDEIFHLDYFSMVMTQKQIEV | |
| | YNAIIGGKSTNDKKIQGLNEYINLYNQKHKDCKLPKLKLLFKQILSDRIAISWLPDNFKDDQEA | |
| | LDSIDTCYKNLLNDGNVLGEGNLKLLLENIDTYNLKGIFIRNDLQLTDISQKMYASWNVIQDAV | |
| | ILDLKKQVSRKKKESAEDYNDRLKKLYTSQESFSIQYLNDCLRAYGKTENIQDYFAKLGAVNNE | |
| | HEQTINLFAQVRNAYTSVQAILTTPYPENANLAQDKETVALIKNLLDSLKRLQRFIKPLLGKGD | |
| | ESDKDERFYGDFTPLWETLNQITPLYNMVRNYMTRKPYSQEKIKLNFENSTLLGGWDLNKEHDN | |
| | TAIILRKNGLYYLAIMKKSANKIFDKDKLDNSGDCYEKMVYKLLPGANKMLPKVFFSKSRIDEF | |
| | KPSENIIENYKKGTHKKGANFNLADCHNLIDFFKSSISKHEDWSKFNFHFSDTSSYEDLSDFYR | |
| | EVEQQGYSISFCDVSVEYINKMVEKGDLYLFQIYNKDFSEFSKGTPNMHTLYWNSLFSKENLNN | |
| | IIYKLNGQAEIFFRKKSLNYKRPTHPAHQAIKNKNKCNEKKESIFDYDLVKDKRYTVDKFQFHV | |
| | PITMNFKSTGNTNINQQVIDYLRTEDDTHIIGIDRGERHLLYLVVIDSHGKIVEQFTLNEIVNE | |
| | YGGNIYRTNYHDLLDTREQNREKARESWQTIENIKELKEGYISQVIHKITDLMQKYHAVVVLED | |
| | LNMGFMRGRQKVEKQVYQKFEEMLINKLNYLVNKKADQNSAGGLLHAYQLTSKFESFQKLGKQS | |
| | GFLFYIPAWNTSKIDPVTGFVNLFDTRYESIDKAKAFFGKFDSIRYNADKDWFEFAFDYNNFTT | |
| | KAEGTRTNWTICTYGSRIRTFRNQAKNSQWDNEEIDLTKAYKAFFAKHGINIYDNIKEAIAMET | |
| | EKSFFEDLLHLLKLTLQMRNSITGTTTDYLISPVHDSKGNFYDSRICDNSLPANADANGAYNIA | |
| | RKGLMLIQQIKDSTSSNRFKFSPITNKDWLIFAQEKPYLND | |
| Mb2Cas12a | MLFQDFTHLYPLSKTVRFELKPIGRTLEHIHAKNFLSQDETMADMYQKVKVILDDYHRDFIADM | 32 |
| Moraxella bovoculi | MGEVKLTKLAEFYDVYLKFRKNPKDDGLQKQLKDLQAVLRKESVKPIGSGGKYKTGYDRLFGAK | |
| AAX08 00205 | LFKDGKELGDLAKFVIAQEGESSPKLAHLAHFEKFSTYFTGFHDNRKNMYSDEDKHTAIAYRLI | |
| | HENLPRFIDNLQILTTIKQKHSALYDQIINELTASGLDVSLASHLDGYHKLLTQEGITAYNRII | |
| | GEVNGYTNKHNQICHKSERIAKLRPLHKQILSDGMGVSFLPSKFADDSEMCQAVNEFYRHYTDV | |
| | FAKVQSLFDGFDDHQKDGIYVEHKNLNELSKQAFGDFALLGRVLDGYYVDVVNPEFNERFAKAK | |
| | TDNAKAKLTKEKDKFIKGVHSLASLEQAIEHHTARHDDESVQAGKLGQYFKHGLAGVDNPIQKI | |
| | HNNHSTIKGFLERERPAGERALPKIKSGKNPEMTQLRQLKELLDNALNVAHFAKLLTTKTTLDN | |
| | QDGNFYGEFGVLYDELAKIPTLYNKVRDYLSQKPFSTEKYKLNFGNPTLLNGWDLNKEKDNFGV | |
| | ILQKDGCYYLALLDKAHKKVFDNAPNTGKNVYQKMVYKLLPGPNKMLPKVFFAKSNLDYYNPSA | |
| | ELLDKYAKGTHKKGDNFNLKDCHALIDFFKAGINKHPEWQHFGFKFSPTSSYRDLSDFYREVEP | |
| | QGYQVKFVDINADYIDELVEQGKLYLFQIYNKDFSPKAHGKPNLHTLYFKALFSEDNLADPIYK | |
| | LNGEAQIFYRKASLDMNETTIHRAGEVLENKNPDNPKKRQFVYDIIKDKRYTQDKFMLHVPITM | |
| | NFGVQGMTIKEFNKKVNQSIQQYDEVNVIGIDRGERHLLYLTVINSKGEILEQRSLNDITTASA | |
| | NGTQVTTPYHKILDKREIERLNARVGWGEIETIKELKSGYLSHVVHQINQLMLKYNAIVVLEDL | |
| | NFGFKRGRFKVEKQIYQNFENALIKKLNHLVLKDKADDEIGSYKNALQLTNNFTDLKSIGKQTG | |
| | FLFYVPAWNTSKIDPETGFVDLLKPRYENIAQSQAFFGKFDKICYNTDKGYFEFHIDYAKFTDK | |
| | AKNSRQKWAICSHGDKRYVYDKTANQNKGAAKGINVNDELKSLFARYHINDKQPNLVMDICQNN | |
| | DKEFHKSLMCLLKTLLALRYSNASSDEDFILSPVANDEGVFFNSALADDTQPQNADANGAYHIA | |
| | LKGLWLLNELKNSDDLNKVKLAIDNQTWLNFAQNR | |
| Mb3Cas12a | MLFQDFTHLYPLSKTVRFELKPIGKTLEHIHAKNFLNQDETMADMYQKVKAILDDYHRDFIADM | 33 |
| Moraxella bovoculi | MGEVKLTKLAEFYDVYLKFRKNPKDDGLQKQLKDLQAVLRKEIVKPIGNGGKYKAGYDRLFGAK | |
| AAX11 00205 | LFKDGKELGDLAKFVIAQEGESSPKLAHLAHFEKFSTYFTGFHDNRKNMYSDEDKHTAIAYRLI | |
| | HENLPRFIDNLQILATIKQKHSALYDQIINELTASGLDVSLASHLDGYHKLLTQEGITAYNTLL | |
| | GGISGEAGSRKIQGINELINSHHNQHCHKSERIAKLRPLHKQILSDGMGVSFLPSKFADDSEVC | |
| | QAVNEFYRHYADVFAKVQSLFDGFDDYQKDGIYVEYKNLNELSKQAFGDFALLGRVLDGYYVDV | |
| | VNPEFNERFAKAKTDNAKAKLTKEKDKFIKGVHSLASLEQAIEHYTARHDDESVQAGKLGQYFK | |
| | HGLAGVDNPIQKIHNNHSTIKGFLERERPAGERALPKIKSDKSPEIRQLKELLDNALNVAHFAK | |
| | LLTTKTTLHNQDGNFYGEFGALYDELAKIATLYNKVRDYLSQKPFSTEKYKLNFGNPTLLNGWD | |
| | LNKEKDNFGVILQKDGCYYLALLDKAHKKVFDNAPNTGKSVYQKMIYKLLPGPNKMLPKVFFAK | |
| | SNLDYYNPSAELLDKYAQGTHKKGDNFNLKDCHALIDFFKAGINKHPEWQHFGFKFSPTSSYQD | |
| | LSDFYREVEPQGYQVKFVDINADYINELVEQGQLYLFQIYNKDFSPKAHGKPNLHTLYFKALFS | |
| | EDNLVNPIYKLNGEAEIFYRKASLDMNETTIHRAGEVLENKNPDNPKKRQFVYDIIKDKRYTQD | |
| | KFMLHVPITMNFGVQGMTIKEFNKKVNQSIQQYDEVNVIGIDRGERHLLYLTVINSKGEILEQR | |
| | SLNDITTASANGTQMTTPYHKILDKREIERLNARVGWGEIETIKELKSGYLSHVVHQISQLMLK | |
| | YNAIVVLEDLNFGFKRGRFKVEKQIYQNFENALIKKLNHLVLKDKADDEIGSYKNALQLTNNFT | |
| | DLKSIGKQTGFLFYVPAWNTSKIDPETGFVDLLKPRYENIAQSQAFFGKFDKICYNADRGYFEF | |
| | HIDYAKFNDKAKNSRQIWKICSHGDKRYVYDKTANQNKGATIGVNVNDELKSLFTRYHINDKQP | |
| | NLVMDICQNNDKEFHKSLMYLLKTLLALRYSNASSDEDFILSPVANDEGVFFNSALADDTQPQN | |
| | ADANGAYHIALKGLWLLNELKNSDDLNKVKLAIDNQTWLNFAQNR | |
| MICas12a | MLFQDFTHLYPLSKTVRFELKPIGKTLEHIHAKNFLSQDETMADMYQKVKAILDDYHRDFITKM | 34 |
| Moraxella lacunata | MSEVTLTKLPEFYEVYLALRKNPKDDTLQKQLTEIQTALREEVVKPIDSGGKYKAGYERLFGAK | |
| | LFKDGKELGDLAKFVIAQEGESSPKLPQIAHFEKFSTYFTGFHDNRKNMYSSDDKHTAIAYRLI | |
| | HENLPRFIDNLQILVTIKQKHSVLYDQIVNELNANGLDVSLASHLDGYHKLLTQEGITAYNRII | |
| | GEVNSYTNKHNQICHKSERIAKLRPLHKQILSDGMGVSFLPSKFADDSEMCQAVNEFYRHYAHV | |
| | FAKVQSLFDRFDDYQKDGIYVEHKNLNELSKQAFGDFALLGRVLDGYYVDVVNPEFNDKFAKAK | |
| | TDNAKEKLTKEKDKFIKGVHSLASLEQAIEHYIAGHDDESVQAGKLGQYFKHGLAGVDNPIQKI | |
| | HNSHSTIKGFLERERPAGERTLPKIKSDKSLEMTQLRQLKELLDNALNVVHFAKLLTTKTTLDN | |
| | QDGNFYGEFGALYDELAKIATLYNKVRDYLSQKPFSTEKYKLNFGNPTLLNGWDLNKEKDNFGV | |
| | ILQKDGCYYLALLDKAHKKVFDNAPNTGKSVYQKMVYKLLPGSNKMLPKVFFAKSNLDYYNPSA | |
| | ELLDKYAQGTHKKGDNFNLKDCHALIDFFKASINKHPEWQHFGFEFSLTSSYQDLSDFYREVEP | |
| | QGYQVKFVDIDADYIDELVEQGQLYLFQIYNKDFSPKAHGKPNLHTLYFKALFSEDNLANPIYK | |
| | LNGEAEIFYRKASLDMNETTIHRAGEVLENKNPDNPKERQFVYDIIKDKRYTQDKFMLHVPITM | |
| | NFGVQGMTIKEFNKKVNQSIQQYDEVNVIGIDRGERHLLYLTVINSKGEILEQRSLNDIITTSA | |
| | NGTQMTTPYHKILDKREIERLNARVGWGEIETIKELKSGYLSHVVHQISQLMLKYNAIVVLEDL | |
| | NFGFKRGRFKVEKQIYQNFENALIKKLNHLVLKDKADNEIGSYKNALQLTNNFTDLKSIGKQTG | |
| | FLFYVPAWNTSKIDPVTGFVDLLKPRYENIAQSQAFFDKFDKICYNADKGYFEFHIDYAKFTDK | |
| | AKNSRQIWTICSHGDKRYVYDKTANQNKGATIGINVNDELKSLFARYRINDKQPNLVMDICQNN | |
| | DKEFHKSLTYLLKALLALRYSNASSDEDFILSPVANDKGVFFNSALADDTQPQNADANGAYHIA | |
| | LKGLWLLNELKNSDDLDKVKLAIDNQTWLNFAQNR | |
| BsCas12a | MYYQNLTKKYPVSKTIRNELIPIGKTLENIRKNNILESDVKRKQDYEHVKGIMDEYHKQLINEA | 35 |
| Butyrivibrio sp. | LDNYMLPSLNQAAEIYLKKHVDVEDREEFKKTQDLLRREVTGRLKEHENYTKIGKKDILDLLEK | |
| NC3005 | LPSISEEDYNALESFRNFYTYFTSYNKVRENLYSDEEKSSTVAYRLINENLPKFLDNIKSYAFV | |
| | KAAGVLADCIEEEEQDALFMVETFNMTLTQEGIDMYNYQIGKVNSAINLYNQKNHKVEEFKKIP | |
| | KMKVLYKQILSDREEVFIGEFKDDETLLSSIGAYGNVLMTYLKSEKINIFFDALRESEGKNVYV | |
| | KNDLSKTTMSNIVFGSWSAFDELLNQEYDLANENKKKDDKYFEKRQKELKKNKSYTLEQMSNLS | |
| | KEDISPIENYIERISEDIEKICIYNGEFEKIVVNEHDSSRKLSKNIKAVKVIKDYLDSIKELEH | |
| | DIKLINGSGQELEKNLVVYVGQEEALEQLRPVDSLYNLTRNYLTKKPFSTEKVKLNFNKSTLLN | |
| | GWDKNKETDNLGILFFKDGKYYLGIMNTTANKAFVNPPAAKTENVFKKVDYKLLPGSNKMLPKV | |
| | FFAKSNIGYYNPSTELYSNYKKGTHKKGPSFSIDDCHNLIDFFKESIKKHEDWSKFGFEFSDTA | |
| | DYRDISEFYREVEKQGYKLTFTDIDESYINDLIEKNELYLFQIYNKDFSEYSKGKLNLHTLYFM | |
| | MLFDQRNLDNVVYKLNGEAEVFYRPASIAENELVIHKAGEGIKNKNPNRAKVKETSTFSYDIVK | |
| | DKRYSKYKFTLHIPITMNFGVDEVRRFNDVINNALRTDDNVNVIGIDRGERNLLYVVVINSEGK | |
| | ILEQISLNSIINKEYDIETNYHALLDEREDDRNKARKDWNTIENIKELKTGYLSQVVNVVAKLV | |
| | LKYNAIICLEDLNFGFKRGRQKVEKQVYQKFEKMLIEKLNYLVIDKSREQVSPEKMGGALNALQ | |
| | LTSKFKSFAELGKQSGIIYYVPAYLTSKIDPTTGFVNLFYIKYENIEKAKQFFDGFDFIRENKK | |
| | DDMFEFSFDYKSFTQKACGIRSKWIVYINGERIIKYPNPEKNNLFDEKVINVTDEIKGLFKQYR | |
| | IPYENGEDIKEIIISKAEADFYKRLFRLLHQTLQMRNSTSDGTRDYIISPVKNDRGEFFCSEFS | |
| | EGTMPKDADANGAYNIARKGLWVLEQIRQKDEGEKVNLSMTNAEWLKYAQLHLLAS | |
| HkCas12a | MFEKLSNIVSISKTIRFKLIPVGKTLENIEKLGKLEKDFERSDFYPILKNISDDYYRQYIKEKL | 36 |
| Helcococcus kunzii | SDLNLDWQKLYDAHELLDSSKKESQKNLEMIQAQYRKVLFNILSGELDKSGEKNSKDLIKNNKA | |
| ATCC 51366 | LYGKLFKKQFILEVLPDFVNNNDSYSEEDLEGLNLYSKFTTRLKNFWETRKNVFTDKDIVTAIP | |
| | FRAVNENFGFYYDNIKIFNKNIEYLENKIPNLENELKEADILDDNRSVKDYFTPNGFNYVITQD | |
| | GIDVYQAIRGGFTKENGEKVQGINEILNLTQQQLRRKPETKNVKLGVLTKLRKQILEYSESTSF | |
| | LIDQIEDDNDLVDRINKFNVSFFESTEVSPSLFEQIERLYNALKSIKKEEVYIDARNTQKFSQM | |
| | LFGQWDVIRRGYTVKITEGSKEEKKKYKEYLELDETSKAKRYLNIREIEELVNLVEGFEEVDVE | |
| | SVLLEKFKMNNIERSEFEAPIYGSPIKLEAIKEYLEKHLEEYHKWKLLLIGNDDLDTDETFYPL | |
| | LNEVISDYYIIPLYNLTRNYLTRKHSDKDKIKVNFDFPTLADGWSESKISDNRSIILRKGGYYY | |
| | LGILIDNKLLINKKNKSKKIYEILIYNQIPEFSKSIPNYPFTKKVKEHFKNNVSDFQLIDGYVS | |
| | PLIITKEIYDIKKEKKYKKDFYKDNNTNKNYLYTIYKWIEFCKQFLYKYKGPNKESYKEMYDFS | |
| | TLKDTSLYVNLNDFYADVNSCAYRVLFNKIDENTIDNAVEDGKLLLFQIYNKDFSPESKGKKNL | |
| | HTLYWLSMFSEENLRTRKLKLNGQAEIFYRKKLEKKPIIHKEGSILLNKIDKEGNTIPENIYHE | |
| | CYRYLNKKIGREDLSDEAIALFNKDVLKYKEARFDIIKDRRYSESQFFFHVPITENWDIKTNKN | |
| | VNQIVQGMIKDGEIKHIIGIDRGERHLLYYSVIDLEGNIVEQGSLNTLEQNRFDNSTVKVDYQN | |
| | KLRTREEDRDRARKNWTNINKIKELKDGYLSHVVHKLSRLIIKYEAIVIMENLNQGFKRGRFKV | |
| | ERQVYQKFELALMNKLSALSFKEKYDERKNLEPSGILNPIQACYPVDAYQELQGQNGIVFYLPA | |
| | AYTSVIDPVTGFTNLFRLKSINSSKYEEFIKKFKNIYFDNEEEDFKFIFNYKDFAKANLVILNN | |
| | IKSKDWKISTRGERISYNSKKKEYFYVQPTEFLINKLKELNIDYENIDIIPLIDNLEEKAKRKI | |
| | LKALFDTFKYSVQLRNYDFENDYIISPTADDNGNYYNSNEIDIDKTNLPNNGDANGAFNIARKG | |
| | LLLKDRIVNSNESKVDLKIKNEDWINFIISAS | |
| LpCas12a | MIMNNVTGDFSEFVAISKVQKTLRNELRPTPLTMKHIKQKGIITEDEYKTQQSLELKRIADGYY | 37 |
| Lachnospira | RDYITHKLNDTNNLDFRNLFEAIEEKYKKNDKDNRDKLDLVEKSKRGEIAKLLSADDNFKSMFE | |
| pectinoschiza strain | AKLITQLLPVYVEQNYIGEDKEKALETIALFKGFTTYFTDYFNIRKNMFKENGGASSICYRIVN | |
| 2789STDY5834836 | VNASIFYDNLKTFMCIKEKAETEIALIEEELTELLDSWRLEHIFSEDYYNELLAQKGIDYYNQI | |
| | CGDVNKHMNLYCQQNKLKANVFKMTKLQKQIMGISEKAFEIPPMYQNDEEVYAAFNGFISRLEE | |
| | VKLIDRLGNVLQNSNIYDTAKIYINARCYTNVSSYVYGGWGVIESAIERYWYNTIAGKGQSKAK | |
| | KIEKAKKDNKFMSVKELDSIVSDYEPDYFNASNMDDDNSGRAFSGHGVLGYFNKMSKLLANMSL | |
| | HTITYDSGDSLIENKETALNIKKDLDDIMSIYHWLQTFIIDEVVEKDNAFYAELEDIYYELENV | |
| | VTLYDRIRNYVTRKPYSTQKFKLNFASPTLASGWSRSKEFDNNAIILLRNNKYYIAIFNVNNKP | |
| | DKQIIKGSEEQRLSTDYKKMVYNLLPGPNKMLPWVFIKSNTGKRDYNPSSYILEGYEKNRHIKS | |
| | SGNFDINYCHDLIDYYKACINKHPEWKNYGFKFKETTQYNDIGQFYKDVEKQGYSISWAYISEA | |
| | DINRLDEEGKIYLFEIYNKDLSSHSTGKDNLHTMYLKNIFSEDNLKNICIELNGNAELFYRKSS | |
| | MKRNITHKKDTVLVNKTYINEAGVRVSLTDEDYIKVYNYYNNDYVIDVEKDKKLVEILERIGHR | |
| | KNPIDIIKDKRYTEDKYFLHFPITINYGVDDENINAKMIEYIAKHNNMNVIGIDRGERNLIYIS | |
| | VINNKGNIIEQKSFNLVNNYDYKNKLKNMEKTRDNARKNWQEIGKIKDVKNGYLSGVISKIARM | |
| | VVDYNAIIVMEDLNRGFKRGRFKVERQVYQKFENMLISKLNYLVFKEKKADENGGILKGYQLTY | |
| | LPKSALQIGKQCGCIFYVPAAYTSKIDPATGFINIFDFKKYSGSAINAKVKDKKEFLMSMNSIR | |
| | YVNEGSAEYEKIGHRQLFAFSFDYNNFKTYNVSIPVNEWTTYTYGERIKKLYKDGRWSGSEVLN | |
| | LTEDLIELMEQYGIEYKDGHDIREDISHMDEMRNADFICNLFEKFKYTVQLRNSKSEAEGDDYD | |
| | RLVSPVLNSHNGFFDSSDYKENEKSDDIIDDKQIMPKDADANGAYCIALKGLYEINKIKENWSD | |
| | DKKLKESELYIGVTEWLDYIQNRRFEAS | |
| CMaCas12a | MDAKEFTGQYPLSKTLRFELRPIGRTWDNLEASGYLAEDRHRAECYPRAKELLDDNHRAFLNRV | 38 |
| Candidatus | LPQIDMDWHPIAEAFCKVHKNPGNKELAQDYNLQLSKRRKEISAYLQDADGYKGLFAKPALDEA | |
| Methanomethylophilus | MKIAKENGNESDIEVLEAFNGFSVYFTGYHESRENIYSDEDMVSVAYRITEDNFPRFVSNALIF | |
| alvus Mx1201 | DKLNESHPDIISEVSGNLGVDDIGKYFDVSNYNNFLSQAGIDDYNHIIGGHTTEDGLIQAFNVV | |
| | LNLRHQKDPGFEKIQFKQLYKQILSVRTSKSYIPKQFDNSKEMVDCICDYVSKIEKSETVERAL | |
| | KLVRNISSFDLRGIFVNKKNLRILSNKLIGDWDAIETALMHSSSSENDKKSVYDSAEAFTLDDI | |
| | FSSVKKFSDASAEDIGNRAEDICRVISETAPFINDLRAVDLDSLNDDGYEAAVSKIRESLEPYM | |
| | DLFHELEIFSVGDEFPKCAAFYSELEEVSEQLIEIIPLENKARSFCTRKRYSTDKIKVNLKFPT | |
| | LADGWDLNKERDNKAAILRKDGKYYLAILDMKKDLSSIRTSDEDESSFEKMEYKLLPSPVKMLP | |
| | KIFVKSKAAKEKYGLTDRMLECYDKGMHKSGSAFDLGFCHELIDYYKRCIAEYPGWDVFDFKER | |
| | ETSDYGSMKEFNEDVAGAGYYMSLRKIPCSEVYRLLDEKSIYLFQIYNKDYSENAHGNKNMHTM | |
| | YWEGLFSPQNLESPVFKLSGGAELFFRKSSIPNDAKTVHPKGSVLVPRNDVNGRRIPDSTYREL | |
| | TRYFNRGDCRISDEAKSYLDKVKTKKADHDIVKDRRFTVDKMMFHVPIAMNFKAISKPNLNKKV | |
| | IDGIIDDQDLKIIGIDRGERNLIYVTMVDRKGNILYQDSLNILNGYDYRKALDVREYDNKEARR | |
| | NWTKVEGIRKMKEGYLSLAVSKLADMIIENNAIIVMEDLNHGFKAGRSKIEKQVYQKFESMLIN | |
| | KLGYMVLKDKSIDQSGGALHGYQLANHVTTLASVGKQCGVIFYIPAAFTSKIDPTTGFADLFAL | |
| | SNVKNVASMREFFSKMKSVIYDKAEGKFAFTFDYLDYNVKSECGRTLWTVYTVGERFTYSRVNR | |
| | EYVRKVPTDIIYDALQKAGISVEGDLRDRIAESDGDTLKSIFYAFKYALDMRVENREEDYIQSP | |
| | VKNASGEFFCSKNAGKSLPQDSDANGAYNIALKGILQLRMLSEQYDPNAESIRLPLITNKAWLT | |
| | FMQSGMKTWKN | |
| PgCas12a | MENIFDQFIGKYSLSKTLRFELKPVGKTEDFLKINKVFEKDQTIDDSYNQAKFYFDSLHQKFID | 39 |
| Parcubacteria | AALASDKTSELSFQNFADVLEKQNKIILDKKREMGALRKRDKNAVGIDRLQKEINDAEDIIQKE | |
| group bacterium | KEKIYKDVRTLFDNEAESWKTYYQEREVDGKKITFSKADLKQKGADELTAAGILKVLKYEFPEE | |
| GW2011 GWC2 44 | KEKEFQAKNQPSLFVEEKENPGQKRYIFDSFDKFAGYLTKFQQTKKNLYAADGTSTAVATRIAD | |
| 17 | NFIIFHQNTKVFRDKYKNNHTDLGFDEENIFEIERYKNCLLQREIEHIKNENSYNKIIGRINKK | |
| | IKEYRDQKAKDTKLTKSDFPFFKNLDKQILGEVEKEKQLIEKTREKTEEDVLIERFKEFIENNE | |
| | ERFTAAKKLMNAFCNGEFESEYEGIYLKNKAINTISRRWFVSDRDFELKLPQQKSKNKSEKNEP | |
| | KVKKFISIAEIKNAVEELDGDIFKAVFYDKKIIAQGGSKLEQFLVIWKYEFEYLFRDIERENGE | |
| | KLLGYDSCLKIAKQLGIFPQEKEAREKATAVIKNYADAGLGIFQMMKYFSLDDKDRKNTPGQLS | |
| | TNFYAEYDGYYKDFEFIKYYNEFRNFITKKPFDEDKIKLNFENGALLKGWDENKEYDFMGVILK | |
| | KEGRLYLGIMHKNHRKLFQSMGNAKGDNANRYQKMIYKQIADASKDVPRLLLTSKKAMEKFKPS | |
| | QEILRIKKEKTFKRESKNFSLRDLHALIEYYRNCIPQYSNWSFYDFQFQDTGKYQNIKEFTDDV | |
| | QKYGYKISFRDIDDEYINQALNEGKMYLFEVVNKDIYNTKNGSKNLHTLYFEHILSAENLNDPV | |
| | FKLSGMAEIFQRQPSVNEREKITTQKNQCILDKGDRAYKYRRYTEKKIMFHMSLVLNTGKGEIK | |
| | QVQFNKIINQRISSSDNEMRVNVIGIDRGEKNLLYYSVVKQNGEIIEQASLNEINGVNYRDKLI | |
| | EREKERLKNRQSWKPVVKIKDLKKGYISHVIHKICQLIEKYSAIVVLEDLNMRFKQIRGGIERS | |
| | VYQQFEKALIDKLGYLVFKDNRDLRAPGGVLNGYQLSAPFVSFEKMRKQTGILFYTQAEYTSKT | |
| | DPITGFRKNVYISNSASLDKIKEAVKKFDAIGWDGKEQSYFFKYNPYNLADEKYKNSTVSKEWA | |
| | IFASAPRIRRQKGEDGYWKYDRVKVNEEFEKLLKVWNFVNPKATDIKQEIIKKEKAGDLQGEKE | |
| | LDGRLRNFWHSFIYLFNLVLELRNSFSLQIKIKAGEVIAVDEGVDFIASPVKPFFTTPNPYIPS | |
| | NLCWLAVENADANGAYNIARKGVMILKKIREHAKKDPEFKKLPNLFISNAEWDEAARDWGKYAG | |
| | TTALNLDH | |
| PrCas12a | MIIGRDFNMYYQNLTKMYPISKTLRNELIPVGKTLENIRKNGILEADIQRKADYEHVKKLMDNY | 40 |
| Pseudobutyrivibrio | HKQLINEALQGVHLSDLSDAYDLYFNLSKEKNSVDAFSKCQDKLRKEIVSLLKNHENFPKIGNK | |
| ruminis CF1b | EIIKLLQSLYDNDTDYKALDSFSNFYTYFSSYNEVRKNLYSDEEKSSTVAYRLINENLPKFLDN | |
| | IKAYAIAKKAGVRAEGLSEEDQDCLFIIETFERTLTQDGIDNYNAAIGKLNTAINLENQQNKKQ | |
| | EGFRKVPQMKCLYKQILSDREEAFIDEFSDDEDLITNIESFAENMNVFLNSEIITDFKIALVES | |
| | DGSLVYIKNDVSKTSFSNIVFGSWNAIDEKLSDEYDLANSKKKKDEKYYEKRQKELKKNKSYDL | |
| | ETIIGLFDDNSDVIGKYIEKLESDITAIAEAKNDFDEIVLRKHDKNKSLRKNTNAVEAIKSYLD | |
| | TVKDFERDIKLINGSGQEVEKNLVVYAEQENILAEIKNVDSLYNMSRNYLTQKPFSTEKFKLNF | |
| | NRATLLNGWDKNKETDNLGILFEKDGMYYLGIMNTKANKIFVNIPKATSNDVYHKVNYKLLPGP | |
| | NKMLPKVFFAQSNLDYYKPSEELLAKYKAGTHKKGDNFSLEDCHALIDFFKASIEKHPDWSSFG | |
| | FEFSETCTYEDLSGFYREVEKQGYKITYTDVDADYITSLVERDELYLFQIYNKDFSPYSKGNLN | |
| | LHTIYLQMLFDQRNLNNVVYKLNGEAEVFYRPASINDEEVIIHKAGEEIKNKNSKRAVDKPTSK | |
| | FGYDIIKDRRYSKDKFMLHIPVTMNFGVDETRRFNDVVNDALRNDEKVRVIGIDRGERNLLYVV | |
| | VVDTDGTILEQISLNSIINNEYSIETDYHKLLDEKEGDRDRARKNWTTIENIKELKEGYLSQVV | |
| | NVIAKLVLKYNAIICLEDLNFGFKRGRQKVEKQVYQKFEKMLIDKLNYLVIDKSRKQDKPEEFG | |
| | GALNALQLTSKFTSFKDMGKQTGIIYYVPAYLTSKIDPTTGFANLFYVKYENVEKAKEFFSRED | |
| | SISYNNESGYFEFAFDYKKFTDRACGARSQWTVCTYGERIIKFRNTEKNNSFDDKTIVLSEEFK | |
| | ELFSIYGISYEDGAELKNKIMSVDEADFFRSLTRLFQQTMQMRNSSNDVTRDYIISPIMNDRGE | |
| | FFNSEACDASKPKDADANGAFNIARKGLWVLEQIRNTPSGDKLNLAMSNAEWLEYAQRNQIAS | |
| FnCas12a | MSIYQEFVNKYSLSKTLRFELIPQGKTLENIKARGLILDDEKRAKDYKKAKQIIDKYHQFFIEE | 41 |
| Francisella | ILSSVCISEDLLQNYSDVYFKLKKSDDDNLQKDFKSAKDTIKKQISEYIKDSEKFKNLFNQNLI | |
| tularensis subsp. | DAKKGQESDLILWLKQSKDNGIELFKANSDITDIDEALEIIKSFKGWTTYFKGFHENRKNVYSS | |
| novicida strain | NDIPTSIIYRIVDDNLPKFLENKAKYESLKDKAPEAINYEQIKKDLAEELTFDIDYKTSEVNQR | |
| U112 | VFSLDEVFEIANFNNYLNQSGITKFNTIIGGKFVNGENTKRKGINEYINLYSQQINDKTLKKYK | |
| | MSVLFKQILSDTESKSFVIDKLEDDSDVVTTMQSFYEQIAAFKTVEEKSIKETLSLLFDDLKAQ | |
| | KLDLSKIYFKNDKSLTDLSQQVFDDYSVIGTAVLEYITQQIAPKNLDNPSKKEQELIAKKTEKA | |
| | KYLSLETIKLALEEFNKHRDIDKQCRFEEILANFAAIPMIFDEIAQNKDNLAQISIKYQNQGKK | |
| | DLLQASAEDDVKAIKDLLDQTNNLLHKLKIFHISQSEDKANILDKDEHFYLVFEECYFELANIV | |
| | PLYNKIRNYITQKPYSDEKFKLNFENSTLANGWDKNKEPDNTAILFIKDDKYYLGVMNKKNNKI | |
| | FDDKAIKENKGEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVENQGYKLTFEN | |
| | ISESYIDSVVNQGKLYLFQIYNKDFSAYSKGRPNLHTLYWKALFDERNLQDVVYKLNGEAELFY | |
| | RKQSIPKKITHPAKEAIANKNKDNPKKESVFEYDLIKDKRFTEDKFFFHCPITINFKSSGANKF | |
| | NDEINLLLKEKANDVHILSIDRGERHLAYYTLVDGKGNIIKQDTFNIIGNDRMKTNYHDKLAAI | |
| | EKDRDSARKDWKKINNIKEMKEGYLSQVVHEIAKLVIEYNAIVVFEDLNFGFKRGRFKVEKQVY | |
| | QKLEKMLIEKLNYLVFKDNEFDKTGGVLRAYQLTAPFETFKKMGKQTGIIYYVPAGFTSKICPV | |
| | TGFVNQLYPKYESVSKSQEFFSKFDKICYNLDKGYFEFSFDYKNFGDKAAKGKWTIASFGSRLI | |
| | NFRNSDKNHNWDTREVYPTKELEKLLKDYSIEYGHGECIKAAICGESDKKFFAKLTSVLNTILQ | |
| | MRNSKTGTELDYLISPVADVNGNFFDSRQAPKNMPQDADANGAYHIGLKGLMLLGRIKNNQEGK | |
| | KLNLVIKNEEYFEFVQNRNNG | |
| BfCas12a | MYYESLTKLYPIKKTIRNELVPIGKTLENIKKNNILEADEDRKIAYIRVKAIMDDYHKRLINEA | 42 |
| Butyrivibrio | LSGFALIDLDKAANLYLSRSKSADDIESFSRFQDKLRKAIAKRLREHENFGKIGNKDIIPLLQK | |
| fibrisolvens | LSENEDDYNALESFKNFYTYFESYNDVRLNLYSDKEKSSTVAYRLINENLPRFLDNIRAYDAVQ | |
| | KAGITSEELSSEAQDGLFLVNTENNVLIQDGINTYNEDIGKLNVAINLYNQKNASVQGFRKVPK | |
| | MKVLYKQILSDREESFIDEFESDTELLDSLESHYANLAKYFGSNKVQLLFTALRESKGVNVYVK | |
| | NDIAKTSFSNVVFGSWSRIDELINGEYDDNNNRKKDEKYYDKRQKELKKNKSYTIEKIITLSTE | |
| | DVDVIGKYIEKLESDIDDIRFKGKNFYEAVLCGHDRSKKLSKNKGAVEAIKGYLDSVKDFERDL | |
| | KLINGSGQELEKNLVVYGEQEAVLSELSGIDSLYNMTRNYLTKKPFSTEKIKLNFNKPTFLDGW | |
| | DYGNEEAYLGFFMIKEGNYFLAVMDANWNKEFRNIPSVDKSDCYKKVIYKQISSPEKSIQNLMV | |
| | IDGKTVKKNGRKEKEGIHSGENLILEELKNTYLPKKINDIRKRRSYLNGDTFSKKDLTEFIGYY | |
| | KQRVIEYYNGYSFYFKSDDDYASFKEFQEDVGRQAYQISYVDVPVSFVDDLINSGKLYLFRVYN | |
| | KDFSEYSKGRLNLHTLYFKMLFDERNLKNVVYKLNGQAEVFYRPSSIKKEELIVHRAGEEIKNK | |
| | NPKRAAQKPTRRLDYDIVKDRRYSQDKFMLHTSIIMNFGAEENVSFNDIVNGVLRNEDKVNVIG | |
| | IDRGERNLLYVVVIDPEGKILEQRSLNCITDSNLDIETDYHRLLDEKESDRKIARRDWTTIENI | |
| | KELKAGYLSQVVHIVAELVLKYNAIICLEDLNFGFKRGRQKVEKQVYQKFEKMLIDKLNYLVMD | |
| | KSREQLSPEKISGALNALQLTPDFKSFKVLGKQTGIIYYVPAYLTSKIDPMTGFANLFYVKYEN | |
| | VDKAKEFFSKFDSIKYNKDGKNWNTKGYFEFAFDYKKFTDRAYGRVSEWTVCTVGERIIKFKNK | |
| | EKNNSYDDKVIDLTNSLKELFDSYKVTYESEVDLKDAILAIDDPAFYRDLTRRLQQTLQMRNSS | |
| | CDGSRDYIISPVKNSKGEFFCSDNNDDTTPNDADANGAFNIARKGLWVLNEIRNSEEGSKINLA | |
| | MSNAQWLEYAQDNTI | |
| SrCas12a | MGNFGEFTHKYQVSKTLRFELIPQGKTLENVAKYGIVDDDKRRSENYKKLKPVIDRIYKYFIDE | 43 |
| Succiniclasticum | SLKNVSIDWQPLYEAIIAYRKEQTTANVVRLKEEQEACRKAIAAWFEGKVPDKGSKDLKEFNKT | |
| ruminis | QSKLFKELFGKELFTESVTQLLPGLSLTEEEKELLASFNKFTSYFKGFYVNRKNVFSADDISTS | |
| | IPHRLVQENFPKFMDNCEAYRRIVEEYPELKAKLEGTAQATGIFIGFKLDNIFKVSFYNHLLQQ | |
| | SQIDLYNQFLCGIAGEEGTMRVQGLNVTLNLAMKQDKVLGQKLKSMPHRFIPLYKQILSDRTTL | |
| | SFIPEAFQNDEEVLLTVEEYRKSLEAERTTGAVSDIFNSLQAADLRHVYVNPAKLTAFSQMLFE | |
| | DWSLCRESLRNWKLRSYGKAATKKVREEIESWLKESAISLDELQAALADGTLSVIINQKVQSVI | |
| | TTLEQELAKPLPKKLKTAEEKESLKSLLDSVQEACHSLEMFAVGENMDTDPCFYVPLREAMEAI | |
| | QPIIPLYNKVRNFATQKPYSIEKFKLNFSNPILASGWDENRERQTCAILFRKGEKYYLGIYNAK | |
| | VKPDFSIIKAVKGGNCFEKVVYRQFPDFSKMMPKCTTQLKEVQQHFASSSEDYVLYNKKFIKPL | |
| | TITKEIYDLNNVLFDGKKKFQIDYLRKTKDEDGYYHALHTWINFAKEFVASYESTSIYDTSTVL | |
| | STEQYVKLNDFYGDLDNLFYRIKFESVSEETISEFVDEGKLFLFQIYNKDFAEGATGAPNLHTI | |
| | YWKAVFDPENMKNVVVKLNGQAELFYRPKSAMDIVRHKVGEKLVNRRLKDGTSLTEELHEELYL | |
| | YANGKLKKKLSEAAAAVLPQAVIYDVHHEIVKDRRFTEDKFFFHVPLTLNYKCDKNAVQFNASV | |
| | QEYLKENPDTYIIGIDRGERNLIYAVVIDPQGNIVEQKSENVINGFDYHNKLEQREKERNKARQ | |
| | DWTTVGKIKELKQGYLSLVVHEITSMMVKYNAIVVLENLNVGFKRIRSGIAEKAVYQQFEKMLI | |
| | NKLNYLMFKDVEGAKPGSVLNAYQLTDRFESFASMRNQTGFLFYIPAAFTSKIDPATGFVDPFC | |
| | WSAIKTLDDKKTFISGFDTLKYDNVTGNFILHFEMKKNKDFQKKLEGFMPEWDIVVEANKDRRD | |
| | AEGKTFISGKRIEFVRENNGHGHYEDYLPCKKLVEILRQYDILFEDGKDVLPLIMKNGDSKLIH | |
| | EVFKVIRLSLQMRNSNAESGEDFISSPVENNEGICFDSRLGVETLPKDADANGAYHIALKGLLL | |
| | LEKIRHDERKLGISNSEWLNHIQSLRG | |
| LbCas12a-MD335 | MHENNGKIADNFIGIYPVSKTLRFELKPVGKTQEYIEKHGILDEDLKRAGDYKSVKKIIDAYHK | 44 |
| Lachnospiraceae | YFIDEALNGIQLDGLKNYYELYEKKRDNNEEKEFQKIQMSLRKQIVKRFSEHPQYKYLFKKELI | |
| bacterium MD335 | KNVLPEFTKDNAEEQTLVKSFQEFTTYFEGFHQNRKNMYSDEEKSTAIAYRVVHQNLPKYIDNM | |
| | RIFSMILNTDIRSDLTELFNNLKTKMDITIVEEYFAIDGFNKVVNQKGIDVYNTILGAFSTDDN | |
| | TKIKGLNEYINLYNQKNKAKLPKLKPLFKQILSDRDKISFIPEQFDSDTEVLEAVDMFYNRLLQ | |
| | FVIENEGQITISKLLTNFSAYDINKIYVKNDTTISAISNDLEDDWSYISKAVRENYDSENVDKN | |
| | KRAAAYEEKKEKALSKIKMYSIEELNFFVKKYSCNECHIEGYFERRILEILDKMRYAYESCKIL | |
| | HDKGLINNISLCQDRQAISELKDFLDSIKEVQWLLKPLMIGQEQADKEEAFYTELLRIWEELEP | |
| | ITLLYNKVRNYVTKKPYTLEKVKLNFYKSTLLDGWDKNKEKDNLGIILLKDGQYYLGIMNRRNN | |
| | KIADDAPLAKTDNVYRKMEYKLLTKVSANLPRIFLKDKYNPSEEMLEKYEKGTHLKGENFCIDD | |
| | CRELIDFFKKGIKQYEDWGQFDFKFSDTESYDDISAFYKEVEHQGYKITFRDIDETYIDSLVNE | |
| | GKLYLFQIYNKDFSPYSKGTKNLHTLYWEMLFSQQNLQNIVYKLNGNAEIFYRKASINQKDVVV | |
| | HKADLPIKNKDPQNSKKESMFDYDIIKDKRFTCDKYQFHVPITMNFKALGENHFNRKVNRLIHD | |
| | AENMHIIGIDRGERNLIYLCMIDMKGNIVKQISLNEIISYDKNKLEHKRNYHQLLKTREDENKS | |
| | ARQSWQTIHTIKELKEGYLSQVIHVITDLMVEYNAIVVLEDLNFGFKQGRQKFERQVYQKFEKM | |
| | LIDKLNYLVDKSKGMDEDGGLLHAYQLTDEFKSFKQLGKQSGFLYYIPAWNTSKLDPTTGFVNL | |
| | FYTKYESVEKSKEFINNFTSILYNQEREYFEFLFDYSAFTSKAEGSRLKWTVCSKGERVETYRN | |
| | PKKNNEWDTQKIDLTFELKKLFNDYSISLLDGDLREQMGKIDKADFYKKFMKLFALIVQMRNSD | |
| | EREDKLISPVLNKYGAFFETGKNERMPLDADANGAYNIARKGLWIIEKIKNTDVEQLDKVKLTI | |
| | SNKEWLQYAQEHIL | |
| CMtCas12a | MNNYDEFTKLYPIQKTIRFELKPQGRTMEHLETENFFEEDRDRAEKYKILKEAIDEYHKKFIDE | 45 |
| Candidatus | HLTNMSLDWNSLKQISEKYYKSREEKDKKVFLSEQKRMRQEIVSEFKKDDRFKDLFSKKLFSEL | |
| Methanoplasma | LKEEIYKKGNHQEIDALKSFDKFSGYFIGLHENRKNMYSDGDEITAISNRIVNENFPKFLDNLQ | |
| termitum | KYQEARKKYPEWIIKAESALVAHNIKMDEVFSLEYFNKVLNQEGIQRYNLALGGYVTKSGEKMM | |
| | GLNDALNLAHQSEKSSKGRIHMTPLFKQILSEKESFSYIPDVFTEDSQLLPSIGGFFAQIENDK | |
| | DGNIFDRALELISSYAEYDTERIYIRQADINRVSNVIFGEWGTLGGLMREYKADSINDINLERT | |
| | CKKVDKWLDSKEFALSDVLEAIKRTGNNDAFNEYISKMRTAREKIDAARKEMKFISEKISGDEE | |
| | SIHIIKTLLDSVQQFLHFFNLFKARQDIPLDGAFYAEFDEVHSKLFAIVPLYNKVRNYLTKNNL | |
| | NTKKIKLNFKNPTLANGWDQNKVYDYASLIFLRDGNYYLGIINPKRKKNIKFEQGSGNGPFYRK | |
| | MVYKQIPGPNKNLPRVFLTSTKGKKEYKPSKEIIEGYEADKHIRGDKFDLDFCHKLIDFFKESI | |
| | EKHKDWSKFNFYFSPTESYGDISEFYLDVEKQGYRMHFENISAETIDEYVEKGDLFLFQIYNKD | |
| | FVKAATGKKDMHTIYWNAAFSPENLQDVVVKLNGEAELFYRDKSDIKEIVHREGEILVNRTYNG | |
| | RTPVPDKIHKKLTDYHNGRTKDLGEAKEYLDKVRYFKAHYDITKDRRYLNDKIYFHVPLTLNFK | |
| | ANGKKNLNKMVIEKFLSDEKAHIIGIDRGERNLLYYSIIDRSGKIIDQQSLNVIDGFDYREKLN | |
| | QREIEMKDARQSWNAIGKIKDLKEGYLSKAVHEITKMAIQYNAIVVMEELNYGFKRGRFKVEKQ | |
| | IYQKFENMLIDKMNYLVFKDAPDESPGGVLNAYQLTNPLESFAKLGKQTGILFYVPAAYTSKID | |
| | PTTGFVNLFNTSSKTNAQERKEFLQKFESISYSAKDGGIFAFAFDYRKFGTSKTDHKNVWTAYT | |
| | NGERMRYIKEKKRNELFDPSKEIKEALTSSGIKYDGGQNILPDILRSNNNGLIYTMYSSFIAAI | |
| | QMRVYDGKEDYIISPIKNSKGEFFRTDPKRRELPIDADANGAYNIALRGELTMRAIAEKFDPDS | |
| | EKMAKLELKHKDWFEFMQTRGD | |
| Cas12a uncultured | MEDKQFLERYKEFIGLNSLSKTLRNSLIPVGSTLKHIQEYGILEEDSLRAQKREELKGIMDDYY | 46 |
| Clostridium sp. | RNYIEMHLRDVHDIDWNELFEALTEVKKNQTDDAKKCLEKIQEKKRKEIYQYLSDDAVFSEMFK | |
| | EKMISGILPDFIRCNEEYSEEEKEEKLKTVALFHRFTSSENDFFLNRKNVFTKEAIATAIGYRV | |
| | VHENAEIFLENMVAFQNIQKSAESQISIIERKNEHYFMEWKLSHIFTADYYMMLMTQKAIEHYN | |
| | EMCGVVNQHMKEYCQKEKKNWNLYRMKRLHKQILSNASTSFKIPEKYENDAEVYESVNSFLQNV | |
| | MEKTVMERIAVLKNNTDNFDLSKIYITAPYYEKISNYLCGSWNTIADCLTHYYEQQIAGKGARK | |
| | DQKVKAAVKADKWKSLSEIEQLLKEYARAEEVKRKPEEYIAEIENIVSLKEVHLLEYHPEVNLI | |
| | ENEKYATEIKDVLDNYMELFHWMKWFYIEEAVEKEVNFYGELDDLYEEIRDIVPLYNKVRNYVT | |
| | QKPYSDTKIKLNFGTPTLANGWSKSKEYDYNAILLQKDGKYYMGIFNPVQKPEKEIIEGHSHPL | |
| | EGNEYKKMVYYYLPSANKMLPKVLLSKKGMEIYQPSEYIINGYKERRHIKSEEKFDLQFCHDLI | |
| | DYFKSGIERNPDWKVFGFHFSDTDTYQDISGFYREVEDQGYKIDWTYIKEADIDRLNEEGKLYL | |
| | FQIYNKDFSEKSTGRENLHTMYLKNLFSEENIREQVLKLNGEAEIFFRKSSVKKPIIHKKGTML | |
| | VNRTYMEEMHGESVKKNIPEKEYQEIYNYMNHRWKGELSAEAKEYLKKAVCHETKKDIVKDYRY | |
| | SVDKFFIHLPITINYRASGKEALNSVAQRYIAHQNDMHVIGIDRGERNLIYVSVINMQGEIIEQ | |
| | KSFNVVNKYNYKEKLKEREQNRDEARKNWKEIGQIKDLKEGYLSGVIHEIAKMMIKYHAIVAME | |
| | DLNYGFKRGRFKVERQVYQKFENMLIQKLNYLVFKDRSADEDGGVLRGYQLAYIPDSVKKLGRQ | |
| | CGMIFYVPAAFTSKIDPATGFVDIFNHKAYTTDQAKREFILSFDEICYDVERQLFRFTEDYANF | |
| | ATHNVTLARNNWTIYTNGTRTQKEFVNRRVRDKKEVFDPTEKMLKLLELEGVEYQSGANLLPKL | |
| | EKISDPHLFHELQRIVRFTVQLRNSKNEENDVDYDHVISPVLNEEGKFFDSSKYENKEEKKESL | |
| | LPVDADANGAYCIALKGLYIMQAIQKNWSEEKALSPDVLRLNNNDWFDYIQNKRYR | |
| Lachnospiraceae | MHENNGKIADNFIGIYPVSKTLRFELKPVGKTQEYIEKHGILDEDLKRAGDYKSVKKIIDAYHK | 47 |
| bacterium COE1 | YFIDEALNGIQLDGLKNYYELYEKKRDNNEEKEFQKIQMSLRKQIVKRFSEHPQYKYLFKKELI | |
| | KNVLPEFTKDNAEEQTLVKSFQEFTTYFEGFHQNRKNMYSDEEKSTAIAYRVVHQNLPKYIDNM | |
| | RIFSMILNTDIRSDLTELFNNLKTKMDITIVEEYFAIDGFNKVVNQKGIDVYNTILGAFSTDDN | |
| | TKIKGLNEYINLYNQKNKAKLPKLKPLFKQILSDRDKISFIPEQFDSDTEVLEAVDMFYNRLLQ | |
| | FVIENEGQITISKLLTNFSAYDLNKIYVKNDTTISAISNDLFDDWSYISKAVRENYDSENVDKN | |
| | KRAAAYEEKKEKALSKIKMYSIEELNFFVKKYSCNECHIEGYFERRILEILDKMRYAYESCKIL | |
| | HDKGLINNISLCQDRQAISELKDFLDSIKEVQWLLKPLMIGQEQADKEEAFYTELLRIWEELEP | |
| | ITLLYNKVRNYVTKKPYTLEKVKLNFYKSTLLDGWDKNKEKDNLGIILLKDGQYYLGIMNRRNN | |
| | KIADDAPLAKTDNVYRKMEYKLLTKVSANLPRIFLKDKYNPSEEMLEKYEKGTHLKGENFCIDD | |
| | CRELIDFFKKGIKQYEDWGQFDFKFSDTESYDDISAFYKEVEHQGYKITFRDIDETYIDSLVNE | |
| | GKLYLFQIYNKDFSPYSKGTKNLHTLYWEMLFSQQNLQNIVYKLNGNAEIFYRKASINQKDVVV | |
| | HKADLPIKNKDP | |
| | QNSKKESMFDYDIIKDKRFTCDKYQFHVPITMNFKALGENHFNRKVNRLIHDAENMHIIGIDRG | |
| | ERNLIYLCMIDMKGNIVKQISLNEIISYDKNKLEHKRNYHQLLKTREDENKSARQSWQTIHTIK | |
| | ELKEGYLSQVIHVITDLMVEYNAIVVLEDLNFGFKQGRQKFERQVYQKFEKMLIDKLNYLVDKS | |
| | KGMDEDGGLLHAYQLTDEFKSFKQLGKQSGFLYYIPAWNTSKLDPTTGFVNLFYTKYESVEKSK | |
| | EFINNFTSILYNQEREYFEFLFDYSAFTSKAEGSRLKWTVCSKGERVETYRNPKKNNEWDTQKI | |
| | DLTFELKKLFNDYSISLLDGDLREQMGKIDKADFYKKFMKLFALIVQMRNSDEREDKLISPVLN | |
| | KYGAFFETGKNERMPLDADANGAYNIARKGLWIIEKIKNTDVEQLDKVKLTISNKEWLQYAQEH | |
| | IL | |

Guide RNA (crRNA)

A guide RNA (gRNA) is an RNA that functions to guide an RNA- or DNA-targeting enzyme to a specific target. Targeting requires a gRNA complementary to the target site as well as a 5′ protospacer adjacent motif (PAM) on the DNA strand opposite the target sequence. The gRNA for a Cas12a endonuclease is relatively short, in some embodiments, about 35-50 nucleotides long (e.g., 35, 36, 37, 38, 39, 40, 41, 42, 43, 44, 45, 46, 47, 48, 49, or 50 nucleotides long). In some embodiments, a gRNA is about 40-44 nucleotides long. The portion of the gRNA that base pairs to the protospacer may be about 15-30 nucleotides long (e.g., 15, 16, 17, 18, 19, 20, 21, 22, 23, 24, 25, 26, 27, 28, 29, or 30 nucleotides long). In some embodiments, the portion of the gRNA that base pairs to the protospacer is about 20-24 nucleotides long, e.g., about 21 nucleotides long. There is also a constant portion that binds to Cas12a, which is about 15-25 nucleotides long. In some embodiments, the constant portion that binds to Cas12a is about 20 nucleotides long).

For Cas12a endonucleases, the target sequence to which a gRNA binds should be next to a PAM sequence—e.g., TTTV, where V can represent A, C, or G. The “V” of the TTTV is typically immediately adjacent to the most 5′ base of the non-targeted strand side of the protospacer element. The PAM sequence may vary, dependent on the variant Cas12a endonuclease.

### II. Variant Cas12a Endonucleases

Provided herein, in some aspects, are engineered variant Cas12a endonucleases that have altered activity relative to a wild-type Case12 endonuclease. An “variant Cas12a endonuclease” herein refers to a non-naturally occurring endonuclease obtained by mutation of a wild-type (e.g., naturally-occurring) Cas12a gene, for example, a Cas12a gene from Table 1 (e.g., any one of SEQ ID NOs: 1-47). Variants of other wild-type Cas12 genes are contemplated herein. Thus, the variant Cas12a endonucleases provided herein are “engineered.”

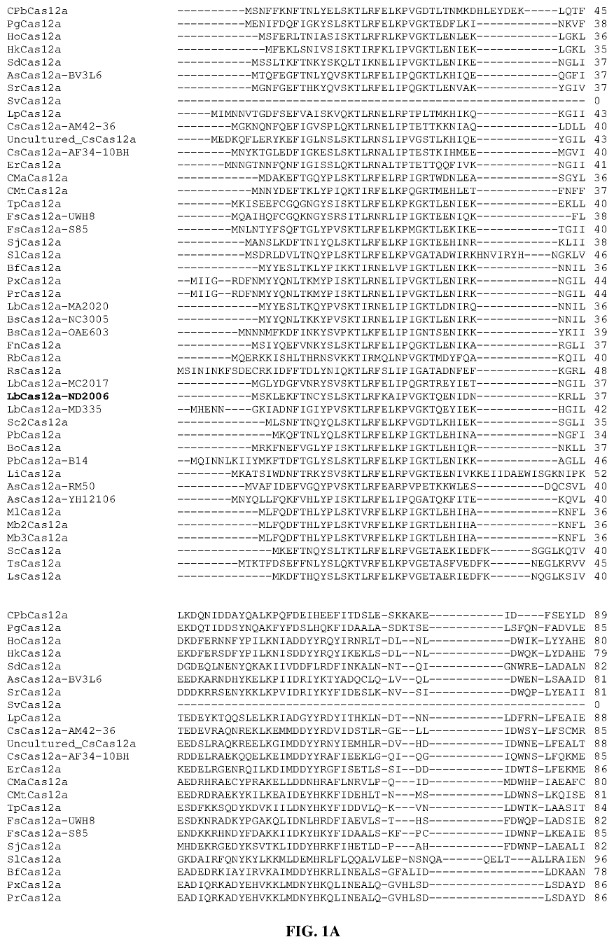

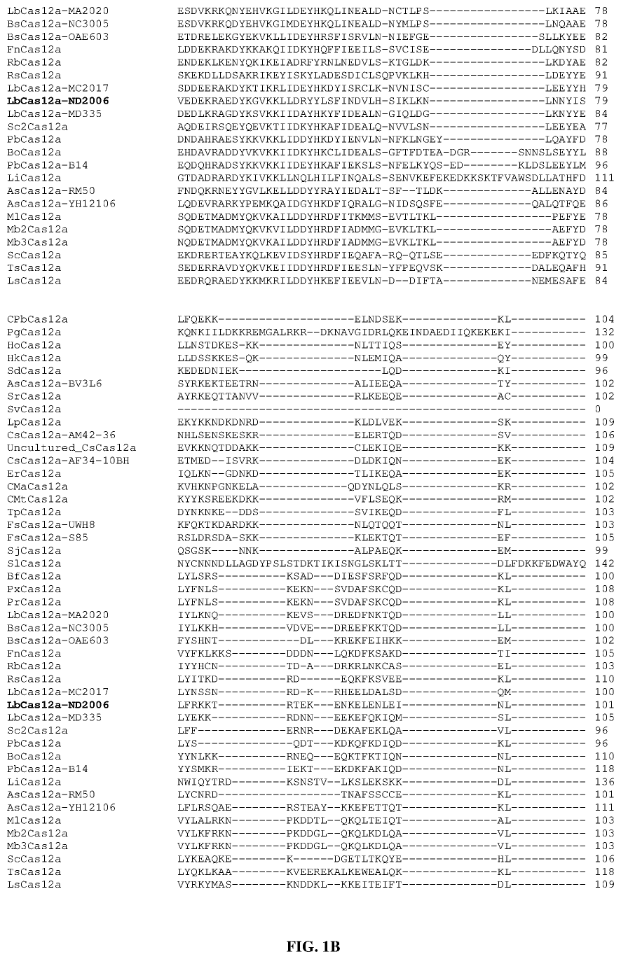

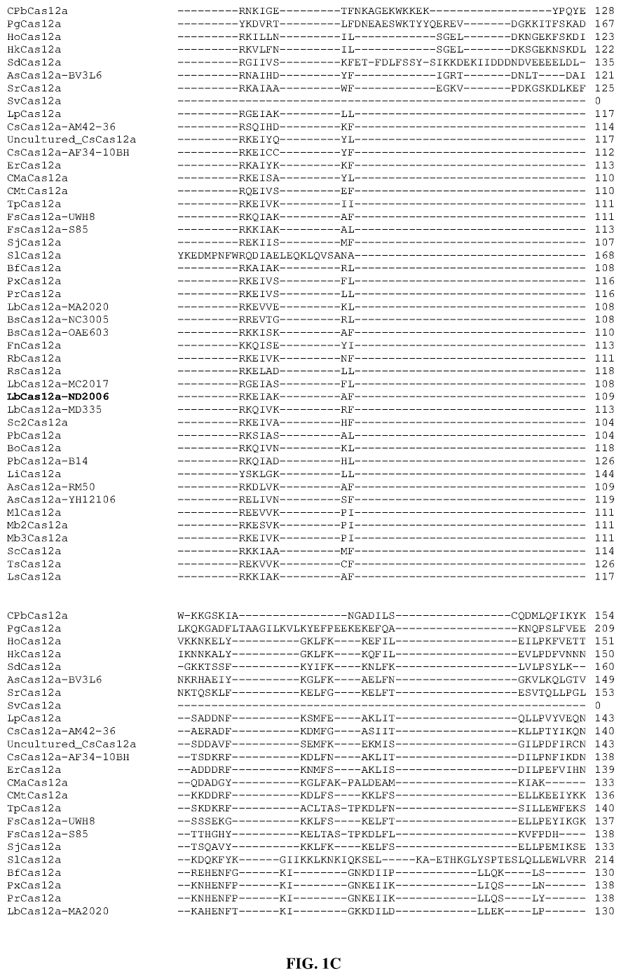

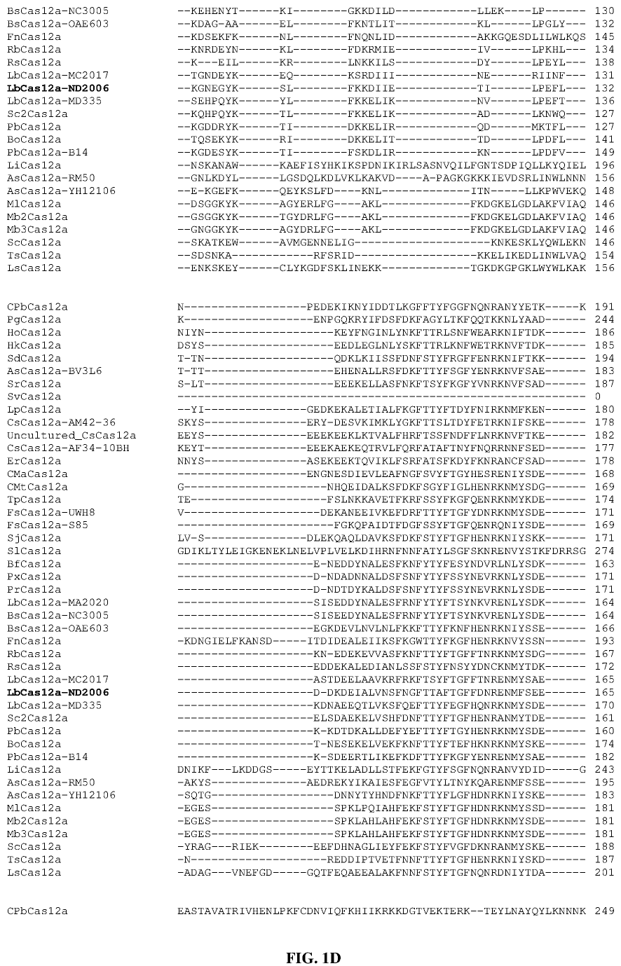

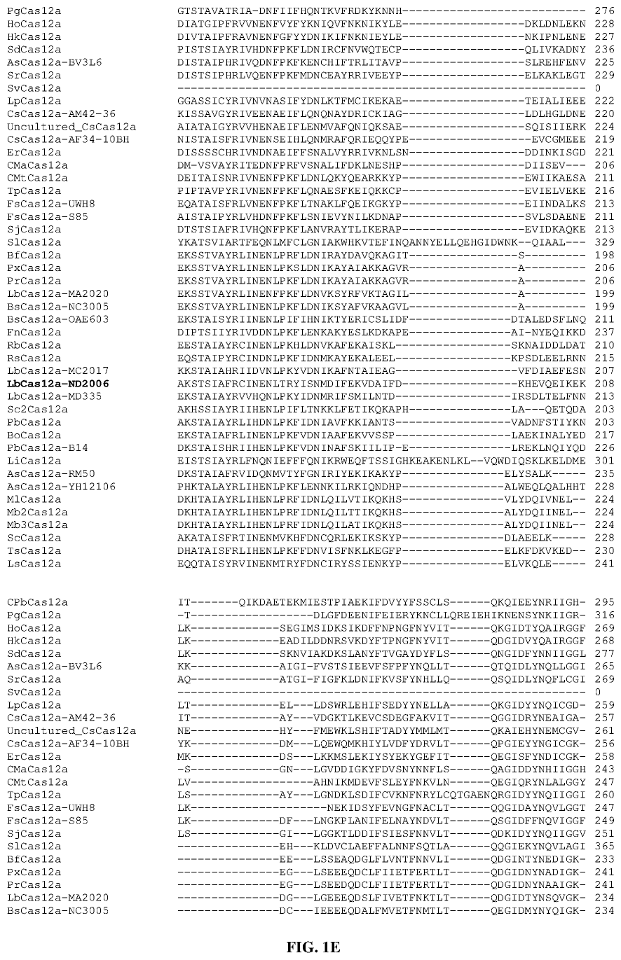

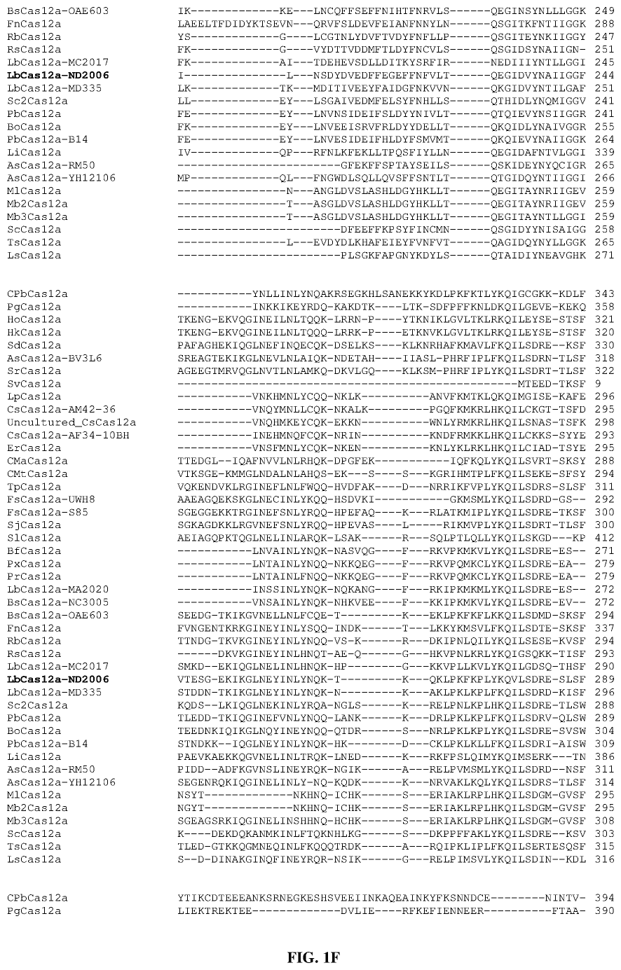

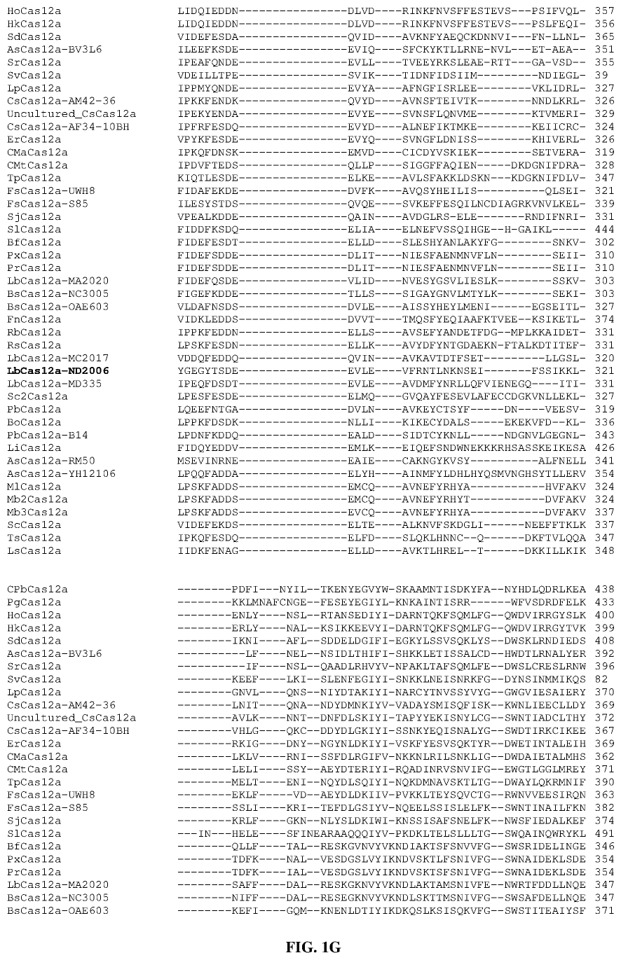

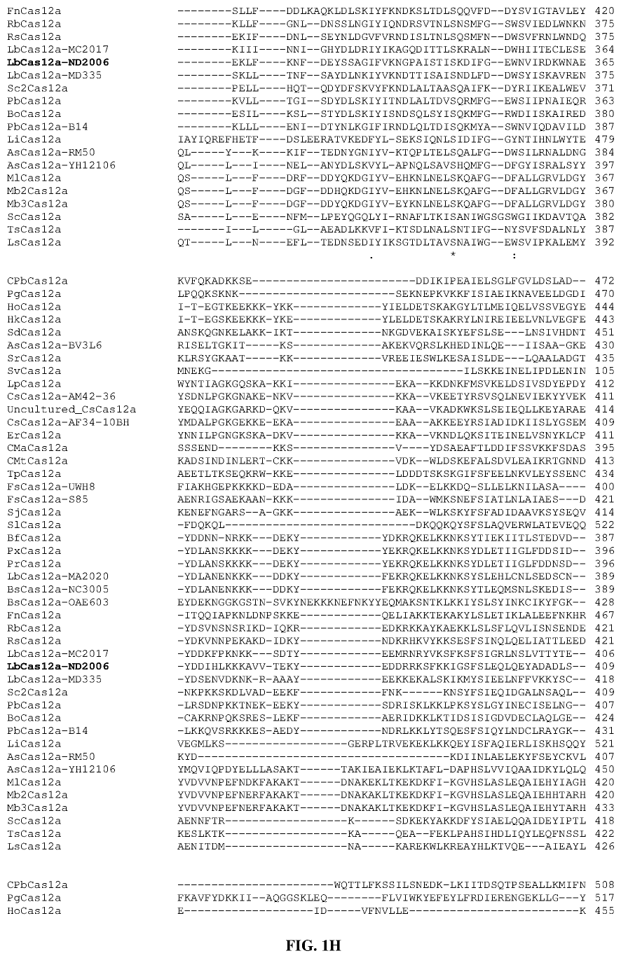

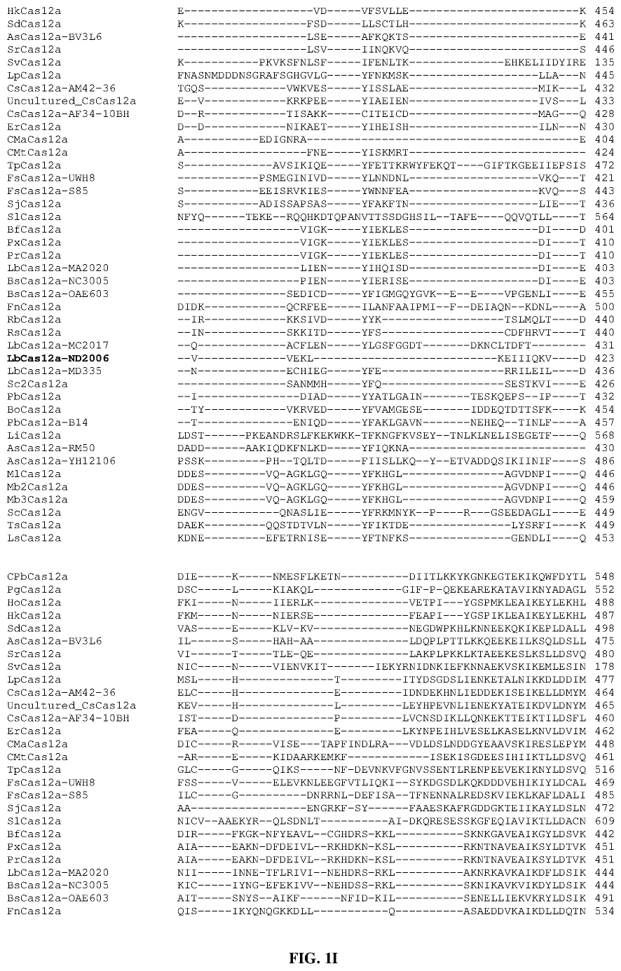

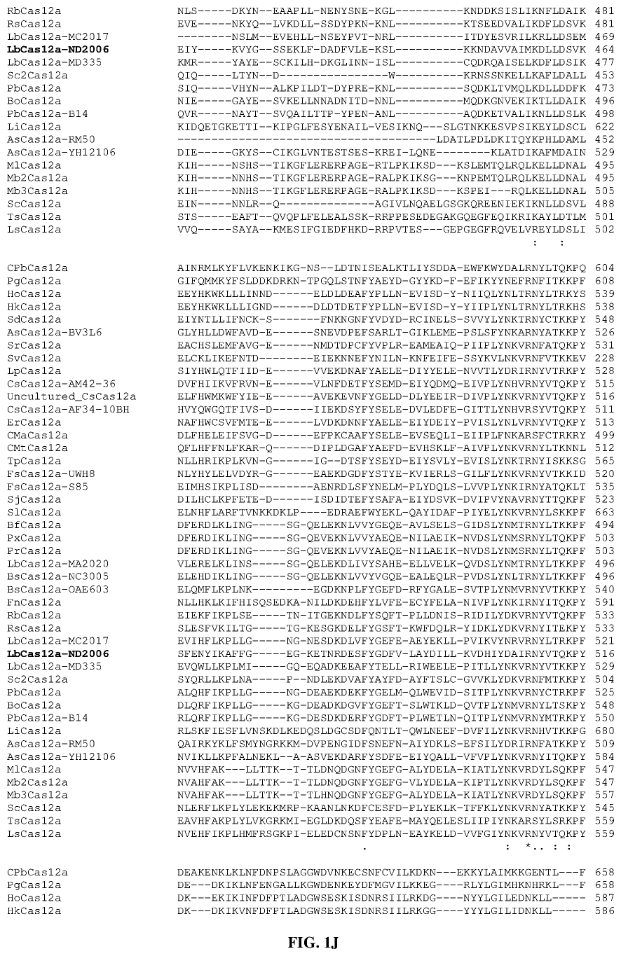

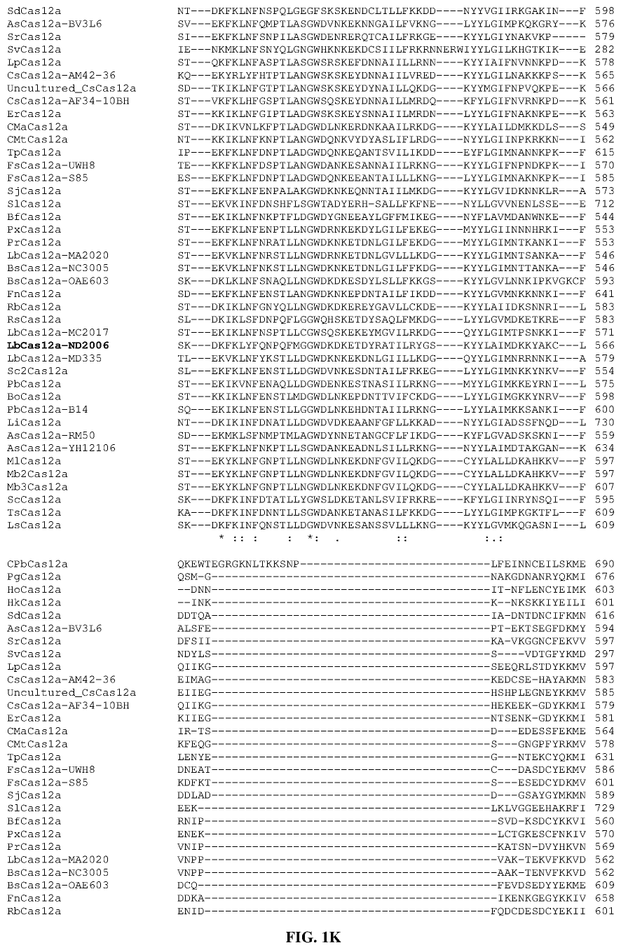

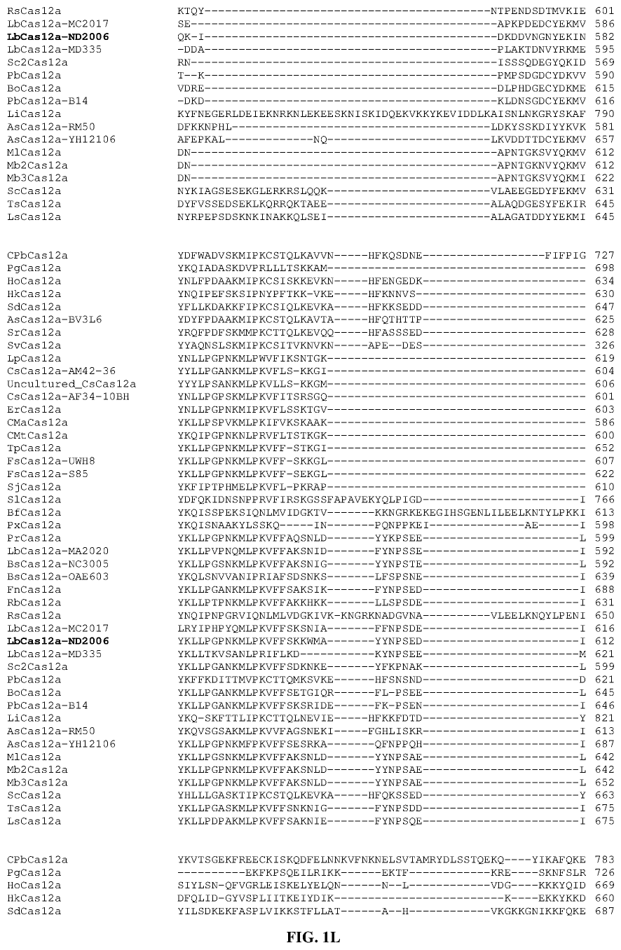

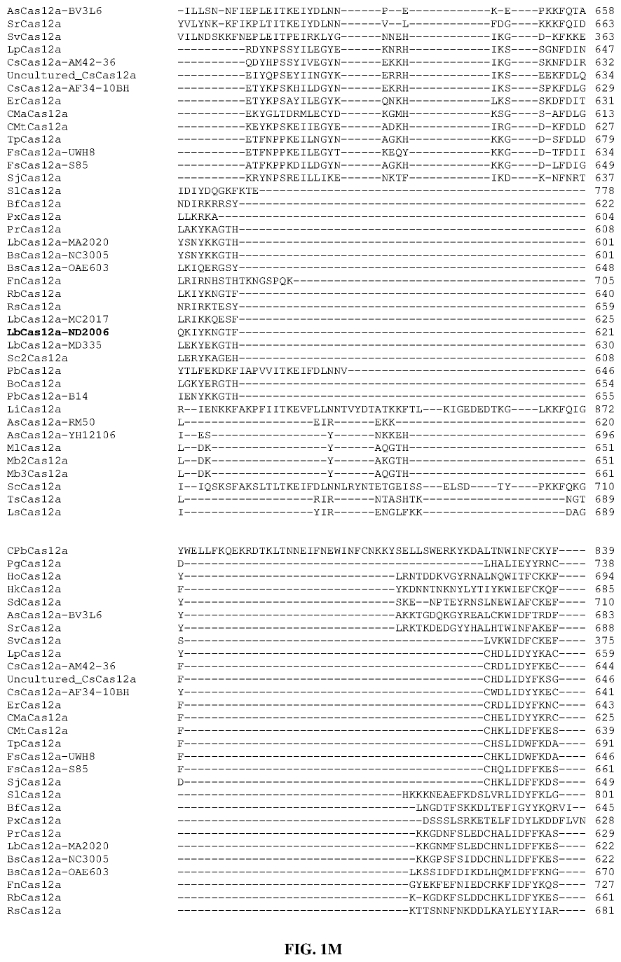

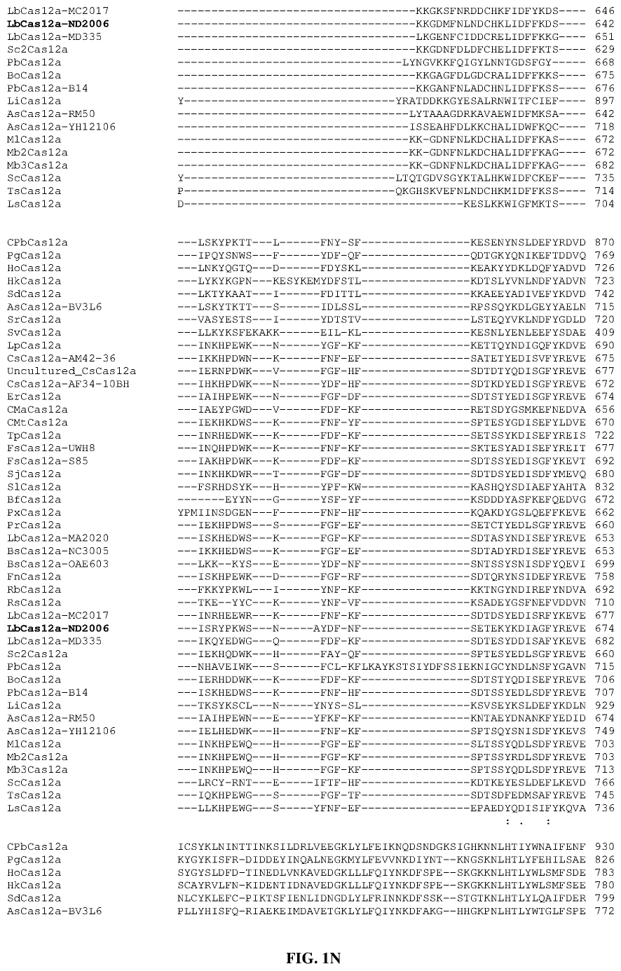

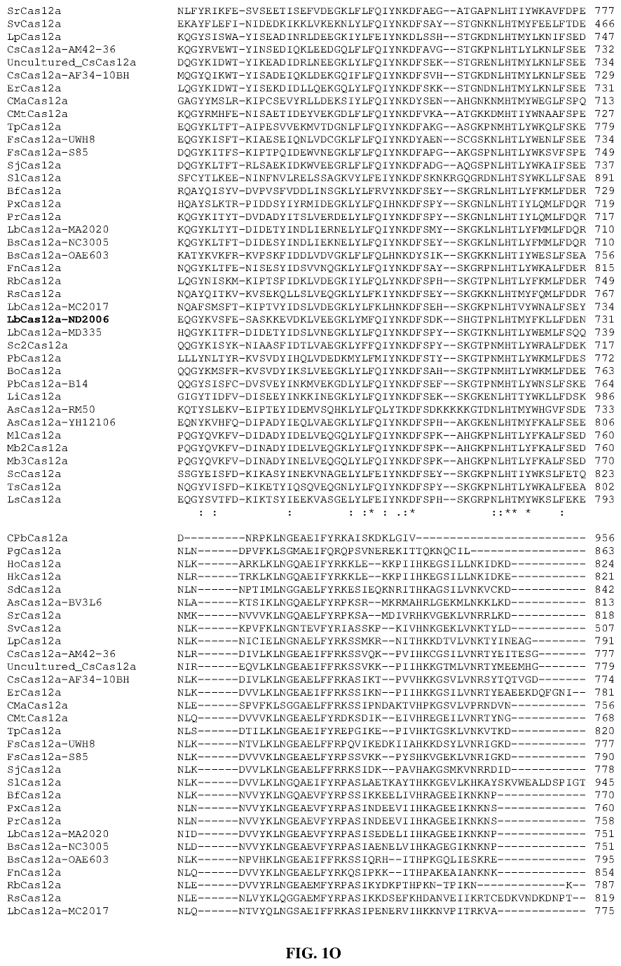

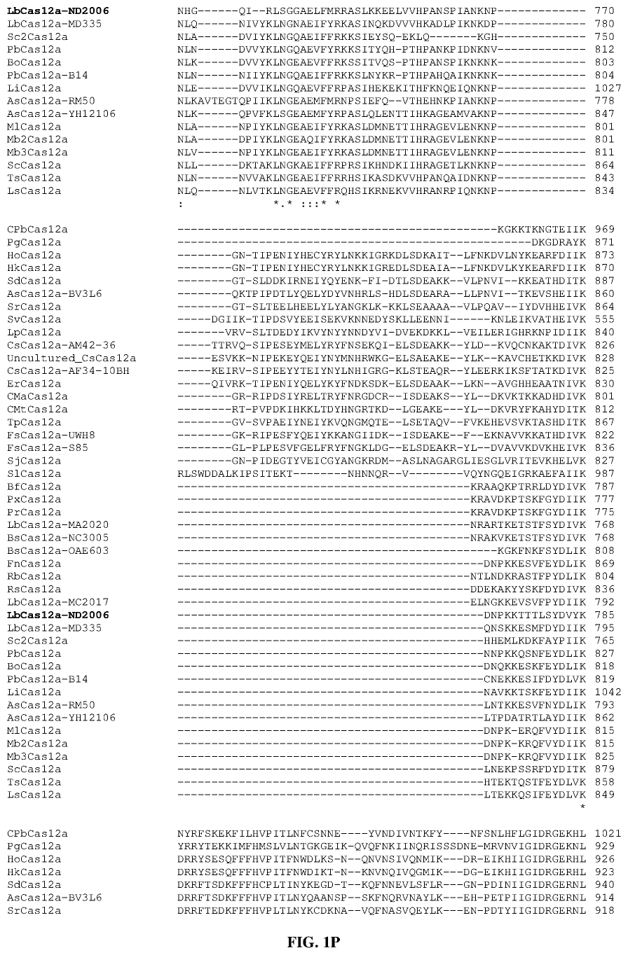

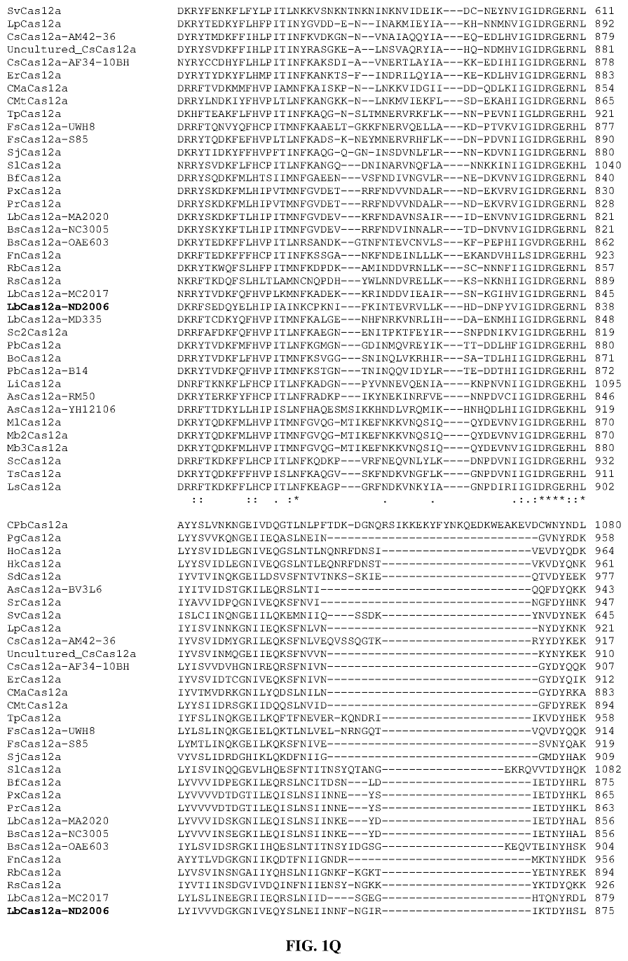

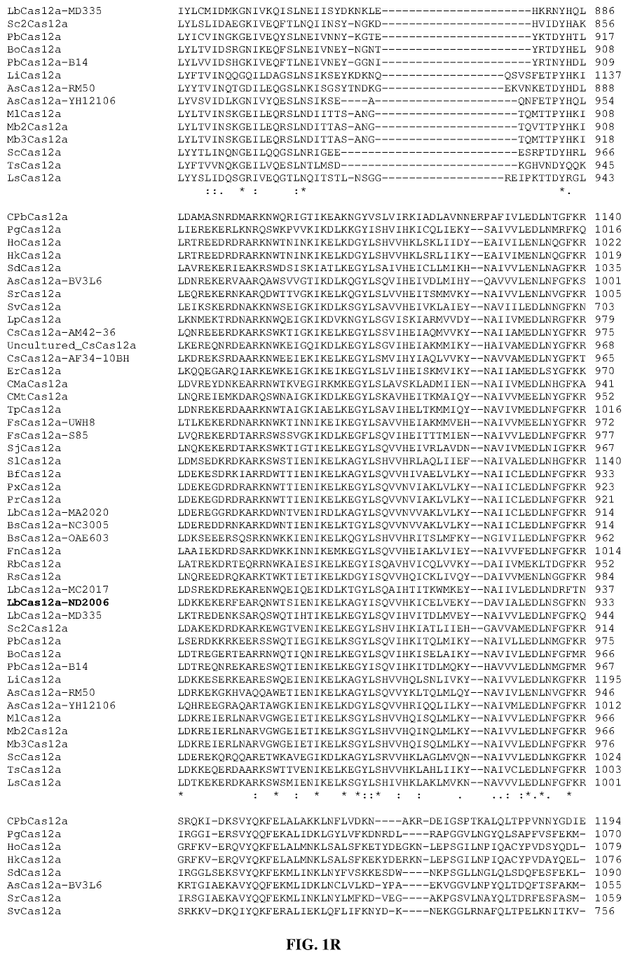

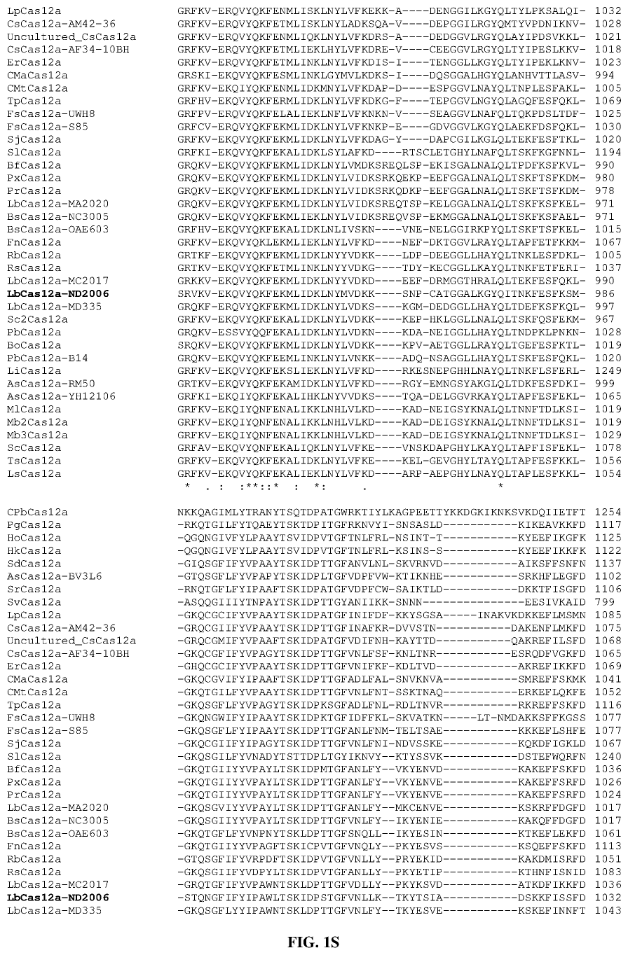

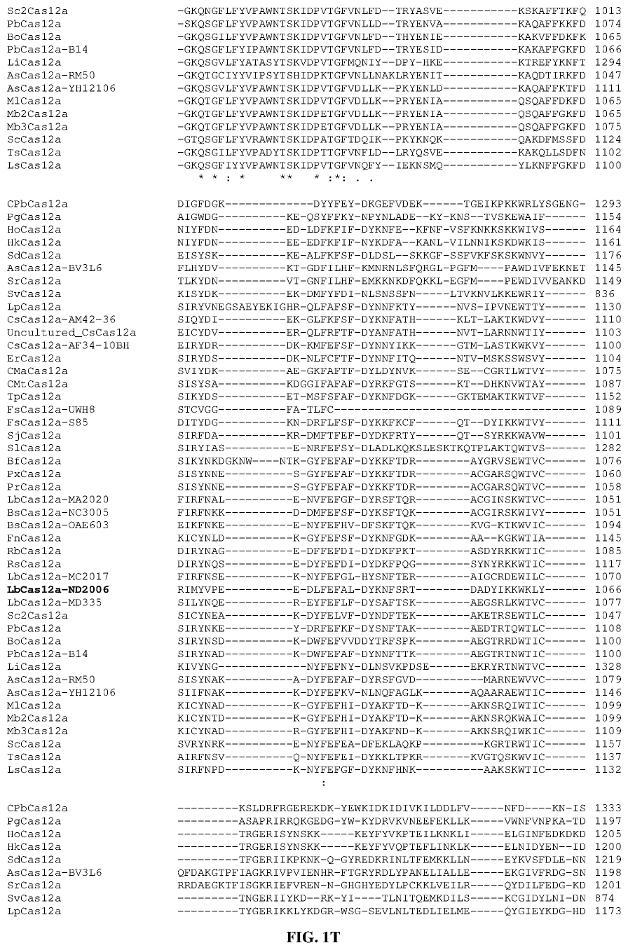

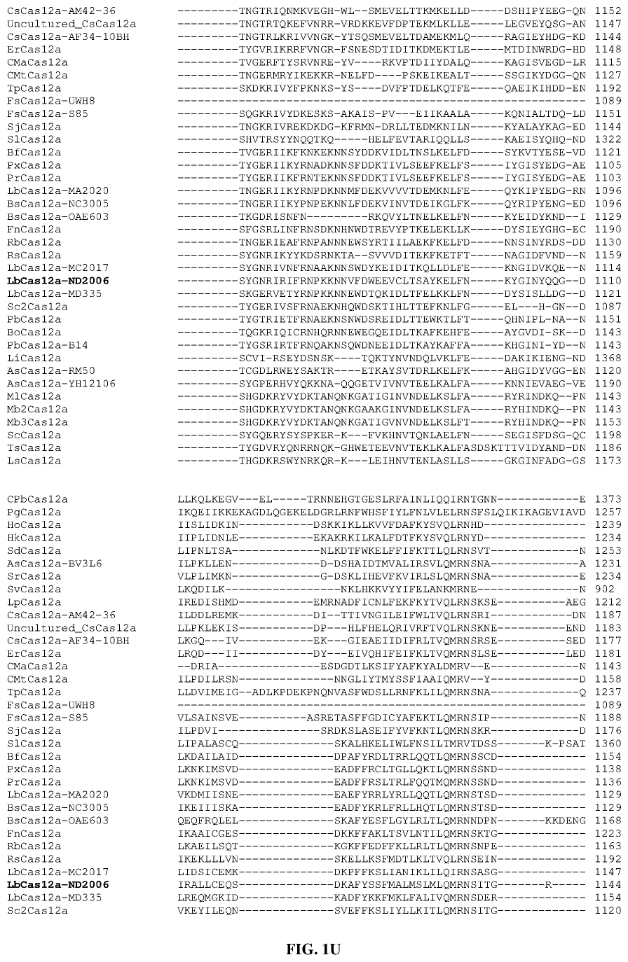

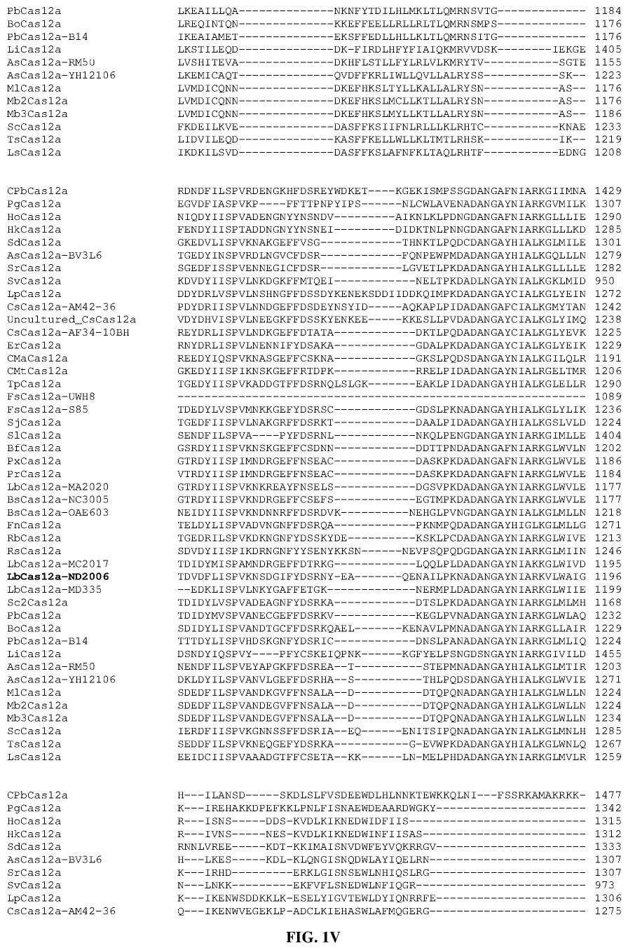

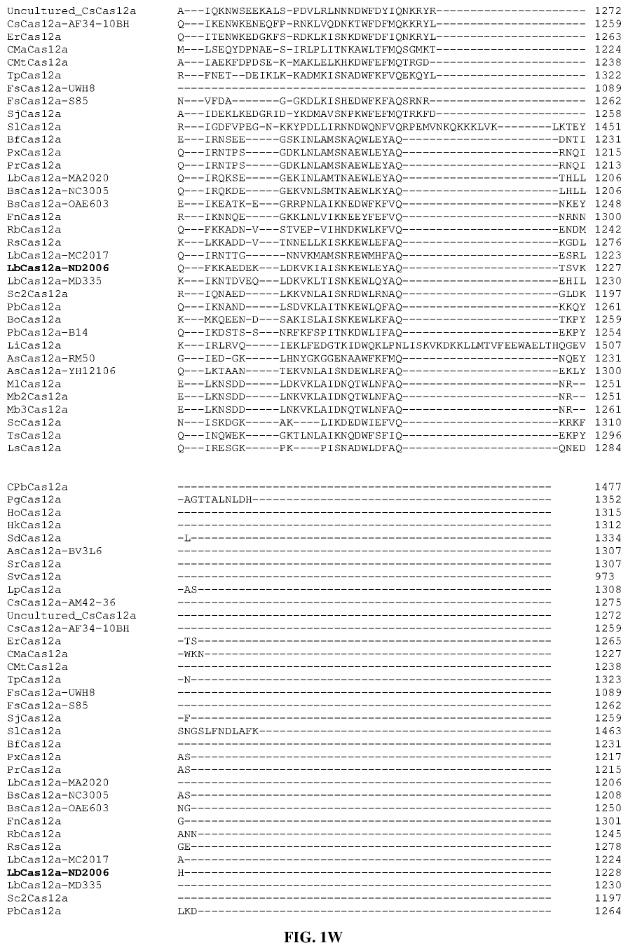

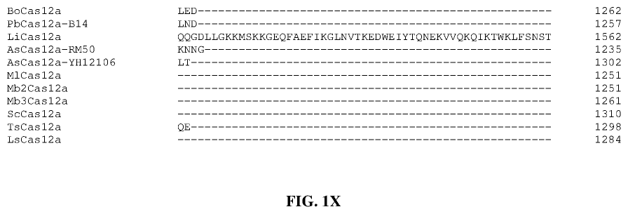

Mutations contemplated herein, with respect to an amino acid sequence, include, without limitation, substitutions, additions, and deletions. An amino acid “substitution” is a change in a single amino acid relative to a reference amino acid sequence. For example, with references to the LbCas12a ND2006 amino acid sequence of SEQ ID NO: 1 of FIGS. 1A-1X, a substitution at position E95 would include any amino acid, other than E, at position 95 (counting from the methionine (M) start codon)—e.g., E95R (R substituted for E) and E95Y (Y substituted for E).

The variant Cas12a endonucleases provided herein, in some aspects, exhibit hyperactivity or low indiscriminate single strand deoxyribonuclease (DNase) activity, described in more detail elsewhere herein.

The activity (e.g., hyperactivity and/or indiscriminate single strand DNase activity) of a variant Cas12a endonuclease may be assessed using any method known in the art. In some embodiments, the activity of a variant Cas12a endonuclease is determined with a gel-based assay. In some embodiments, the activity of a variant Cas12a endonuclease is determined using fluorophores and/or a fluorophore-quencher system. In some embodiments, the activity of a variant Cas12a endonuclease may be assessed using short, labelled oligonucleotides, which measure the activity of Cas9 and CasΦ, respectively (see, e.g., Jinek et al., Science, 2012, 337 (6096): 816-821 and Pausche et al., Science, 2020, 396 (6501): 333-337). In some embodiments, fluorophore-labeled short oligonucleotides are used to assess cleavage on both strands (see, e.g., Stella et al., Cell, 2018, 175:1856-1871). In some embodiments, nickase activity is determined using optical tweezers (see, e.g., Paul et al., bioRxiv, 2021, doi.org/10.1101/2021.06.09.447528). In some embodiments, longer fluorophore-labeled oligonucleotides are used to assess Cas12a cleavage on both strands (see, e.g., Yamano et al., Cell, 2016, 165 (4): 494-962; Cofsky et al., eLife, 2020, 9: e55143). In some embodiments, quencher-fluorophore-labeled single-stranded DNA is used to assess ssDNase activity (see, e.g., Chen et al., Science, 2018, 360 (6387): 436-439). In some embodiments, variant Cas12a endonuclease activity is assessed using single molecule fluorescence resonance energy transfer (FRET) (see, e.g., Son et al., PNAS, 2021, 118 (49): e2113747118). Other methods are contemplated herein.

In embodiments in which an amino acid substitution is exemplified (e.g., E95R), the present disclosure contemplates alternative substitutions having an “equivalent” charge, polarity, and or chemical class (defined by the amino acid side chain). Table 2 provides the 20 naturally-occurring amino acids with a description of corresponding charge, polarity, and chemical class. For example, arginine has an equivalent charge to histidine and lysine; an equivalent polarity to asparagine, glutamine, serine, threonine, tyrosine, aspartic acid, glutamic acid, arginine, histidine, and lysine; and an equivalent chemical class/side chain to histidine and lysine. Thus, using the E95R substitution as an example, E95H and E95K are examples of amino acid substitutions having an equivalent charge; E95N, E95Q, E95S, E95T, E95Y, E95D, E95E, E95H, and E95K are examples of amino acid substitutions having an equivalent polarity, and E95H and E95K are examples of amino acid substitutions having an equivalent chemical class. In some embodiments, a given amino acid substitution is equivalent in charge, polarity, and chemical class. Again, using the E95R substitution as an example, E95H and E95K are examples of amino acid substitutions having an equivalent charge (i.e., positive), an equivalent polarity (i.e., polar), and an equivalent chemical class (i.e., basic).

TABLE 2
Amino Acids

| | | | | | Chemical |
|---|---|---|---|---|---|
| Amino acid | Abbreviation | | Charge | Polarity | Class/Side Chain |
| Alanine | Ala | A | uncharged | nonpolar | aliphatic |
| Glycine | Gly | G | uncharged | nonpolar | aliphatic |
| Isoleucine | Ile | I | uncharged | nonpolar | aliphatic |
| Leucine | Leu | L | uncharged | nonpolar | aliphatic |
| Proline | Pro | P | uncharged | nonpolar | aliphatic |
| Valine | Val | V | uncharged | nonpolar | aliphatic |
| Phenylalanine | Phe | F | uncharged | nonpolar | aromatic |
| Tryptophan | Trp | W | uncharged | nonpolar | aromatic |
| Cysteine | Cys | C | uncharged | nonpolar | sulfur |
| Methionine | Met | M | uncharged | nonpolar | sulfur |
| Asparagine | Asn | N | uncharged | polar | amide |
| Glutamine | Gln | Q | uncharged | polar | acidic |
| Serine | Ser | S | uncharged | polar | hydroxyl |
| Threonine | Thr | T | uncharged | polar | hydroxyl |
| Tyrosine | Tyr | Y | uncharged | polar | aromatic |
| Aspartic acid | Asp | D | negative | polar | acidic |
| Glutamic acid | Glu | E | negative | polar | amide |
| Arginine | Arg | R | positive | polar | basic |
| Histidine | His | H | positive | polar | basic |
| Lysine | Lys | K | positive | polar | basic |

In some embodiments, a variant Cas12a endonuclease comprises a polypeptide sequence that comprises a mutation at an amino acid position corresponding to position E95, E125, N256, R747, H759, N813, K932, N933, S934, V936, S982, or K984 with reference to amino acid position numbering of LbCas12a ND2006 (e.g., SEQ ID NO: 1), optionally wherein the variant Cas12a endonuclease exhibits hyperactivity. In other embodiments, a variant Cas12a endonuclease comprises a polypeptide sequence that comprise a mutation at an amino acid position corresponding to position N256, 1831, K932, N933, S934, V936, Q944, S982, F983, K984, M986, or T988 with reference to amino acid position numbering of LbCas12a ND2006 (e.g., SEQ ID NO: 1), optionally wherein the variant Cas12a endonuclease exhibits hypoactivity. In yet other embodiments, a variant Cas12a endonuclease comprises a polypeptide sequence that comprise a mutation at an amino acid position corresponding to position N813, 1831, K932, N933, S934, V936, Q944, S982, F983, K984, M986, or T988 with reference to amino acid position numbering of LbCas12a ND2006 (e.g., SEQ ID NO: 1), optionally wherein the variant Cas12a endonuclease exhibits low (or no) indiscriminate ssDNase activity. It should be understood that a variant Cas12a endonuclease comprising a mutation at an amino acid position corresponding to a specific position with reference to amino acid position numbering of LbCas12a ND2006 encompasses variants of LbCas12a ND2006 (e.g., SEQ ID NO: 1), as well as variants of Cas12a orthologs of LbCas12a ND2006, including without limitation, variants of any one of the Cas12a endonucleases in Table 1 (e.g., SEQ ID NOs: 2-47). Identification of such “corresponding” amino acid positions can be readily performed by aligning any Cas12a endonuclease amino acid sequence to those examples provided herein, and in particular, with an LbCas12a ND2006 sequence, such as the amino acid sequence of SEQ ID NO: 1, as shown in FIGS. 1A-1X.

FIG. 1A, for example, shows an alignment of various Cas12a homologs, highlighting that a variant Cas12a endonuclease comprising a mutation at an amino acid position corresponding to E95 with reference to amino acid position numbering of LbCas12a ND2006 includes: variant Cas12a endonucleases comprising a mutation at position 196 with reference to amino acid position numbering of AsCas12a BV3L6, and variant Cas12a endonucleases comprising a mutation at position K99 with reference to amino acid position numbering of FnCas12a.

The variant Cas12a endonucleases of the present disclosure may share a certain percent identity relative to a wild-type Cas12a endonuclease. For example, a variant Cas12a endonuclease may comprise an amino acid sequence that includes any one or more mutation(s) (e.g., amino acid substitution(s)) described herein and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence.

In some embodiments, a variant Cas12a endonuclease comprises a polypeptide sequence that comprise a mutation at an amino acid position corresponding to position E95, E125, N256, R747, H759, N813, K932, N933, S934, V936, S982, or K984 with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits hyperactivity.

In some embodiments, a variant Cas12a endonuclease comprises a polypeptide sequence that comprise a mutation at an amino acid position corresponding to position E95R, E95Y, E125A, E125W, N256A, R747Y, H759V, H759D, N813R, N813H, K932L, N933E, N933V, S934Q, V936E, V936M, V936K, S982N, or K984R with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits hyperactivity.

In some embodiments, a variant Cas12a endonuclease comprises a polypeptide sequence that comprise a mutation at an amino acid position corresponding to position N256, 1831, K932, N933, S934, V936, Q944, S982, F983, K984, M986, or T988 with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits hypoactivity.

In some embodiments, a variant Cas12a endonuclease comprises a polypeptide sequence that comprise a mutation at an amino acid position corresponding to position N256K, I831A, I831Y, K932A, K932F, K932H, K932M, K932N, K932Q, K932R, K932S, K932T, K932W, K932Y, N933L, S934W, V936G, Q944D, Q944E, Q944K, Q944M, S982T, S982W, F983G, F983L, K984F, M986G, M986L, M986S, or T988F with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits hypoactivity.

In some embodiments, a variant Cas12a endonuclease comprises a polypeptide sequence that comprise mutations at amino acid positions corresponding to positions K932F and F983L; K932F and T988F; K932R and Q944D; K932R and F983L; K932R and T988F; K932Y and F983L; K932Y and T988F; N933L and Q944M; V936G and Q944D; V936G and S982W; V936G and M986G; V936G and T988F; Q944D and S982W; Q944D and F983L; Q944D and T988F; S982W and F983L; S982W and T988F; or F983G and M986G with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits hypoactivity.

In some embodiments, a variant Cas12a endonuclease comprises a polypeptide sequence that comprise a mutation at an amino acid position corresponding to position N813, 1831, K932, N933, S934, V936, Q944, S982, F983, K984, M986, or T988 with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits low (or no) ssDNase activity.

In some embodiments, a variant Cas12a endonuclease comprises a polypeptide sequence that comprise a mutation at an amino acid position corresponding to position N813H, N813R, N813W, I831A, I831Y, K932A, K932F, K932H, K932M, K932N, K932Q, K932R, K932S, K932T, K932W, K932Y, N933E, N933L, S934K, S934Q, V936E, V936G, Q944D, Q944E, Q944K, S982W, F983G, F983L, K984F, M986F, M986G, or T988F with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits low (or no) ssDNase activity.

In some embodiments, a variant Cas12a endonuclease comprises a polypeptide sequence that comprise mutations at amino acid positions corresponding to positions: N933L and Q944M; or F983G and M986G with reference to amino acid position numbering of LbCas12a and has at least 80% (e.g., at least 80%, at least 85%, at least 90%, at least 95%, or at least 98%) identity to the amino acid sequence of any one of the Cas12a endonucleases in Table 1 (e.g., SEQ ID NOs: 1-47), an ortholog thereof, or other wild-type Cas12a protein sequence. In some embodiments, any one or more of the foregoing variant Cas12a endonucleases exhibits low (or no) ssDNase activity.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids or strings of nucleotides. Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to proteins or genes, for example, such as the Cas12a endonucleases described herein, is defined as the percentage of residues (amino acid or nucleic acid residues) in a first protein or gene sequence that are identical with the residues in a second protein or gene sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular protein or gene have at least 90%, 91%, 92%, 93%, 94%, 95%, 96%, 97%, 98%, 99% but less than 100% sequence identity to that particular wild-type, native, or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and amino acid sequences faster than other optimal global alignment methods, including the Needleman-Wunsch algorithm.

An alignment of the non-limiting examples of wild-type Cas12a endonuclease sequences is provided in FIGS. 1A-1X.

A Cas12 “homolog” refers to a Cas12a endonuclease that has at least some sequence identity (e.g., at least 30%, at least 40%, at least 50%, at least 60%, at least 70%, at least 80%, or at least 90% identity) to a wild-type reference Cas12a endonuclease and exhibits at least one activity exhibited by the wild-type reference Cas12a endonuclease (e.g., cleavage of a double strand or single strand polynucleotide, binding to a crRNA, etc.). For example, the wild-type Cas12a endonuclease exhibits indiscriminate ssDNase activity, cuts ˜14 bp away from the PAM, and possesses RNase activity to self-process pre-crRNA. Further, its cleavage activity results in 5′ staggered overhangs, and its PAM site is 3′ to the targeting binding site. By contrast, a wild-type Cas9 endonuclease does not exhibit indiscriminate ssDNase activity, cuts ˜3-4 bp away from the PAM, and does not possess RNase activity to self-process pre-crRNA (it requires accessory proteins to mediate pre-crRNA processing). Further, Cas9 cleavage activity results in blunt ends, and its PAM site is 5′ to the targeting binding site.

A Cas12a “ortholog” refers to Cas12a genes (and proteins encoded by the genes) inferred to be descended from the same ancestral sequence separated by a speciation event: when a species diverges into two separate species, the copies of a single gene in the two resulting species are said to be orthologous. Orthologs, or orthologous genes, are genes in different species that originated by vertical descent from a single gene of the last common ancestor. Cas12a ortholog can be identified and characterized based on sequence similarities to the present Cas12a system, as has been described with Type II systems, for example. For example, orthologs of Cas12a include the Cas12a endonucleases of Table 1.

### III. Cas12a Endonuclease Hyperactive Variants

Some aspects of the present disclosure relate to hyperactive variant Cas12a endonucleases, i.e., variant Cas12a endonucleases that exhibit hyperactivity. “Hyperactivity” herein refers to polynucleotide cleavage activity of a variant endonuclease that is at least 10% greater than polynucleotide cleavage activity of the wild-type or other reference endonuclease. A hyperactive variant Cas12a endonuclease has a higher reaction speed or initiates a cleavage reaction faster than the corresponding wild-type Cas12a endonuclease. In some embodiments, a hyperactive variant Cas12a endonucleases exhibits cleavage activity that is at least 15%, at least 20%, at least 25%, at least 30%, at least 35%, at least 40%, at least 45%, or at least 50% greater than polynucleotide cleavage activity of the wild-type or other reference endonuclease. See, e.g., Zhang, L. et al. Nat Commun. 2021 Jun. 23; 12 (1): 3908.

In some embodiments, a variant Cas12a endonuclease (a) comprises a mutation at an amino acid position corresponding to position E95, E125, N256, R747, H759, N813, K932, N933, S934, V936, S982, or K984 with reference to amino acid position numbering of LbCas12a and (b) exhibits hyperactivity, optionally wherein the variant Cas12a endonuclease has at least 85%, at least 90%, at least 95%, or at least 98% identity with a wild-type reference Cas12a endonuclease.

In some embodiments, the variant Cas12a endonuclease comprises a polypeptide sequence that comprises a mutation at an amino acid position corresponding to position E95 with reference to amino acid position numbering of LbCas12a ND2006 (e.g., SEQ ID NO: 1). In some embodiments, the mutation is E95R or E95Y. In some embodiments, a variant LbCas12a endonuclease comprises a mutation at position E95 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position E95 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position E125 with reference to amino acid position numbering of LbCas12a ND2006 (e.g., SEQ ID NO: 1). In some embodiments, the mutation is E125A or E125W. In some embodiments, a variant LbCas12a endonuclease comprises a mutation at position E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position N256 with reference to amino acid position numbering of LbCas12a ND2006 (e.g., SEQ ID NO: 1). In some embodiments, the mutation is N256A. In some embodiments, a variant LbCas12a endonuclease comprises a mutation at position N256 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N256 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position R747 with reference to amino acid position numbering of LbCas12a ND2006 (e.g., SEQ ID NO: 1). In some embodiments, the mutation is R747Y. In some embodiments, a variant LbCas12a endonuclease comprises a mutation at position R747 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position R747 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position H759 with reference to amino acid position numbering of LbCas12a ND2006 (e.g., SEQ ID NO: 1). In some embodiments, the mutation is H759V or H759D. In some embodiments, a variant LbCas12a endonuclease comprises a mutation at position H759 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position H759 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position N813 with reference to amino acid position numbering of LbCas12a ND2006 (e.g., SEQ ID NO: 1). In some embodiments, the mutation is N813R or N813H. In some embodiments, a variant LbCas12a endonuclease comprises a mutation at position N813 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N813 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position K932 with reference to amino acid position numbering of LbCas12a ND2006 (e.g., SEQ ID NO: 1). In some embodiments, the mutation is K932L. In some embodiments, a variant LbCas12a endonuclease comprises a mutation at position K932 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K932 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position N933 with reference to amino acid position numbering of LbCas12a ND2006 (e.g., SEQ ID NO: 1). In some embodiments, the mutation is N933E or N933V. In some embodiments, a variant LbCas12a endonuclease comprises a mutation at position N933 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N933 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position S934 with reference to amino acid position numbering of LbCas12a ND2006 (e.g., SEQ ID NO: 1). In some embodiments, the mutation is S934Q. In some embodiments, a variant LbCas12a endonuclease comprises a mutation at position S934 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S934 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position V936 with reference to amino acid position numbering of LbCas12a ND2006 (e.g., SEQ ID NO: 1). In some embodiments, the mutation is V936E, V936M, or V936K. In some embodiments, a variant LbCas12a endonuclease comprises a mutation at position V936 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position V936 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position S982 with reference to amino acid position numbering of LbCas12a ND2006 (e.g., SEQ ID NO: 1). In some embodiments, the mutation is S982N. In some embodiments, a variant LbCas12a endonuclease comprises a mutation at position S982 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S982 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

In some embodiments, the variant Cas12a endonuclease comprises a polypeptide sequence that comprises a mutation at an amino acid position corresponding to position K984 with reference to amino acid position numbering of LbCas12a ND2006 (e.g., SEQ ID NO: 1). In some embodiments, the mutation is K984R. In some embodiments, a variant LbCas12a endonuclease comprises a mutation at position K984 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K984 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hyperactivity.

### Additional Engineered Variant Cas12a Endonucleases With Hyperactivity

In some embodiments, a variant LbCas12a endonuclease comprises an E95 (e.g., E95R or E95Y) substitution and has at least 80% identity to the amino acid sequence of SEQ ID NO: 1. In some embodiments, a variant LbCas12a endonuclease comprises an E95 (e.g., E95R or E95Y) substitution and has at least 85% identity to the amino acid sequence of SEQ ID NO: 1. In some embodiments, a variant LbCas12a endonuclease comprises an E95 (e.g., E95R or E95Y) substitution and has at least 90% identity to the amino acid sequence of SEQ ID NO: 1. In some embodiments, a variant LbCas12a endonuclease comprises an E95 (e.g., E95R or E95Y) substitution and has at least 95% identity to the amino acid sequence of SEQ ID NO: 1. In some embodiments, a variant LbCas12a endonuclease comprises an E95 (e.g., E95R or E95Y) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an E125 (e.g., E125A or E125W) substitution and has at least 80% identity to the amino acid sequence of SEQ ID NO: 1. In some embodiments, a variant LbCas12a endonuclease comprises an E125 (e.g., E125A or E125W) substitution and has at least 85% identity to the amino acid sequence of SEQ ID NO: 1. In some embodiments, a variant LbCas12a endonuclease comprises an E125 (e.g., E125A or E125W) substitution and has at least 90% identity to the amino acid sequence of SEQ ID NO: 1. In some embodiments, a variant LbCas12a endonuclease comprises an E125 (e.g., E125A or E125W) substitution and has at least 95% identity to the amino acid sequence of SEQ ID NO: 1. In some embodiments, a variant LbCas12a endonuclease comprises an E125 (e.g., E125A or E125W) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an N256 (e.g., N256A) substitution and has at least 80% identity to the amino acid sequence of SEQ ID NO: 1. In some embodiments, a variant LbCas12a endonuclease comprises an N256 (e.g., N256A) substitution and has at least 85% identity to the amino acid sequence of SEQ ID NO: 1. In some embodiments, a variant LbCas12a endonuclease comprises an N256 (e.g., N256A) substitution and has at least 90% identity to the amino acid sequence of SEQ ID NO: 1. In some embodiments, a variant LbCas12a endonuclease comprises an N256 (e.g., N256A) substitution and has at least 95% identity to the amino acid sequence of SEQ ID NO: 1. In some embodiments, a variant LbCas12a endonuclease comprises an N256 (e.g., N256A) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an R747 (e.g., R747Y) substitution and has at least 80% identity to the amino acid sequence of SEQ ID NO: 1. In some embodiments, a variant LbCas12a endonuclease comprises an R747 (e.g., R747Y) substitution and has at least 85% identity to the amino acid sequence of SEQ ID NO: 1. In some embodiments, a variant LbCas12a endonuclease comprises an R747 (e.g., R747Y) substitution and has at least 90% identity to the amino acid sequence of SEQ ID NO: 1. In some embodiments, a variant LbCas12a endonuclease comprises an R747 (e.g., R747Y) substitution and has at least 95% identity to the amino acid sequence of SEQ ID NO: 1. In some embodiments, a variant LbCas12a endonuclease comprises an R747 (e.g., R747Y) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an H759 (e.g., H759V or H759D) substitution and has at least 80% identity to the amino acid sequence of SEQ ID NO: 1. In some embodiments, a variant LbCas12a endonuclease comprises an H759 (e.g., H759V or H759D) substitution and has at least 85% identity to the amino acid sequence of SEQ ID NO: 1. In some embodiments, a variant LbCas12a endonuclease comprises an H759 (e.g., H759V or H759D) substitution and has at least 90% identity to the amino acid sequence of SEQ ID NO: 1. In some embodiments, a variant LbCas12a endonuclease comprises an H759 (e.g., H759V or H759D) substitution and has at least 95% identity to the amino acid sequence of SEQ ID NO: 1. In some embodiments, a variant LbCas12a endonuclease comprises an H759 (e.g., H759V or H759D) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an N813 (e.g., N813R or N813H) substitution and has at least 80% identity to the amino acid sequence of SEQ ID NO: 1. In some embodiments, a variant LbCas12a endonuclease comprises an N813 (e.g., N813R or N813H) substitution and has at least 85% identity to the amino acid sequence of SEQ ID NO: 1. In some embodiments, a variant LbCas12a endonuclease comprises an N813 (e.g., N813R or N813H) substitution and has at least 90% identity to the amino acid sequence of SEQ ID NO: 1. In some embodiments, a variant LbCas12a endonuclease comprises an N813 (e.g., N813R or N813H) substitution and has at least 95% identity to the amino acid sequence of SEQ ID NO: 1. In some embodiments, a variant LbCas12a endonuclease comprises an N813 (e.g., N813R or N813H) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an K932 (e.g., K932L) substitution and has at least 80% identity to the amino acid sequence of SEQ ID NO: 1. In some embodiments, a variant LbCas12a endonuclease comprises an K932 (e.g., K932L) substitution and has at least 85% identity to the amino acid sequence of SEQ ID NO: 1. In some embodiments, a variant LbCas12a endonuclease comprises an K932 (e.g., K932L) substitution and has at least 90% identity to the amino acid sequence of SEQ ID NO: 1. In some embodiments, a variant LbCas12a endonuclease comprises an K932 (e.g., K932L) substitution and has at least 95% identity to the amino acid sequence of SEQ ID NO: 1. In some embodiments, a variant LbCas12a endonuclease comprises an K932 (e.g., K932L) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an N933 (e.g., N933E or N933V) substitution and has at least 80% identity to the amino acid sequence of SEQ ID NO: 1. In some embodiments, a variant LbCas12a endonuclease comprises an N933 (e.g., N933E or N933V) substitution and has at least 85% identity to the amino acid sequence of SEQ ID NO: 1. In some embodiments, a variant LbCas12a endonuclease comprises an N933 (e.g., N933E or N933V) substitution and has at least 90% identity to the amino acid sequence of SEQ ID NO: 1. In some embodiments, a variant LbCas12a endonuclease comprises an N933 (e.g., N933E or N933V) substitution and has at least 95% identity to the amino acid sequence of SEQ ID NO: 1. In some embodiments, a variant LbCas12a endonuclease comprises an N933 (e.g., N933E or N933V) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an S934Q (e.g., S934Q) substitution and has at least 80% identity to the amino acid sequence of SEQ ID NO: 1. In some embodiments, a variant LbCas12a endonuclease comprises an S934Q (e.g., S934Q) substitution and has at least 85% identity to the amino acid sequence of SEQ ID NO: 1. In some embodiments, a variant LbCas12a endonuclease comprises an S934Q (e.g., S934Q) substitution and has at least 90% identity to the amino acid sequence of SEQ ID NO: 1. In some embodiments, a variant LbCas12a endonuclease comprises an S934Q (e.g., S934Q) substitution and has at least 95% identity to the amino acid sequence of SEQ ID NO: 1. In some embodiments, a variant LbCas12a endonuclease comprises an S934Q (e.g., S934Q) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an V936 (e.g., V936E, V936M, or V936K) substitution and has at least 80% identity to the amino acid sequence of SEQ ID NO: 1. In some embodiments, a variant LbCas12a endonuclease comprises an V936 (e.g., V936E, V936M, or V936K) substitution and has at least 85% identity to the amino acid sequence of SEQ ID NO: 1. In some embodiments, a variant LbCas12a endonuclease comprises an V936 (e.g., V936E, V936M, or V936K) substitution and has at least 90% identity to the amino acid sequence of SEQ ID NO: 1. In some embodiments, a variant LbCas12a endonuclease comprises an V936 (e.g., V936E, V936M, or V936K) substitution and has at least 95% identity to the amino acid sequence of SEQ ID NO: 1. In some embodiments, a variant LbCas12a endonuclease comprises an V936 (e.g., V936E, V936M, or V936K) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an S982 (e.g., S982N) substitution and has at least 80% identity to the amino acid sequence of SEQ ID NO: 1. In some embodiments, a variant LbCas12a endonuclease comprises an S982 (e.g., S982N) substitution and has at least 85% identity to the amino acid sequence of SEQ ID NO: 1. In some embodiments, a variant LbCas12a endonuclease comprises an S982 (e.g., S982N) substitution and has at least 90% identity to the amino acid sequence of SEQ ID NO: 1. In some embodiments, a variant LbCas12a endonuclease comprises an S982 (e.g., S982N) substitution and has at least 95% identity to the amino acid sequence of SEQ ID NO: 1. In some embodiments, a variant LbCas12a endonuclease comprises an S982 (e.g., S982N) substitution and has at least 98% identity to the amino acid sequence of SEQ ID NO: 1. Any one or more of the foregoing variant Cas12a endonucleases may exhibit hyperactivity.

In some embodiments, a variant LbCas12a endonuclease comprises an K984 (e.g., K984R) substitution and has at least 80% identity to the amino acid sequence of SEQ ID NO: 1. In some embodiments, a variant LbCas12a endonuclease comprises an K984 (e.g., K984R) substitution and has at least 85% identity to the amino acid sequence of SEQ ID NO: 1. In some embodiments, a variant LbCas12a endonuclease comprises an K984 (e.g., K984R) substitution and has at least 90% identity to the amino acid sequence of SEQ ID NO: 1. In some embodiments, a variant LbCas12a endonuclease comprises an K984 (e.g., K984R) substitution and has at least 95% identity to the amino acid sequence of SEQ ID NO: 1. In some embodiments, a variant LbCas12a endonuclease comprises an K984 (e.g., K984R) substitution and has at least 98% identity to the amino acid sequence of SEQ ID NO: 1. Any one or more of the foregoing variant Cas12a endonucleases may exhibit hyperactivity.

### IV. Cas12a Endonuclease Hypoactive Variants

Other aspects of the present disclosure provide hypoactive variant Cas12a endonucleases, i.e., variant Cas12a endonucleases that exhibit hypoactivity. “Hypoactivity” herein refers to polynucleotide cleavage activity of a variant endonuclease that is at least 10% lower than polynucleotide cleavage activity of the wild-type or other reference endonuclease. In some embodiments, a hypoactive variant Cas12a endonucleases exhibits cleavage activity that is at least 15%, at least 20%, at least 25%, at least 30%, at least 35%, at least 40%, at least 45%, or at least 50% lower than polynucleotide cleavage activity of the wild-type or other reference endonuclease.

In some embodiments, the variant Cas12a endonuclease comprises a polypeptide sequence that comprises a mutation at an amino acid position corresponding to position N256 with reference to amino acid position numbering of LbCas12a ND2006 (e.g., SEQ ID NO: 1). In some embodiments, the mutation is N256K. In some embodiments, a variant LbCas12a endonuclease comprises a mutation at position N256 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N256 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position I831 with reference to amino acid position numbering of LbCas12a ND2006 (e.g., SEQ ID NO: 1). In some embodiments, the mutation is I831A or I831Y. In some embodiments, a variant LbCas12a endonuclease comprises a mutation at position I831 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position I831 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position K932 with reference to amino acid position numbering of LbCas12a ND2006 (e.g., SEQ ID NO: 1). In some embodiments, the mutation is K932A, K932F, K932H, K932M, K932N, K932Q, K932R, K932S, K932T, K932W, or K932Y. In some embodiments, a variant LbCas12a endonuclease comprises a mutation at position K932 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K932 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position N933 with reference to amino acid position numbering of LbCas12a ND2006 (e.g., SEQ ID NO: 1). In some embodiments, the mutation is N933L. In some embodiments, a variant LbCas12a endonuclease comprises a mutation at position N933 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N933 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position S934 with reference to amino acid position numbering of LbCas12a ND2006 (e.g., SEQ ID NO: 1). In some embodiments, the mutation is S934W. In some embodiments, a variant LbCas12a endonuclease comprises a mutation at position S934 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S934 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position V936 with reference to amino acid position numbering of LbCas12a ND2006 (e.g., SEQ ID NO: 1). In some embodiments, the mutation is V936G. In some embodiments, a variant LbCas12a endonuclease comprises a mutation at position V936 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position V936 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position Q944 with reference to amino acid position numbering of LbCas12a ND2006 (e.g., SEQ ID NO: 1). In some embodiments, the mutation is Q944D, Q944E, Q944K, or Q944M. In some embodiments, a variant LbCas12a endonuclease comprises a mutation at position Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position S982 with reference to amino acid position numbering of LbCas12a ND2006 (e.g., SEQ ID NO: 1). In some embodiments, the mutation is S982T or S982W. In some embodiments, a variant LbCas12a endonuclease comprises a mutation at position S982 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S982 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position F983 with reference to amino acid position numbering of LbCas12a ND2006 (e.g., SEQ ID NO: 1). In some embodiments, the mutation is F983G or F983L. In some embodiments, a variant LbCas12a endonuclease comprises a mutation at position F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position K984 with reference to amino acid position numbering of LbCas12a ND2006 (e.g., SEQ ID NO: 1). In some embodiments, the mutation is K984F. In some embodiments, a variant LbCas12a endonuclease comprises a mutation at position K984 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K984 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position M986 with reference to amino acid position numbering of LbCas12a ND2006 (e.g., SEQ ID NO: 1). In some embodiments, the mutation is M986G, M986L, or M986S. In some embodiments, a variant LbCas12a endonuclease comprises a mutation at position M986 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position M986 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises a mutation at an amino acid position corresponding to position T988 with reference to amino acid position numbering of LbCas12a ND2006 (e.g., SEQ ID NO: 1). In some embodiments, the mutation is T988F. In some embodiments, a variant LbCas12a endonuclease comprises a mutation at position T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and F983 with reference to amino acid position numbering of LbCas12a ND2006 (e.g., SEQ ID NO: 1). In some embodiments, the mutations are K932F and F983L. In some embodiments, a variant LbCas12a endonuclease comprises mutations at positions K932 and F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and T988 with reference to amino acid position numbering of LbCas12a ND2006 (e.g., SEQ ID NO: 1). In some embodiments, the mutations are K932F and T988F. In some embodiments, a variant LbCas12a endonuclease comprises mutations at positions K932 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and Q944 with reference to amino acid position numbering of LbCas12a ND2006 (e.g., SEQ ID NO: 1). In some embodiments, the mutations are K932R and Q944D. In some embodiments, a variant LbCas12a endonuclease comprises mutations at positions K932 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and F983 with reference to amino acid position numbering of LbCas12a ND2006 (e.g., SEQ ID NO: 1). In some embodiments, the mutations are K932R and F983L. In some embodiments, a variant LbCas12a endonuclease comprises mutations at positions K932 and F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and T988 with reference to amino acid position numbering of LbCas12a ND2006 (e.g., SEQ ID NO: 1). In some embodiments, the mutations are K932R and T988F. In some embodiments, a variant LbCas12a endonuclease comprises mutations at positions K932 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and F983 with reference to amino acid position numbering of LbCas12a ND2006 (e.g., SEQ ID NO: 1). In some embodiments, the mutations are K932Y and F983L. In some embodiments, a variant LbCas12a endonuclease comprises mutations at positions K932 and F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions K932 and T988 with reference to amino acid position numbering of LbCas12a ND2006 (e.g., SEQ ID NO: 1). In some embodiments, the mutations are K932Y and T988F. In some embodiments, a variant LbCas12a endonuclease comprises mutations at positions K932 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions N933 and Q944 with reference to amino acid position numbering of LbCas12a ND2006 (e.g., SEQ ID NO: 1). In some embodiments, the mutations are N933L and Q944M. In some embodiments, a variant LbCas12a endonuclease comprises mutations at positions N933 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions N933 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions V936 and Q944 with reference to amino acid position numbering of LbCas12a ND2006 (e.g., SEQ ID NO: 1). In some embodiments, the mutations are V936G and Q944D. In some embodiments, a variant LbCas12a endonuclease comprises mutations at positions V936 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions V936 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions V936 and S982 with reference to amino acid position numbering of LbCas12a ND2006 (e.g., SEQ ID NO: 1). In some embodiments, the mutations are V936G and S982W. In some embodiments, a variant LbCas12a endonuclease comprises mutations at positions V936 and S982 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions V936 and S982 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions V936 and M986 with reference to amino acid position numbering of LbCas12a ND2006 (e.g., SEQ ID NO: 1). In some embodiments, the mutations are V936G and M986G. In some embodiments, a variant LbCas12a endonuclease comprises mutations at positions V936 and M986 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions V936 and M986 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions V936 and T988 with reference to amino acid position numbering of LbCas12a ND2006 (e.g., SEQ ID NO: 1). In some embodiments, the mutations are V936G and T988F. In some embodiments, a variant LbCas12a endonuclease comprises mutations at positions V936 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions V936 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions Q944 and S982 with reference to amino acid position numbering of LbCas12a ND2006 (e.g., SEQ ID NO: 1). In some embodiments, the mutations are Q944D and S982W. In some embodiments, a variant LbCas12a endonuclease comprises mutations at positions Q944 and S982 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions Q944 and S982 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions Q944 and F983 with reference to amino acid position numbering of LbCas12a ND2006 (e.g., SEQ ID NO: 1). In some embodiments, the mutations are Q944D and F983L. In some embodiments, a variant LbCas12a endonuclease comprises mutations at positions Q944 and F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions Q944 and F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions Q944 and T988 with reference to amino acid position numbering of LbCas12a ND2006 (e.g., SEQ ID NO: 1). In some embodiments, the mutations are Q944D and T988F. In some embodiments, a variant LbCas12a endonuclease comprises mutations at positions Q944 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions Q944 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions S982 and F983 with reference to amino acid position numbering of LbCas12a ND2006 (e.g., SEQ ID NO: 1). In some embodiments, the mutations are S982W and F983L. In some embodiments, a variant LbCas12a endonuclease comprises mutations at positions S982 and F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions S982 and F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions S982 and T988 with reference to amino acid position numbering of LbCas12a ND2006 (e.g., SEQ ID NO: 1). In some embodiments, the mutations are S982W and T988F. In some embodiments, a variant LbCas12a endonuclease comprises mutations at positions S982 and T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions S982 and T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

In some embodiments, the variant Cas12a endonuclease comprises a polypeptide sequence that comprises mutations at amino acid positions corresponding to positions F983 and M986 with reference to amino acid position numbering of LbCas12a ND2006 (e.g., SEQ ID NO: 1). In some embodiments, the mutations are F983G and M986G. In some embodiments, a variant LbCas12a endonuclease comprises mutations at positions F983 and M986 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions F983 and M986 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit hypoactivity.

### Additional Engineered Variant Cas12a Endonucleases With Hypoactivity

In some embodiments, a variant LbCas12a endonuclease comprises an N256 (e.g., N256K) substitution and has at least 80% identity to the amino acid sequence of SEQ ID NO: 1. In some embodiments, a variant LbCas12a endonuclease comprises an N256 (e.g., N256K) substitution and has at least 85% identity to the amino acid sequence of SEQ ID NO: 1. In some embodiments, a variant LbCas12a endonuclease comprises an N256 (e.g., N256K) substitution and has at least 90% identity to the amino acid sequence of SEQ ID NO: 1. In some embodiments, a variant LbCas12a endonuclease comprises an N256 (e.g., N256K) substitution and has at least 95% identity to the amino acid sequence of SEQ ID NO: 1. In some embodiments, a variant LbCas12a endonuclease comprises an N256 (e.g., N256K) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an I831 (e.g., I831A or I831Y) substitution and has at least 80% identity to the amino acid sequence of SEQ ID NO: 1. In some embodiments, a variant LbCas12a endonuclease comprises an I831 (e.g., I831A or I831Y) substitution and has at least 85% identity to the amino acid sequence of SEQ ID NO: 1. In some embodiments, a variant LbCas12a endonuclease comprises an I831 (e.g., I831A or I831Y) substitution and has at least 90% identity to the amino acid sequence of SEQ ID NO: 1. In some embodiments, a variant LbCas12a endonuclease comprises an I831 (e.g., I831A or I831Y) substitution and has at least 95% identity to the amino acid sequence of SEQ ID NO: 1. In some embodiments, a variant LbCas12a endonuclease comprises an I831 (e.g., I831A or I831Y) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932 (e.g., K932A, K932F, K932H, K932M, K932N, K932Q, K932R, K932S, K932T, K932W, or K932Y) substitution and has at least 80% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 85% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 90% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 95% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an N933 (e.g., N933L) substitution and has at least 80% identity to the amino acid sequence of SEQ ID NO: 1. In some embodiments, a variant LbCas12a endonuclease comprises an N933 (e.g., N933L) substitution and has at least 85% identity to the amino acid sequence of SEQ ID NO: 1. In some embodiments, a variant LbCas12a endonuclease comprises an N933 (e.g., N933L) substitution and has at least 90% identity to the amino acid sequence of SEQ ID NO: 1. In some embodiments, a variant LbCas12a endonuclease comprises an N933 (e.g., N933L) substitution and has at least 95% identity to the amino acid sequence of SEQ ID NO: 1. In some embodiments, a variant LbCas12a endonuclease comprises an N933 (e.g., N933L) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an S934 (e.g., S934W) substitution and has at least 80% identity to the amino acid sequence of SEQ ID NO: 1. In some embodiments, a variant LbCas12a endonuclease comprises an S934 (e.g., S934W) substitution and has at least 85% identity to the amino acid sequence of SEQ ID NO: 1. In some embodiments, a variant LbCas12a endonuclease comprises an S934 (e.g., S934W) substitution and has at least 90% identity to the amino acid sequence of SEQ ID NO: 1. In some embodiments, a variant LbCas12a endonuclease comprises an S934 (e.g., S934W) substitution and has at least 95% identity to the amino acid sequence of SEQ ID NO: 1. In some embodiments, a variant LbCas12a endonuclease comprises an S934 (e.g., S934W) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an V936 (e.g., V936G) substitution and has at least 80% identity to the amino acid sequence of SEQ ID NO: 1. In some embodiments, a variant LbCas12a endonuclease comprises an V936 (e.g., V936G) substitution and has at least 85% identity to the amino acid sequence of SEQ ID NO: 1. In some embodiments, a variant LbCas12a endonuclease comprises an V936 (e.g., V936G) substitution and has at least 90% identity to the amino acid sequence of SEQ ID NO: 1. In some embodiments, a variant LbCas12a endonuclease comprises an V936 (e.g., V936G) substitution and has at least 95% identity to the amino acid sequence of SEQ ID NO: 1. In some embodiments, a variant LbCas12a endonuclease comprises an V936 (e.g., V936G) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an Q944 (e.g., Q944D, Q944E, Q944K, or Q944M) substitution and has at least 80% identity to the amino acid sequence of SEQ ID NO: 1. In some embodiments, a variant LbCas12a endonuclease comprises an Q944 (e.g., Q944D, Q944E, Q944K, or Q944M) substitution and has at least 85% identity to the amino acid sequence of SEQ ID NO: 1. In some embodiments, a variant LbCas12a endonuclease comprises an Q944 (e.g., Q944D, Q944E, Q944K, or Q944M) substitution and has at least 90% identity to the amino acid sequence of SEQ ID NO: 1. In some embodiments, a variant LbCas12a endonuclease comprises an Q944 (e.g., Q944D, Q944E, Q944K, or Q944M) substitution and has at least 95% identity to the amino acid sequence of SEQ ID NO: 1. In some embodiments, a variant LbCas12a endonuclease comprises an Q944 (e.g., Q944D, Q944E, Q944K, or Q944M) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an S982 (e.g., S982T or S982W) substitution and has at least 80% identity to the amino acid sequence of SEQ ID NO: 1. In some embodiments, a variant LbCas12a endonuclease comprises an S982 (e.g., S982T or S982W) substitution and has at least 85% identity to the amino acid sequence of SEQ ID NO: 1. In some embodiments, a variant LbCas12a endonuclease comprises an S982 (e.g., S982T or S982W) substitution and has at least 90% identity to the amino acid sequence of SEQ ID NO: 1. In some embodiments, a variant LbCas12a endonuclease comprises an S982 (e.g., S982T or S982W) substitution and has at least 95% identity to the amino acid sequence of SEQ ID NO: 1. In some embodiments, a variant LbCas12a endonuclease comprises an S982 (e.g., S982T or S982W) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an F983 (e.g., F983G or F983L) substitution and has at least 80% identity to the amino acid sequence of SEQ ID NO: 1. In some embodiments, a variant LbCas12a endonuclease comprises an F983 (e.g., F983G or F983L) substitution and has at least 85% identity to the amino acid sequence of SEQ ID NO: 1. In some embodiments, a variant LbCas12a endonuclease comprises an F983 (e.g., F983G or F983L) substitution and has at least 90% identity to the amino acid sequence of SEQ ID NO: 1. In some embodiments, a variant LbCas12a endonuclease comprises an F983 (e.g., F983G or F983L) substitution and has at least 95% identity to the amino acid sequence of SEQ ID NO: 1. In some embodiments, a variant LbCas12a endonuclease comprises an F983 (e.g., F983G or F983L) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K984 (e.g., K984F) substitution and has at least 80% identity to the amino acid sequence of SEQ ID NO: 1. In some embodiments, a variant LbCas12a endonuclease comprises an K984 (e.g., K984F) substitution and has at least 85% identity to the amino acid sequence of SEQ ID NO: 1. In some embodiments, a variant LbCas12a endonuclease comprises an K984 (e.g., K984F) substitution and has at least 90% identity to the amino acid sequence of SEQ ID NO: 1. In some embodiments, a variant LbCas12a endonuclease comprises an K984 (e.g., K984F) substitution and has at least 95% identity to the amino acid sequence of SEQ ID NO: 1. In some embodiments, a variant LbCas12a endonuclease comprises an K984 (e.g., K984F) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an M986 (e.g., M986G, M986L, or M986S) substitution and has at least 80% identity to the amino acid sequence of SEQ ID NO: 1. In some embodiments, a variant LbCas12a endonuclease comprises an M986 (e.g., M986G, M986L, or M986S) substitution and has at least 85% identity to the amino acid sequence of SEQ ID NO: 1. In some embodiments, a variant LbCas12a endonuclease comprises an M986 (e.g., M986G, M986L, or M986S) substitution and has at least 90% identity to the amino acid sequence of SEQ ID NO: 1. In some embodiments, a variant LbCas12a endonuclease comprises an M986 (e.g., M986G, M986L, or M986S) substitution and has at least 95% identity to the amino acid sequence of SEQ ID NO: 1. In some embodiments, a variant LbCas12a endonuclease comprises an M986 (e.g., M986G, M986L, or M986S) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an T988 (e.g., T988F) substitution and has at least 80% identity to the amino acid sequence of SEQ ID NO: 1. In some embodiments, a variant LbCas12a endonuclease comprises an T988 (e.g., T988F) substitution and has at least 85% identity to the amino acid sequence of SEQ ID NO: 1. In some embodiments, a variant LbCas12a endonuclease comprises an T988 (e.g., T988F) substitution and has at least 90% identity to the amino acid sequence of SEQ ID NO: 1. In some embodiments, a variant LbCas12a endonuclease comprises an T988 (e.g., T988F) substitution and has at least 95% identity to the amino acid sequence of SEQ ID NO: 1. In some embodiments, a variant LbCas12a endonuclease comprises an T988 (e.g., T988F) substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932F and F983L substitution and has at least 80% identity to the amino acid sequence of SEQ ID NO: 1. In some embodiments, a variant LbCas12a endonuclease comprises an K932F and F983L substitution and has at least 85% identity to the amino acid sequence of SEQ ID NO: 1. In some embodiments, a variant LbCas12a endonuclease comprises an K932F and F983L substitution and has at least 90% identity to the amino acid sequence of SEQ ID NO: 1. In some embodiments, a variant LbCas12a endonuclease comprises an K932F and F983L substitution and has at least 95% identity to the amino acid sequence of SEQ ID NO: 1. In some embodiments, a variant LbCas12a endonuclease comprises an K932F and F983L substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932F and T988F substitution and has at least 80% identity to the amino acid sequence of SEQ ID NO: 1. In some embodiments, a variant LbCas12a endonuclease comprises an K932F and T988F substitution and has at least 85% identity to the amino acid sequence of SEQ ID NO: 1. In some embodiments, a variant LbCas12a endonuclease comprises an K932F and T988F substitution and has at least 90% identity to the amino acid sequence of SEQ ID NO: 1. In some embodiments, a variant LbCas12a endonuclease comprises an K932F and T988F substitution and has at least 95% identity to the amino acid sequence of SEQ ID NO: 1. In some embodiments, a variant LbCas12a endonuclease comprises an K932F and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932R and Q944D substitution and has at least 80% identity to the amino acid sequence of SEQ ID NO: 1. In some embodiments, a variant LbCas12a endonuclease comprises an K932R and Q944D substitution and has at least 85% identity to the amino acid sequence of SEQ ID NO: 1. In some embodiments, a variant LbCas12a endonuclease comprises an K932R and Q944D substitution and has at least 90% identity to the amino acid sequence of SEQ ID NO: 1. In some embodiments, a variant LbCas12a endonuclease comprises an K932R and Q944D substitution and has at least 95% identity to the amino acid sequence of SEQ ID NO: 1. In some embodiments, a variant LbCas12a endonuclease comprises an K932R and Q944D substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932R and F983L substitution and has at least 80% identity to the amino acid sequence of SEQ ID NO: 1. In some embodiments, a variant LbCas12a endonuclease comprises an K932R and F983L substitution and has at least 85% identity to the amino acid sequence of SEQ ID NO: 1. In some embodiments, a variant LbCas12a endonuclease comprises an K932R and F983L substitution and has at least 90% identity to the amino acid sequence of SEQ ID NO: 1. In some embodiments, a variant LbCas12a endonuclease comprises an K932R and F983L substitution and has at least 95% identity to the amino acid sequence of SEQ ID NO: 1. In some embodiments, a variant LbCas12a endonuclease comprises an K932R and F983L substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932R and T988F substitution and has at least 80% identity to the amino acid sequence of SEQ ID NO: 1. In some embodiments, a variant LbCas12a endonuclease comprises an K932R and T988F substitution and has at least 85% identity to the amino acid sequence of SEQ ID NO: 1. In some embodiments, a variant LbCas12a endonuclease comprises an K932R and T988F substitution and has at least 90% identity to the amino acid sequence of SEQ ID NO: 1. In some embodiments, a variant LbCas12a endonuclease comprises an K932R and T988F substitution and has at least 95% identity to the amino acid sequence of SEQ ID NO: 1. In some embodiments, a variant LbCas12a endonuclease comprises an K932R and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932Y and F983L substitution and has at least 80% identity to the amino acid sequence of SEQ ID NO: 1. In some embodiments, a variant LbCas12a endonuclease comprises an K932Y and F983L substitution and has at least 85% identity to the amino acid sequence of SEQ ID NO: 1. In some embodiments, a variant LbCas12a endonuclease comprises an K932Y and F983L substitution and has at least 90% identity to the amino acid sequence of SEQ ID NO: 1. In some embodiments, a variant LbCas12a endonuclease comprises an K932Y and F983L substitution and has at least 95% identity to the amino acid sequence of SEQ ID NO: 1. In some embodiments, a variant LbCas12a endonuclease comprises an K932Y and F983L substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an K932Y and T988F substitution and has at least 80% identity to the amino acid sequence of SEQ ID NO: 1. In some embodiments, a variant LbCas12a endonuclease comprises an K932Y and T988F substitution and has at least 85% identity to the amino acid sequence of SEQ ID NO: 1. In some embodiments, a variant LbCas12a endonuclease comprises an K932Y and T988F substitution and has at least 90% identity to the amino acid sequence of SEQ ID NO: 1. In some embodiments, a variant LbCas12a endonuclease comprises an K932Y and T988F substitution and has at least 95% identity to the amino acid sequence of SEQ ID NO: 1. In some embodiments, a variant LbCas12a endonuclease comprises an K932Y and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an N933L and Q944M substitution and has at least 80% identity to the amino acid sequence of SEQ ID NO: 1. In some embodiments, a variant LbCas12a endonuclease comprises an N933L and Q944M substitution and has at least 85% identity to the amino acid sequence of SEQ ID NO: 1. In some embodiments, a variant LbCas12a endonuclease comprises an N933L and Q944M substitution and has at least 90% identity to the amino acid sequence of SEQ ID NO: 1. In some embodiments, a variant LbCas12a endonuclease comprises an N933L and Q944M substitution and has at least 95% identity to the amino acid sequence of SEQ ID NO: 1. In some embodiments, a variant LbCas12a endonuclease comprises an N933L and Q944M substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an V936G and Q944D substitution and has at least 80% identity to the amino acid sequence of SEQ ID NO: 1. In some embodiments, a variant LbCas12a endonuclease comprises an V936G and Q944D substitution and has at least 85% identity to the amino acid sequence of SEQ ID NO: 1. In some embodiments, a variant LbCas12a endonuclease comprises an V936G and Q944D substitution and has at least 90% identity to the amino acid sequence of SEQ ID NO: 1. In some embodiments, a variant LbCas12a endonuclease comprises an V936G and Q944D substitution and has at least 95% identity to the amino acid sequence of SEQ ID NO: 1. In some embodiments, a variant LbCas12a endonuclease comprises an V936G and Q944D substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an V936G and S982W substitution and has at least 80% identity to the amino acid sequence of SEQ ID NO: 1. In some embodiments, a variant LbCas12a endonuclease comprises an V936G and S982W substitution and has at least 85% identity to the amino acid sequence of SEQ ID NO: 1. In some embodiments, a variant LbCas12a endonuclease comprises an V936G and S982W substitution and has at least 90% identity to the amino acid sequence of SEQ ID NO: 1. In some embodiments, a variant LbCas12a endonuclease comprises an V936G and S982W substitution and has at least 95% identity to the amino acid sequence of SEQ ID NO: 1. In some embodiments, a variant LbCas12a endonuclease comprises an V936G and S982W substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an V936G and M986G substitution and has at least 80% identity to the amino acid sequence of SEQ ID NO: 1. In some embodiments, a variant LbCas12a endonuclease comprises an V936G and M986G substitution and has at least 85% identity to the amino acid sequence of SEQ ID NO: 1. In some embodiments, a variant LbCas12a endonuclease comprises an V936G and M986G substitution and has at least 90% identity to the amino acid sequence of SEQ ID NO: 1. In some embodiments, a variant LbCas12a endonuclease comprises an V936G and M986G substitution and has at least 95% identity to the amino acid sequence of SEQ ID NO: 1. In some embodiments, a variant LbCas12a endonuclease comprises an V936G and M986G substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an V936G and T988F substitution and has at least 80% identity to the amino acid sequence of SEQ ID NO: 1. In some embodiments, a variant LbCas12a endonuclease comprises an V936G and T988F substitution and has at least 85% identity to the amino acid sequence of SEQ ID NO: 1. In some embodiments, a variant LbCas12a endonuclease comprises an V936G and T988F substitution and has at least 90% identity to the amino acid sequence of SEQ ID NO: 1. In some embodiments, a variant LbCas12a endonuclease comprises an V936G and T988F substitution and has at least 95% identity to the amino acid sequence of SEQ ID NO: 1. In some embodiments, a variant LbCas12a endonuclease comprises an V936G and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an Q944D and S982W substitution and has at least 80% identity to the amino acid sequence of SEQ ID NO: 1. In some embodiments, a variant LbCas12a endonuclease comprises an Q944D and S982W substitution and has at least 85% identity to the amino acid sequence of SEQ ID NO: 1. In some embodiments, a variant LbCas12a endonuclease comprises an Q944D and S982W substitution and has at least 90% identity to the amino acid sequence of SEQ ID NO: 1. In some embodiments, a variant LbCas12a endonuclease comprises an Q944D and S982W substitution and has at least 95% identity to the amino acid sequence of SEQ ID NO: 1. In some embodiments, a variant LbCas12a endonuclease comprises an Q944D and S982W substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an Q944D and F983L substitution and has at least 80% identity to the amino acid sequence of SEQ ID NO: 1. In some embodiments, a variant LbCas12a endonuclease comprises an Q944D and F983L substitution and has at least 85% identity to the amino acid sequence of SEQ ID NO: 1. In some embodiments, a variant LbCas12a endonuclease comprises an Q944D and F983L substitution and has at least 90% identity to the amino acid sequence of SEQ ID NO: 1. In some embodiments, a variant LbCas12a endonuclease comprises an Q944D and F983L substitution and has at least 95% identity to the amino acid sequence of SEQ ID NO: 1. In some embodiments, a variant LbCas12a endonuclease comprises an Q944D and F983L substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an Q944D and T988F substitution and has at least 80% identity to the amino acid sequence of SEQ ID NO: 1. In some embodiments, a variant LbCas12a endonuclease comprises an Q944D and T988F substitution and has at least 85% identity to the amino acid sequence of SEQ ID NO: 1. In some embodiments, a variant LbCas12a endonuclease comprises an Q944D and T988F substitution and has at least 90% identity to the amino acid sequence of SEQ ID NO: 1. In some embodiments, a variant LbCas12a endonuclease comprises an Q944D and T988F substitution and has at least 95% identity to the amino acid sequence of SEQ ID NO: 1. In some embodiments, a variant LbCas12a endonuclease comprises an Q944D and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an S982W and F983L substitution and has at least 80% identity to the amino acid sequence of SEQ ID NO: 1. In some embodiments, a variant LbCas12a endonuclease comprises an S982W and F983L substitution and has at least 85% identity to the amino acid sequence of SEQ ID NO: 1. In some embodiments, a variant LbCas12a endonuclease comprises an S982W and F983L substitution and has at least 90% identity to the amino acid sequence of SEQ ID NO: 1. In some embodiments, a variant LbCas12a endonuclease comprises an S982W and F983L substitution and has at least 95% identity to the amino acid sequence of SEQ ID NO: 1. In some embodiments, a variant LbCas12a endonuclease comprises an S982W and F983L substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an S982W and T988F substitution and has at least 80% identity to the amino acid sequence of SEQ ID NO: 1. In some embodiments, a variant LbCas12a endonuclease comprises an S982W and T988F substitution and has at least 85% identity to the amino acid sequence of SEQ ID NO: 1. In some embodiments, a variant LbCas12a endonuclease comprises an S982W and T988F substitution and has at least 90% identity to the amino acid sequence of SEQ ID NO: 1. In some embodiments, a variant LbCas12a endonuclease comprises an S982W and T988F substitution and has at least 95% identity to the amino acid sequence of SEQ ID NO: 1. In some embodiments, a variant LbCas12a endonuclease comprises an S982W and T988F substitution and has at least 98% identity to the amino acid sequence of SEQ ID NO: 1. Any one or more of the foregoing variant Cas12a endonucleases may exhibit hypoactivity.

In some embodiments, a variant LbCas12a endonuclease comprises an F983G and M986G substitution and has at least 80% identity to the amino acid sequence of SEQ ID NO: 1. In some embodiments, a variant LbCas12a endonuclease comprises an F983G and M986G substitution and has at least 85% identity to the amino acid sequence of SEQ ID NO: 1. In some embodiments, a variant LbCas12a endonuclease comprises an F983G and M986G substitution and has at least 90% identity to the amino acid sequence of SEQ ID NO: 1. In some embodiments, a variant LbCas12a endonuclease comprises an F983G and M986G substitution and has at least 95% identity to the amino acid sequence of SEQ ID NO: 1. In some embodiments, a variant LbCas12a endonuclease comprises an F983G and M986G substitution and has at least 98% identity to the amino acid sequence of SEQ ID NO: 1. Any one or more of the foregoing variant Cas12a endonucleases may exhibit hypoactivity.

V. Cas12a Endonuclease Low Indiscriminate ssDNase Variants

In addition to high-specific double strand DNA (dsDNA) cleavage, Cas12a has also been shown to exhibit indiscriminate single strand DNA (ssDNA) degradation activity upon activation with a ssDNA complementary to the crRNA guide as well as with dsDNA complementary to the crRNA guide. This activity is displayed by all Cas12a orthologs and degrades any available ssDNA molecule into single/double nucleotides. Comparisons of the structures of Cas12a before, during and after cleavage reveal the structural changes that result in such an indiscriminate activity. The lid region, which is involved in the checkpoints for accurate target recognition is responsible for this action. Before the crRNA-DNA hybrid is formed, the lid occludes the cleft where the catalytic residues reside. Upon formation of the hybrid, the lid changes conformation to form an a helix, thus interacting with the crRNA of the hybrid assembly, thus dissociating the polar interactions and making available the catalytic pocket. In the R-loop structure after cleavage, this region appears disordered indicating that the catalytic site is accessible after the distal part of the dsDNA substrate dissociates from the complex. Therefore, the catalytic cleft is open and able to sever ssDNA indiscriminately. This molecular mechanism would explain how ssDNA molecules are degraded by Cas12a after being activated by the presence of the RNA-DNA hybrid. In addition, recent studies have reported non-specific nicking of target sequences bearing mismatches in distal regions of the target DNA, suggesting that this could be a problem for potential applications. See Paul, B. & Montoya, G. et al.

Several of the variant Cas12a endonucleases provided herein, surprisingly, exhibit low to no indiscriminate ssDNA degradation activity, also referred to as indiscriminate single strand deoxyribonuclease (ssDNase) activity. This activity was unexpected in part because the mutations made in the parent wild-type enzyme are outside of the lid region—the region thought to be responsible for indiscriminate ssDNase activity. “Low ssDNase activity” herein refers to indiscriminate ssDNA degradation activity of a variant endonuclease that is at least 10% lower than indiscriminate ssDNA degradation activity of the wild-type or other reference endonuclease. In some embodiments, a variant Cas12a endonucleases exhibits indiscriminate ssDNase activity that is at least 15%, at least 20%, at least 25%, at least 30%, at least 35%, at least 40%, at least 45%, at least 50%, at least 60%, at least 70%, at least 80%, at least 90%, at least 95%, at least 96%, at least 97%, at least 98%, or at least 99% lower than indiscriminate ssDNA degradation activity of the wild-type or other reference endonuclease. In some embodiments, a variant Cas12a endonucleases exhibits no (no measurable) indiscriminate ssDNase activity.

In some embodiments, a variant Cas12a endonuclease (a) comprises a mutation at an amino acid position corresponding to position N813, 1831, K932, N933, S934, V936, Q944, S982, F983, K984, M986, or T988 with reference to amino acid position numbering of LbCas12a and (b) exhibits low (or no) single indiscriminate ssDNase, optionally wherein the variant Cas12a endonuclease has at least 85%, at least 90%, at least 95%, or at least 98% identity with a wild-type reference Cas12a endonuclease.

In some embodiments, the variant Cas12a endonuclease comprises a polypeptide sequence that comprises a mutation at an amino acid position corresponding to position N813 with reference to amino acid position numbering of LbCas12a ND2006 (e.g., SEQ ID NO: 1). In some embodiments, the mutation is N813H, N813R, or N813W. In some embodiments, a variant LbCas12a endonuclease comprises a mutation at position N813 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N813 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position I831 with reference to amino acid position numbering of LbCas12a ND2006 (e.g., SEQ ID NO: 1). In some embodiments, the mutation is I831A or I831Y. In some embodiments, a variant LbCas12a endonuclease comprises a mutation at position I831 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position I831 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position K932 with reference to amino acid position numbering of LbCas12a ND2006 (e.g., SEQ ID NO: 1). In some embodiments, the mutation is K932A, K932F, K932H, K932M, K932N, K932Q, K932R, K932S, K932T, K932W, or K932Y. In some embodiments, a variant LbCas12a endonuclease comprises a mutation at position K932 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K932 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position N933 with reference to amino acid position numbering of LbCas12a ND2006 (e.g., SEQ ID NO: 1). In some embodiments, the mutation is N933E or N933L. In some embodiments, a variant LbCas12a endonuclease comprises a mutation at position N933 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position N933 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position S934 with reference to amino acid position numbering of LbCas12a ND2006 (e.g., SEQ ID NO: 1). In some embodiments, the mutation is S934K or S934Q. In some embodiments, a variant LbCas12a endonuclease comprises a mutation at position S934 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S934 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position V936 with reference to amino acid position numbering of LbCas12a ND2006 (e.g., SEQ ID NO: 1). In some embodiments, the mutation is V936E or V936G. In some embodiments, a variant LbCas12a endonuclease comprises a mutation at position V936 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position V936 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position Q944 with reference to amino acid position numbering of LbCas12a ND2006 (e.g., SEQ ID NO: 1). In some embodiments, the mutation is Q944D, Q944E, or Q944K. In some embodiments, a variant LbCas12a endonuclease comprises a mutation at position Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position S982 with reference to amino acid position numbering of LbCas12a ND2006 (e.g., SEQ ID NO: 1). In some embodiments, the mutation is S982W. In some embodiments, a variant LbCas12a endonuclease comprises a mutation at position S982 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position S982 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position F983 with reference to amino acid position numbering of LbCas12a ND2006 (e.g., SEQ ID NO: 1). In some embodiments, the mutation is F983G or F983L. In some embodiments, a variant LbCas12a endonuclease comprises a mutation at position F983 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position F983 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position K984 with reference to amino acid position numbering of LbCas12a ND2006 (e.g., SEQ ID NO: 1). In some embodiments, the mutation is K984F. In some embodiments, a variant LbCas12a endonuclease comprises a mutation at position K984 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K984 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position M986 with reference to amino acid position numbering of LbCas12a ND2006 (e.g., SEQ ID NO: 1). In some embodiments, the mutation is M986F or M986G. In some embodiments, a variant LbCas12a endonuclease comprises a mutation at position M986 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position M986 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises a mutation at an amino acid position corresponding to position T988 with reference to amino acid position numbering of LbCas12a ND2006 (e.g., SEQ ID NO: 1). In some embodiments, the mutation is T988F. In some embodiments, a variant LbCas12a endonuclease comprises a mutation at position T988 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position T988 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises mutations at amino acid positions corresponding to positions N933 and Q944 with reference to amino acid position numbering of LbCas12a ND2006 (e.g., SEQ ID NO: 1). In some embodiments, the mutations are N933L and Q944M. In some embodiments, a variant LbCas12a endonuclease comprises mutations at positions N933 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions N933 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

In some embodiments, the variant Cas12a endonuclease comprises a polypeptide sequence that comprises mutations at amino acid positions corresponding to positions F983 and M986 with reference to amino acid position numbering of LbCas12a ND2006 (e.g., SEQ ID NO: 1). In some embodiments, the mutations are F983G and M986G. In some embodiments, a variant LbCas12a endonuclease comprises mutations at positions F983 and M986 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions F983 and M986 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1). Any one or more of the foregoing variant Cas12a endonucleases may exhibit low (or no) indiscriminate ssDNase activity.

Additional Engineered Variant Cas12a Endonucleases With Low ssDNase Activity

In some embodiments, a variant LbCas12a endonuclease comprises an N813 (e.g., N813H, N813R, or N813W) substitution and has at least 80% identity to the amino acid sequence of SEQ ID NO: 1. In some embodiments, a variant LbCas12a endonuclease comprises an N813 (e.g., N813H, N813R, or N813W) substitution and has at least 85% identity to the amino acid sequence of SEQ ID NO: 1. In some embodiments, a variant LbCas12a endonuclease comprises an N813 (e.g., N813H, N813R, or N813W) substitution and has at least 90% identity to the amino acid sequence of SEQ ID NO: 1. In some embodiments, a variant LbCas12a endonuclease comprises an N813 (e.g., N813H, N813R, or N813W) substitution and has at least 95% identity to the amino acid sequence of SEQ ID NO: 1. In some embodiments, a variant LbCas12a endonuclease comprises an N813 (e.g., N813H, N813R, or N813W) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an I831 (e.g., I831A or I831Y) substitution and has at least 80% identity to the amino acid sequence of SEQ ID NO: 1. In some embodiments, a variant LbCas12a endonuclease comprises an I831 (e.g., I831A or I831Y) substitution and has at least 85% identity to the amino acid sequence of SEQ ID NO: 1. In some embodiments, a variant LbCas12a endonuclease comprises an I831 (e.g., I831A or I831Y) substitution and has at least 90% identity to the amino acid sequence of SEQ ID NO: 1. In some embodiments, a variant LbCas12a endonuclease comprises an I831 (e.g., I831A or I831Y) substitution and has at least 95% identity to the amino acid sequence of SEQ ID NO: 1. In some embodiments, a variant LbCas12a endonuclease comprises an I831 (e.g., I831A or I831Y) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an K932 (e.g., K932A, K932F, K932H, K932M, K932N, K932Q, K932R, K932S, K932T, K932W, or K932Y) substitution and has at least 80% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 85% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 90% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 95% identity to the amino acid sequence of SEQ ID NO: 1. In some embodiments, a variant LbCas12a endonuclease comprises an K932 (e.g., K932A, K932F, K932H, K932M, K932N, K932Q, K932R, K932S, K932T, K932W, or K932Y) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an N933 (e.g., N933E or N933L) substitution and has at least 80% identity to the amino acid sequence of SEQ ID NO: 1. In some embodiments, a variant LbCas12a endonuclease comprises an N933 (e.g., N933E or N933L) substitution and has at least 85% identity to the amino acid sequence of SEQ ID NO: 1. In some embodiments, a variant LbCas12a endonuclease comprises an N933 (e.g., N933E or N933L) substitution and has at least 90% identity to the amino acid sequence of SEQ ID NO: 1. In some embodiments, a variant LbCas12a endonuclease comprises an N933 (e.g., N933E or N933L) substitution and has at least 95% identity to the amino acid sequence of SEQ ID NO: 1. In some embodiments, a variant LbCas12a endonuclease comprises an N933 (e.g., N933E or N933L) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an S934 (e.g., S934K or S934Q) substitution and has at least 80% identity to the amino acid sequence of SEQ ID NO: 1. In some embodiments, a variant LbCas12a endonuclease comprises an S934 (e.g., S934K or S934Q) substitution and has at least 85% identity to the amino acid sequence of SEQ ID NO: 1. In some embodiments, a variant LbCas12a endonuclease comprises an S934 (e.g., S934K or S934Q) substitution and has at least 90% identity to the amino acid sequence of SEQ ID NO: 1. In some embodiments, a variant LbCas12a endonuclease comprises an S934 (e.g., S934K or S934Q) substitution and has at least 95% identity to the amino acid sequence of SEQ ID NO: 1. In some embodiments, a variant LbCas12a endonuclease comprises an S934 (e.g., S934K or S934Q) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an V936 (e.g., V936E or V936G) substitution and has at least 80% identity to the amino acid sequence of SEQ ID NO: 1. In some embodiments, a variant LbCas12a endonuclease comprises an V936 (e.g., V936E or V936G) substitution and has at least 85% identity to the amino acid sequence of SEQ ID NO: 1. In some embodiments, a variant LbCas12a endonuclease comprises an V936 (e.g., V936E or V936G) substitution and has at least 90% identity to the amino acid sequence of SEQ ID NO: 1. In some embodiments, a variant LbCas12a endonuclease comprises an V936 (e.g., V936E or V936G) substitution and has at least 95% identity to the amino acid sequence of SEQ ID NO: 1. In some embodiments, a variant LbCas12a endonuclease comprises an V936 (e.g., V936E or V936G) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an Q944 (e.g., Q944D, Q944E, or Q944K) substitution and has at least 80% identity to the amino acid sequence of SEQ ID NO: 1. In some embodiments, a variant LbCas12a endonuclease comprises an Q944 (e.g., Q944D, Q944E, or Q944K) substitution and has at least 85% identity to the amino acid sequence of SEQ ID NO: 1. In some embodiments, a variant LbCas12a endonuclease comprises an Q944 (e.g., Q944D, Q944E, or Q944K) substitution and has at least 90% identity to the amino acid sequence of SEQ ID NO: 1. In some embodiments, a variant LbCas12a endonuclease comprises an Q944 (e.g., Q944D, Q944E, or Q944K) substitution and has at least 95% identity to the amino acid sequence of SEQ ID NO: 1. In some embodiments, a variant LbCas12a endonuclease comprises an Q944 (e.g., Q944D, Q944E, or Q944K) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an S982 (e.g., S982W) substitution and has at least 80% identity to the amino acid sequence of SEQ ID NO: 1. In some embodiments, a variant LbCas12a endonuclease comprises an S982 (e.g., S982W) substitution and has at least 85% identity to the amino acid sequence of SEQ ID NO: 1. In some embodiments, a variant LbCas12a endonuclease comprises an S982 (e.g., S982W) substitution and has at least 90% identity to the amino acid sequence of SEQ ID NO: 1. In some embodiments, a variant LbCas12a endonuclease comprises an S982 (e.g., S982W) substitution and has at least 95% identity to the amino acid sequence of SEQ ID NO: 1. In some embodiments, a variant LbCas12a endonuclease comprises an S982 (e.g., S982W) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an F983 (e.g., F983G or F983L) substitution and has at least 80% identity to the amino acid sequence of SEQ ID NO: 1. In some embodiments, a variant LbCas12a endonuclease comprises an F983 (e.g., F983G or F983L) substitution and has at least 85% identity to the amino acid sequence of SEQ ID NO: 1. In some embodiments, a variant LbCas12a endonuclease comprises an F983 (e.g., F983G or F983L) substitution and has at least 90% identity to the amino acid sequence of SEQ ID NO: 1. In some embodiments, a variant LbCas12a endonuclease comprises an F983 (e.g., F983G or F983L) substitution and has at least 95% identity to the amino acid sequence of SEQ ID NO: 1. In some embodiments, a variant LbCas12a endonuclease comprises an F983 (e.g., F983G or F983L) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an K984 (e.g., K984F) substitution and has at least 80% identity to the amino acid sequence of SEQ ID NO: 1. In some embodiments, a variant LbCas12a endonuclease comprises an K984 (e.g., K984F) substitution and has at least 85% identity to the amino acid sequence of SEQ ID NO: 1. In some embodiments, a variant LbCas12a endonuclease comprises an K984 (e.g., K984F) substitution and has at least 90% identity to the amino acid sequence of SEQ ID NO: 1. In some embodiments, a variant LbCas12a endonuclease comprises an K984 (e.g., K984F) substitution and has at least 95% identity to the amino acid sequence of SEQ ID NO: 1. In some embodiments, a variant LbCas12a endonuclease comprises an K984 (e.g., K984F) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an M986 (e.g., M986F or M986G) substitution and has at least 80% identity to the amino acid sequence of SEQ ID NO: 1. In some embodiments, a variant LbCas12a endonuclease comprises an M986 (e.g., M986F or M986G) substitution and has at least 85% identity to the amino acid sequence of SEQ ID NO: 1. In some embodiments, a variant LbCas12a endonuclease comprises an M986 (e.g., M986F or M986G) substitution and has at least 90% identity to the amino acid sequence of SEQ ID NO: 1. In some embodiments, a variant LbCas12a endonuclease comprises an M986 (e.g., M986F or M986G) substitution and has at least 95% identity to the amino acid sequence of SEQ ID NO: 1. In some embodiments, a variant LbCas12a endonuclease comprises an M986 (e.g., M986F or M986G) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an T988 (e.g., T988F) substitution and has at least 80% identity to the amino acid sequence of SEQ ID NO: 1. In some embodiments, a variant LbCas12a endonuclease comprises an T988 (e.g., T988F) substitution and has at least 85% identity to the amino acid sequence of SEQ ID NO: 1. In some embodiments, a variant LbCas12a endonuclease comprises an T988 (e.g., T988F) substitution and has at least 90% identity to the amino acid sequence of SEQ ID NO: 1. In some embodiments, a variant LbCas12a endonuclease comprises an T988 (e.g., T988F) substitution and has at least 95% identity to the amino acid sequence of SEQ ID NO: 1. In some embodiments, a variant LbCas12a endonuclease comprises an T988 (e.g., T988F) substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an N933L and Q944M substitution and has at least 80% identity to the amino acid sequence of SEQ ID NO: 1. In some embodiments, a variant LbCas12a endonuclease comprises an N933L and Q944M substitution and has at least 85% identity to the amino acid sequence of SEQ ID NO: 1. In some embodiments, a variant LbCas12a endonuclease comprises an N933L and Q944M substitution and has at least 90% identity to the amino acid sequence of SEQ ID NO: 1. In some embodiments, a variant LbCas12a endonuclease comprises an N933L and Q944M substitution and has at least 95% identity to the amino acid sequence of SEQ ID NO: 1. In some embodiments, a variant LbCas12a endonuclease comprises an N933L and Q944M substitution and has at least 98% identity to the amino acid sequence of SEQ ID NO: 1. Any one or more of the foregoing variant Cas12a endonucleases may exhibit low (or no) ssDNase activity.

In some embodiments, a variant LbCas12a endonuclease comprises an F983G and M986G substitution and has at least 80% identity to the amino acid sequence of SEQ ID NO: 1. In some embodiments, a variant LbCas12a endonuclease comprises an F983G and M986G substitution and has at least 85% identity to the amino acid sequence of SEQ ID NO: 1. In some embodiments, a variant LbCas12a endonuclease comprises an F983G and M986G substitution and has at least 90% identity to the amino acid sequence of SEQ ID NO: 1. In some embodiments, a variant LbCas12a endonuclease comprises an F983G and M986G substitution and has at least 95% identity to the amino acid sequence of SEQ ID NO: 1. In some embodiments, a variant LbCas12a endonuclease comprises an F983G and M986G substitution and has at least 98% identity to the amino acid sequence of SEQ ID NO: 1. Any one or more of the foregoing variant Cas12a endonucleases may exhibit low (or no) ssDNase activity.

TABLE 3
Variant Cas12a Endonucleases

| Variant | Sequence | SEQ ID NO: |
|---|---|---|
| E95R | MSKLEKFTNC YSLSKTLRFK AIPVGKTQEN IDNKRLLVED EKRAEDYKGV KKLLDRYYLS | 48 |
| | FINDVLHSIK LKNLNNYISL FRKKTRTEKE NKELRNLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| E95Y | MSKLEKFTNC YSLSKTLRFK AIPVGKTQEN IDNKRLLVED EKRAEDYKGV KKLLDRYYLS | 49 |
| | FINDVLHSIK LKNLNNYISL FRKKTRTEKE NKELYNLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| E125A | MSKLEKFTNC YSLSKTLRFK AIPVGKTQEN IDNKRLLVED EKRAEDYKGV KKLLDRYYLS | 50 |
| | FINDVLHSIK LKNLNNYISL FRKKTRTEKE NKELENLEIN LRKEIAKAFK GNEGYKSLFK | |
| | KDIIATILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| E125W | MSKLEKFTNC YSLSKTLRFK AIPVGKTQEN IDNKRLLVED EKRAEDYKGV KKLLDRYYLS | 51 |
| | FINDVLHSIK LKNLNNYISL FRKKTRTEKE NKELENLEIN LRKEIAKAFK GNEGYKSLFK | |
| | KDIIWTILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDES DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N256A | MSKLEKFTNC YSLSKTLRFK AIPVGKTQEN IDNKRLLVED EKRAEDYKGV KKLLDRYYLS | 52 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLAEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N256K | MSKLEKFTNC YSLSKTLRFK AIPVGKTQEN IDNKRLLVED EKRAEDYKGV KKLLDRYYLS | 53 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLKEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R747Y | MSKLEKFTNC YSLSKILRFK AIPVGKTQEN IDNKRLLVED EKRAEDYKGV KKLLDRYYLS | 54 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMYRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| H759V | MSKLEKFTNC YSLSKTLRFK AIPVGKTQEN IDNKRLLVED EKRAEDYKGV KKLLDRYYLS | 55 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVVP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| H759D | MSKLEKFTNC YSLSKTLRFK AIPVGKTQEN IDNKRLLVED EKRAEDYKGV KKLLDRYYLS | 56 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVDP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDINSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N813W | MSKLEKFTNC YSLSKTLRFK AIPVGKTQEN IDNKRLLVED EKRAEDYKGV KKLLDRYYLS | 57 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KIWTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N813R | MSKLEKFTNC YSLSKTLRFK AIPVGKTQEN IDNKRLLVED EKRAEDYKGV KKLLDRYYLS | 58 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KIRTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N813H | MSKLEKFTNC YSLSKTLRFK AIPVGKTQEN IDNKRLLVED EKRAEDYKGV KKLLDRYYLS | 59 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MESEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KIHTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| I831A | MSKLEKFTNC YSLSKTLRFK AIPVGKTQEN IDNKRLLVED EKRAEDYKGV KKLLDRYYLS | 60 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG ADRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| I831Y | MSKLEKFTNC YSLSKTLRFK AIPVGKTQEN IDNKRLLVED EKRAEDYKGV KKLLDRYYLS | 61 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG YDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932L | MSKLEKFTNC YSLSKTLRFK AIPVGKTQEN IDNKRLLVED EKRAEDYKGV KKLLDRYYLS | 62 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FLNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932I | MSKLEKFTNC YSLSKTLRFK AIPVGKTQEN IDNKRLLVED EKRAEDYKGV KKLLDRYYLS | 63 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FINSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932V | MSKLEKFTNC YSLSKTLRFK AIPVGKTQEN IDNKRLLVED EKRAEDYKGV KKLLDRYYLS | 64 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FVNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932M | MSKLEKFTNC YSLSKTLRFK AIPVGKTQEN IDNKRLLVED EKRAEDYKGV KKLLDRYYLS | 65 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FMNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932F | MSKLEKFTNC YSLSKTLRFK AIPVGKTQEN IDNKRLLVED EKRAEDYKGV KKLLDRYYLS | 66 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FFNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932R | MSKLEKFTNC YSLSKILRFK AIPVGKTQEN IDNKRLLVED EKRAEDYKGV KKLLDRYYLS | 67 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FRNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932A | MSKLEKFTNC YSLSKTLRFK AIPVGKTQEN IDNKRLLVED EKRAEDYKGV KKLLDRYYLS | 68 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FANSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932H | MSKLEKFTNC YSLSKTLRFK AIPVGKTQEN IDNKRLLVED EKRAEDYKGV KKLLDRYYLS | 69 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FHNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932N | MSKLEKFTNC YSLSKTLRFK AIPVGKTQEN IDNKRLLVED EKRAEDYKGV KKLLDRYYLS | 70 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENI | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FNNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932Q | MSKLEKFTNC YSLSKTLRFK AIPVGKTQEN IDNKRLLVED EKRAEDYKGV KKLLDRYYLS | 71 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FQNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932S | MSKLEKFTNC YSLSKTLRFK AIPVGKTQEN IDNKRLLVED EKRAEDYKGV KKLLDRYYLS | 72 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FSNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932T | MSKLEKFTNC YSLSKTLRFK AIPVGKTQEN IDNKRLLVED EKRAEDYKGV KKLLDRYYLS | 73 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FTNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932Y | MSKLEKFTNC YSLSKTLRFK AIPVGKTQEN IDNKRLLVED EKRAEDYKGV KKLLDRYYLS | 74 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FYNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932W | MSKLEKFTNC YSLSKTLRFK AIPVGKTQEN IDNKRLLVED EKRAEDYKGV KKLLDRYYLS | 75 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FWNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N933E | MSKLEKFTNC YSLSKTLRFK AIPVGKTQEN IDNKRLLVED EKRAEDYKGV KKLLDRYYLS | 76 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKESRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N933V | MSKLEKFTNC YSLSKTLRFK AIPVGKTQEN IDNKRLLVED EKRAEDYKGV KKLLDRYYLS | 77 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKVSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N933L | MSKLEKFTNC YSLSKTLRFK AIPVGKTQEN IDNKRLLVED EKRAEDYKGV KKLLDRYYLS | 78 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKLSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S934Q | MSKLEKFTNC YSLSKTLRFK AIPVGKTQEN IDNKRLLVED EKRAEDYKGV KKLLDRYYLS | 79 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNQRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S934K | MSKLEKFTNC YSLSKTLRFK AIPVGKTQEN IDNKRLLVED EKRAEDYKGV KKLLDRYYLS | 80 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNKRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S934W | MSKLEKFTNC YSLSKTLRFK AIPVGKTQEN IDNKRLLVED EKRAEDYKGV KKLLDRYYLS | 81 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNWRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936E | MSKLEKFTNC YSLSKTLRFK AIPVGKTQEN IDNKRLLVED EKRAEDYKGV KKLLDRYYLS | 82 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSREKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936M | MSKLEKFTNC YSLSKTLRFK AIPVGKTQEN IDNKRLLVED EKRAEDYKGV KKLLDRYYLS | 83 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRMKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936K | MSKLEKFTNC YSLSKTLRFK AIPVGKTQEN IDNKRLLVED EKRAEDYKGV KKLLDRYYLS | 84 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRKKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936G | MSKLEKFTNC YSLSKTLRFK AIPVGKTQEN IDNKRLLVED EKRAEDYKGV KKLLDRYYLS | 85 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRGKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944D | MSKLEKFTNC YSLSKTLRFK AIPVGKTQEN IDNKRLLVED EKRAEDYKGV KKLLDRYYLS | 86 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDINSG FKNSRXKVEK QVYDKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944E | MSKLEKFTNC YSLSKTLRFK AIPVGKTQEN IDNKRLLVED EKRAEDYKGV KKLLDRYYLS | 87 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYEKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944K | MSKLEKFTNC YSLSKTLRFK AIPVGKTQEN IDNKRLLVED EKRAEDYKGV KKLLDRYYLS | 88 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944M | MSKLEKFTNC YSLSKTLRFK AIPVGKTQEN IDNKRLLVED EKRAEDYKGV KKLLDRYYLS | 89 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYMKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S982W | MSKLEKFTNC YSLSKTLRFK AIPVGKTQEN IDNKRLLVED EKRAEDYKGV KKLLDRYYLS | 90 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF EWFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S982T | MSKLEKFTNC YSLSKTLRFK AIPVGKTQEN IDNKRLLVED EKRAEDYKGV KKLLDRYYLS | 91 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ETFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S982N | MSKLEKFTNC YSLSKTLRFK AIPVGKTQEN IDNKRLLVED EKRAEDYKGV KKLLDRYYLS | 92 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ENFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| F983L | MSKLEKFTNC YSLSKTLRFK AIPVGKTQEN IDNKRLLVED EKRAEDYKGV KKLLDRYYLS | 93 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| F983G | MSKLEKFTNC YSLSKILRFK AIPVGKTQEN IDNKRLLVED EKRAEDYKGV KKLLDRYYLS | 94 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MESEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESGKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K984F | MSKLEKFTNC YSLSKTLRFK AIPVGKTQEN IDNKRLLVED EKRAEDYKGV KKLLDRYYLS | 95 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFFSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |

| | | |
|---|---|---|
| K984R | MSKLEKFTNC YSLSKTLRFK AIPVGKTQEN IDNKRLLVED EKRAEDYKGV KKLLDRYYLS | 96 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ERFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| M986G | MSKLEKFTNC YSLSKTLRFK AIPVGKTQEN IDNKRLLVED EKRAEDYKGV KKLLDRYYLS | 97 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNQRVKVEK QVYQKFEKML IDKLNYMVDK | |
| | KSNPCATGGA LKGYQITNKF ESFKSGSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| M986F | MSKLEKFTNC YSLSKTLRFK AIPVGKTQEN IDNKRLLVED EKRAEDYKGV KKLLDRYYLS | 98 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNQRVKVEK QVYQKFEKML IDKLNYMVDK | |
| | KSNPCATGGA LKGYQITNKF ESFKSFSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| M986L | MSKLEKFTNC YSLSKTLRFK AIPVGKTQEN IDNKRLLVED EKRAEDYKGV KKLLDRYYLS | 99 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNQRVKVEK QVYQKFEKML IDKLNYMVDK | |
| | KSNPCATGGA LKGYQITNKF ESFKSLSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| M986S | MSKLEKFTNC YSLSKTLRFK AIPVGKTQEN IDNKRLLVED EKRAEDYKGV KKLLDRYYLS | 100 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNQRVKVEK QVYQKFEKML IDKLNYMVDK | |
| | KSNPCATGGA LKGYQITNKF ESFKSSSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| T988F | MSKLEKFTNC YSLSKTLRFK AIPVGKTQEN IDNKRLLVED EKRAEDYKGV KKLLDRYYLS | 101 |
| | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFFSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932F, | MSKLEKFTNC YSLSKTLRFK AIPVGKTQEN IDNKRLLVED EKRAEDYKGV KKLLDRYYLS | 102 |
| F983L | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FFNSRVKVEK QVYQKFEKML IDKLNYMVDK | |
| | KSNPCATGGA LKGYQITNKF ESLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932F, | MSKLEKFTNC YSLSKTLRFK AIPVGKTQEN IDNKRLLVED EKRAEDYKGV KKLLDRYYLS | 103 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FFNSRVKVEK QVYQKFEKML IDKLNYMVDK | |
| | KSNPCATGGA LKGYQITNKF ESFKSMSFQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932R | MSKLEKFTNC YSLSKTLRFK AIPVGKTQEN IDNKRLLVED EKRAEDYKGV KKLLDRYYLS | 104 |
| Q944D | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FRNSRVKVEK QVYDKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932R, | MSKLEKFTNC YSLSKTLRFK AIPVGKTQEN IDNKRLLVED EKRAEDYKGV KKLLDRYYLS | 105 |
| F983L | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FRNSRVKVEK QVYQKFEKML IDKLNYMVDK | |
| | KSNPCATGGA LKGYQITNKF ESLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932R, | MSKLEKFTNC YSLSKTLRFK AIPVGKTQEN IDNKRLLVED EKRAEDYKGV KKLLDRYYLS | 106 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FRNSRVKVEK QVYQKFEKML IDKLNYMVDK | |
| | KSNPCATGGA LKGYQITNKF ESFKSMSFQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932Y, | MSKLEKFTNC YSLSKTLRFK AIPVGKTQEN IDNKRLLVED EKRAEDYKGV KKLLDRYYLS | 107 |
| F983L | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FYNSRVKVEK QVYQKFEKML IDKLNYMVDK | |
| | KSNPCATGGA LKGYQITNKF ESLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932Y, | MSKLEKFTNC YSLSKTLRFK AIPVGKTQEN IDNKRLLVED EKRAEDYKGV KKLLDRYYLS | 108 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FYNSRVKVEK QVYQKFEKML IDKLNYMVDK | |
| | KSNPCATGGA LKGYQITNKF ESFKSMSFQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| N933L, | MSKLEKFTNC YSLSKTLRFK AIPVGKTQEN IDNKRLLVED EKRAEDYKGV KKLLDRYYLS | 109 |
| Q944M | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKLSRVKVEK QVYMKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936G, | MSKLEKFTNC YSLSKILRFK AIPVGKTQEN IDNKRLLVED EKRAEDYKGV KKLLDRYYLS | 110 |
| Q944D | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRGKVEK QVYDKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936G, | MSKLEKFTNC YSLSKTLRFK AIPVGKTQEN IDNKRLLVED EKRAEDYKGV KKLLDRYYLS | 111 |
| S982W | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRGKVEK QVYQKFEKML IDKLNYMVDK | |
| | KSNPCATGGA LKGYQITNKF EWFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936G, | MSKLEKFTNC YSLSKTLRFK AIPVGKTQEN IDNKRLLVED EKRAEDYKGV KKLLDRYYLS | 112 |
| M986G | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRGKVEK QVYQKFEKML IDKLNYMVDK | |
| | KSNPCATGGA LKGYQITNKF ESFKSGSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| V936G, | MSKLEKFTNC YSLSKTLRFK AIPVGKTQEN IDNKRLLVED EKRAEDYKGV KKLLDRYYLS | 113 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRGKVEK QVYQKFEKML IDKLNYMVDK | |
| | KSNPCATGGA LKGYQITNKF ESFKSMSFQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAI GQFKK | |
| | AEDEKLDKVK IAISNKEWLE YAQTSVKH | |
| Q944D, | MSKLEKFTNC YSLSKTLRFK AIPVGKTQEN IDNKRLLVED EKRAEDYKGV KKLLDRYYLS | 114 |
| S982W | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYDKFEKML IDKLNYMVDK | |
| | KSNPCATGGA LKGYQITNKF EWFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944D, | MSKLEKFTNC YSLSKTLRFK AIPVGKTQEN IDNKRLLVED EKRAEDYKGV KKLLDRYYLS | 115 |
| F983L | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYDKFEKML IDKLNYMVDK | |
| | KSNPCATGGA LKGYQITNKF ESLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944D, | MSKLEKFTNC YSLSKTLRFK AIPVGKTQEN IDNKRLLVED EKRAEDYKGV KKLLDRYYLS | 116 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRXKVEK QVYDKFEKML IDKLNYMVDK | |
| | KSNPCATGGA LKGYQITNKF ESFKSMSFQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S982W, | MSKLEKFTNC YSLSKTLRFK AIPVGKTQEN IDNKRLLVED EKRAEDYKGV KKLLDRYYLS | 117 |
| F983L | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF EWLKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| S982W, | MSKLEKFTNC YSLSKTLRFK AIPVGKTQEN IDNKRLLVED EKRAEDYKGV KKLLDRYYLS | 118 |
| T988F | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDINSG FKNSRVKVEK QVYQKFEKML IDKLNYMVDK | |
| | KSNPCATGGA LKGYQITNKF EWFKSMSFQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| F983G, | MSKLEKFTNC YSLSKTLRFK AIPVGKTQEN IDNKRLLVED EKRAEDYKGV KKLLDRYYLS | 119 |
| M986G | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESGKSGSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932G, | MSKLEKFTNC YSLSKTLRFK AIPVGKTQEN IDNKRLLVED EKRAEDYKGV KKLLDRYYLS | 367 |
| N933G | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FGGSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833L | MSKLEKFTNC YSLSKTLRFK AIPVGKTQEN IDNKRLLVED EKRAEDYKGV KKLLDRYYLS | 368 |
| (LbAA9) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDLGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833K | MSKLEKFTNC YSLSKTLRFK AIPVGKTQEN IDNKRLLVED EKRAEDYKGV KKLLDRYYLS | 369 |
| (LbAA19) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDKGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M | MSKLEKFTNC YSLSKILRFK AIPVGKTQEN IDNKRLLVED EKRAEDYKGV KKLLDRYYLS | 370 |
| (LbEF1s9) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDINSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932E | MSKLEKFTNC YSLSKTLRFK AIPVGKTQEN IDNKRLLVED EKRAEDYKGV KKLLDRYYLS | 371 |
| (LbAA23) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FENSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932G | MSKLEKFTNC YSLSKTLRFK AIPVGKTQEN IDNKRLLVED EKRAEDYKGV KKLLDRYYLS | 372 |
| (LbMS07) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FGNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K940G | MSKLEKFTNC YSLSKTLRFK AIPVGKTQEN IDNKRLLVED EKRAEDYKGV KKLLDRYYLS | 373 |
| (LbAA49) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEG QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Q944K | MSKLEKFTNC YSLSKTLRFK AIPVGKTQEN IDNKRLLVED EKRAEDYKGV KKLLDRYYLS | 374 |
| (LbAC10) | FINDVLHSIK LKNLNNYISL FRKKTRTEKE NKELENLEIN LRKEIAKAFK GNEGYKSLFK | |
| | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K932G, | MSKLEKFTNC YSLSKTLRFK AIPVGKTQEN IDNKRLLVED EKRAEDYKGV KKLLDRYYLS | 375 |
| N933G, | FINDVLHSIK LKNLNNYISL FRKKTRTEKE NKELENLEIN LRKEIAKAFK GNEGYKSLFK | |
| V936G, | KDIIETILPE FLDDKDEIAL VNSFNGFTTA FTGFFDNREN MFSEEAKSTS IAFRCINENL | |
| S929G | TRYISNMDIF EKVDAIFDKH EVQEIKEKIL NSDYDVEDFF EGEFFNFVLT QEGIDVYNAI | |
| (LbMS3n5) | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNGG FGGSRGKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| K940G, | MSKLEKFTNC YSLSKTLRFK AIPVGKTQEN IDNKRLLVED EKRAEDYKGV KKLLDRYYLS | 376 |
| Q944K | FINDVLHSIK LKNLNNYISL FRKKTRTEKE NKELENLEIN LRKEIAKAFK GNEGYKSLFK | |
| (LbTN37) | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEG QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRIDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R836G, | MSKLEKFTNC YSLSKTLRFK AIPVGKTQEN IDNKRLLVED EKRAEDYKGV KKLLDRYYLS | 377 |
| Q944K | FINDVLHSIK LKNLNNYISL FRKKTRTEKE NKELENLEIN LRKEIAKAFK GNEGYKSLFK | |
| (LbTN39) | KDIIETILPE FLDDKDEIAL VNSFNGFTTA FTGFFDNREN MFSEEAKSTS IAFRCINENL | |
| | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDRGEGNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKTLRFK AIPVGKTQEN IDNKRLLVED EKRAEDYKGV KKLLDRYYLS | 378 |
| E835D, | FINDVLHSIK LKNLNNYISL FRKKTRTEKE NKELENLEIN LRKEIAKAFK GNEGYKSLFK | |
| Y943T | KDIIETILPE FLDDKDEIAL VNSFNGFTTA FTGFFDNREN MFSEEAKSTS IAFRCINENL | |
| (LbTN2) | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDES DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVTQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R836G, | MSKLEKFTNC YSLSKTLRFK AIPVGKTQEN IDNKRLLVED EKRAEDYKGV KKLLDRYYLS | 379 |
| Q944K, | FINDVLHSIK LKNLNNYISL FRKKTRTEKE NKELENLEIN LRKEIAKAFK GNEGYKSLFK | |
| R935G | KDIIETILPE FLDDKDEIAL VNSFNGFTTA FTGFFDNREN MFSEEAKSTS IAFRCINENL | |
| (LbFM14) | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGEGNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSGVKVEK QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKTLRFK AIPVGKTQEN IDNKRLLVED EKRAEDYKGV KKLLDRYYLS | 380 |
| E835D, | FINDVLHSIK LKNLNNYISL FRKKTRTEKE NKELENLEIN LRKEIAKAFK GNEGYKSLFK | |
| Y943T, | KDIIETILPE FLDDKDEIAL VNSFNGFTTA FTGFFDNREN MFSEEAKSTS IAFRCINENL | |
| R935G | TRYISNMDIF EKVDAIFDKH EVQEIKEKIL NSDYDVEDFF EGEFFNFVLT QEGIDVYNAI | |
| (LbFM17) | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSGVKVEK QVTQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKILRFK AIPVGKTQEN IDNKRLLVED EKRAEDYKGV KKLLDRYYLS | 381 |
| E835D, | FINDVLHSIK LKNLNNYISL FRKKTRTEKE NKELENLEIN LRKEIAKAFK GNEGYKSLFK | |
| Y943T, | KDIIETILPE FLDDKDEIAL VNSFNGFTTA FTGFFDNREN MFSEEAKSTS IAFRCINENL | |
| Q941K | TRYISNMDIF EKVDAIFDKH EVQEIKEKIL NSDYDVEDFF EGEFFNFVLT QEGIDVYNAI | |
| (LbFM28) | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK KVTQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKTLRFK AIPVGKTQEN IDNKRLLVED EKRAEDYKGV KKLLDRYYLS | 382 |
| E835D | FINDVLHSIK LKNLNNYISL FRKKTRTEKE NKELENLEIN LRKEIAKAFK GNEGYKSLFK | |
| E125A | KDIIATILPE FLDDKDEIAL VNSFNGFTTA FTGFFDNREN MFSEEAKSTS IAFRCINENL | |
| (LbFM44) | TRYISNMDIF EKVDAIFDKH EVQEIKEKIL NSDYDVEDFF EGEFFNFVLT QEGIDVYNAI | |
| | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| Y943F, | MSKLEKFTNC YSLSKTLRFK AIPVGKTQEN IDNKRLLVED EKRAEDYKGV KKLLDRYYLS | 383 |
| Q944K | FINDVLHSIK LKNLNNYISL FRKKTRTEKE NKELENLEIN LRKEIAKAFK GNEGYKSLFK | |
| K932G- | KDIIATILPE FLDDKDEIAL VNSFNGFTTA FTGFFDNREN MFSEEAKSTS IAFRCINENL | |
| N933G, | TRYISNMDIF EKVDAIFDKH EVQEIKEKIL NSDYDVEDFF EGEFFNFVLT QEGIDVYNAI | |
| E125A | IGGFVTESGE KIKGLNEYIN LYNQKTKQKL PKFKPLYKQV LSDRESLSFY GEGYTSDEEV | |
| (LbFM51) | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FGGSRVKVEK QVFKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R836G, | MSKLEKFTNC YSLSKTLRFK AIPVGKTQEN IDNKRLLVED EKRAEDYKGV KKLLDRYYLS | 384 |
| Q944K, | FINDVLHSIK LKNLNNYISL FRKKTRTEKE NKELENLEIN LRKEIAKAFK GNEGYKSLFK | |
| R935G, | KDIIATILPE FLDDKDEIAL VNSFNGFTTA FTGFFDNREN MFSEEAKSTS IAFRCINENL | |
| E125A | TRYISNMDIF EKVDAIFDKH EVQEIKEKIL NSDYDVEDFF EGEFFNFVLT QEGIDVYNAI | |
| (LbFM64) | IGGFVTESGE KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDRGEGNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSGVKVEK QVYKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKTLRFK AIPVGKTQEN IDNKRLLVED EKRAEDYKGV KKLLDRYYLS | 385 |
| E835D, | FINDVLHSIK LKNLNNYISL FRKKTRTEKE NKELENLEIN LRKEIAKAFK GNEGYKSLFK | |
| Y943T, | KDIIATILPE FLDDKDEIAL VNSFNGFTTA FTGFFDNREN MFSEEAKSTS IAFRCINENL | |
| R935G, | TRYISNMDIF EKVDAIFDKH EVQEIKEKIL NSDYDVEDFF EGEFFNFVLT QEGIDVYNAI | |
| E125A | IGGFVTESGE KIKGLNEYIN LYNQKTKQKL PKFKPLYKQV LSDRESLSFY GEGYTSDEEV | |
| (LbFM65) | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKITTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSGVKVEK QVTQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| R833M, | MSKLEKFTNC YSLSKTLRFK AIPVGKTQEN IDNKRLLVED EKRAEDYKGV KKLLDRYYLS | 386 |
| E835D, | FINDVLHSIK LKNLNNYISL FRKKTRTEKE NKELENLEIN LRKEIAKAFK GNEGYKSLFK | |
| Y943T, | KDIIATILPE FLDDKDEIAL VNSFNGFTTA FTGFFDNREN MFSEEAKSTS IAFRCINENL | |
| Q941K, | TRYISNMDIF EKVDAIFDKH EVQEIKEKIL NSDYDVEDFF EGEFFNFVLT QEGIDVYNAI | |
| E125A | IGGFVTESGE KIKGLNEYIN LYNQKTKQKL PKFKPLYKQV LSDRESLSFY GEGYTSDEEV | |
| (LbFM67) | LEVFRNTLNK NSEIFSSIKK LEKLFKNFDE YSSAGIFVKN GPAISTISKD IFGEWNVIRD | |
| | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMENLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IDMGDRNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FKNSRVKVEK KVTQKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELEN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |
| D832A, | MSKLEKFTNC YSLSKTLRFK AIPVGKTQEN IDNKRLLVED EKRAEDYKGV KKLLDRYYLS | 387 |
| Y943F, | FINDVLHSIK LKNLNNYISL FRKKTRTEKE NKELENLEIN LRKEIAKAFK GNEGYKSLFK | |
| Q944K, | KDIIATILPE FLDDKDEIAL VNSFNGFTTA FTGFFDNREN MFSEEAKSTS IAFRCINENL | |
| K932G- | TRYISNMDIF EKVDAIFDKH EVQEIKEKIL NSDYDVEDFF EGEFFNFVLT QEGIDVYNAI | |
| N933G, | IGGFVTESGE KIKGLNEYIN LYNQKTKQKL PKFKPLYKQV LSDRESLSFY GEGYTSDEEV | |
| E125A | LEVFRNTLNK NSEIFSSIKK LEKLFKNFDE YSSAGIFVKN GPAISTISKD IFGEWNVIRD | |
| (LbFM76) | KWNAEYDDIH LKKKAVVTEK YEDDRRKSFK KIGSFSLEQL QEYADADLSV VEKLKEIIIQ | |
| | KVDEIYKVYG SSEKLFDADF VLEKSLKKND AVVAIMKDLL DSVKSFENYI KAFFGEGKET | |
| | NRDESFYGDF VLAYDILLKV DHIYDAIRNY VTQKPYSKDK FKLYFQNPQF MGGWDKDKET | |
| | DYRATILRYG SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS NAYDFNFSET | |
| | EKYKDIAGFY REVEEQGYKV SFESASKKEV DKLVEEGKLY MFQIYNKDFS DKSHGTPNLH | |
| | TMYFKLLFDE NNHGQIRLSG GAELFMRRAS LKKEELVVHP ANSPIANKNP DNPKKTTTLS | |
| | YDVYKDKRFS EDQYELHIPI AINKCPKNIF KINTEVRVLL KHDDNPYVIG IARGERNLLY | |
| | IVVVDGKGNI VEQYSLNEII NNFNGIRIKT DYHSLLDKKE KERFEARQNW TSIENIKELK | |
| | AGYISQVVHK ICELVEKYDA VIALEDLNSG FGGSRVKVEK QVFKKFEKML IDKLNYMVDK | |
| | KSNPCATGGA LKGYQITNKF ESFKSMSTQN GFIFYIPAWL TSKIDPSTGF VNLLKTKYTS | |
| | IADSKKFISS FDRIMYVPEE DLFEFALDYK NFSRTDADYI KKWKLYSYGN RIRIFRNPKK | |
| | NNVFDWEEVC LTSAYKELFN KYGINYQQGD IRALLCEQSD KAFYSSFMAL MSLMLQMRNS | |
| | ITGRTDVDFL ISPVKNSDGI FYDSRNYEAQ ENAILPKNAD ANGAYNIARK VLWAIGQFKK | |
| | AEDEKLDKVK IAISNKEWLE YAQTSVKH | |

### VI. Polynucleotides

The present disclosure provides, in some aspects, polynucleotides encoding the variant Cas12a endonucleases. Nucleic acids comprise a polymer of nucleotides (nucleotide monomers). Thus, nucleic acids are also referred to as polynucleotides. Nucleic acids may be or may include, for example, deoxyribonucleic acid (DNA), ribonucleic acid (RNA), threose nucleic acid (TNA), glycol nucleic acid (GNA), peptide nucleic acid (PNA), locked nucleic acid (LNA, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acid (ENA), cyclohexenyl nucleic acid (CeNA) and/or chimeras and/or combinations thereof.

In some embodiments, the polynucleotide encoding the variant Cas12a endonuclease is an RNA, such as an mRNA. Messenger RNA (mRNA) is RNA that encodes a (at least one) protein (a naturally-occurring, non-naturally-occurring, or modified polymer of amino acids) and can be translated to produce the encoded protein in vitro, in vivo, in situ, or ex vivo. An mRNA provided herein typically comprises an open reading frame (ORF) encoding a variant Cas12a endonuclease. In some embodiments, an mRNA also comprises an ORF encoding a crRNA or multiple crRNAs. In some embodiments, an mRNA further comprises a 5′ cap, a 5′ untranslated region (UTR), a 3′ UTR, and a poly(A) tail.

An ORF is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and/or 3′ UTRs, but that those elements, unlike the ORF, need not necessarily be present in an RNA (e.g., mRNA) of the present disclosure.

In some embodiments, an ORF encoding a variant Cas12a endonuclease of the disclosure is codon optimized. Codon optimization methods are known in the art. An open reading frame of any one or more of the variant Cas12a endonucleases provided herein may be codon optimized. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame sequence is optimized using optimization algorithms.

A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the stop codon (i.e., the codon of an mRNA transcript that signals a termination of translation) that does not encode a polypeptide. When RNA transcripts are being generated, the 5′ UTR may comprise a promoter sequence.

In some embodiments, an RNA (e.g., mRNA) comprises a 5′ terminal cap. 5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA (e.g., mRNA) may be completed post-transcriptionally using, for example, a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs may be used.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. It can, in some instances, comprise up to about 400 adenine nucleotides. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation. In some embodiments, the length of the 3′-poly(A) tail may be an essential element with respect to the stability of the individual mRNA. In some embodiments, a poly(A) tail has a length of about 50, about 100, about 150, about 200, about 250, about 300, about 350, or about 400 nucleotides. In some embodiments, a poly(A) tail has a length of 100 nucleotides.

### VII. Fusion Proteins

Some aspects relate to fusion proteins comprising any one or more of the variant Cas12a endonucleases provided herein and one or more effector proteins (e.g., a protein, such as an enzyme, that regulates a biological activity). Non-limiting examples include proteins that exhibit deaminase activity (e.g., adenosine deaminase and/or cytidine deaminase), reverse transcriptase, endonuclease (e.g., FokI), exonuclease activity (e.g., T5 exonuclease), methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

Provided herein, in some aspects, are base editing fusion proteins comprising one or more base editing enzyme(s). A “base editing enzyme” is an enzyme that is capable of converting a target nucleobase or base pair into a different nucleobase or base pair (e.g. conversion from adenine to thymine (A to T), cytosine to thymine (C to T), adenine to guanine (A to G), cytosine to guanine (C to G)) without requiring the creation and/or repair of double-stranded breaks in a polynucleotide chain. Base editing enzymes can be specific for DNA bases or specific for RNA bases. Any base editing enzyme may be utilized in a fusion protein as described herein.

A base editing enzyme may be capable of converting adenine to guanine; adenine to thymine; adenine to uracil; adenine to cytosine; guanine to adenine; guanine to thymine; guanine to uracil; guanine to cytosine; thymine to adenine; thymine to guanine; thymine to uracil; thymine to cytosine; uracil to adenine; uracil to guanine; uracil to thymine; uracil to cytosine; cytosine to adenine; cytosine to guanine; cytosine to thymine; or cytosine to uracil. In some embodiments, a base editing enzyme is capable converting a standard nucleobase (e.g., A, C, G, T, U) into a modified nucleobase (e.g., hypoxanthine, xanthine 7-methylguanine, 5,6-dihydrouracil, 5-methylcytosine, 5-hydroxymethylcytosine). In some embodiments, a base editing enzyme is capable converting a modified nucleobase (e.g., hypoxanthine, xanthine 7-methylguanine, 5,6-dihydrouracil, 5-methylcytosine, 5-hydroxymethylcytosine) into a standard nucleobase (e.g., A, C, G, T, U). In some embodiments, a base editing enzyme is capable of converting a modified nucleobase into a different modified nucleobase.

In some embodiments, a base editing enzyme converts a target base pair. For example, in some embodiments, a base editing enzyme converts C-G base pairs to T-A base pairs. In some embodiments, a base editing enzyme converts A-T base pairs to G-C base pairs.

In some embodiments, a base editing enzyme is a deaminase (e.g., cytidine deaminase or adenosine deaminase) that is capable of removing an amino group from a molecule. Cytidine deaminases are capable of removing an amino group from a cytidine; adenosine deaminases are capable of removing an amino group from an adenosine. A cytidine deaminase may be an apolipoprotein B mRNA editing enzyme complex (APOBEC1) family protein. In some embodiments, the deaminase is an APOBEC1 polypeptide, an APOBEC2 polypeptide, an APOBEC3 polypeptide, an APOBEC3A polypeptide, an APOBEC3B polypeptide, an APOBEC3C polypeptide, an APOBEC3D polypeptide, an APOBEC3E polypeptide, an APOBEC3F polypeptide, an APOBEC3G deaminase polypeptide, an APOBEC3H polypeptide, an APOBEC4 polypeptide, or an activation-induced deaminase (AID). In some embodiments, an adenosine deaminase is a TadA polypeptide.

In some embodiments, a base editing enzyme is an oxidase (e.g., a guanine oxidase) that is capable of oxidizing a certain nucleobase. For example, a guanine oxidase functions to oxidize a certain guanine nucleobase in a target gene to form 8-oxoguanine (8-oxo-G). 8-oxo-G induces steric rotation of the nucleobase around the glycosidic bond, forcing base pairing in the Hoogsteen orientation of 8-oxo-G. Cellular recognition of the mismatched 8-oxo-G/cytosine paring leads to natural repair of the cytosine to an adenine. After an additional replication or mismatch repair, the 8-oxo-G is converted to a thymine, thereby producing a guanine-to-thymine conversion. In some embodiments, a guanine oxidase is a wild-type guanine oxidase, a xanthine dehydrogenase (XHD), a cytochrome P450 enzyme (e.g., CYP1A2, CYP2A6 or CYP3A6), a TET-oxidase (e.g., TET1, TET 1-CD, TET2 or TET3), an alpha-ketoglutarate-dependent hydroxylase (e.g., AlkB). In some embodiments, a xanthine dehydrogenase is a Streptomyces cyanogenus xanthine dehydrogenase, C. capitata xanthine dehydrogenase, N. crassa xanthine dehydrogenase, M. hansupus xanthine dehydrogenase, E. cloacae xanthine dehydrogenase, S. snoursei xanthine dehydrogenase, S. albulus xanthine dehydrogenase, S. himastatinicus xanthine dehydrogenase, or a S. lividans xanthine dehydrogenase.

In some embodiments, a base editing enzyme is a methyltransferase (e.g., a guanine methyltransferase) that is capable of methylating a certain nucleobase. For example, a guanine methyltransferase functions to methylate a certain guanine nucleobase in a target gene to form N2,N2-dimethyl-guanine or N-methyl-guanine. These methylated bases disrupt the hydrogen bonding interactions with the paired cytosine. Cellular recognition of the mismatched pairing leads to natural repair of the cytosine to an adenine. After an additional replication or mismatch repair, the methylated guanine is converted to a thymine, thereby producing a guanine-to-thymine conversion. In some embodiments, a guanine methyltransferase is a wild-type RlmA, Escherichia coli RlmA, human TrmTIOA, Escherichia coli TrmD, M. Jannaschii Trm5b, P. Abyssi Trm5a, a Trm5c from an archaeon, or a Staphylococcus scirui Cfr.

In some embodiments, a base editing enzyme (e.g., a deaminase) is a base editing enzyme derived from a human, chimpanzee, gorilla, monkey, cow, dog, rat, or mouse deaminase. In some embodiments, a base editing enzyme is a human base editing enzyme (e.g., a human deaminase, e.g., hAPOBEC polypeptide). In some embodiments, a base editing enzyme is a rat base editing enzyme (e.g., a rat deaminase, e.g., rAPOBEC1 polypeptide). In some embodiments, a base editing enzyme (e.g., a deaminase) is an evolved variant of a wild-type base editing enzyme. For example, in some embodiments, a base editing enzyme is an evolved APOBEC polypeptide (e.g., evoAPOBEC1 polypeptide), an evolved cytidine deaminase polypeptide (e.g., evoCDA polypeptide), or an evolved FERNY polypeptide (e.g., evoFERNY polypeptide). In some embodiments, a base editing enzyme is as described in Thuronyi, et. al. Nat Biotechnol. 2019 September; 37 (9): 1070-1079, the contents of which are incorporated herein by reference. In some embodiments, a base editing enzyme is as described in US20200172931, the contents of which are incorporated herein by reference.

Exemplary, non-limiting, base editing enzyme sequences are provided in Table 4. In some embodiments, a base editing enzyme comprises the amino acid sequence of any one of SEQ ID NOs: 27-69. In some embodiments, a base editing enzyme comprises an amino acid sequence that includes one or more mutation(s) (e.g., amino acid substitution(s)) relative to the amino acid sequence of any one of SEQ ID NOs: 27-69 and has at least 70% (e.g., at least 70%, at least 80%, at least 85%, at least 90%, at least 95%, or at least 98%) identity to the amino acid sequence of any one of the base editing enzymes in Table 4 (e.g., SEQ ID NOS: 120-162), an ortholog thereof, or other base editing enzyme sequence.

TABLE 4
Non-limiting Examples of Base Editing Enzyme Sequences

| Name | Sequence | SEQ ID NO: |
|---|---|---|
| rAPOBEC1 | SSETGPVAVDPTLRRRIEPHEFEVFFDPRELRKETCLLYEINWGGRHSIWRHTSQNTNKHVEVNF | 120 |
| | IEKFTTERYFCPNTRCSITWFLSWSPCGECSRAITEFLSRYPHVTLFIYIARLYHHADPRNRQGL | |
| | RDLISSGVTIQIMTEQESGYCWRNFVNYSPSNEAHWPRYPHLWVRLYVLELYCIILGLPPCLNIL | |
| | RRKQPQLTFFTIALQSCHYQRLPPHILWATGLK | |
| evoCDA | STDAEYVRIHEKLDIYTFKKQFSNNKKSVSHRCYVLFELKRRGERRACFWGYAVNKPQSGTERGI | 121 |
| | HAEIFSIRKVEEYLRDNPGQFTINWYSSWSPCADCAEKILEWYNQELRGNGHTLKIWVCKLYYEK | |
| | NARNQIGLWNLRDNGVGLNVMVSEHYQCCRKIFIQSSHNQLNENRWLEKTLKRAEKRRSELSIMF | |
| | QVKILHTTKSPAV | |
| evoFERNY | SFERNYDPRELRKETYLLYEIKWGKSGKLWRHWCQNNRTQHAEVYFLENIFNARRFNPSTHCSIT | 122 |
| | WYLSWSPCAECSQKIVDFLKEHPNVNLEIYVARLYYPENERNRQGLRDLVNSGVTIRIMDLPDYN | |
| | YCWKTFVSDQGGDEDYWPGHFAPWIKQYSLKL | |
| evoAPOBEC1 | SSKTGPVAVDPTLRRRIEPHEFEVFFDPRELRKETCLLYEINWGGRHSIWRHTSQNTNKHVEVNF | 123 |
| | IEKFTTERYFCPNTRCSITWFLSWSPCGECSRAITEFLSRYPNVTLFIYIARLYHLANPRNRQGL | |
| | RDLISSGVTIQIMTEQESGYCWHNFVNYSPSNESHWPRYPHLWVRLYVLELYCIILGLPPCLNIL | |
| | RRKQSQLTSFTIALQSCHYQRLPPHILWATGLK | |
| hAPOBEC3A | EASPASGPRHLMDPHIFTSNFNNGIGRHKTYLCYEVERLDNGTSVKMDQHRGFLHNQAKNLLCGF | 124 |
| | YGRHAELRFLDLVPSLQLDPAQIYRVTWFISWSPCFSWGCAGEVRAFLQENTHVRLRIFAARIYD | |
| | YDPLYKEALQMLRDAGAQVSIMTYDEFKHCWDTFVDHQGCPFQPWDGLDEHSQALSGRLRAILQN | |
| | QGN | |
| TadA | SEVEFSHEYWMRHALTLAKRARDEREVPVGAVLVLNNRVIGEGWNRAIGLHDPTAHAEIMALRQG | 125 |
| Sequence A | GLVMQNYRLIDATLYVTFEPCVMCAGAMIHSRIGRVVFGVRNSKRGAAGSLMNVLNYPGMNHRVE | |
| | ITEGILADECAALLCDFYRMPRQVENAQKKAQSSIN | |
| TadA | SEVEFSHEYWMRHALTLAKRAWDEREVPVGAVLVHNNRVIGEGWNRPIGRHDPTAHAEIMALRQG | 126 |
| Sequence B | GLVMQNYRLIDATLYVTLEPCVMCAGAMIHSRIGRVVFGARDAKTGAAGSLMDVLHHPGMNHRVE | |
| | ITEGILADECAALLSDFFRMRRQEIKAQKKAQSSTD | |
| E. coli TadA | RRAFITGVFFLSEVEFSHEYWMRHALTLAKRAWDEREVPVGAVLVHNNRVIGEGWNRPIGRHDPT | 127 |
| | AHAEIMALRQGGLVMQNYRLIDATLYVTLEPCVMCAGAMIHSRIGRVVFGARDAKTGAAGSLMDV | |
| | LHHPGMNHRVEITEGILADECAALLSDFFRMRRQEIKAQKKAQSSTD | |
| Staphylococcus | GSHMTNDIYFMTLAIEEAKKAAQLGEVPIGAIITKDDEVIARAHNLRETLQQPTAHAEHIAIERA | 128 |
| aureus TadA | AKVLGSWRLEGCTLYVTLEPCVMCAGTIVMSRIPRVVYGADDPKGGCSGSLMNLLQQSNFNHRAI | |
| | VDKGVLKEACSTLLTTFFKNLRANKKSTN | |
| Bacillus subtilis | TQDELYMKEAIKEAKKAEEKGEVPIGAVLVINGEIIARAHNLRETEQRSIAHAEMLVIDEACKAL | 129 |
| TadA | GTWRLEGATLYVTLEPCPMCAGAVVLSRVEKVVFGAFDPKGGCSGTLMNLLQEERFNHQAEVVSG | |
| | VLEEECGGMLSAFFRELRKKKKAARKNLS | |
| Salmonella | PPAFITGVTSLSDVELDHEYWMRHALTLAKRAWDEREVPVGAVLVHNHRVIGEGWNRPIGRHDPT | 130 |
| typhimurium TadA | AHAEIMALRQGGLVLQNYRLLDTTLYVTLEPCVMCAGAMVHSRIGRVVFGARDAKTGAAGSLIDV | |
| | LHHPGMNHRVEIIEGVLRDECATLLSDFFRMRRQEIKALKKADRAEGAGPAV | |
| Shewanella | DEYWMQVAMQMAEKAEAAGEVPVGAVLVKDGQQIATGYNLSISQHDPTAHAEILCLRSAGKKLEN | 131 |
| putrefaciens TadA | YRLLDATLYITLEPCAMCAGAMVHSRIARVVYGARDEKTGAAGTVVNLLQHPAFNHQVEVTSGVL | |
| | AEACSAQLSRFFKRRRDEKKALKLAQRAQQGIE | |
| Haemophilus | DAAKVRSEFDEKMMRYALELADKAEALGEIPVGAVLVDDARNIIGEGWNLSIVQSDPTAHAEIIA | 132 |
| influenzae F3031 | LRNGAKNIQNYRLLNSTLYVTLEPCTMCAGAILHSRIKRLVFGASDYKTGAIGSRFHFFDDYKMN | |
| TadA | HTLEITSGVLAEECSQKLSTFFQKRREEKKIEKALLKSLSDK | |
| Caulobacter | RTDESEDQDHRMMRLALDAARAAAEAGETPVGAVILDPSTGEVIATAGNGPIAAHDPTAHAEIAA | 133 |
| crescentus TadA | MRAAAAKLGNYRLTDLTLVVTLEPCAMCAGAISHARIGRVVFGADDPKGGAVVHGPKFFAQPTCH | |
| | WRPEVTGGVLADESADLLRGFFRRRKAKI | |
| Geobacter | SSLKKTPIRDDAYWMGKAIREAAKAAARDEVPIGAVIVRDGAVIGRGHNLREGSNDPSAHAEMIA | 134 |
| sulfurreducens | IRQAARRSANWRLTGATLYVTLEPCLMCMGAIILARLERVVFGCYDPKGGAAGSLYDLSADPRLN | |
| TadA | HQVRLSPGVCQEECGTMLSDFFRDLRRRKKAKATPALFIDERKVPPEP | |
| S. cyanogenus XDH | MSHLSERPEKPVVGVSMPHESAVQHVTGAALYTDDLVQRTKDVLHAYPVQVMKARGRVTALRTGA | 135 |
| | ALAVPGVVRVLTGADVPGVNDAGMKHDEPLFPDEVMFHGHAVAWVLGETLEAARIGAAAVEVDLE | |
| | ELPSVITLQDAIAADSYHGARPVMTHGDVDAGFADSAHVFTGEFQFSGQEHFYLETHAALAQVDE | |
| | NGQVFIQSSTQHPSETQEIVSHVLGVPAHEVTVQCLRMGGGFGGKEMQPHGFAAIAALGAKLTGR | |
| | PVRFRLNRTQDLTMSGKRHGFHATWKIGFDTEGRIQALDATLTADGGWSLDLSEPVLARALCHID | |
| | NTYWIPNARVAGRIARTNTVSNTAFRGFGGPQGMLVIEDILGRCAPRLGVDAKELRERNFYRPGQ | |
| | GQTTPYGQPVTQPERIAAVWQQVQDNGHIADREREIAAFNAAHPHTKRALAVTGVKFGISFNLTA | |
| | FNQGGALVLIYKDGSVLINHGGTEMGQGLHTKMLQVAATTLGIPLHKVRLAPTRTDKVPNTSATA | |
| | ASSGADLNGGAVKNACEQLRERLLRVAASQLGTNASDVRIVEGVARSLGSDQELAWDDLVRTAYF | |
| | QRVQLSAAGYYRTEGLHWDAKSFRGSPFKYFAIGAAATEVEVDGFTGAYRIRRVDIVHDVGDSLS | |
| | PLIDIGQVEGGFVQGAGWLTLEDLRWDTGDGPNRGRLLTQAASTYKLPSFSEMPEEFNVTLLENA | |
| | TEEGAVFGSKAVGEPPLMLAFSVREALRQAAAAFGPRGTAVELASPATPEAVYWAIESARQGGTA | |
| | GDGRTHGAAASDAVAVRTGVEALSGA | |
| C. capitata XDH | MTTNGNSFIVPVEKESPLIFFVNGKKVIDPTPDPECTLLTYLREKLRLCGTKLGCGEGGCGACTV | 136 |
| | MLSRVDRATNSVKHLAVNACLMPVCAMHGCAVTTIEGIGSTRTRLHPVQERLAKAHGSQCGFCTP | |
| | GIVMSMYALLRSMPLPSMKDLEVAFQGNLCRCTGYRPILEGYKTFTKEFSCGMGEKCCKLQSNGN | |
| | DVEKNGDDKLFERSAFLPFDPSQEPIFPPELHLNSQFDAENLLFKGPRSTWYRPVELSDLLKLKS | |
| | ENPHGKIIVGNTEVGVEMKFKQFLYTVHINPIKVPELNEMQELEDSILFGSAVTLMDIEEYLRER | |
| | IAKLPEHETRFFRCAVKMLHYFAGKQIRNVASLGGNIMTGSPISDMNPILTAACAKLKVCSLVEG | |
| | RIETREVCMGPGFFTGYRKNTIQPHEVLVAIHFPKSKKDQHFVAFKQARRRDDDIAIVNAAVNVT | |
| | FESNTNIVRQIYMAFGGMAPTTVMVPKTSQIMAKQKWNRVLVERVSESLCAELPLAPTAPGGMIA | |
| | YRRSLVVSLFFKAYLAISQELVKSNVIEEDAIPEREQSGAAIFHTPILKSAQLFERVCVEQSTCD | |
| | PIGRPKVHASAFKQATGEAIYCDDIPRHENELYLALVLSTKAHAKIVSVDESDALKQAGVHAFFS | |
| | SKDITEYENKVGSVFHDEEVFASERVYCQGQVIGAIVADSQVLAQRAARLVHIKYEELTPVIITI | |
| | EQAIKHKSYFPNYPQYIVQGDVATAFEEADHVYENSCRMGGQEHFYLETNACVATPRDSDEIELF | |
| | CSTQNPTEVQKLVAHVLSVPCHRVVCRSKRLGGGFGGKESRSIILALPVALASYRLRRPVRCMLD | |
| | RDEDMMTTGTRHPFLFKYKVGFTKEGLITACDIECYNNAGCSMDLSFSVLDRAMNHFENCYRIPN | |
| | VKVAGWVCRTNLPSNTAFRGFGGPQGMFAAEHIVRDVARIVGKDYLDIMQMNFYKTGDYTHYNQK | |
| | LENFPIEKCFTDCLNQSEFHKKRLAIEEFNKKNRWRKRGIALVPTKYGIAFGAMHLNQAGALINI | |
| | YGDGSVLLSHGGVEIGQGLHTKMIQCCARALGIPTELIHIAETATDKVPNTSPTAASVGSDINGM | |
| | AVLDACEKLNQRLKPIREANPKATWQECISKAYFDRISLSASGFYKMPDVGDDPKTNPNARTYNY | |
| | FTNGVGVSVVEIDCLTGDHQVLSTDIVMDIGSSLNPAIDIGQIEGAFMQGYGLFVLEELIYSPQG | |
| | ALYSRGPGMYKLPGFADIPGEFNVSLLTGAPNPRAVYSSKAVGEPPLFIGSTVFFAIKQAIAAAR | |
| | AERGLSITFELDAPATAARIRMACQDEFTDLIEQPSPGTYTPWNVVP | |
| N. crassa XDH | MTTNGNSFIVPVEKESPLIFFVNGKKVIDPTPDPECTLLTYLREKLRLCGTKLGCGEGGCGACTV | 137 |
| | MLSRVDRATNSVKHLAVNACLMPVCAMHGCAVTTIEGIGSTRTRLHPVQERLAKAHGSQCGFCTP | |
| | GIVMSMYALLRSMPLPSMKDLEVAFQGNLCRCTGYRPILEGYKTFTKEFSCGMGEKCCKLQSNGN | |
| | DVEKNGDDKLFERSAFLPFDPSQEPIFPPELHLNSQFDAENLLFKGPRSTWYRPVELSDLLKLKS | |
| | ENPHGKIIVGNTEVGVEMKFKQFLYTVHINPIKVPELNEMQELEDSILFGSAVTLMDIEEYLRER | |
| | IAKLPEHETRFFRCAVKMLHYFAGKQIRNVASLGGNIMTGSPISDMNPILTAACAKLKVCSLVEG | |
| | RIETREVCMGPGFFTGYRKNTIQPHEVLVAIHFPKSKKDQHFVAFKQARRRDDDIAIVNAAVNVT | |
| | FESNTNIVRQIYMAFGGMAPTTVMVPKTSQIMAKQKWNRVLVERVSESLCAELPLAPTAPGGMIA | |
| | YRRSLVVSLFFKAYLAISQELVKSNVIEEDAIPEREQSGAAIFHTPILKSAQLFERVCVEQSTCD | |
| | PIGRPKVHASAFKQATGEAIYCDDIPRHENELYLALVLSTKAHAKIVSVDESDALKQAGVHAFFS | |
| | SKDITEYENKVGSVFHDEEVFASERVYCQGQVIGAIVADSQVLAQRAARLVHIKYEELTPVIITI | |
| | EQAIKHKSYFPNYPQYIVQGDVATAFEEADHVYENSCRMGGQEHFYLETNACVATPRDSDEIELF | |
| | CSTQNPTEVQKLVAHVLSVPCHRVVCRSKRLGGGFGGKESRSIILALPVALASYRLRRPVRCMLD | |
| | RDEDMMTTGTRHPFLFKYKVGFTKEGLITACDIECYNNAGCSMDLSFSVLDRAMNHFENCYRIPN | |
| | VKVAGWVCRTNLPSNTAFRGFGGPQGMFAAEHIVRDVARIVGKDYLDIMQMNFYKTGDYTHYNQK | |
| | LENFPIEKCFTDCLNQSEFHKKRLAIEEFNKKNRWRKRGIALVPTKYGIAFGAMHLNQAGALINI | |
| | YGDGSVLLSHGGVEIGQGLHTKMIQCCARALGIPTELIHIAETATDKVPNTSPTAASVGSDINGM | |
| | AVLDACEKLNQRLKPIREANPKATWQECISKAYFDRISLSASGFYKMPDVGDDPKTNPNARTYNY | |
| | FTNGVGVSVVEIDCLTGDHQVLSTDIVMDIGSSLNPAIDIGQIEGAFMQGYGLFVLEELIYSPQG | |
| | ALYSRGPGMYKLPGFADIPGEFNVSLLTGAPNPRAVYSSKAVGEPPLFIGSTVFFAIKQAIAAAR | |
| | AERGLSITFELDAPATAARIRMACQDEFTDLIEQPSPGTYTPWNVVP | |
| M. Hansupus XDH | MSNMFEFRLNGATVRVDGVSPNTTLLDFLRNRGLTGTKQGCAEGDCGACTVALVDRDAQGNRCLR | 138 |
| | AFNACIALVPMVAGRELVTVEGVGSSEKPHPVQQAMVKHYGSQCGFCTPGFIVSMAEGYSRKDVC | |
| | TPSSVADQLCGNLCRCTGYRPIRDAMMEALAERDADASPATAIPSAPLGGPAEPLSALHYEATGQ | |
| | TFLRPTSWKELLDLRARHPEAHLVAGATELGVDITKKARRFPFLISTEGVESLREVRREKDCWYV | |
| | GGAASLVALEEALGDALPEVTKMLNVFASRQIRQRATLAGNLVTASPIGDMAPVLLALDARLVLG | |
| | SVRGERTVALSEFFLAYRKTALQADEVVRHIVIPHPAVPERGQRLSDSFKVSKRRELDISIVAAG | |
| | FRVELDAHGVVSLARLGYGGVAATPVRAVRAEAALTGQPWTRETVDQVLPVLAEEITPISDQRGS | |
| | AEYRRGLVAGLFEKFFAGTYSPVLDAAPGFEKGDAQVPADAGRALRHESAMGHVTGSARYVDDFA | |
| | QRQPMFEVWPVCAPHAHARIFKRDPTAARKVPGVVRVFMAEDIPGTNDTGPIRHDEPFFADREVF | |
| | FHGQIVAFVVGESVEACRAGARAVEVEYEPFPAIFTVEDAMAQGSYHTEPHVIRRGDVDAAFASS | |
| | PHRFSGTMAIGGQEHFYFETQAAFAERGDDGDITVVSSTQHPSEVQAIISHVFHFPRSRVVVKSP | |
| | RMGGGFGGKETQGNSPAAFVAFASWHTGRPTRWMMDRDVDMVVTGKRHPFHAAYEVGFDDEGKFF | |
| | AFRVQFVSNGGWSFDFSESETDRAFFHEDNAYYVPAFTYTGRVAKTHFVSNTAFRGFGGPQGMLV | |
| | TEEVLAHVARSVGVPADVVRERNLYRGTGETNTTHYGQELEDERIHRVWEELKRTSDFEQRRAEV | |
| | DAFNARSPFIKRGLAITPMKFGISFTATFLNQAGALVHLYRDGSVMVSHGGTEMGQGFHTKVQGV | |
| | AMREFGVEASAVRIAKTATDKVPNTSATAASSGSDENGAAVRFACITFRERFAPVAVRFFADRHG | |
| | RTVAPEAFFFSEGKVGFRGEPEVSLPFANVVEAAYLARVGLSATGYYQTPGIGYDKAKGRGRPFL | |
| | YFAYGASVCEVEVDGHTGVKRVLRVDLLEDVGDSLNPGVDRGQIEGGFVQGLGWLTGEELRWDAN | |
| | GRLLTHSASTYAVPAFSDAPIDFRVRLLERAHQHNTIHGSKAVGEPPLMLAMSAREALRDAVGAF | |
| | GQAGGGVALASPATHEALFLAIQKRLSRGAREDGREAA | |
| E. cloacae XDH | MKFDKPATTNPIDTLRVVGQPHTRIDGPRKTTGSAHYAYEWHDIAPNAAYGH | 139 |
| | VVGAPIAKGRITAIDTKAAEAAPGVLAVITADNAGPLGKGEKNTATLLGGPEIEHYHQAVALVVA | |
| | ETFEQARAAAALVKVTCKRAQGAYDLAAEKASVTEPPEDTPDKNVGDVATAFASAAVKLDAIYTT | |
| | PDQSHMAMEPHASMAVWEGDNVTVWTSNQMIDWCRTDLALTLKIPPENVRIVSPYIGGGFGGKLF | |
| | LRSDALLAALGARAVKRPVKVMLPRPTIPNNTTHRPATLQHIRIGTDTEGKIVAIAHDSWSGNLP | |
| | GGTPETAVQQTELLYAGANRHTGLRLATLDLPEGNAMRAPGEAPGLMALEIAIDEIADKAGVDPV | |
| | AFRILNDTQVDPANPERRFSRRQLVECLQTGAERFGWQKRHAQPGQVRDGRWLVGMGMAAGFRNN | |
| | LVATSGARVHLNADGSVAVETDMTDIGTGSYTIIAQTAAEMLGLPLEKVDVRLGDSRFPVSAGSG | |
| | GQWGANTSTAGVYAACVKLREAIARQLGFDPATAEFADETISAQGRSAPLAEAAKSGVLTAEDSI | |
| | EFGDLDKEYQQSTFAGHFVEVGVDSATGEVRVRRMLAVCAAGRILNPITARSQVIGAMTMGLGAA | |
| | LMEELAVDTRLGYFVNHDMAAYEVPVHADIPEQEVIFLEDTDPISSPMKAKGVGELGLCGVSAAI | |
| | ANAIYNATGVRVRDYPITLDKLIDALPDAV | |
| S. snoursei XDH | MSHDPVPHLPPAAPLPHPLGAPSVRREGREKVTGAARYAAEHTPPGCAYAWPVPATVARGRITEL | 140 |
| | DTAAALALPGVIAVLTHENAPRLASTGDPTLAVLQEDRVPHRGWYVALAVADTLEAARDAAEAVH | |
| | VGYATEPHDVRITADHPRLYVPEEVFGGPGARERGDFDAAFAAAPATVDVAYTVPPLHNHPMEPH | |
| | AATAQWTDGHLTVHDSSQGATRVCEDLAALFKLGTDEITVVSEHVGGGFGAKGTPRPQVVLAAMA | |
| | ARHTGRPVKLALPRRQLPGVVGHRAPTLHRVRIGAGHDGVITALAHEIVTHTSTVTEFVEQAAIP | |
| | ARMMYTSPHSRTVHRLAALDVPTPSWMRAPGEAPGMYALESALDELAVVLDIDPVELRIRNDPAT | |
| | EPDTGRPFSSRHLVECLRAGAERFGWLPRDPRPAVRRRGDLLLGTGVAAATYPVQISETEAEAHA | |
| | AADGGYRIRVNATDIGTGARTVLTQIAAAVLGAPEDRVRVDIGSSDLPPAVLAGGSTGTASWGWA | |
| | VHKACTSLLARLRAHHGPLPAEGIMAELSEWAPMALRAWRIISGLGLPTKYGSTPVALVMRAATE | |
| | PVAGSGPSVEGPVSSGLVAMKRAPFSMSRMALVSASKL | |
| S. albulus XDH | MTPPPTTRTRAMSHPPEEAPFPPGPPPHPLGDPLVRREGREKVTGTARYAAEHTPDGCAYAWPVP | 141 |
| | ATVVRGRITELDTGAALALPGVIAVLTHENAPRLAPTGDPTLALLQEDRVPHRGWYVALAVADTL | |
| | EAARDAAEAVHVSYATEPHDVTLTADHPRLYVPAEVFGGPGARERGDFDTAFAAAPATVDVTYTV | |
| | PPLHNHPMEPHAATALWTHGHLTVHDSSQGATRVREDLAALFKLGQDQITVHSEHVGGGFGSKGT | |
| | PRPQVVLAAMAARHTGRPVKLALPRRHLPAVVGHRAPTLHRVRLGAGPDGVITALAHEIVTHTST | |
| | VAEFVEQAAMPARIMYTSPHSRTVHRLAALDVPTPSWMRAPGEAPGMYALESAVDELAVVLDLDP | |
| | IDLRIRNEPGTEPDTGRPFSSRHLVDCLRAGAARFGWSSRDPRPAVRRQGDLLLGTGVAAATYPV | |
| | QISATDAEAHAAADGTFRVRVNATDIGTGARTVLAQIAAAALGAPADRVRVEIGSSDLPPAVLAG | |
| | GSTGTASWGWAVHKACTVLLARLREHRGPLPAEGVTVTEDTRRETEQPSPYSRHAFGAVFAEVQV | |
| | DTRTGEVRARRLLGQYAAGHILNPRTARSQFVGGMVMGLGMALTEDSALDPVYGDFTARDLAAYH | |
| | VPACADVPAIEAHWLDEEDPHLNPMGSKGIGEIGIVGTPAAIGNAVWHATGVRLRDLPLTPDRIL | |
| | TARTVPLT | |
| S. himastatinicus | MTRVDGLDKVTGAATYAYEFPTPDVGYVWPVQATIARGRVTEVDGAPALARPGVLAVLDSGNAPR | 142 |
| XDH | LNTEAQAGPDLFVLQSPEVAYHGQIVAAVVATSLEAAREGAAAVRVSYEQEPHDVVLREDDERAQ | |
| | VAETVTDGSPGFVEHGDAEGALAAAPVRTEAMYTTPVEHTSPMEPHATIAAWDEDRLTLYNADQG | |
| | PFMSSQLLAAVFGLDQGAVEVVAEYIGGGFGSKGIPRSPAVLAALAAKHLGRPVKIALTRQQMFQ | |
| | LIPYRAPTIQRIRLGAERDGRLTAIDHEVVQQRSAMAEFADQTGSSTRVMYAAPNIRTTVKTAPL | |
| | DVLTPAWFRAPGHTPGMFALESAMDELATELEIDPVELRIRNDTGVDPDSGKPFSSRGLVACLRE | |
| | GAARFDWALRDPKPGIRREGRWLVGTGVASAHHPDYVFPSSATARAEADGTFTVRVGAVDIGTGG | |
| | RTALTQLAADALGIPVERLRLEIGRASLGPAPFAGGSLGTASWGWAVDKACRALLAELDTYGGAV | |
| | PDGGLEVRADTTEDVELRASFSRHSFGAHFAQVRVDTDTGEIRVDRMLGVFAAGRIVNPKTARSQ | |
| | FVGAMTMGLSMALLEIGEVDPVFGDFANHDFAGYHVAANADVPKLEALWLDEQDDNPNPVRGKGI | |
| | GELGIVGAAAAVTNAFHHATGQRVRDLPIRVERSREALRAARAEAQKRGPGAAEQGKPVG | |
| S. lividans XDH | MSHLSERPEKPVVGVSMPHESAVQHVTGAALYTDDLVQRTKDVLHAYPVQVMKARGRVTALRTGA | 143 |
| | ALAVPGVVRVLTGADVPGVNDAGMKHDEPLFPDEVMFHGHAVAWVLGETLEAARIGAAAVEVDLE | |
| | ELPSVITLQDAIAADSYHGARPVMTHGDVDAGFADSAHVFTGEFQFSGQEHFYLETHAALAQVDE | |
| | NGQVFIQSSTQHPSETQEIVSHVFGVPAHEVTVQCFRMGGGFGGKEMQPHGFAAIAAFGAKFTGR | |
| | PVRFRFNRTQDFTMSGKRHGFHATWKIGFDTEGRIQAFDATFTADGGWSFDFSEPVFARAFCHID | |
| | NTYWIPNARVAGRIARTNTVSNTAFRGFGGPQGMFVIEDIFGRCAPRFGVDAKEFRERNFYRPGQ | |
| | GQTTPYGQPVTQPERIAAVWQQVQDNGHIADREREIAAFNAAHPHTKRAFAVTGVKFGISFNFTA | |
| | FNQGGAFVFIYKDGSVFINHGGTEMGQGFHTKMFQVAATTFGIPFHKVRFAPTRTDKVPNTSATA | |
| | ASSGADENGGAVKNACEQFRERFFRVAASQFGTNASDVRIVEGVARSFGSDQEFAWDDFVRTAYF | |
| | QRVQFSAAGYYRTEGFHWDAKSFRGSPFKYFAIGAAATEVEVDGFTGAYRIRRVDIVHDVGDSFS | |
| | PFIDIGQVEGGFVQGAGWFTFEDFRWDTGDGPNRGRFFTQAASTYKFPSFSEMPEEFNVTFFENA | |
| | TEEGAVFGSKAVGEPPFMFAFSVREAFRQAAAAFGPRGTAVEFASPATPEAVYWAIESARQGGTA | |
| | GDGRTHGAAASDAVAVRTGVEAESGA | |
| Cytochrome P1A2 | MLASGMLLVALLVCLTVMVLMSVWQQRKSKGKLPPGPTPLPFIGNYLQLNTEQMYNSLMKISERY | 144 |
| | GPVFTIHLGPRRVVVLCGHDAVREALVDQAEEFSGRGEQATFDWVFKGYGVVFSNGERAKQLRRF | |
| | SIATLRDFGVGKRGIEERIQEEAGFLIDALRGTGGANIDPTFFLSRTVSNVISSIVFGDREDYKD | |
| | KEFLSLLRMMLGIFQFTSTSTGQLYEMFSSVMKHLPGPQQQAFQLLQGLEDFIAKKVEHNQRTLD | |
| | PNSPRDFIDSFLIRMQEEEKNPNTEFYLKNLVMTTLNLFIGGTETVSTTLRYGFLLLMKHPEVEA | |
| | KVHEEIDRVIGKNRQPKFEDRAKMPYMEAVIHEIQRFGDVIPMSLARRVKKDTKFRDFFLPKGTE | |
| | VFPMLGSVLRDPSFFSNPQDFNPQHFLNEKGQFKKSDAFVPFSIGKRNCFGEGLARMELFLFFTT | |
| | VMQNFRFKSSQSPKDIDVSPKHVGFATIPRNYTMSFFPR | |
| Cytochrome P2A6 | MLASGMLLVALLVCLTVMVLMSVWQQRKSKGKLPPGPTPLPFIGNYLQLNTEQMYNSLMKISERY | 145 |
| | GPVFTIHLGPRRVVVLCGHDAVREALVDQAEEFSGRGEQATFDWVFKGYGVVFSNGERAKQLRRF | |
| | SIATLRDFGVGKRGIEERIQEEAGFLIDALRGTGGANIDPTFFLSRTVSNVISSIVFGDREDYKD | |
| | KEFLSLLRMMLGIFQFTSTSTGQLYEMFSSVMKHLPGPQQQAFQLLQGLEDFIAKKVEHNQRTLD | |
| | PNSPRDFIDSFLIRMQEEEKNPNTEFYLKNLVMTTLNLFIGGTETVSTTLRYGFLLLMKHPEVEA | |
| | KVHEEIDRVIGKNRQPKFEDRAKMPYMEAVIHEIQRFGDVIPMSLARRVKKDTKFRDFFLPKGTE | |
| | VFPMLGSVLRDPSFFSNPQDFNPQHFLNEKGQFKKSDAFVPFSIGKRNCFGEGLARMELFLFFTT | |
| | VMQNFRLKSSQSPKDIDVSPKHVGFATIPRNYTMSFLPR | |
| Cytochrome P3A4 | MALIPDLAMETWLLLAVSLVLLYLYGTHSHGLFKKLGIPGPTPLPFLGNILSYHKGFCMEDMECH | 146 |
| | KKYGKVWGFYDGQQPVLAITDPDMIKTVLVKECYSVFTNRRPFGPVGFMKSAISIAEDEEWKRLR | |
| | SLLSPTFTSGKLKEMVPIIAQYGDVLVRNLRREAETGKPVTLKDVFGAYSMDVITSTSFGVNIDS | |
| | LNNPQDPFVENTKKLLRFDFLDPFFLSITVFPFLIPILEVLNICVFPREVINFLRKSVKRMKESR | |
| | LEDTQKHRVDFLQLMIDSQNSKETESHKALSDLELVAQSIIFIFAGYETTSSVLSFIMYELATHP | |
| | DVQQKLQEEIDAVLPNKAPPTYDTVLQMEYLDMVVNETLRLFPIAMRLERVCKKDVEINGMFIPK | |
| | GVVVMIPSYALHRDPKYWTEPEKFLPERFSKKNKDNIDPYIYTPFGSGPRNCIGMRFALMNMKLA | |
| | LIRVLQNFSFKPCKETQIPLKLSLGGLLQPEKPVVLKVESRDGTVSGA | |
| TET1 | MSRSRHARPSRLVRKEDVNKKKKNSQLRKTTKGANKNVASVKTLSPGKLKQLIQERDVKKKTEPK | 147 |
| | PPVPVRSLLTRAGAARMNLDRTEVLFQNPESLTCNGFTMALRSTSLSRRLSQPPLVVAKSKKVPL | |
| | SKGLEKQHDCDYKILPALGVKHSENDSVPMQDTQVLPDIETLIGVQNPSLLKGKSQETTQFWSQR | |
| | VEDSKINIPTHSGPAAEILPGPLEGTRCGEGLFSEETLNDTSGSPKMFAQDTVCAPFPQRATPKV | |
| | TSQGNPSIQLEELGSRVESLKLSDSYLDPIKSEHDCYPTSSLNKVIPDLNLRNCLALGGSTSPTS | |
| | VIKFLLAGSKQATLGAKPDHQEAFEATANQQEVSDTTSFLGQAFGAIPHQWELPGADPVHGEALG | |
| | ETPDLPEIPGAIPVQGEVFGTILDQQETLGMSGSVVPDLPVFLPVPPNPIATFNAPSKWPEPQST | |
| | VSYGLAVQGAIQILPLGSGHTPQSSSNSEKNSLPPVMAISNVENEKQVHISFLPANTQGFPLAPE | |
| | RGLFHASLGIAQLSQAGPSKSDRGSSQVSVTSTVHVVNTTVVTMPVPMVSTSSSSYTTLLPTLEK | |
| | KKRKRCGVCEPCQQKTNCGECTYCKNRKNSHQICKKRKCEELKKKPSVVVPLEVIKENKRPQREK | |
| | KPKVLKADFDNKPVNGPKSESMDYSRCGHGEEQKLELNPHTVENVTKNEDSMTGIEVEKWTQNKK | |
| | SQLTDHVKGDFSANVPEAEKSKNSEVDKKRTKSPKLFVQTVRNGIKHVHCLPAETNVSFKKFNIE | |
| | EFGKTLENNSYKFLKDTANHKNAMSSVATDMSCDHLKGRSNVLVFQQPGFNCSSIPHSSHSIINH | |
| | HASIHNEGDQPKTPENIPSKEPKDGSPVQPSLLSLMKDRRLTLEQVVAIEALTQLSEAPSENSSP | |
| | SKSEKDEESEQRTASLLNSCKAILYTVRKDLQDPNLQGEPPKLNHCPSLEKQSSCNTVVENGQTT | |
| | TLSNSHINSATNQASTKSHEYSKVINSLSLFIPKSNSSKIDTNKSIAQGIITLDNCSNDLHQLPP | |
| | RNNEVEYCNQLLDSSKKLDSDDLSCQDATHTQIEEDVATQLTQLASIIKINYIKPEDKKVESTPT | |
| | SLVTCNVQQKYNQEKGTIQQKPPSSVHNNHGSSLTKQKNPTQKKTKSTPSRDRRKKKPTVVSYQE | |
| | NDRQKWEKLSYMYGTICDIWIASKFQNFGQFCPHDFPTVFGKISSSTKIWKPLAQTRSIMQPKTV | |
| | FPPLTQIKLQRYPESAEEKVKVEPLDSLSLFHLKTESNGKAFTDKAYNSQVQLTVNANQKAHPLT | |
| | QPSSPPNQCANVMAGDDQIRFQQVVKEQLMHQRLPTLPGISHETPLPESALTLRNVNVVCSGGIT | |
| | VVSTKSEEEVCSSSFGTSEFSTVDSAQKNFNDYAMNFFTNPTKNLVSITKDSELPTCSCLDRVIQ | |
| | KDKGPYYTHLGAGPSVAAVREIMENRYGQKGNAIRIEIVVYTGKEGKSSHGCPIAKWVLRRSSDE | |
| | EKVLCLVRQRTGHHCPTAVMVVLIMVWDGIPLPMADRLYTELTENLKSYNGHPTDRRCTLNENRT | |
| | CTCQGIDPETCGASFSFGCSWSMYFNGCKFGRSPSPRRFRIDPSSPLHEKNLEDNLQSLATRLAP | |
| | IYKQYAPVAYQNQVEYENVARECRLGSKEGRPFSGVTACLDFCAHPHRDIHNMNNGSTVVCTLTR | |
| | EDNRSLGVIPQDEQLHVLPLYKLSDTDEFGSKEGMEAKIKSGAIEVLAPRRKKRTCFTQPVPRSG | |
| | KKRAAMMTEVLAHKIRAVEKKPIPRIKRKNNSTTTNNSKPSSLPTLGSNTETVQPEVKSETEPHF | |
| | ILKSSDNTKTYSLMPSAPHPVKEASPGFSWSPKTASATPAPLKNDATASCGFSERSSTPHCTMPS | |
| | GRLSGANAAAADGPGISQLGEVAPLPTLSAPVMEPLINSEPSTGVTEPLTPHQPNHQPSFLTSPQ | |
| | DLASSPMEEDEQHSEADEPPSDEPLSDDPLSPAEEKLPHIDEYWSDSEHIFLDANIGGVAIAPAH | |
| | GSVLIECARRELHATTPVEHPNRNHPTRLSLVFYQHKNLNKPQHGFELNKIKFEAKEAKNKKMKA | |
| | SEQKDQAANEGPEQSSEVNELNQIPSHKALTLTHDNVVTVSPYALTHVAGPYNHWV | |
| TET1 catalytic | MGSLPTCSCLDRVIQKDKGPYYTHLGAGPSVAAVREIMENRYGQKGNAIRIEIVVYTGKEGKSSH | 148 |
| domain | GCPIAKWVLRRSSDEEKVLCLVRQRTGHHCPTAVMVVLIMVWDGIPLPMADRLYTELTENLKSYN | |
| | GHPTDRRCTLNENRTCTCQGIDPETCGASFSFGCSWSMYFNGCKFGRSPSPRRFRIDPSSPLHEK | |
| | NLEDNLQSLATRLAPIYKQYAPVAYQNQVEYENVARECRLGSKEGRPFSGVTACLDFCAHPHRDI | |
| | HNMNNGSTVVCTLTREDNRSLGVIPQDEQLHVLPLYKLSDTDEFGSKEGMEAKIKSGAIEVLAPR | |
| | RKKRTCFTQPVPRSGKKRAAMMTEVLAHKIRAVEKKPIPRIKRKNNSTTTNNSKPSSLPTLGSNT | |
| | ETVQPEVKSETEPHFILKSSDNTKTYSLMPSAPHPVKEASPGFSWSPKTASATPAPLKNDATASC | |
| | GFSERSSTPHCTMPSGRLSGANAAAADGPGISQLGEVAPLPTLSAPVMEPLINSEPSTGVTEPLT | |
| | PHQPNHQPSFLTSPQDLASSPMEEDEQHSEADEPPSDEPLSDDPLSPAEEKLPHIDEYWSDSEHI | |
| | FLDANIGGVAIAPAHGSVLIECARRELHATTPVEHPNRNHPTRLSLVFYQHKNLNKPQHGFELNK | |
| | IKFEAKEAKNKKMKASEQKDQAANEGPEQSSEVNELNQIPSHKALTLTHDNVVTVSPYALTHVAG | |
| | PYNHWV | |
| TET2 | MEQDRTNHVEGNRLSPFLIPSPPICQTEPLATKLQNGSPLPERAHPEVNGDTKWHSFKSYYGIPC | 149 |
| | MKGSQNSRVSPDFTQESRGYSKCLQNGGIKRTVSEPSLSGLLQIKKLKQDQKANGERRNFGVSQE | |
| | RNPGESSQPNVSDLSDKKESVSSVAQENAVKDFTSFSTHNCSGPENPELQILNEQEGKSANYHDK | |
| | NIVLLKNKAVLMPNGATVSASSVEHTHGELLEKTLSQYYPDCVSIAVQKTTSHINAINSQATNEL | |
| | SCEITHPSHTSGQINSAQTSNSELPPKPAAVVSEACDADDADNASKLAAMLNTCSFQKPEQLQQQ | |
| | KSVFEICPSPAENNIQGTTKLASGEEFCSGSSSNLQAPGGSSERYLKQNEMNGAYFKQSSVFTKD | |
| | SFSATTTPPPPSQLLLSPPPPLPQVPQLPSEGKSTLNGGVLEEHHHYPNQSNTTLLREVKIEGKP | |
| | EAPPSQSPNPSTHVCSPSPMLSERPQNNCVNRNDIQTAGTMTVPLCSEKTRPMSEHLKHNPPIFG | |
| | SSGELQDNCQQLMRNKEQEILKGRDKEQTRDLVPPTQHYLKPGWIELKAPRFHQAESHLKRNEAS | |
| | LPSILQYQPNLSNQMTSKQYTGNSNMPGGLPRQAYTQKTTQLEHKSQMYQVEMNQGQSQGTVDQH | |
| | LQFQKPSHQVHFSKTDHLPKAHVQSLCGTRFHFQQRADSQTEKLMSPVLKQHLNQQASETEPFSN | |
| | SHLLQHKPHKQAAQTQPSQSSHLPQNQQQQQKLQIKNKEEILQTFPHPQSNNDQQREGSFFGQTK | |
| | VEECFHGENQYSKSSEFETHNVQMGFEEVQNINRRNSPYSQTMKSSACKIQVSCSNNTHFVSENK | |
| | EQTTHPEFFAGNKTQNFHHMQYFPNNVIPKQDFFHRCFQEQEQKSQQASVFQGYKNRNQDMSGQQ | |
| | AAQFAQQRYFIHNHANVFPVPDQGGSHTQTPPQKDTQKHAAFRWHFFQKQEQQQTQQPQTESCHS | |
| | QMHRPIKVEPGCKPHACMHTAPPENKTWKKVTKQENPPASCDNVQQKSIIETMEQHFKQFHAKSF | |
| | FDHKAFTFKSQKQVKVEMSGPVTVFTRQTTAAEFDSHTPAFEQQTTSSEKTPTKRTAASVENNFI | |
| | ESPSKFFDTPIKNFFDTPVKTQYDFPSCRCVEQIIEKDEGPFYTHFGAGPNVAAIREIMEERFGQ | |
| | KGKAIRIERVIYTGKEGKSSQGCPIAKWVVRRSSSEEKFFCFVRERAGHTCEAAVIVIFIFVWEG | |
| | IPFSFADKFYSEFTETFRKYGTFTNRRCAFNEERTCACQGFDPETCGASFSFGCSWSMYYNGCKF | |
| | ARSKIPRKFKFFGDDPKEEEKFESHFQNFSTFMAPTYKKFAPDAYNNQIEYEHRAPECRFGFKEG | |
| | RPFSGVTACFDFCAHAHRDFHNMQNGSTFVCTFTREDNREFGGKPEDEQFHVFPFYKVSDVDEFG | |
| | SVEAQEEKKRSGAIQVFSSFRRKVRMFAEPVKTCRQRKFEAKKAAAEKLSSLENSSNKNEKEKSA | |
| | PSRTKQTENASQAKQLAELLRLSGPVMQQSQQPQPLQKQPPQPQQQQRPQQQQPHHPQTESVNSY | |
| | SASGSTNPYMRRPNPVSPYPNSSHTSDIYGSTSPMNFYSTSSQAAGSYLNSSNPMNPYPGLLNQN | |
| | TQYPSYQCNGNLSVDNCSPYLGSYSPQSQPMDLYRYPSQDPLSKLSLPPIHTLYQPREGNSQSFT | |
| | SKYLGYGNQNMQGDGFSSCTIRPNVHHVGKLPPYPTHEMDGHFMGATSRLPPNLSNPNMDYKNGE | |
| | HHSPSHIIHNYSAAPGMFNSSLHALHLQNKENDMLSHTANGLSKMLPALNHDRTACVQGGLHKLS | |
| | DANGQEKQPLALVQGVASGAEDNDEVWSDSEQSFLDPDIGGVAVAPTHGSILIECAKRELHATTP | |
| | LKNPNRNHPTRISLVFYQHKSMNEPKHGLALWEAKMAEKAREKEEECEKYGPDYVPQKSHGKKVK | |
| | REPAEPHETSEPTYLRFIKSLAERTMSVTTDSTVTTSPYAFTRVTGPYNRYI | |
| TET3 | MDSGPVYHGDSRQLSASGVPVNGAREPAGPSLLGTGGPWRVDQKPDWEAAPGPAHTARLEDAHDL | 150 |
| | VAFSAVAEAVSSYGALSTRLYETFNREMSREAGNNSRGPRPGPEGCSAGSEDLDTLQTALALARH | |
| | GMKPPNCNCDGPECPDYLEWLEGKIKSVVMEGGEERPRLPGPLPPGEAGLPAPSTRPLLSSEVPQ | |
| | ISPQEGLPLSQSALSIAKEKNISLQTAIAIEALTQLSSALPQPSHSTPQASCPLPEALSPPAPER | |
| | SPQSYLRAPSWPVVPPEEHSSFAPDSSAFPPATPRTEFPEAWGTDTPPATPRSSWPMPRPSPDPM | |
| | AELEQLLGSASDYIQSVFKRPEALPTKPKVKVEAPSSSPAPAPSPVLQREAPTPSSEPDTHQKAQ | |
| | TALQQHLHHKRSLFLEQVHDTSFPAPSEPSAPGWWPPPSSPVPRLPDRPPKEKKKKLPTPAGGPV | |
| | GTEKAAPGIKPSVRKPIQIKKSRPREAQPLFPPVRQIVLEGLRSPASQEVQAHPPAPLPASQGSA | |
| | VPLPPEPSLALFAPSPSRDSLLPPTQEMRSPSPMTALQPGSTGPLPPADDKLEELIRQFEAEFGD | |
| | SFGLPGPPSVPIQDPENQQTCLPAPESPFATRSPKQIKIESSGAVTVLSTTCFHSEEGGQEATPT | |
| | KAENPLTPTLSGFLESPLKYLDTPTKSLLDTPAKRAQAEFPTCDCVEQIVEKDEGPYYTHLGSGP | |
| | TVASIRELMEERYGEKGKAIRIEKVIYTGKEGKSSRGCPIAKWVIRRHTLEEKLLCLVRHRAGHH | |
| | CQNAVIVILILAWEGIPRSLGDTLYQELTDTLRKYGNPTSRRCGLNDDRTCACQGKDPNTCGASF | |
| | SFGCSWSMYFNGCKYARSKTPRKFRLAGDNPKEEEVLRKSFQDLATEVAPLYKRLAPQAYQNQVT | |
| | NEEIAIDCRLGLKEGRPFAGVTACMDFCAHAHKDQHNLYNGCTVVCTLTKEDNRCVGKIPEDEQL | |
| | HVLPLYKMANTDEFGSEENQNAKVGSGAIQVLTAFPREVRRLPEPAKSCRQRQLEARKAAAEKKK | |
| | IQKEKLSTPEKIKQEALELAGITSDPGLSLKGGLSQQGLKPSLKVEPQNHFSSFKYSGNAWESYS | |
| | VLGNCRPSDPYSMNSVYSYHSYYAQPSLTSVNGFHSKYALPSFSYYGFPSSNPVFPSQFLGPGAW | |
| | GHSGSSGSFEKKPDLHALHNSLSPAYGGAEFAELPSQAVPTDAHHPTPHHQQPAYPGPKEYLLPK | |
| | APLLHSVSRDPSPFAQSSNCYNRSIKQEPVDPLTQAEPVPRDAGKMGKTPLSEVSQNGGPSHLWG | |
| | QYSGGPSMSPKRTNGVGGSWGVFSSGESPAIVPDKLSSFGASCLAPSHFTDGQWGLFPGEGQQAA | |
| | SHSGGRLRGKPWSPCKFGNSTSALAGPSLTEKPWALGAGDFNSALKGSPGFQDKLWNPMKGEEGR | |
| | IPAAGASQLDRAWQSFGLPLGSSEKLFGALKSEEKLWDPFSLEEGPAEEPPSKGAVKEEKGGGGA | |
| | EEEEEELWSDSEHNFLDENIGGVAVAPAHGSILIECARRELHATTPLKKPNRCHPTRISLVFYQH | |
| | KNLNQPNHGLALWEAKMKQLAERARARQEEAARLGLGQQEAKLYGKKRKWGGTVVAEPQQKEKKG | |
| | VVPTRQALAVPTDSAVTVSSYAYTKVTGPYSRWI | |
| E. coli AlkB | MLDLFADAEPWQEPLAAGAVILRRFAFNAAEQLIRDINDVASQSPFRQMVTPGGYTMSVAMTNCG | 151 |
| | HLGWTTHRQGYLYSPIDPQTNKPWPAMPQSFHNLCQRAATAAGYPDFQPDACLINRYAPGAKLSL | |
| | HQDKDEPDLRAPIVSVSLGLPAIFQFGGLKRNDPLKRLLLEHGDVVVWGGESRLFYHGIQPLKAG | |
| | FHPLTIDCRYNLTFRQAGKKE | |
| Human ABH3 | MEEKRRRARVQGAWAAPVKSQAIAQPATTAKSHLHQKPGQTWKNKEHHLSDREFVFKEPQQVVRR | 152 |
| | APEPRVIEEGVYEISLSPTGVSRVCLYPGFVDVKEADWILEQLCQDVPWKQRTGIREDSILQLTF | |
| | KKSAPVSGTATAPQSCWYERPSPPHIPGPAILTRTRLWAP | |
| E. coli GMP | MTENIHKHRILILDFGSQYTQLVARRVRELGVYCELWAWDVTEAQIRDENPSGIILSGGPESTTE | 153 |
| Synthase | ENSPRAPQYVFEAGVPVFGVCYGMQTMAMQLGGHVEASNEREFGYAQVEVVNDSALVRGIEDALT | |
| | ADGKPLLDVWMSHGDKVTAIPSDFITVASTESCPFAIMANEEKRFYGVQFHPEVTHTRQGMRMLE | |
| | RFVRDICQCEALWTPAKIIDDAVARIREQVGDDKVILGLSGGVDSSVTAMLLHRAIGKNLTCVFV | |
| | DNGLLRLNEAEQVLDMFGDHFGLNIVHVPAEDRFLSALAGENDPEAKRKIIGRVFVEVFDEEALK | |
| | LEDVKWLAQGTIYPDVIESAASATGKAHVIKSHHNVGGLPKEMKMGLVEPLKELFKDEVRKIGLE | |
| | LGLPYDMLYRHPFPGPGLGVRVLGEVKKEYCDLLRRADAIFIEELRKADLYDKVSQAFTVFLPVR | |
| | SVGVMGDGRKYDWVVSLRAVETIDFMTAHWAHLPYDFLGRVSNRIINEVNGISRVVYDISGKPPA | |
| | TIEWE | |
| S. scirui Cfr | MNFNNKTKYGKIQEFLRSNNEPDYRIKQITNAIFKQRISRFEDMKVLPKLLREDLINNFGETVLN | 154 |
| | IKLLAEQNSEQVTKVLFEVSKNERVETVNMKYKAGWESFCISSQCGCNFGCKFCATGDIGLKKNL | |
| | TVDEITDQVLYFHLLGHQIDSISFMGMGEALANRQVFDALDSFTDPNLFALSPRRLSISTIGIIP | |
| | SIKKITQEYPQVNLTFSLHSPYSEERSKLMPINDRYPIDEVMNILDEHIRLTSRKVYIAYIMLPG | |
| | VNDSLEHANEVVSLLKSRYKSGKLYHVNLIRYNPTISAPEMYGEANEGQVEAFYKVLKSAGIHVT | |
| | IRSQFGIDIDAACGQLYGNYQNSQ | |
| A. aeolicus Trm1 | MEIVQEGIAKIIVPEIPKTVSSDMPVFYNPRMRVNRDLAVLGLEYLCKKLGRPVKVADPLSASGI | 155 |
| | RAIRFLLETSCVEKAYANDISSKAIEIMKENFKLNNIPEDRYEIHGMEANFFLRKEWGFGFDYVD | |
| | LDPFGTPVPFIESVALSMKRGGILSLTATDTAPLSGTYPKTCMRRYMARPLRNEFKHEVGIRILI | |
| | KKVIELAAQYDIAMIPIFAYSHLHYFKLFFVKERGVEKVDKLIEQFGYIQYCFNCMNREVVTDLY | |
| | KFKEKCPHCGSKFHIGGPLWIGKLWDEEFTNFLYEEAQKREEIEKETKRILKLIKEESQLQTVGF | |
| | YVLSKLAEKVKLPAQPPIRIAVKFFNGVRTHFVGDGFRTNLSFEEVMKKMEELKEKQKEFLEKKK | |
| | QG | |
| S. cerevisiae Trm1 | MEGFFRIPLKRANLHGMLKAAISKIKANFTAYGAPRINIEDFNIVKEGKAEILFPKKETVFYNPI | 156 |
| | QQFNRDLSVTCIKAWDNLYGEECGQKRNNKKSKKKRCAETNDDSSKRQKMGNGSPKEAVGNSNRN | |
| | EPYINILEALSATGLRAIRYAHEIPHVREVIANDLLPEAVESIKRNVEYNSVENIVKPNLDDANV | |
| | LMYRNKATNNKFHVIDLDPYGTVTPFVDAAIQSIEEGGLMLVTCTDLSVLAGNGYPEKCFALYGG | |
| | ANMVSHESTHESALRLVLNLLKQTAAKYKKTVEPLLSLSIDFYVRVFVKVKTSPIEVKNVMSSTM | |
| | TTYHCSRCGSYHNQPLGRISQREGRNNKTFTKYSVAQGPPVDTKCKFCEGTYHLAGPMYAGPLHN | |
| | KEFIEEVLRINKEEHRDQDDTYGTRKRIEGMLSLAKNELSDSPFYFSPNHIASVIKLQVPPLKKV | |
| | VAGLGSLGFECSLTHAQPSSLKTNAPWDAIWYVMQKCDDEKKDLSKMNPNTTGYKILSAMPGWLS | |
| | GTVKSEYDSKLSFAPNEQSGNIEKLRKLKIVRYQENPTKNWGPKARPNTS | |
| Human TRM1 | MQGSSLWLSLTFRSARVLSRARFFEWQSPGLPNTAAMENGTGPYGEERPREVQETTVTEGAAKIA | 157 |
| | FPSANEVFYNPVQEFNRDLTCAVITEFARIQLGAKGIQIKVPGEKDTQKVVVDLSEQEEEKVELK | |
| | ESENLASGDQPRTAAVGEICEEGLHVLEGLAASGLRSIRFALEVPGLRSVVANDASTRAVDLIRR | |
| | NVQLNDVAHLVQPSQADARMLMYQHQRVSERFDVIDLDPYGSPATFLDAAVQAVSEGGLLCVTCT | |
| | DMAVLAGNSGETCYSKYGAMALKSRACHEMALRIVLHSLDLRANCYQRFVVPLLSISADFYVRVF | |
| | VRVFTGQAKVKASASKQALVFQCVGCGAFHLQRLGKASGVPSGRAKFSAACGPPVTPECEHCGQR | |
| | HQLGGPMWAEPIHDLDFVGRVLEAVSANPGRFHTSERIRGVLSVITEELPDVPLYYTLDQLSSTI | |
| | HCNTPSLLQLRSALLHADFRVSLSHACKNAVKTDAPASALWDIMRCWERECPVKRERLSETSPAF | |
| | RILSVEPRLQANFTIREDANPSSRQRGLKRFQANPEANWGPRPRARPGGKAADEAMEERRRLLQN | |
| | KRKEPPEDVAQRAARLKTFPCKRFKEGTCQRGDQCCYSHSPPTPRVSADAAPDCPETSNQTPPGP | |
| | GAAAGPGID | |
| E. coli RlmA | MSFSCPLCHQPLSREKNSYICPQRHQFDMAKEGYVNLLPVQHKRSRDPGDSAEMMQARRAFLDAG | 158 |
| | HYQPLRDAIVAQLRERLDDKATAVLDIGCGEGYYTHAFADALPEITTFGLDVSKVAIKAAAKRYP | |
| | QVTFCVASSHRLPFSDTSMDAIIRIYAPCKAEELARVVKPGGWVITATPGPRHLMELKGLIYNEV | |
| | HLHAPHAEQLEGFTLQQSAELCYPMRLRGDEAVALLQMTPFAWRAKPEVWQTLAAKEVFDCQTDF | |
| | NIHLWQRSY | |
| E. coli TrmD | MWIGIISLFPEMFRAITDYGVTGRAVKNGLLSIQSWSPRDFTHDRHRTVDDRPYGGGPGMLMMVQ | 159 |
| | PLRDAIHAAKAAAGEGAKVIYLSPQGRKLDQAGVSELATNQKLILVCGRYEGIDERVIQTEIDEE | |
| | WSIGDYVLSGGELPAMTLIDSVSRFIPGVLGHEASATEDSFAEGLLDCPHYTRPEVLEGMEVPPV | |
| | LLSGNHAEIRRWRLKQSLGRTWLRRPELLENLALTEEQARLLAEFKTEHAQQQHKHDGMA | |
| Human TRMT10A | MSSEMLPAFIETSNVDKKQGINEDQEESQKPRLGEGCEPISKRQMKKLIKQKQWEEQRELRKQKR | 160 |
| | KEKRKRKKLERQCQMEPNSDGHDRKRVRRDVVHSTLRLIIDCSFDHLMVLKDIKKLHKQIQRCYA | |
| | ENRRALHPVQFYLTSHGGQLKKNMDENDKGWVNWKDIHIKPEHYSELIKKEDLIYLTSDSPNILK | |
| | ELDESKAYVIGGLVDHNHHKGLTYKQASDYGINHAQLPLGNFVKMNSRKVLAVNHVFEIILEYLE | |
| | TRDWQEAFFTILPQRKGAVPTDRACESASHDNQSVRMEEGGSDSDSSEEEYSRNELDSPHEEKQD | |
| | KENHTESTVNSLPH | |
| M. Jannaschii | MPLCLKINKKHGEQTRRILIENNLLNKDYKITSEGNYLYLPIKDVDEDILKSILNIEFELVDKEL | 161 |
| Trm5b | EEKKIIKKPSFREIISKKYRKEIDEGLISLSYDVVGDLVILQISDEVDEKIRKEIGELAYKLIPC | |
| | KGVFRRKSEVKGEFRVRELEHLAGENRTLTIHKENGYRLWVDIAKVYFSPRLGGERARIMKKVSL | |
| | NDVVVDMFAGVGPFSIACKNAKKIYAIDINPHAIELLKKNIKLNKLEHKIIPILSDVREVDVKGN | |
| | RVIMNLPKFAHKFIDKALDIVEEGGVIHYYTIGKDFDKAIKLFEKKCDCEVLEKRIVKSYAPREY | |
| | ILALDEKINKK | |
| P. Abyssi Trm5a | MTLAVKVPLKEGEIVRRRLIELGALDNTYKIKREGNFLLIPVKFPVKGFEVVEAELEQVSRRPNS | 162 |
| | YREIVNVPQELRRFLPTSFDIIGNIAIIEIPEELKGYAKEIGRAIVEVHKNVKAVYMKGSKIEGE | |
| | YRTRELIHIAGENITETIHRENGIRLKLDVAKVYFSPRLATERMRVFKMAQEGEVVFDMFAGVGP | |
| | FSILLAKKAELVFACDINPWAIKYLEENIKLNKVNNVVPILGDSREIEVKADRIIMNLPKYAHEF | |
| | LEHAISCINDGGVIHYYGFGPEGDPYGWHLERIRELANKFGVKVEVLGKRVIRNYAPRQYNIAID | |
| | FRVSF | |

In some embodiments, the fusion protein comprises one or more variant Cas12a endonuclease, one or more base editing enzymes, and one or more additional proteins (e.g., a protein, such as an enzyme, that regulates a biological activity). Non-limiting examples of additional protein elements include a polypeptide having uracil glycosylase inhibitor (UGI) activity, a polypeptide having uracil DNA glycosylase activity, a DNA binding domain (e.g., a Rad51 DNA binding domain), reverse transcriptase, endonuclease (e.g., FokI), a polypeptide having exonuclease activity (e.g., T5 exonuclease), a polypeptide having methyltransferase activity, a polypeptide having demethylase activity, a polypeptide having acetyltransferase activity, a polypeptide having deacetylase activity, a polypeptide having kinase activity, a polypeptide having phosphatase activity, a polypeptide having ubiquitin ligase activity, a polypeptide having deubiquitinating activity, a polypeptide having adenylation activity, a polypeptide having deadenylation activity, a polypeptide having SUMOylating activity, a polypeptide having deSUMOylating activity, a polypeptide having ribosylation activity, a polypeptide having deribosylation activity, a polypeptide having myristoylation activity, or a polypeptide having demyristoylation activity.

A fusion protein may comprise any one of the variant Cas12a endonucleases described herein (e.g., any one of SEQ ID NOs: 48-119 and 367-387, preferably any one of SEQ ID NOs: 367-387). In some embodiments, a fusion protein comprises any one of the base editing enzymes described herein.

In some embodiments, the variant Cas12a endonuclease comprises a polypeptide sequence that comprises a mutation at an amino acid position corresponding to position R833 with reference to amino acid position numbering of LbCas12a ND2006 (e.g., SEQ ID NO: 1). In some embodiments, the mutation is R833L, R833K, or R833M. In some embodiments, a variant LbCas12a endonuclease comprises a mutation at position R833 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position R833 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises a mutation at an amino acid position corresponding to position K932 with reference to amino acid position numbering of LbCas12a ND2006 (e.g., SEQ ID NO: 1). In some embodiments, the mutation is K932E or K932G. In some embodiments, a variant LbCas12a endonuclease comprises a mutation at position K932 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K932 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises a mutation at an amino acid position corresponding to position Q944 with reference to amino acid position numbering of LbCas12a ND2006 (e.g., SEQ ID NO: 1). In some embodiments, the mutation is Q944K. In some embodiments, a variant LbCas12a endonuclease comprises a mutation at position Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises a mutation at an amino acid position corresponding to position K940 with reference to amino acid position numbering of LbCas12a ND2006 (e.g., SEQ ID NO: 1). In some embodiments, the mutation is K940G. In some embodiments, a variant LbCas12a endonuclease comprises a mutation at position K940 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises a mutation at position K940 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions K932, N933, V936, and S929 with reference to amino acid position numbering of LbCas12a ND2006 (e.g., SEQ ID NO: 1). In some embodiments, the mutations are K932G, N933G, V936G, and S929G. In some embodiments, a variant LbCas12a endonuclease comprises mutations at positions K932, N933, V936, and S929 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K932, N933, V936, and S929 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions K940 and Q944 with reference to amino acid position numbering of LbCas12a ND2006 (e.g., SEQ ID NO: 1). In some embodiments, the mutations are K940G and Q944K. In some embodiments, a variant LbCas12a endonuclease comprises mutations at positions K940 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions K940 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R836 and Q944 with reference to amino acid position numbering of LbCas12a ND2006 (e.g., SEQ ID NO: 1). In some embodiments, the mutations are R836G and Q944K. In some embodiments, a variant LbCas12a endonuclease comprises mutations at positions R836 and Q944 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R836 and Q944 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, and Y943 with reference to amino acid position numbering of LbCas12a ND2006 (e.g., SEQ ID NO: 1). In some embodiments, the mutations are R833M, E835D, and Y943T. In some embodiments, a variant LbCas12a endonuclease comprises mutations at positions R833, E835, and Y943 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, and Y943 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R836, Q944, and R935 with reference to amino acid position numbering of LbCas12a ND2006 (e.g., SEQ ID NO: 1). In some embodiments, the mutations are R836G, Q944K, and R935G. In some embodiments, a variant LbCas12a endonuclease comprises mutations at positions R836, Q944, and R935 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R836, Q944, and R935 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, Y943, and R935 with reference to amino acid position numbering of LbCas12a ND2006 (e.g., SEQ ID NO: 1). In some embodiments, the mutations are R833M, E835D, Y943T, and R935G. In some embodiments, a variant LbCas12a endonuclease comprises mutations at positions R833, E835, Y943, and R935 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, Y943, and R935 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, Y943, and Q941 with reference to amino acid position numbering of LbCas12a ND2006 (e.g., SEQ ID NO: 1). In some embodiments, the mutations are R833M, E835D, Y943T, and Q941K. In some embodiments, a variant LbCas12a endonuclease comprises mutations at positions R833, E835, Y943, and Q941 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, Y943, and Q941 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, and E125 with reference to amino acid position numbering of LbCas12a ND2006 (e.g., SEQ ID NO: 1). In some embodiments, the mutations are R833M, E835D, and E125A. In some embodiments, a variant LbCas12a endonuclease comprises mutations at positions R833, E835, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions Y943, Q944, K932, N933, and E125 with reference to amino acid position numbering of LbCas12a ND2006 (e.g., SEQ ID NO: 1). In some embodiments, the mutations are Y943F, Q944K, K932G, N933G, and E125A. In some embodiments, a variant LbCas12a endonuclease comprises mutations at positions Y943, Q944, K932, N933, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions Y943, Q944, K932, N933, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R836, Q944, R935, and E125 with reference to amino acid position numbering of LbCas12a ND2006 (e.g., SEQ ID NO: 1). In some embodiments, the mutations are R836G, Q944K, R935G, and E125A. In some embodiments, a variant LbCas12a endonuclease comprises mutations at positions R836, Q944, R935, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R836, Q944, R935, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, Y943, R935, and E125 with reference to amino acid position numbering of LbCas12a ND2006 (e.g., SEQ ID NO: 1). In some embodiments, the mutations are R833M, E835D, Y943T, R935G, and E125A. In some embodiments, a variant LbCas12a endonuclease comprises mutations at positions R833, E835, Y943, R935, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, Y943, R935, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions R833, E835, Y943, Q941, and E125 with reference to amino acid position numbering of LbCas12a ND2006 (e.g., SEQ ID NO: 1). In some embodiments, the mutations are R833M, E835D, Y943T, Q941K, and E125A. In some embodiments, a variant LbCas12a endonuclease comprises mutations at positions R833, E835, Y943, Q941, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions R833, E835, Y943, Q941, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, the variant Cas12a endonuclease comprises a polypeptide sequence that comprises mutations at amino acid positions corresponding to positions D832, Y943, Q944, K932, N933, and E125 with reference to amino acid position numbering of LbCas12a ND2006 (e.g., SEQ ID NO: 1). In some embodiments, the mutations are D832A, Y943F, Q944K, K932G, N933G, and E125A. In some embodiments, a variant LbCas12a endonuclease comprises mutations at positions D832, Y943, Q944, K932, N933, and E125 with reference to amino acid position numbering of LbCas12a ND2006 endonuclease (e.g., SEQ ID NO: 1) and has at least 80%, at least 85%, at least 90%, at least 95%, or at least 98% identity to a wild-type reference LbCas12a ND2006 endonuclease (e.g., SEQ ID NO: 1). In some embodiments, a variant LbCas12a endonuclease comprises mutations at positions D832, Y943, Q944, K932, N933, and E125 with reference to amino acid position numbering of LbCas12a ND2006 endonuclease (e.g., SEQ ID NO: 1) and has no more than 1, 2, 3, 4, or 5 additional substitutions relative to a wild-type reference LbCas12a ND2006 endonuclease (e.g., SEQ ID NO: 1).

In some embodiments, a fusion protein comprises a variant Cas12a endonuclease that is located at or near the N-terminal end of the protein. In some embodiments, a fusion protein comprises a variant Cas12a endonuclease that is located at or near the C-terminal end of the protein. In some embodiments, a fusion protein comprises a base editing enzyme that is located at or near the N-terminal end of the protein. In some embodiments, a fusion protein comprises a base editing enzyme that is located at or near the C-terminal end of the protein. In some embodiments, a fusion protein comprises an N-terminal variant Cas12a endonuclease and a C-terminal base editing enzyme (i.e., variant Cas12a endonuclease is located closer to the N-terminus of the protein than the base editing enzyme). In some embodiments, a fusion protein comprises an N-terminal base editing enzyme and a C-terminal variant Cas12a endonuclease (i.e., base editing enzyme is located closer to the N-terminus of the protein than the variant Cas12a endonuclease).

A fusion protein may comprise one or more nuclear localization signals (NLSs). In some embodiments, a fusion protein comprises 1, 2, 3, 4, 5, or more NLSs. An NLS is an amino acid sequence that directs the fusion protein for import into the cell nucleus by nuclear transport. In some embodiments, an NLS is a positively charged amino acid sequence that comprises several lysine and/or arginine amino acids. In some embodiments, a fusion protein comprises an NLS that is located at or near the N-terminal end of the protein. In some embodiments, a fusion protein comprises an NLS that is located at or near the C-terminal end of the protein. In some embodiments, a fusion protein comprises an NLS that is located at or near the N-terminal end of the protein and an NLS that is located at or near the C-terminal end of the protein.

Non-limiting examples of an NLS include an SV40 NLS, a nucleoprotein (NP) NLS, and a bipartite (BP) NLS. In some embodiments, an SV40 NLS comprises the amino acid sequence of PKKKRKV (SEQ ID NO: 193). In some embodiments, a nucleoprotein NLS comprises the amino acid sequence of KRPAATKKAGQAKKKK (SEQ ID NO: 194). In some embodiments, a bipartite NLS comprises the amino acid sequence of KRTADGSEFESPKKKRKV (SEQ ID NO: 195). In some embodiments, a fusion protein comprises an SV40 NLS that is located at or near the N-terminal end of the protein and/or an SV40 NLS that is located at or near the C-terminal end of the protein. In some embodiments, a fusion protein comprises an SV40 NLS that is located at or near the N-terminal end of the protein, an SV40 NLS that is located at or near the C-terminal end of the protein, and an NP NLS that is located at or near the C-terminal end of the protein. In some embodiments, a fusion protein comprises an NP NLS that is located at or near the N-terminal end of the protein, an NP NLS that is located at or near the C-terminal end of the protein, and an SV40 NLS that is located at or near the C-terminal end of the protein. In some embodiments, a fusion protein comprises a BP NLS that is located at or near the N-terminal end of the protein, an SV40 NLS that is located at or near the C-terminal end of the protein, and an NP NLS that is located at or near the C-terminal end of the protein. In some embodiments, a fusion protein comprises a BP NLS that is located at or near the N-terminal end of the protein and a BP NLS that is located at or near the C-terminal end of the protein. In some embodiments, a fusion protein comprises a BP NLS that is located at or near the N-terminal end of the protein and an NP NLS that is located at or near the C-terminal end of the protein.

A fusion protein may comprise one or more linkers. A linker for use in a fusion protein of the disclosure is generally an amino acid linker. In some embodiments, a linker functions to provide separation between different protein elements of the fusion protein (e.g., variant Cas12a endonuclease and base editing enzyme). In some embodiments, the presence of a linker between two protein elements of the fusion protein provides flexibility between the two elements of the fusion protein (e.g., to allow for each protein to fold and perform its function, e.g., enzymatic function). In some embodiments, a fusion protein comprises a linker between a variant Cas12a endonuclease and a base editing enzyme.

In some embodiments, a linker is a flexible linker. In some embodiments, a linker is a flexible linker comprising serine and/or glycine amino acids. In some embodiments, a linker is an amino acid sequence, wherein the majority of the amino acids of the linker are serine and/or glycine amino acids. In some embodiments, a linker comprises the amino acid sequence of (GS)n (SEQ ID NO: 196), (GGS)n (SEQ ID NO: 197), (GSS)n (SEQ ID NO: 198), (GGSS)n (SEQ ID NO: 199), (SGGGS)n (SEQ ID NO: 200) or (SGGS)n (SEQ ID NO: 201) wherein n is 1-10. In some embodiments, a linker comprises the amino acid sequence of GSSGGSGGSGGSGS (SEQ ID NO: 202). In some embodiments, a linker comprises the amino acid sequence of SGSETPGTSESATPES (SEQ ID NO: 203). In other embodiments, a linker comprises the amino acid sequence of SGGSSGGSSGSETPGTSESATPESSGGSSGGS (SEQ ID NO: 204). In some embodiments, a linker comprises the amino acid sequence of SGGSGGSGGS (SEQ ID NO: 205). In some embodiments, a linker comprises the amino acid sequence of GGGGGGS (SEQ ID NO: 206); GSSGGSGGSGGS (SEQ ID NO: 207); or SGGS (SEQ ID NO: 208).

In various embodiments, the base editor fusion protein further comprises an inhibitor of base excision repair (“iBER”) that covalently or non-covalently binds to a mutated nucleobase to prevent its excision during subsequent mismatch repair. Use of an iBER in the base editor fusion protein may increase base editing efficiency for the deamination-oxidation and other strategies. In certain embodiments, the iBER is an 8-oxo-guanine glycosylase (OGG or OGGI) inhibitor (“OGG inhibitor”), a thymine-DNA glycosylase (TDG) inhibitor, a uracil-DNA glycosylase (UDG) inhibitor, a Methyl-CpG Binding Domain 4 (MBD4) inhibitor. In certain embodiments, the iBER comprises a catalytically inactive OGG that binds 8-oxo-inosine to prevent its excision during subsequent mismatch repair.

A fusion protein may comprise one or more uracil glycosylase inhibitors (UGI). In some embodiments, a fusion protein comprises 1, 2, 3, 4, or 5 UGI polypeptides. In some embodiments, a UGI is a polypeptide that is capable of inhibiting a uracil-DNA glycosylase base-excision repair enzyme (e.g., from the uracil base excision repair (UBER) pathway). In some embodiments, a UGI is capable of inhibiting repair machinery such that the UGI polypeptide increases the efficiency of a C to T conversion. In some embodiments, a UGI polypeptide reduces the rate of conversions that are not the C to T conversion. A UGI polypeptide may be a wild-type UGI polypeptide or a variant UGI polypeptide. In some embodiments, a UGI is a UGI from Bacillus subtilis bacteriophage PBS1. In some embodiments, a UGI polypeptide comprises the amino acid sequence of TNLSDIIEKETGKQLVIQESILMLPEEVEEVIGNKPESDILVHTAYDESTDENVML LTSDAPEYKPWALVIQDSNGENKIKML (SEQ ID NO: 209). In some embodiments, a UGI polypeptide comprises at least 70%, 75%, 80%, 85%, 90%, 95%, or 97% identity to SEQ ID NO: 209.

A fusion protein may comprise one or more DNA glycosylases. In some embodiments, a fusion protein comprises 1, 2, 3, 4, or 5 DNA glycosylases. In some embodiments, a fusion protein comprises a uracil DNA glycosylase (UNG). In some embodiments, a fusion protein comprises a N-methyl purine glycosylase (MPG). In some embodiments, an N-methyl purine glycosylase (MPG) functions in recognition and repair of base pairs comprising deoxyinosine. Hypoxanthine (the nucleobase of deoxyinosine) is recognized and excised by MPG, which results in the generation of an abasic site (AP site). The abasic site is then processed by the base excision repair pathway. Accordingly, MPG is useful, in some embodiments, for A-to-C or A-to-T conversions, particularly when in combination with an adenosine deaminase (e.g., TadA deaminase).

In some embodiments, a fusion protein comprises an MPG and an adenosine deaminase (e.g., TadA deaminase). In such embodiments, the adenosine deaminase converts adenine to inosine; and the MPG subsequently removes the inosine to produce an abasic site. The abasic site may subsequently be processed (e.g., to place a cytosine at that position). In some embodiments, a fusion protein comprises an MPG and a cytidine deaminase (e.g., APOBEC1 deaminase).

In some embodiments, an MPG polypeptide comprises the amino acid sequence of SEQ ID NO: 210. In some embodiments, an MPG polypeptide comprises at least 70%, 75%, 80%, 85%, 90%, 95%, or 97% identity to SEO ID NO: 210.

(SEQ ID NO: 210)
VTPALQMKKPKQFCRRMGQKKQRPARAGQPHSSSDAAQAPAEQPHSSSD
AAQAPCPRERCLGPPTTPGPYRSIYFSSPKGHLTRLGLEFFDQPAVPLA
RAFLGQVLVRRLPNGTELRGRIVETEAYLGPEDEAAHSRGGRQTPRNRG
MEMKPGTLYVYIIYGMYFCMNISSQGDGACVLLRALEPLEGLETMRQLR
STLRKGTASRVLKDRELCSGPSKLCQALAINKSFDQRDLAQDEAVWLER
GPLEPSEPAVVAAARVGVGHAGEWARKPLRFYVRGSPWVSVVDRVAEQD
TQA

In some embodiments, a UNG is a polypeptide that is capable of recognition and excision of uracil from DNA strands. A UNG polypeptide is able to remove unwanted uracil bases from DNA molecules by cleaving the N-glycosidic bond and initiating the base-excision repair (BER) pathway. In some embodiments, a UNG is capable of increasing the efficiency of a C to G conversion. A UNG polypeptide may be a human UNG (hUNG) or an Escherichia coli UNG (eUNG). In some embodiments, a hUNG polypeptide is a mitochondrial UNG1 or the nuclear UNG2A. A UNG polypeptide may be a wild-type UNG polypeptide or a variant UNG polypeptide. In some embodiments, an UNG polypeptide comprises the amino acid sequence of ANELTWHDVLAEEKOOPYFLNTLQTVASERQS GVTIYPPQKDVFNAFRFTELGDVKVVILGQDPYHGPGQAHGLAFSVRPGIAIPPSLLNMYKE LENTIPGFTRPNHGYLESWARQGVLLLNTVLTVRAGQAHSHASLGWETFTDKVISLINQHRE GVVFLLWGSHAQKKGAIIDKQRHHVLKAPHPSPLSAHRGFFGCNHFVLANQWLEQRGETPID WMPVLPAESE (SEQ ID NO: 211). In some embodiments, a UNG polypeptide comprises the amino acid sequence of IGQKTLYSFFSPSPARKRHAPSPEPA VQGTGVAGVPEESGDAAAIPAKKAPAGQEEPGTPPSSPLSAEQLDRIORNKAAALLRLAARN VPVGFGESWKKHLSGEFGKPYFIKLMGFVAEERKHYTVYPPPHQVFTWTQMCDIKDVKVVIL GQDPYHGPNQAHGLCFSVQRPVPPPPSLENIYKELSTDIEDFVHPGHGDLSGWAKQGVLLLN AVLTVRAHQANSHKERGWEQFTDAVVSWLNQNSNGLVFLLWGSYAQKKGSAI DRKRHHVLQT AHPSPLSVYRGFFGCRHFSKTNELLOKSGKKPIDWKEL (SEQ ID NO: 212). In some embodiments, an UNG polypeptide comprises at least 70%, 75%, 80%, 85%, 90%, 95%, or 97% identity to SEQ ID NO: 211 or 212.

A fusion protein may comprise one or more DNA binding domains (DBD). In some embodiments, a fusion protein comprises 1, 2, 3, 4, or 5 DBDs. In some embodiments, a DBD is a DBD that recognizes a sequence-specific single-stranded DNA molecule. In some embodiments, a DBD is a DBD that recognizes a non-sequence-specific single-stranded DNA molecule. In some embodiments, a DBD is a DBD that recognizes a sequence-specific double-stranded DNA molecule. In some embodiments, a DBD is a DBD that recognizes a non-sequence-specific double-stranded DNA molecule. In some embodiments, a DBD comprises a Rad51 DNA binding domain (DBD). A DBD may be a wild-type DBD polypeptide or a variant DBD polypeptide. In some embodiments, a DBD polypeptide comprises the amino acid sequence of MAMQMQLEANADTSVEEESFGPOPISRLEQ CGINANDVKKLEEAGFHTVEAVAYAPKKELINIKGISEAKADKILAEAAKLVPMGFTTATEF HORRSEIIQITTGSKELDKLLQ (SEQ ID NO: 213). In some embodiments, a DBD polypeptide comprises at least 70%, 75%, 80%, 85%, 90%, 95%, or 97% identity to SEQ ID NO: 213.

A fusion protein can be designed to be specific for a certain type of enzymatic conversion. For example, certain fusion proteins are designed for C to T conversion, A to G conversion, or C to G conversion.

In some embodiments, a fusion protein comprising a variant Cas12a endonuclease, a cytidine deaminase (e.g., APOBEC1), and a UGI is a C to T base editor (i.e., enzymatically converts C to T). In some embodiments, a fusion protein comprising a variant Cas12a endonuclease, a cytidine deaminase (e.g., APOBEC1), a UGI, and a Rad51 DBD is a C to T base editor (i.e., enzymatically converts C to T). In some embodiments, a fusion protein comprising a variant Cas12a endonuclease and a base editing enzyme (e.g., TadA) is an A to G base editor (i.e., enzymatically converts A to G). In some embodiments, a fusion protein comprising a variant Cas12a endonuclease, an adenosine deaminase (e.g., TadA), and a Rad51 DBD is an A to G base editor (i.e., enzymatically converts A to G). In some embodiments, a fusion protein comprising a variant Cas12a endonuclease and a cytidine deaminase (e.g., APOBEC1) is a C to G base editor (i.e., enzymatically converts C to G). In some embodiments, a fusion protein comprising a variant Cas12a endonuclease, a cytidine deaminase (e.g., APOBEC1), and a UNG is a C to G base editor (i.e., enzymatically converts C to G).

Exemplary, non-limiting, fusion protein sequences are provided in Table 5.

TABLE 5
Non-limiting Examples of Fusion Proteins Comprising Variant Cas12a
Endonucleases and Base Editing Enzyme

| | | SEQ |
|---|---|---|
| Name* | Sequence | ID NO: |
| LbBEv2 | MGSSETGPVAVDPTLRRRIEPHEFEVFFDPRELRKETCLLYEINWGGRHSIWRHTSQNTNKHVEV | 163 |
| (rAPOBEC1, | NFIEKFTTERYFCPNTRCSITWFLSWSPCGECSRAITEFLSRYPHVTLFIYIARLYHHADPRNRQ | |
| Linker, Cas12a, | GLRDLISSGVTIQIMTEQESGYCWRNFVNYSPSNEAHWPRYPHLWVRLYVLELYCIILGLPPCLN | |
| UGI, sv40 NLSs, NP | ILRRKQPQLTFFTIALQSCHYQRLPPHILWATGLKSGGSSGGSSGSETPGTSESATPESSGGSSG | |
| NLS, BP NLS) | GSSKLEKFTNCYSLSKTLRFKAIPVGKTQENIDNKRLLVEDEKRAEDYKGVKKLLDRYYLSFIND | |
| C to T base editor | VLHSIKLKNLNNYISLFRKKTRTEKENKELENLEINLRKEIAKAFKGNEGYKSLFKKDIIETILP | |
| | EFLDDKDEIALVNSFNGFTTAFTGFFDNRENMFSEEAKSTSIAFRCINENLTRYISNMDIFEKVD | |
| | AIFDKHEVQEIKEKILNSDYDVEDFFEGEFFNFVLTQEGIDVYNAIIGGFVTESGEKIKGLNEYI | |
| | NLYNQKTKQKLPKFKPLYKQVLSDRESLSFYGEGYTSDEEVLEVFRNTLNKNSEIFSSIKKLEKL | |
| | FKNFDEYSSAGIFVKNGPAISTISKDIFGEWNVIRDKWNAEYDDIHLKKKAVVTEKYEDDRRKSF | |
| | KKIGSFSLEQLQEYADADLSVVEKLKEIIIQKVDEIYKVYGSSEKLFDADFVLEKSLKKNDAVVA | |
| | IMKDLLDSVKSFENYIKAFFGEGKETNRDESFYGDFVLAYDILLKVDHIYDAIRNYVTQKPYSKD | |
| | KFKLYFQNPQFMGGWDKDKETDYRATILRYGSKYYLAIMDKKYAKCLQKIDKDDVNGNYEKINYK | |
| | LLPGPNKMLPKVFFSKKWMAYYNPSEDIQKIYKNGTFKKGDMFNLNDCHKLIDFFKDSISRYPKW | |
| | SNAYDFNFSETEKYKDIAGFYREVEEQGYKVSFESASKKEVDKLVEEGKLYMFQIYNKDFSDKSH | |
| | GTPNLHTMYFKLLFDENNHGQIRLSGGAELFMRRASLKKEELVVHPANSPIANKNPDNPKKTTTL | |
| | SYDVYKDKRFSEDQYELHIPIAINKCPKNIFKINTEVRVLLKHDDNPYVIGIARGERNLLYIVVV | |
| | DGKGNIVEQYSLNEIINNFNGIRIKTDYHSLLDKKEKERFEARQNWTSIENIKELKAGYISQVVH | |
| | KICELVEKYDAVIALEDLNSGFKNSRVKVEKQVYQKFEKMLIDKLNYMVDKKSNPCATGGALKGY | |
| | QITNKFESFKSMSTQNGFIFYIPAWLTSKIDPSTGFVNLLKTKYTSIADSKKFISSFDRIMYVPE | |
| | EDLFEFALDYKNFSRTDADYIKKWKLYSYGNRIRIFRNPKKNNVFDWEEVCLTSAYKELFNKYGI | |
| | NYQQGDIRALLCEQSDKAFYSSFMALMSLMLQMRNSITGRTDVDFLISPVKNSDGIFYDSRNYEA | |
| | QENAILPKNADANGAYNIARKVLWAIGQFKKAEDEKLDKVKIAISNKEWLEYAQTSVKKRPAATK | |
| | KAGQAKKKKGSSGGSGGSGGSTNLSDIIEKETGKQLVIQESILMLPEEVEEVIGNKPESDILVHT | |
| | AYDESTDENVMLLTSDAPEYKPWALVIQDSNGENKIKMLSGGSPKKKRKV | |
| LbBEv3 | MPKKKRKVGSSETGPVAVDPTLRRRIEPHEFEVFFDPRELRKETCLLYEINWGGRHSIWRHTSQN | 164 |
| (rAPOBEC1, | TNKHVEVNFIEKFTTERYFCPNTRCSITWFLSWSPCGECSRAITEFLSRYPHVTLFIYIARLYHH | |
| Linker, Cas12a, | ADPRNRQGLRDLISSGVTIQIMTEQESGYCWRNFVNYSPSNEAHWPRYPHLWVRLYVLELYCIIL | |
| UGI, sv40 NLSs, NP | GLPPCLNILRRKQPQLTFFTIALQSCHYQRLPPHILWATGLKSGGSSGGSSGSETPGTSESATPE | |
| NLS, BP NLS) | SSGGSSGGSSKLEKFTNCYSLSKTLRFKAIPVGKTQENIDNKRLLVEDEKRAEDYKGVKKLLDRY | |
| C to T base editor | YLSFINDVLHSIKLKNLNNYISLFRKKTRTEKENKELENLEINLRKEIAKAFKGNEGYKSLFKKD | |
| | IIETILPEFLDDKDEIALVNSFNGFTTAFTGFFDNRENMFSEEAKSTSIAFRCINENLTRYISNM | |
| | DIFEKVDAIFDKHEVQEIKEKILNSDYDVEDFFEGEFFNFVLTQEGIDVYNAIIGGFVTESGEKI | |
| | KGLNEYINLYNQKTKQKLPKFKPLYKQVLSDRESLSFYGEGYTSDEEVLEVFRNTLNKNSEIFSS | |
| | IKKLEKLFKNFDEYSSAGIFVKNGPAISTISKDIFGEWNVIRDKWNAEYDDIHLKKKAVVTEKYE | |
| | DDRRKSFKKIGSFSLEQLQEYADADLSVVEKLKEIIIQKVDEIYKVYGSSEKLFDADFVLEKSLK | |
| | KNDAVVAIMKDLLDSVKSFENYIKAFFGEGKETNRDESFYGDFVLAYDILLKVDHIYDAIRNYVT | |
| | QKPYSKDKFKLYFQNPQFMGGWDKDKETDYRATILRYGSKYYLAIMDKKYAKCLQKIDKDDVNGN | |
| | YEKINYKLLPGPNKMLPKVFFSKKWMAYYNPSEDIQKIYKNGTFKKGDMFNLNDCHKLIDFFKDS | |
| | ISRYPKWSNAYDFNFSETEKYKDIAGFYREVEEQGYKVSFESASKKEVDKLVEEGKLYMFQIYNK | |
| | DFSDKSHGTPNLHTMYFKLLFDENNHGQIRLSGGAELFMRRASLKKEELVVHPANSPIANKNPDN | |
| | PKKTTTLSYDVYKDKRFSEDQYELHIPIAINKCPKNIFKINTEVRVLLKHDDNPYVIGIARGERN | |
| | LLYIVVVDGKGNIVEQYSLNEIINNFNGIRIKTDYHSLLDKKEKERFEARQNWTSIENIKELKAG | |
| | YISQVVHKICELVEKYDAVIALEDLNSGFKNSRVKVEKQVYQKFEKMLIDKLNYMVDKKSNPCAT | |
| | GGALKGYQITNKFESFKSMSTQNGFIFYIPAWLTSKIDPSTGFVNLLKTKYTSIADSKKFISSFD | |
| | RIMYVPEEDLFEFALDYKNFSRTDADYIKKWKLYSYGNRIRIFRNPKKNNVFDWEEVCLTSAYKE | |
| | LFNKYGINYQQGDIRALLCEQSDKAFYSSFMALMSLMLQMRNSITGRTDVDFLISPVKNSDGIFY | |
| | DSRNYEAQENAILPKNADANGAYNIARKVLWAIGQFKKAEDEKLDKVKIAISNKEWLEYAQTSVK | |
| | KRPAATKKAGQAKKKKGSSGGSGGSGGSTNLSDIIEKETGKQLVIQESILMLPEEVEEVIGNKPE | |
| | SDILVHTAYDESTDENVMLLTSDAPEYKPWALVIQDSNGENKIKMLSGGSPKKKRKV | |
| LbBEv4 | MKRPAATKKAGQAKKKKGSSETGPVAVDPTLRRRIEPHEFEVFFDPRELRKETCLLYEINWGGRH | 165 |
| (rAPOBEC1, | SIWRHTSQNTNKHVEVNFIEKFTTERYFCPNTRCSITWFLSWSPCGECSRAITEFLSRYPHVTLF | |
| Linker, Cas12a, | IYIARLYHHADPRNRQGLRDLISSGVTIQIMTEQESGYCWRNFVNYSPSNEAHWPRYPHLWVRLY | |
| UGI, sv40 NLSs, NP | VLELYCIILGLPPCLNILRRKQPQLTFFTIALQSCHYQRLPPHILWATGLKSGGSSGGSSGSETP | |
| NLS, BP NLS) | GTSESATPESSGGSSGGSSKLEKFTNCYSLSKTLRFKAIPVGKTQENIDNKRLLVEDEKRAEDYK | |
| C to T base editor | GVKKLLDRYYLSFINDVLHSIKLKNLNNYISLFRKKTRTEKENKELENLEINLRKEIAKAFKGNE | |
| | GYKSLFKKDIIETILPEFLDDKDEIALVNSFNGFTTAFTGFFDNRENMFSEEAKSTSIAFRCINE | |
| | NLTRYISNMDIFEKVDAIFDKHEVQEIKEKILNSDYDVEDFFEGEFFNFVLTQEGIDVYNAIIGG | |
| | FVTESGEKIKGLNEYINLYNQKTKQKLPKFKPLYKQVLSDRESLSFYGEGYTSDEEVLEVERNTL | |
| | NKNSEIFSSIKKLEKLFKNFDEYSSAGIFVKNGPAISTISKDIFGEWNVIRDKWNAEYDDIHLKK | |
| | KAVVTEKYEDDRRKSFKKIGSFSLEQLQEYADADLSVVEKLKEIIIQKVDEIYKVYGSSEKLFDA | |
| | DFVLEKSLKKNDAVVAIMKDLLDSVKSFENYIKAFFGEGKETNRDESFYGDFVLAYDILLKVDHI | |
| | YDAIRNYVTQKPYSKDKFKLYFQNPQFMGGWDKDKETDYRATILRYGSKYYLAIMDKKYAKCLQK | |
| | IDKDDVNGNYEKINYKLLPGPNKMLPKVFFSKKWMAYYNPSEDIQKIYKNGTFKKGDMFNLNDCH | |
| | KLIDFFKDSISRYPKWSNAYDFNFSETEKYKDIAGFYREVEEQGYKVSFESASKKEVDKLVEEGK | |
| | LYMFQIYNKDFSDKSHGTPNLHTMYFKLLFDENNHGQIRLSGGAELFMRRASLKKEELVVHPANS | |
| | PIANKNPDNPKKTTTLSYDVYKDKRFSEDQYELHIPIAINKCPKNIFKINTEVRVLLKHDDNPYV | |
| | IGIARGERNLLYIVVVDGKGNIVEQYSLNEIINNENGIRIKTDYHSLLDKKEKERFEARQNWTSI | |
| | ENIKELKAGYISQVVHKICELVEKYDAVIALEDLNSGFKNSRVKVEKQVYQKFEKMLIDKLNYMV | |
| | DKKSNPCATGGALKGYQITNKFESFKSMSTQNGFIFYIPAWLTSKIDPSTGFVNLLKTKYTSIAD | |
| | SKKFISSFDRIMYVPEEDLFEFALDYKNFSRTDADYIKKWKLYSYGNRIRIFRNPKKNNVEDWEE | |
| | VCLTSAYKELFNKYGINYQQGDIRALLCEQSDKAFYSSFMALMSLMLQMRNSITGRTDVDFLISP | |
| | VKNSDGIFYDSRNYEAQENAILPKNADANGAYNIARKVLWAIGQFKKAEDEKLDKVKIAISNKEW | |
| | LEYAQTSVKKRPAATKKAGQAKKKKGSSGGSGGSGGSTNLSDIIEKETGKQLVIQESILMLPEEV | |
| | EEVIGNKPESDILVHTAYDESTDENVMLLTSDAPEYKPWALVIQDSNGENKIKMLSGGSPKKKRK | |
| | V | |
| LbBEv5 | MGSKRTADGSEFESPKKKRKVGSSETGPVAVDPTLRRRIEPHEFEVFFDPRELRKETCLLYEINW | 166 |
| (rAPOBEC1, | GGRHSIWRHTSQNTNKHVEVNFIEKFTTERYFCPNTRCSITWFLSWSPCGECSRAITEFLSRYPH | |
| Linker, Cas12a, | VTLFIYIARLYHHADPRNRQGLRDLISSGVTIQIMTEQESGYCWRNFVNYSPSNEAHWPRYPHLW | |
| UGI, sv40 NLSs, NP | VRLYVLELYCIILGLPPCLNILRRKQPQLTFFTIALQSCHYQRLPPHILWATGLKSGGSSGGSSG | |
| NLS, BP NLS) | SETPGTSESATPESSGGSSGGSSKLEKFTNCYSLSKTLRFKAIPVGKTQENIDNKRLLVEDEKRA | |
| C to T base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSTNLSDIIEKETGKQLVIQESILML | |
| | PEEVEEVIGNKPESDILVHTAYDESTDENVMLLTSDAPEYKPWALVIQDSNGENKIKMLSGGSPK | |
| | KKRKV | |
| LbBEv6 | MGSKRTADGSEFESPKKKRKVGSSETGPVAVDPTLRRRIEPHEFEVFFDPRELRKETCLLYEINW | 167 |
| (rAPOBEC1, | GGRHSIWRHTSQNTNKHVEVNFIEKFTTERYFCPNTRCSITWFLSWSPCGECSRAITEFLSRYPH | |
| Linker, Cas12a, | VTLFIYIARLYHHADPRNRQGLRDLISSGVTIQIMTEQESGYCWRNFVNYSPSNEAHWPRYPHLW | |
| UGI, sv40 NLSs, NP | VRLYVLELYCIILGLPPCLNILRRKQPQLTFFTIALQSCHYQRLPPHILWATGLKSGGSSGGSSG | |
| NLS, BP NLS) | SETPGTSESATPESSGGSSGGSSKLEKFTNCYSLSKTLRFKAIPVGKTQENIDNKRLLVEDEKRA | |
| C to T base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSENGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSTNLSDIIEKETGKQLVIQESILML | |
| | PEEVEEVIGNKPESDILVHTAYDESTDENVMLLTSDAPEYKPWALVIQDSNGENKIKMLSGGSGG | |
| | SGGSTNLSDIIEKETGKQLVIQESILMLPEEVEEVIGNKPESDILVHTAYDESTDENVMLLTSDA | |
| | PEYKPWALVIQDSNGENKIKMLSGGSPKKKRKV | |
| LbBEv16 | MGSKRTADGSEFESPKKKRKVGSGTNLSDIIEKETGKQLVIQESILMLPEEVEEVIGNKPESDIL | 168 |
| (rAPOBEC1, | VHTAYDESTDENVMLLTSDAPEYKPWALVIQDSNGENKIKMLGSSGGSGGSGGSGSSETGPVAVD | |
| Linker, Cas12a, | PTLRRRIEPHEFEVFFDPRELRKETCLLYEINWGGRHSIWRHTSQNTNKHVEVNFIEKFTTERYF | |
| UGI, sv40 NLSs, NP | CPNTRCSITWFLSWSPCGECSRAITEFLSRYPHVTLFIYIARLYHHADPRNRQGLRDLISSGVTI | |
| NLS, BP NLS) | QIMTEQESGYCWRNFVNYSPSNEAHWPRYPHLWVRLYVLELYCIILGLPPCLNILRRKQPQLTFF | |
| C to T base editor | TIALQSCHYQRLPPHILWATGLKSGGSSGGSSGSETPGTSESATPESSGGSSGGSSKLEKFTNCY | |
| | SLSKTLRFKAIPVGKTQENIDNKRLLVEDEKRAEDYKGVKKLLDRYYLSFINDVLHSIKLKNLNN | |
| | YISLFRKKTRTEKENKELENLEINLRKEIAKAFKGNEGYKSLFKKDIIETILPEFLDDKDEIALV | |
| | NSFNGFTTAFTGFFDNRENMFSEEAKSTSIAFRCINENLTRYISNMDIFEKVDAIFDKHEVQEIK | |
| | EKILNSDYDVEDFFEGEFFNFVLTQEGIDVYNAIIGGFVTESGEKIKGLNEYINLYNQKTKQKLP | |
| | KFKPLYKQVLSDRESLSFYGEGYTSDEEVLEVFRNTLNKNSEIFSSIKKLEKLFKNFDEYSSAGI | |
| | FVKNGPAISTISKDIFGEWNVIRDKWNAEYDDIHLKKKAVVTEKYEDDRRKSFKKIGSFSLEQLQ | |
| | EYADADLSVVEKLKEIIIQKVDEIYKVYGSSEKLFDADFVLEKSLKKNDAVVAIMKDLLDSVKSF | |
| | ENYIKAFFGEGKETNRDESFYGDFVLAYDILLKVDHIYDAIRNYVTQKPYSKDKFKLYFQNPQFM | |
| | GGWDKDKETDYRATILRYGSKYYLAIMDKKYAKCLQKIDKDDVNGNYEKINYKLLPGPNKMLPKV | |
| | FFSKKWMAYYNPSEDIQKIYKNGTFKKGDMFNLNDCHKLIDFFKDSISRYPKWSNAYDENFSETE | |
| | KYKDIAGFYREVEEQGYKVSFESASKKEVDKLVEEGKLYMFQIYNKDFSDKSHGTPNLHTMYFKL | |
| | LFDENNHGQIRLSGGAELFMRRASLKKEELVVHPANSPIANKNPDNPKKTTTLSYDVYKDKRFSE | |
| | DQYELHIPIAINKCPKNIFKINTEVRVLLKHDDNPYVIGIARGERNLLYIVVVDGKGNIVEQYSL | |
| | NEIINNFNGIRIKTDYHSLLDKKEKERFEARQNWTSIENIKELKAGYISQVVHKICELVEKYDAV | |
| | IALEDLNSGFKNSRVKVEKQVYQKFEKMLIDKLNYMVDKKSNPCATGGALKGYQITNKFESFKSM | |
| | STQNGFIFYIPAWLTSKIDPSTGFVNLLKTKYTSIADSKKFISSFDRIMYVPEEDLFEFALDYKN | |
| | FSRTDADYIKKWKLYSYGNRIRIFRNPKKNNVFDWEEVCLTSAYKELFNKYGINYQQGDIRALLC | |
| | EQSDKAFYSSFMALMSLMLQMRNSITGRTDVDFLISPVKNSDGIFYDSRNYEAQENAILPKNADA | |
| | NGAYNIARKVLWAIGQFKKAEDEKLDKVKIAISNKEWLEYAQTSVKKRPAATKKAGQAKKKKGSS | |
| | GGSGGSGGSPKKKRKV | |
| LbBEv17 | MGSKRTADGSEFESPKKKRKVGSSETGPVAVDPTLRRRIEPHEFEVFFDPRELRKETCLLYEINW | 169 |
| (rAPOBEC1, | GGRHSIWRHTSQNTNKHVEVNFIEKFTTERYFCPNTRCSITWFLSWSPCGECSRAITEFLSRYPH | |
| Linker, Cas12a, | VTLFIYIARLYHHADPRNRQGLRDLISSGVTIQIMTEQESGYCWRNFVNYSPSNEAHWPRYPHLW | |
| UGI, sv40 NLSs, NP | VRLYVLELYCIILGLPPCLNILRRKQPQLTFFTIALQSCHYQRLPPHILWATGLKSGGSSGGSSG | |
| NLS, BP NLS) | SETPGTSESATPESSGGSSGGSTNLSDIIEKETGKQLVIQESILMLPEEVEEVIGNKPESDILVH | |
| C to T base editor | TAYDESTDENVMLLTSDAPEYKPWALVIQDSNGENKIKMLGSSGGSGGSGGSSKLEKFTNCYSLS | |
| | KTLRFKAIPVGKTQENIDNKRLLVEDEKRAEDYKGVKKLLDRYYLSFINDVLHSIKLKNLNNYIS | |
| | LFRKKTRTEKENKELENLEINLRKEIAKAFKGNEGYKSLFKKDIIETILPEFLDDKDEIALVNSF | |
| | NGFTTAFTGFFDNRENMFSEEAKSTSIAFRCINENLTRYISNMDIFEKVDAIFDKHEVQEIKEKI | |
| | LNSDYDVEDFFEGEFFNFVLTQEGIDVYNAIIGGFVTESGEKIKGLNEYINLYNQKTKQKLPKFK | |
| | PLYKQVLSDRESLSFYGEGYTSDEEVLEVFRNTLNKNSEIFSSIKKLEKLFKNFDEYSSAGIFVK | |
| | NGPAISTISKDIFGEWNVIRDKWNAEYDDIHLKKKAVVTEKYEDDRRKSFKKIGSFSLEQLQEYA | |
| | DADLSVVEKLKEIIIQKVDEIYKVYGSSEKLFDADFVLEKSLKKNDAVVAIMKDLLDSVKSFENY | |
| | IKAFFGEGKETNRDESFYGDFVLAYDILLKVDHIYDAIRNYVTQKPYSKDKFKLYFQNPQFMGGW | |
| | DKDKETDYRATILRYGSKYYLAIMDKKYAKCLQKIDKDDVNGNYEKINYKLLPGPNKMLPKVFFS | |
| | KKWMAYYNPSEDIQKIYKNGTFKKGDMFNLNDCHKLIDFFKDSISRYPKWSNAYDENFSETEKYK | |
| | DIAGFYREVEEQGYKVSFESASKKEVDKLVEEGKLYMFQIYNKDFSDKSHGTPNLHTMYFKLLED | |
| | ENNHGQIRLSGGAELFMRRASLKKEELVVHPANSPIANKNPDNPKKTTTLSYDVYKDKRFSEDQY | |
| | ELHIPIAINKCPKNIFKINTEVRVLLKHDDNPYVIGIARGERNLLYIVVVDGKGNIVEQYSLNEI | |
| | INNFNGIRIKTDYHSLLDKKEKERFEARQNWTSIENIKELKAGYISQVVHKICELVEKYDAVIAL | |
| | EDLNSGFKNSRVKVEKQVYQKFEKMLIDKLNYMVDKKSNPCATGGALKGYQITNKFESFKSMSTQ | |
| | NGFIFYIPAWLTSKIDPSTGFVNLLKTKYTSIADSKKFISSFDRIMYVPEEDLFEFALDYKNFSR | |
| | TDADYIKKWKLYSYGNRIRIFRNPKKNNVFDWEEVCLTSAYKELFNKYGINYQQGDIRALLCEQS | |
| | DKAFYSSFMALMSLMLQMRNSITGRTDVDFLISPVKNSDGIFYDSRNYEAQENAILPKNADANGA | |
| | YNIARKVLWAIGQFKKAEDEKLDKVKIAISNKEWLEYAQTSVKKRPAATKKAGQAKKKKGSSGGS | |
| | GGSGGSPKKKRKV | |
| LbBEv7 | MGSKRTADGSEFESPKKKRKVGSSETGPVAVDPTLRRRIEPHEFEVFFDPRELRKETCLLYEINW | 170 |
| (rAPOBEC1, | GGRHSIWRHTSQNTNKHVEVNFIEKFTTERYFCPNTRCSITWFLSWSPCGECSRAITEFLSRYPH | |
| Linker, Cas12a, | VTLFIYIARLYHHADPRNRQGLRDLISSGVTIQIMTEQESGYCWRNFVNYSPSNEAHWPRYPHLW | |
| UGI, sv40 NLSs, NP | VRLYVLELYCIILGLPPCLNILRRKQPQLTFFTIALQSCHYQRLPPHILWATGLKSGGSSGGSSG | |
| NLS, BP NLS, | SETPGTSESATPESSGGSSGGSMAMQMQLEANADTSVEEESFGPQPISRLEQCGINANDVKKLEE | |
| Rad51 DBD) | AGFHTVEAVAYAPKKELINIKGISEAKADKILAEAAKLVPMGFTTATEFHQRRSEIIQITTGSKE | |
| C to T base editor | LDKLLQSGGSSGGSSGSETPGTSESATPESSGGSSGGSSKLEKFTNCYSLSKTLRFKAIPVGKTQ | |
| | ENIDNKRLLVEDEKRAEDYKGVKKLLDRYYLSFINDVLHSIKLKNLNNYISLFRKKTRTEKENKE | |
| | LENLEINLRKEIAKAFKGNEGYKSLFKKDIIETILPEFLDDKDEIALVNSFNGFTTAFTGFFDNR | |
| | ENMFSEEAKSTSIAFRCINENLTRYISNMDIFEKVDAIFDKHEVQEIKEKILNSDYDVEDFFEGE | |
| | FFNFVLTQEGIDVYNAIIGGFVTESGEKIKGLNEYINLYNQKTKQKLPKFKPLYKQVLSDRESLS | |
| | FYGEGYTSDEEVLEVFRNTLNKNSEIFSSIKKLEKLFKNFDEYSSAGIFVKNGPAISTISKDIFG | |
| | EWNVIRDKWNAEYDDIHLKKKAVVTEKYEDDRRKSFKKIGSFSLEQLQEYADADLSVVEKLKEII | |
| | IQKVDEIYKVYGSSEKLFDADFVLEKSLKKNDAVVAIMKDLLDSVKSFENYIKAFFGEGKETNRD | |
| | ESFYGDFVLAYDILLKVDHIYDAIRNYVTQKPYSKDKFKLYFQNPQFMGGWDKDKETDYRATILR | |
| | YGSKYYLAIMDKKYAKCLQKIDKDDVNGNYEKINYKLLPGPNKMLPKVFFSKKWMAYYNPSEDIQ | |
| | KIYKNGTFKKGDMFNLNDCHKLIDFFKDSISRYPKWSNAYDFNFSETEKYKDIAGFYREVEEQGY | |
| | KVSFESASKKEVDKLVEEGKLYMFQIYNKDFSDKSHGTPNLHTMYFKLLFDENNHGQIRLSGGAE | |
| | LFMRRASLKKEELVVHPANSPIANKNPDNPKKTTTLSYDVYKDKRFSEDQYELHIPIAINKCPKN | |
| | IFKINTEVRVLLKHDDNPYVIGIARGERNLLYIVVVDGKGNIVEQYSLNEIINNENGIRIKTDYH | |
| | SLLDKKEKERFEARQNWTSIENIKELKAGYISQVVHKICELVEKYDAVIALEDLNSGFKNSRVKV | |
| | EKQVYQKFEKMLIDKLNYMVDKKSNPCATGGALKGYQITNKFESFKSMSTQNGFIFYIPAWLTSK | |
| | IDPSTGFVNLLKTKYTSIADSKKFISSFDRIMYVPEEDLFEFALDYKNFSRTDADYIKKWKLYSY | |
| | GNRIRIFRNPKKNNVFDWEEVCLTSAYKELFNKYGINYQQGDIRALLCEQSDKAFYSSFMALMSL | |
| | MLQMRNSITGRTDVDFLISPVKNSDGIFYDSRNYEAQENAILPKNADANGAYNIARKVLWAIGQF | |
| | KKAEDEKLDKVKIAISNKEWLEYAQTSVKKRPAATKKAGQAKKKKGSSGGSGGSGGSTNLSDIIE | |
| | KETGKQLVIQESILMLPEEVEEVIGNKPESDILVHTAYDESTDENVMLLTSDAPEYKPWALVIQD | |
| | SNGENKIKMLSGGSPKKKRKV | |
| LbBEv8 | MGSKRTADGSEFESPKKKRKVGSSETGPVAVDPTLRRRIEPHEFEVFFDPRELRKETCLLYEINW | 171 |
| (rAPOBEC1, | GGRHSIWRHTSQNTNKHVEVNFIEKFTTERYFCPNTRCSITWFLSWSPCGECSRAITEFLSRYPH | |
| Linker, Cas12a, | VTLFIYIARLYHHADPRNRQGLRDLISSGVTIQIMTEQESGYCWRNFVNYSPSNEAHWPRYPHLW | |
| UGI, sv40 NLSs, NP | VRLYVLELYCIILGLPPCLNILRRKQPQLTFFTIALQSCHYQRLPPHILWATGLKSGGSSGGSSG | |
| NLS, BP NLS, | SETPGTSESATPESSGGSSGGSSKLEKFTNCYSLSKTLRFKAIPVGKTQENIDNKRLLVEDEKRA | |
| Rad51 DBD) | EDYKGVKKLLDRYYLSFINDVLHSIKLKNLNNYISLFRKKTRTEKENKELENLEINLRKEIAKAF | |
| C to T base editor | KGNEGYKSLFKKDIIETILPEFLDDKDEIALVNSENGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSTNLSDIIEKETGKQLVIQESILML | |
| | PEEVEEVIGNKPESDILVHTAYDESTDENVMLLTSDAPEYKPWALVIQDSNGENKIKMLSGGSPK | |
| | KKRKVSGGSMAMQMQLEANADTSVEEESFGPQPISRLEQCGINANDVKKLEEAGFHTVEAVAYAP | |
| | KKELINIKGISEAKADKILAEAAKLVPMGFTTATEFHQRRSEIIQITTGSKELDKLLQ | |
| LbBEv9 | MGSKRTADGSEFESPKKKRKVGSGAMQMQLEANADTSVEEESFGPQPISRLEQCGINANDVKKLE | 172 |
| (rAPOBEC1, | EAGFHTVEAVAYAPKKELINIKGISEAKADKILAEAAKLVPMGFTTATEFHQRRSEIIQITTGSK | |
| Linker, Cas12a, | ELDKLLQSGGSSGGSSGSETPGTSESATPESSGGSSGGSGSETGPVAVDPTLRRRIEPHEFEVFF | |
| UGI, sv40 NLSs, NP | DPRELRKETCLLYEINWGGRHSIWRHTSQNTNKHVEVNFIEKFTTERYFCPNTRCSITWFLSWSP | |
| NLS, BP NLS, | CGECSRAITEFLSRYPHVTLFIYIARLYHHADPRNRQGLRDLISSGVTIQIMTEQESGYCWRNFV | |
| Rad51 DBD) | NYSPSNEAHWPRYPHLWVRLYVLELYCIILGLPPCLNILRRKQPQLTFFTIALQSCHYQRLPPHI | |
| C to T base editor | LWATGLKSGGSSGGSSGSETPGTSESATPESSGGSSGGSSKLEKFTNCYSLSKTLRFKAIPVGKT | |
| | QENIDNKRLLVEDEKRAEDYKGVKKLLDRYYLSFINDVLHSIKLKNLNNYISLFRKKTRTEKENK | |
| | ELENLEINLRKEIAKAFKGNEGYKSLFKKDIIETILPEFLDDKDEIALVNSFNGFTTAFTGFFDN | |
| | RENMFSEEAKSTSIAFRCINENLTRYISNMDIFEKVDAIFDKHEVQEIKEKILNSDYDVEDFFEG | |
| | EFFNFVLTQEGIDVYNAIIGGFVTESGEKIKGLNEYINLYNQKTKQKLPKFKPLYKQVLSDRESL | |
| | SFYGEGYTSDEEVLEVFRNTLNKNSEIFSSIKKLEKLFKNFDEYSSAGIFVKNGPAISTISKDIF | |
| | GEWNVIRDKWNAEYDDIHLKKKAVVTEKYEDDRRKSFKKIGSFSLEQLQEYADADLSVVEKLKEI | |
| | IIQKVDEIYKVYGSSEKLFDADFVLEKSLKKNDAVVAIMKDLLDSVKSFENYIKAFFGEGKETNR | |
| | DESFYGDFVLAYDILLKVDHIYDAIRNYVTQKPYSKDKFKLYFQNPQFMGGWDKDKETDYRATIL | |
| | RYGSKYYLAIMDKKYAKCLQKIDKDDVNGNYEKINYKLLPGPNKMLPKVFFSKKWMAYYNPSEDI | |
| | QKIYKNGTFKKGDMFNLNDCHKLIDFFKDSISRYPKWSNAYDFNFSETEKYKDIAGFYREVEEQG | |
| | YKVSFESASKKEVDKLVEEGKLYMFQIYNKDFSDKSHGTPNLHTMYFKLLFDENNHGQIRLSGGA | |
| | ELFMRRASLKKEELVVHPANSPIANKNPDNPKKTTTLSYDVYKDKRFSEDQYELHIPIAINKCPK | |
| | NIFKINTEVRVLLKHDDNPYVIGIARGERNLLYIVVVDGKGNIVEQYSLNEIINNENGIRIKTDY | |
| | HSLLDKKEKERFEARQNWTSIENIKELKAGYISQVVHKICELVEKYDAVIALEDLNSGFKNSRVK | |
| | VEKQVYQKFEKMLIDKLNYMVDKKSNPCATGGALKGYQITNKFESFKSMSTQNGFIFYIPAWLTS | |
| | KIDPSTGFVNLLKTKYTSIADSKKFISSFDRIMYVPEEDLFEFALDYKNFSRTDADYIKKWKLYS | |
| | YGNRIRIFRNPKKNNVFDWEEVCLTSAYKELFNKYGINYQQGDIRALLCEQSDKAFYSSFMALMS | |
| | LMLQMRNSITGRTDVDFLISPVKNSDGIFYDSRNYEAQENAILPKNADANGAYNIARKVLWAIGQ | |
| | FKKAEDEKLDKVKIAISNKEWLEYAQTSVKKRPAATKKAGQAKKKKGSSGGSGGSGGSTNLSDII | |
| | EKETGKQLVIQESILMLPEEVEEVIGNKPESDILVHTAYDESTDENVMLLTSDAPEYKPWALVIQ | |
| | DSNGENKIKMLSGGSPKKKRKV | |
| LbBEv10 | MGSKRTADGSEFESPKKKRKVGSGSTDAEYVRIHEKLDIYTFKKQFSNNKKSVSHRCYVLFELKR | 173 |
| (evoCDA, Linker, | RGERRACFWGYAVNKPQSGTERGIHAEIFSIRKVEEYLRDNPGQFTINWYSSWSPCADCAEKILE | |
| Cas12a, UGI, sv40 | WYNQELRGNGHTLKIWVCKLYYEKNARNQIGLWNLRDNGVGLNVMVSEHYQCCRKIFIQSSHNQL | |
| NLSs, NPNLS, BP | NENRWLEKTLKRAEKRRSELSIMFQVKILHTTKSPAVSGGSSGGSSGSETPGTSESATPESSGGS | |
| NLS) | SGGSSKLEKFTNCYSLSKTLRFKAIPVGKTQENIDNKRLLVEDEKRAEDYKGVKKLLDRYYLSFI | |
| | NDVLHSIKLKNLNNYISLFRKKTRTEKENKELENLEINLRKEIAKAFKGNEGYKSLFKKDIIETI | |
| | LPEFLDDKDEIALVNSFNGFTTAFTGFFDNRENMFSEEAKSTSIAFRCINENLTRYISNMDIFEK | |
| | VDAIFDKHEVQEIKEKILNSDYDVEDFFEGEFFNFVLTQEGIDVYNAIIGGFVTESGEKIKGLNE | |
| C to T base editor | YINLYNQKTKQKLPKFKPLYKQVLSDRESLSFYGEGYTSDEEVLEVFRNTLNKNSEIFSSIKKLE | |
| | KLFKNFDEYSSAGIFVKNGPAISTISKDIFGEWNVIRDKWNAEYDDIHLKKKAVVTEKYEDDRRK | |
| | SFKKIGSFSLEQLQEYADADLSVVEKLKEIIIQKVDEIYKVYGSSEKLFDADFVLEKSLKKNDAV | |
| | VAIMKDLLDSVKSFENYIKAFFGEGKETNRDESFYGDFVLAYDILLKVDHIYDAIRNYVTQKPYS | |
| | KDKFKLYFQNPQFMGGWDKDKETDYRATILRYGSKYYLAIMDKKYAKCLQKIDKDDVNGNYEKIN | |
| | YKLLPGPNKMLPKVFFSKKWMAYYNPSEDIQKIYKNGTFKKGDMFNLNDCHKLIDFFKDSISRYP | |
| | KWSNAYDFNFSETEKYKDIAGFYREVEEQGYKVSFESASKKEVDKLVEEGKLYMFQIYNKDFSDK | |
| | SHGTPNLHTMYFKLLFDENNHGQIRLSGGAELFMRRASLKKEELVVHPANSPIANKNPDNPKKTT | |
| | TLSYDVYKDKRFSEDQYELHIPIAINKCPKNIFKINTEVRVLLKHDDNPYVIGIARGERNLLYIV | |
| | VVDGKGNIVEQYSLNEIINNFNGIRIKTDYHSLLDKKEKERFEARQNWTSIENIKELKAGYISQV | |
| | VHKICELVEKYDAVIALEDLNSGFKNSRVKVEKQVYQKFEKMLIDKLNYMVDKKSNPCATGGALK | |
| | GYQITNKFESFKSMSTQNGFIFYIPAWLTSKIDPSTGFVNLLKTKYTSIADSKKFISSFDRIMYV | |
| | PEEDLFEFALDYKNFSRTDADYIKKWKLYSYGNRIRIFRNPKKNNVFDWEEVCLTSAYKELFNKY | |
| | GINYQQGDIRALLCEQSDKAFYSSFMALMSLMLQMRNSITGRTDVDFLISPVKNSDGIFYDSRNY | |
| | EAQENAILPKNADANGAYNIARKVLWAIGQFKKAEDEKLDKVKIAISNKEWLEYAQTSVKKRPAA | |
| | TKKAGQAKKKKGSSGGSGGSGGSTNLSDIIEKETGKQLVIQESILMLPEEVEEVIGNKPESDILV | |
| | HTAYDESTDENVMLLTSDAPEYKPWALVIQDSNGENKIKMLSGGSPKKKRKV | |
| LbBEvll | MGSKRTADGSEFESPKKKRKVGSGSFERNYDPRELRKETYLLYEIKWGKSGKLWRHWCQNNRTQH | 174 |
| (evoFERNY, Linker, | AEVYFLENIFNARRFNPSTHCSITWYLSWSPCAECSQKIVDFLKEHPNVNLEIYVARLYYPENER | |
| Cas12a, UGI, sv40 | NRQGLRDLVNSGVTIRIMDLPDYNYCWKTFVSDQGGDEDYWPGHFAPWIKQYSLKLSGGSSGGSS | |
| NLSs, NPNLS, BP | GSETPGTSESATPESSGGSSGGSSKLEKFTNCYSLSKTLRFKAIPVGKTQENIDNKRLLVEDEKR | |
| NLS) | AEDYKGVKKLLDRYYLSFINDVLHSIKLKNLNNYISLFRKKTRTEKENKELENLEINLRKEIAKA | |
| C to T base editor | FKGNEGYKSLFKKDIIETILPEFLDDKDEIALVNSFNGFTTAFTGFFDNRENMFSEEAKSTSIAF | |
| | RCINENLTRYISNMDIFEKVDAIFDKHEVQEIKEKILNSDYDVEDFFEGEFFNFVLTQEGIDVYN | |
| | AIIGGFVTESGEKIKGLNEYINLYNQKTKQKLPKFKPLYKQVLSDRESLSFYGEGYTSDEEVLEV | |
| | FRNTLNKNSEIFSSIKKLEKLFKNFDEYSSAGIFVKNGPAISTISKDIFGEWNVIRDKWNAEYDD | |
| | IHLKKKAVVTEKYEDDRRKSFKKIGSFSLEQLQEYADADLSVVEKLKEIIIQKVDEIYKVYGSSE | |
| | KLFDADFVLEKSLKKNDAVVAIMKDLLDSVKSFENYIKAFFGEGKETNRDESFYGDFVLAYDILL | |
| | KVDHIYDAIRNYVTQKPYSKDKFKLYFQNPQFMGGWDKDKETDYRATILRYGSKYYLAIMDKKYA | |
| | KCLQKIDKDDVNGNYEKINYKLLPGPNKMLPKVFFSKKWMAYYNPSEDIQKIYKNGTFKKGDMEN | |
| | LNDCHKLIDFFKDSISRYPKWSNAYDENFSETEKYKDIAGFYREVEEQGYKVSFESASKKEVDKL | |
| | VEEGKLYMFQIYNKDFSDKSHGTPNLHTMYFKLLFDENNHGQIRLSGGAELFMRRASLKKEELVV | |
| | HPANSPIANKNPDNPKKTTTLSYDVYKDKRFSEDQYELHIPIAINKCPKNIFKINTEVRVLLKHD | |
| | DNPYVIGIARGERNLLYIVVVDGKGNIVEQYSLNEIINNFNGIRIKTDYHSLLDKKEKERFEARQ | |
| | NWTSIENIKELKAGYISQVVHKICELVEKYDAVIALEDLNSGFKNSRVKVEKQVYQKFEKMLIDK | |
| | LNYMVDKKSNPCATGGALKGYQITNKFESFKSMSTQNGFIFYIPAWLTSKIDPSTGFVNLLKTKY | |
| | TSIADSKKFISSFDRIMYVPEEDLFEFALDYKNFSRTDADYIKKWKLYSYGNRIRIFRNPKKNNV | |
| | FDWEEVCLTSAYKELFNKYGINYQQGDIRALLCEQSDKAFYSSFMALMSLMLQMRNSITGRTDVD | |
| | FLISPVKNSDGIFYDSRNYEAQENAILPKNADANGAYNIARKVLWAIGQFKKAEDEKLDKVKIAI | |
| | SNKEWLEYAQTSVKKRPAATKKAGQAKKKKGSSGGSGGSGGSTNLSDIIEKETGKQLVIQESILM | |
| | LPEEVEEVIGNKPESDILVHTAYDESTDENVMLLTSDAPEYKPWALVIQDSNGENKIKMLSGGSP | |
| | KKKRKV | |
| LbBEv12 | MGSKRTADGSEFESPKKKRKVGSGSSKTGPVAVDPTLRRRIEPHEFEVFFDPRELRKETCLLYEI | 175 |
| (evoAPOBEC1, | NWGGRHSIWRHTSQNTNKHVEVNFIEKFTTERYFCPNTRCSITWFLSWSPCGECSRAITEFLSRY | |
| Linker, Cas12a, | PNVTLFIYIARLYHLANPRNRQGLRDLISSGVTIQIMTEQESGYCWHNFVNYSPSNESHWPRYPH | |
| UGI, sv40 NLSs, NP | LWVRLYVLELYCIILGLPPCLNILRRKQSQLTSFTIALQSCHYQRLPPHILWATGLKSGGSSGGS | |
| NLS, BP NLS) | SGSETPGTSESATPESSGGSSGGSSKLEKFTNCYSLSKTLRFKAIPVGKTQENIDNKRLLVEDEK | |
| C to T base editor | RAEDYKGVKKLLDRYYLSFINDVLHSIKLKNLNNYISLFRKKTRTEKENKELENLEINLRKEIAK | |
| | AFKGNEGYKSLFKKDIIETILPEFLDDKDEIALVNSFNGFTTAFTGFFDNRENMFSEEAKSTSIA | |
| | FRCINENLTRYISNMDIFEKVDAIFDKHEVQEIKEKILNSDYDVEDFFEGEFFNFVLTQEGIDVY | |
| | NAIIGGFVTESGEKIKGLNEYINLYNQKTKQKLPKFKPLYKQVLSDRESLSFYGEGYTSDEEVLE | |
| | VFRNTLNKNSEIFSSIKKLEKLFKNFDEYSSAGIFVKNGPAISTISKDIFGEWNVIRDKWNAEYD | |
| | DIHLKKKAVVTEKYEDDRRKSFKKIGSFSLEQLQEYADADLSVVEKLKEIIIQKVDEIYKVYGSS | |
| | EKLFDADFVLEKSLKKNDAVVAIMKDLLDSVKSFENYIKAFFGEGKETNRDESFYGDFVLAYDIL | |
| | LKVDHIYDAIRNYVTQKPYSKDKFKLYFQNPQFMGGWDKDKETDYRATILRYGSKYYLAIMDKKY | |
| | AKCLQKIDKDDVNGNYEKINYKLLPGPNKMLPKVFFSKKWMAYYNPSEDIQKIYKNGTFKKGDMF | |
| | NLNDCHKLIDFFKDSISRYPKWSNAYDFNFSETEKYKDIAGFYREVEEQGYKVSFESASKKEVDK | |
| | LVEEGKLYMFQIYNKDFSDKSHGTPNLHTMYFKLLFDENNHGQIRLSGGAELFMRRASLKKEELV | |
| | VHPANSPIANKNPDNPKKTTTLSYDVYKDKRFSEDQYELHIPIAINKCPKNIFKINTEVRVLLKH | |
| | DDNPYVIGIARGERNLLYIVVVDGKGNIVEQYSLNEIINNENGIRIKTDYHSLLDKKEKERFEAR | |
| | QNWTSIENIKELKAGYISQVVHKICELVEKYDAVIALEDLNSGFKNSRVKVEKQVYQKFEKMLID | |
| | KLNYMVDKKSNPCATGGALKGYQITNKFESFKSMSTQNGFIFYIPAWLTSKIDPSTGFVNLLKTK | |
| | YTSIADSKKFISSFDRIMYVPEEDLFEFALDYKNFSRTDADYIKKWKLYSYGNRIRIFRNPKKNN | |
| | VFDWEEVCLTSAYKELFNKYGINYQQGDIRALLCEQSDKAFYSSFMALMSLMLQMRNSITGRTDV | |
| | DFLISPVKNSDGIFYDSRNYEAQENAILPKNADANGAYNIARKVLWAIGQFKKAEDEKLDKVKIA | |
| | ISNKEWLEYAQTSVKKRPAATKKAGQAKKKKGSSGGSGGSGGSTNLSDIIEKETGKQLVIQESIL | |
| | MLPEEVEEVIGNKPESDILVHTAYDESTDENVMLLTSDAPEYKPWALVIQDSNGENKIKMLSGGS | |
| | PKKKRKV | |
| LbBEv13 | MGSKRTADGSEFESPKKKRKVGSGEASPASGPRHLMDPHIFTSNFNNGIGRHKTYLCYEVERLDN | 176 |
| (hAPOBEC3A, | GTSVKMDQHRGFLHNQAKNLLCGFYGRHAELRFLDLVPSLQLDPAQIYRVTWFISWSPCFSWGCA | |
| Linker, Cas12a, | GEVRAFLQENTHVRLRIFAARIYDYDPLYKEALQMLRDAGAQVSIMTYDEFKHCWDTFVDHQGCP | |
| UGI, sv40 NLSs, NP | FQPWDGLDEHSQALSGRLRAILQNQGNSGGSSGGSSGSETPGTSESATPESSGGSSGGSSKLEKF | |
| NLS, BP NLS) | TNCYSLSKTLRFKAIPVGKTQENIDNKRLLVEDEKRAEDYKGVKKLLDRYYLSFINDVLHSIKLK | |
| C to T base editor | NLNNYISLFRKKTRTEKENKELENLEINLRKEIAKAFKGNEGYKSLFKKDIIETILPEFLDDKDE | |
| | IALVNSFNGFTTAFTGFFDNRENMFSEEAKSTSIAFRCINENLTRYISNMDIFEKVDAIFDKHEV | |
| | QEIKEKILNSDYDVEDFFEGEFFNFVLTQEGIDVYNAIIGGFVTESGEKIKGLNEYINLYNQKTK | |
| | QKLPKFKPLYKQVLSDRESLSFYGEGYTSDEEVLEVFRNTLNKNSEIFSSIKKLEKLFKNFDEYS | |
| | SAGIFVKNGPAISTISKDIFGEWNVIRDKWNAEYDDIHLKKKAVVTEKYEDDRRKSFKKIGSFSL | |
| | EQLQEYADADLSVVEKLKEIIIQKVDEIYKVYGSSEKLFDADFVLEKSLKKNDAVVAIMKDLLDS | |
| | VKSFENYIKAFFGEGKETNRDESFYGDFVLAYDILLKVDHIYDAIRNYVTQKPYSKDKFKLYFQN | |
| | PQFMGGWDKDKETDYRATILRYGSKYYLAIMDKKYAKCLQKIDKDDVNGNYEKINYKLLPGPNKM | |
| | LPKVFFSKKWMAYYNPSEDIQKIYKNGTFKKGDMFNLNDCHKLIDFFKDSISRYPKWSNAYDENF | |
| | SETEKYKDIAGFYREVEEQGYKVSFESASKKEVDKLVEEGKLYMFQIYNKDFSDKSHGTPNLHTM | |
| | YFKLLFDENNHGQIRLSGGAELFMRRASLKKEELVVHPANSPIANKNPDNPKKTTTLSYDVYKDK | |
| | RFSEDQYELHIPIAINKCPKNIFKINTEVRVLLKHDDNPYVIGIARGERNLLYIVVVDGKGNIVE | |
| | QYSLNEIINNFNGIRIKTDYHSLLDKKEKERFEARQNWTSIENIKELKAGYISQVVHKICELVEK | |
| | YDAVIALEDLNSGFKNSRVKVEKQVYQKFEKMLIDKLNYMVDKKSNPCATGGALKGYQITNKFES | |
| | FKSMSTQNGFIFYIPAWLTSKIDPSTGFVNLLKTKYTSIADSKKFISSFDRIMYVPEEDLFEFAL | |
| | DYKNFSRTDADYIKKWKLYSYGNRIRIFRNPKKNNVFDWEEVCLTSAYKELFNKYGINYQQGDIR | |
| | ALLCEQSDKAFYSSFMALMSLMLQMRNSITGRTDVDFLISPVKNSDGIFYDSRNYEAQENAILPK | |
| | NADANGAYNIARKVLWAIGQFKKAEDEKLDKVKIAISNKEWLEYAQTSVKKRPAATKKAGQAKKK | |
| | KGSSGGSGGSGGSTNLSDIIEKETGKQLVIQESILMLPEEVEEVIGNKPESDILVHTAYDESTDE | |
| | NVMLLTSDAPEYKPWALVIQDSNGENKIKMLSGGSPKKKRKV | |
| LbABE8e | MKRTADGSEFESPKKKRKVSEVEFSHEYWMRHALTLAKRARDEREVPVGAVLVLNNRVIGEGWNR | 177 |
| (TadA, Linker, | AIGLHDPTAHAEIMALRQGGLVMQNYRLIDATLYVTFEPCVMCAGAMIHSRIGRVVFGVRNSKRG | |
| Cas12a, BP NLS) | AAGSLMNVLNYPGMNHRVEITEGILADECAALLCDFYRMPRQVFNAQKKAQSSINSGGSSGGSSG | |
| A to G base editor | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbABE8ev11 | MKRTADGSEFESPKKKRKVSEVEFSHEYWMRHALTLAKRARDEREVPVGAVLVLNNRVIGEGWNR | 178 |
| (TadA, Linker, | AIGLHDPTAHAEIMALRQGGLVMQNYRLIDATLYVTFEPCVMCAGAMIHSRIGRVVFGVRNSKRG | |
| Cas12a, BP NLS, | AAGSLMNVLNYPGMNHRVEITEGILADECAALLCDFYRMPRQVFNAQKKAQSSINSGGSSGGSSG | |
| Rad51 DBD) | SETPGTSESATPESSGGSSGGSMAMQMQLEANADTSVEEESFGPQPISRLEQCGINANDVKKLEE | |
| A to G base editor | AGFHTVEAVAYAPKKELINIKGISEAKADKILAEAAKLVPMGFTTATEFHQRRSEIIQITTGSKE | |
| | LDKLLQSGGSSGGSSGSETPGTSESATPESSGGSSGGSSKLEKFTNCYSLSKTLRFKAIPVGKTQ | |
| | ENIDNKRLLVEDEKRAEDYKGVKKLLDRYYLSFINDVLHSIKLKNLNNYISLFRKKTRTEKENKE | |
| | LENLEINLRKEIAKAFKGNEGYKSLFKKDIIETILPEFLDDKDEIALVNSFNGFTTAFTGFFDNR | |
| | ENMFSEEAKSTSIAFRCINENLTRYISNMDIFEKVDAIFDKHEVQEIKEKILNSDYDVEDFFEGE | |
| | FFNFVLTQEGIDVYNAIIGGFVTESGEKIKGLNEYINLYNQKTKQKLPKFKPLYKQVLSDRESLS | |
| | FYGEGYTSDEEVLEVFRNTLNKNSEIFSSIKKLEKLFKNFDEYSSAGIFVKNGPAISTISKDIFG | |
| | EWNVIRDKWNAEYDDIHLKKKAVVTEKYEDDRRKSFKKIGSFSLEQLQEYADADLSVVEKLKEII | |
| | IQKVDEIYKVYGSSEKLFDADFVLEKSLKKNDAVVAIMKDLLDSVKSFENYIKAFFGEGKETNRD | |
| | ESFYGDFVLAYDILLKVDHIYDAIRNYVTQKPYSKDKFKLYFQNPQFMGGWDKDKETDYRATILR | |
| | YGSKYYLAIMDKKYAKCLQKIDKDDVNGNYEKINYKLLPGPNKMLPKVFFSKKWMAYYNPSEDIQ | |
| | KIYKNGTFKKGDMFNLNDCHKLIDFFKDSISRYPKWSNAYDFNFSETEKYKDIAGFYREVEEQGY | |
| | KVSFESASKKEVDKLVEEGKLYMFQIYNKDFSDKSHGTPNLHTMYFKLLFDENNHGQIRLSGGAE | |
| | LFMRRASLKKEELVVHPANSPIANKNPDNPKKTTTLSYDVYKDKRFSEDQYELHIPIAINKCPKN | |
| | IFKINTEVRVLLKHDDNPYVIGIARGERNLLYIVVVDGKGNIVEQYSLNEIINNENGIRIKTDYH | |
| | SLLDKKEKERFEARQNWTSIENIKELKAGYISQVVHKICELVEKYDAVIALEDLNSGFKNSRVKV | |
| | EKQVYQKFEKMLIDKLNYMVDKKSNPCATGGALKGYQITNKFESFKSMSTQNGFIFYIPAWLTSK | |
| | IDPSTGFVNLLKTKYTSIADSKKFISSFDRIMYVPEEDLFEFALDYKNFSRTDADYIKKWKLYSY | |
| | GNRIRIFRNPKKNNVFDWEEVCLTSAYKELFNKYGINYQQGDIRALLCEQSDKAFYSSFMALMSL | |
| | MLQMRNSITGRTDVDFLISPVKNSDGIFYDSRNYEAQENAILPKNADANGAYNIARKVLWAIGQF | |
| | KKAEDEKLDKVKIAISNKEWLEYAQTSVKSGGSKRTADGSEFEPKKKRKVEFGSG | |
| LbABE8ev12 | MKRTADGSEFESPKKKRKVSEVEFSHEYWMRHALTLAKRARDEREVPVGAVLVLNNRVIGEGWNR | 179 |
| (TadA, Linker, | AIGLHDPTAHAEIMALRQGGLVMQNYRLIDATLYVTFEPCVMCAGAMIHSRIGRVVFGVRNSKRG | |
| Cas12a, BP NLS, | AAGSLMNVLNYPGMNHRVEITEGILADECAALLCDFYRMPRQVFNAQKKAQSSINSGGSSGGSSG | |
| Rad51 DBD) | SETPGTSESATPESSGGSSGGSSKLEKFTNCYSLSKTLRFKAIPVGKTQENIDNKRLLVEDEKRA | |
| A to G base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKVSGGSMAMQMQLEANADTSVEEESFGPQPISR | |
| | LEQCGINANDVKKLEEAGFHTVEAVAYAPKKELINIKGISEAKADKILAEAAKLVPMGFTTATEF | |
| | HQRRSEIIQITTGSKELDKLLQ | |
| LbABE8ev13 | MKRTADGSEFESPKKKRKVGSGAMQMQLEANADTSVEEESFGPQPISRLEQCGINANDVKKLEEA | 180 |
| (TadA, Linker, | GFHTVEAVAYAPKKELINIKGISEAKADKILAEAAKLVPMGFTTATEFHQRRSEIIQITTGSKEL | |
| Cas12a, BP NLS, | DKLLQSGGSSGGSSGSETPGTSESATPESSGGSSGGSSEVEFSHEYWMRHALTLAKRARDEREVP | |
| Rad51 DBD) | VGAVLVLNNRVIGEGWNRAIGLHDPTAHAEIMALRQGGLVMQNYRLIDATLYVTFEPCVMCAGAM | |
| A to G base editor | IHSRIGRVVFGVRNSKRGAAGSLMNVLNYPGMNHRVEITEGILADECAALLCDFYRMPRQVENAQ | |
| | KKAQSSINSGGSSGGSSGSETPGTSESATPESSGGSSGGSSKLEKFTNCYSLSKTLRFKAIPVGK | |
| | TQENIDNKRLLVEDEKRAEDYKGVKKLLDRYYLSFINDVLHSIKLKNLNNYISLFRKKTRTEKEN | |
| | KELENLEINLRKEIAKAFKGNEGYKSLFKKDIIETILPEFLDDKDEIALVNSFNGFTTAFTGFFD | |
| | NRENMFSEEAKSTSIAFRCINENLTRYISNMDIFEKVDAIFDKHEVQEIKEKILNSDYDVEDFFE | |
| | GEFFNFVLTQEGIDVYNAIIGGFVTESGEKIKGLNEYINLYNQKTKQKLPKFKPLYKQVLSDRES | |
| | LSFYGEGYTSDEEVLEVFRNTLNKNSEIFSSIKKLEKLFKNFDEYSSAGIFVKNGPAISTISKDI | |
| | FGEWNVIRDKWNAEYDDIHLKKKAVVTEKYEDDRRKSFKKIGSFSLEQLQEYADADLSVVEKLKE | |
| | IIIQKVDEIYKVYGSSEKLFDADFVLEKSLKKNDAVVAIMKDLLDSVKSFENYIKAFFGEGKETN | |
| | RDESFYGDFVLAYDILLKVDHIYDAIRNYVTQKPYSKDKFKLYFQNPQFMGGWDKDKETDYRATI | |
| | LRYGSKYYLAIMDKKYAKCLQKIDKDDVNGNYEKINYKLLPGPNKMLPKVFFSKKWMAYYNPSED | |
| | IQKIYKNGTFKKGDMFNLNDCHKLIDFFKDSISRYPKWSNAYDFNFSETEKYKDIAGFYREVEEQ | |
| | GYKVSFESASKKEVDKLVEEGKLYMFQIYNKDFSDKSHGTPNLHTMYFKLLFDENNHGQIRLSGG | |
| | AELFMRRASLKKEELVVHPANSPIANKNPDNPKKTTTLSYDVYKDKRFSEDQYELHIPIAINKCP | |
| | KNIFKINTEVRVLLKHDDNPYVIGIARGERNLLYIVVVDGKGNIVEQYSLNEIINNENGIRIKTD | |
| | YHSLLDKKEKERFEARQNWTSIENIKELKAGYISQVVHKICELVEKYDAVIALEDINSGFKNSRV | |
| | KVEKQVYQKFEKMLIDKLNYMVDKKSNPCATGGALKGYQITNKFESFKSMSTQNGFIFYIPAWLT | |
| | SKIDPSTGFVNLLKTKYTSIADSKKFISSFDRIMYVPEEDLFEFALDYKNFSRTDADYIKKWKLY | |
| | SYGNRIRIFRNPKKNNVFDWEEVCLTSAYKELFNKYGINYQQGDIRALLCEQSDKAFYSSFMALM | |
| | SLMLQMRNSITGRTDVDFLISPVKNSDGIFYDSRNYEAQENAILPKNADANGAYNIARKVLWAIG | |
| | QFKKAEDEKLDKVKIAISNKEWLEYAQTSVKSGGSKRTADGSEFEPKKKRKV | |
| LbCGBEv0 | MGSKRTADGSEFESPKKKRKVGSSETGPVAVDPTLRRRIEPHEFEVFFDPRELRKETCLLYEINW | 181 |
| (rAPOBEC1, | GGRHSIWRHTSQNTNKHVEVNFIEKFTTERYFCPNTRCSITWFLSWSPCGECSRAITEFLSRYPH | |
| Linker, LbCas12a, | VTLFIYIARLYHHADPRNRQGLRDLISSGVTIQIMTEQESGYCWRNFVNYSPSNEAHWPRYPHLW | |
| sv40 NLSs, NPNLS, | VRLYVLELYCIILGLPPCLNILRRKQPQLTFFTIALQSCHYQRLPPHILWATGLKSGGSSGGSSG | |
| BP NLS) | SETPGTSESATPESSGGSSGGSSKLEKFTNCYSLSKTLRFKAIPVGKTQENIDNKRLLVEDEKRA | |
| C to G base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSPKKKRKV | |
| LbCGBEv1 | MGSKRTADGSEFESPKKKRKVGSSETGPVAVDPTLRRRIEPHEFEVFFDPRELRKETCLLYEINW | 182 |
| (rAPOBEC1, | GGRHSIWRHTSQNTNKHVEVNFIEKFTTERYFCPNTRCSITWFLSWSPCGECSRAITEFLSRYPH | |
| Linker, LbCas12a, | VTLFIYIARLYHHADPRNRQGLRDLISSGVTIQIMTEQESGYCWRNFVNYSPSNEAHWPRYPHLW | |
| sv40 NLSs, NPNLS, | VRLYVLELYCIILGLPPCLNILRRKQPQLTFFTIALQSCHYQRLPPHILWATGLKSGGSSGGSSG | |
| BP NLS, eUNG) | SETPGTSESATPESSGGSSGGSSKLEKFTNCYSLSKTLRFKAIPVGKTQENIDNKRLLVEDEKRA | |
| C to G base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLISKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSANELTWHDVLAEEKQQPYFLNTLQ | |
| | TVASERQSGVTIYPPQKDVFNAFRFTELGDVKVVILGQDPYHGPGQAHGLAFSVRPGIAIPPSLL | |
| | NMYKELENTIPGFTRPNHGYLESWARQGVLLLNTVLTVRAGQAHSHASLGWETFTDKVISLINQH | |
| | REGVVFLLWGSHAQKKGAIIDKQRHHVLKAPHPSPLSAHRGFFGCNHFVLANQWLEQRGETPIDW | |
| | MPVLPAESESGGSPKKKRKV | |
| LbCGBEv2 | MGSKRTADGSEFESPKKKRKVGSGANELTWHDVLAEEKQQPYFLNTLQTVASERQSGVTIYPPQK | 183 |
| (rAPOBEC1, | DVFNAFRFTELGDVKVVILGQDPYHGPGQAHGLAFSVRPGIAIPPSLLNMYKELENTIPGFTRPN | |
| Linker, LbCas12a, | HGYLESWARQGVLLLNTVLTVRAGQAHSHASLGWETFTDKVISLINQHREGVVFLLWGSHAQKKG | |
| sv40 NLSs, NPNLS, | AIIDKQRHHVLKAPHPSPLSAHRGFFGCNHFVLANQWLEQRGETPIDWMPVLPAESESGGSSGGS | |
| BP NLS, eUNG) | SGSETPGTSESATPESSGGSSGGSGSSETGPVAVDPTLRRRIEPHEFEVFFDPRELRKETCLLYE | |
| C to G base editor | INWGGRHSIWRHTSQNTNKHVEVNFIEKFTTERYFCPNTRCSITWFLSWSPCGECSRAITEFLSR | |
| | YPHVTLFIYIARLYHHADPRNRQGLRDLISSGVTIQIMTEQESGYCWRNFVNYSPSNEAHWPRYP | |
| | HLWVRLYVLELYCIILGLPPCLNILRRKQPQLTFFTIALQSCHYQRLPPHILWATGLKSGGSSGG | |
| | SSGSETPGTSESATPESSGGSSGGSSKLEKFTNCYSLSKTLRFKAIPVGKTQENIDNKRLLVEDE | |
| | KRAEDYKGVKKLLDRYYLSFINDVLHSIKLKNLNNYISLFRKKTRTEKENKELENLEINLRKEIA | |
| | KAFKGNEGYKSLFKKDIIETILPEFLDDKDEIALVNSFNGFTTAFTGFFDNRENMFSEEAKSTSI | |
| | AFRCINENLTRYISNMDIFEKVDAIFDKHEVQEIKEKILNSDYDVEDFFEGEFFNFVLTQEGIDV | |
| | YNAIIGGFVTESGEKIKGLNEYINLYNQKTKQKLPKFKPLYKQVLSDRESLSFYGEGYTSDEEVL | |
| | EVFRNTLNKNSEIFSSIKKLEKLFKNFDEYSSAGIFVKNGPAISTISKDIFGEWNVIRDKWNAEY | |
| | DDIHLKKKAVVTEKYEDDRRKSFKKIGSFSLEQLQEYADADLSVVEKLKEIIIQKVDEIYKVYGS | |
| | SEKLFDADFVLEKSLKKNDAVVAIMKDLLDSVKSFENYIKAFFGEGKETNRDESFYGDFVLAYDI | |
| | LLKVDHIYDAIRNYVTQKPYSKDKFKLYFQNPQFMGGWDKDKETDYRATILRYGSKYYLAIMDKK | |
| | YAKCLQKIDKDDVNGNYEKINYKLLPGPNKMLPKVFFSKKWMAYYNPSEDIQKIYKNGTFKKGDM | |
| | FNLNDCHKLIDFFKDSISRYPKWSNAYDFNFSETEKYKDIAGFYREVEEQGYKVSFESASKKEVD | |
| | KLVEEGKLYMFQIYNKDFSDKSHGTPNLHTMYFKLLFDENNHGQIRLSGGAELFMRRASLKKEEL | |
| | VVHPANSPIANKNPDNPKKTTTLSYDVYKDKRFSEDQYELHIPIAINKCPKNIFKINTEVRVLLK | |
| | HDDNPYVIGIARGERNLLYIVVVDGKGNIVEQYSLNEIINNFNGIRIKTDYHSLLDKKEKERFEA | |
| | RQNWTSIENIKELKAGYISQVVHKICELVEKYDAVIALEDLNSGFKNSRVKVEKQVYQKFEKMLI | |
| | DKLNYMVDKKSNPCATGGALKGYQITNKFESFKSMSTQNGFIFYIPAWLTSKIDPSTGFVNLLKT | |
| | KYTSIADSKKFISSFDRIMYVPEEDLFEFALDYKNFSRTDADYIKKWKLYSYGNRIRIFRNPKKN | |
| | NVFDWEEVCLTSAYKELFNKYGINYQQGDIRALLCEQSDKAFYSSFMALMSLMLQMRNSITGRTD | |
| | VDFLISPVKNSDGIFYDSRNYEAQENAILPKNADANGAYNIARKVLWAIGQFKKAEDEKLDKVKI | |
| | AISNKEWLEYAQTSVKKRPAATKKAGQAKKKKGSSGGSGGSGGSPKKKRKV | |
| LbCGBEv3 | MGSKRTADGSEFESPKKKRKVGSSETGPVAVDPTLRRRIEPHEFEVFFDPRELRKETCLLYEINW | 184 |
| (rAPOBEC1, | GGRHSIWRHTSQNTNKHVEVNFIEKFTTERYFCPNTRCSITWFLSWSPCGECSRAITEFLSRYPH | |
| Linker, LbCas12a, | VTLFIYIARLYHHADPRNRQGLRDLISSGVTIQIMTEQESGYCWRNFVNYSPSNEAHWPRYPHLW | |
| sv40 NLSs, NPNLS, | VRLYVLELYCIILGLPPCLNILRRKQPQLTFFTIALQSCHYQRLPPHILWATGLKSGGSSGGSSG | |
| BP NLS, hUNG) | SETPGTSESATPESSGGSSGGSSKLEKFTNCYSLSKTLRFKAIPVGKTQENIDNKRLLVEDEKRA | |
| C to G base editor | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSENGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDENFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKKRPAATKKAGQAKKKKGSSGGSGGSGGSIGQKTLYSFFSPSPARKRHAPSPE | |
| | PAVQGTGVAGVPEESGDAAAIPAKKAPAGQEEPGTPPSSPLSAEQLDRIQRNKAAALLRLAARNV | |
| | PVGFGESWKKHLSGEFGKPYFIKLMGFVAEERKHYTVYPPPHQVFTWTQMCDIKDVKVVILGQDP | |
| | YHGPNQAHGLCFSVQRPVPPPPSLENIYKELSTDIEDFVHPGHGDLSGWAKQGVLLLNAVLTVRA | |
| | HQANSHKERGWEQFTDAVVSWLNQNSNGLVFLLWGSYAQKKGSAIDRKRHHVLQTAHPSPLSVYR | |
| | GFFGCRHFSKTNELLQKSGKKPIDWKELSGGSPKKKRKV | |
| LbCGBEv4 | MGSKRTADGSEFESPKKKRKVGSGIGQKTLYSFFSPSPARKRHAPSPEPAVQGTGVAGVPEESGD | 185 |
| (rAPOBEC1, | AAAIPAKKAPAGQEEPGTPPSSPLSAEQLDRIQRNKAAALLRLAARNVPVGFGESWKKHLSGEFG | |
| Linker, LbCas12a, | KPYFIKLMGFVAEERKHYTVYPPPHQVFTWTQMCDIKDVKVVILGQDPYHGPNQAHGLCFSVQRP | |
| sv40 NLSs, NPNLS, | VPPPPSLENIYKELSTDIEDFVHPGHGDLSGWAKQGVLLLNAVLTVRAHQANSHKERGWEQFTDA | |
| BP NLS, hUNG) | VVSWLNQNSNGLVFLLWGSYAQKKGSAIDRKRHHVLQTAHPSPLSVYRGFFGCRHFSKTNELLQK | |
| C to G base editor | SGKKPIDWKELSGGSSGGSSGSETPGTSESATPESSGGSSGGSGSSETGPVAVDPTLRRRIEPHE | |
| | FEVFFDPRELRKETCLLYEINWGGRHSIWRHTSQNTNKHVEVNFIEKFTTERYFCPNTRCSITWF | |
| | LSWSPCGECSRAITEFLSRYPHVTLFIYIARLYHHADPRNRQGLRDLISSGVTIQIMTEQESGYC | |
| | WRNFVNYSPSNEAHWPRYPHLWVRLYVLELYCIILGLPPCLNILRRKQPQLTFFTIALQSCHYQR | |
| | LPPHILWATGLKSGGSSGGSSGSETPGTSESATPESSGGSSGGSSKLEKFTNCYSLSKTLRFKAI | |
| | PVGKTQENIDNKRLLVEDEKRAEDYKGVKKLLDRYYLSFINDVLHSIKLKNLNNYISLFRKKTRT | |
| | EKENKELENLEINLRKEIAKAFKGNEGYKSLFKKDIIETILPEFLDDKDEIALVNSENGFTTAFT | |
| | GFFDNRENMFSEEAKSTSIAFRCINENLTRYISNMDIFEKVDAIFDKHEVQEIKEKILNSDYDVE | |
| | DFFEGEFFNFVLTQEGIDVYNAIIGGFVTESGEKIKGLNEYINLYNQKTKQKLPKFKPLYKQVLS | |
| | DRESLSFYGEGYTSDEEVLEVFRNTLNKNSEIFSSIKKLEKLFKNFDEYSSAGIFVKNGPAISTI | |
| | SKDIFGEWNVIRDKWNAEYDDIHLKKKAVVTEKYEDDRRKSFKKIGSFSLEQLQEYADADLSVVE | |
| | KLKEIIIQKVDEIYKVYGSSEKLFDADFVLEKSLKKNDAVVAIMKDLLDSVKSFENYIKAFFGEG | |
| | KETNRDESFYGDFVLAYDILLKVDHIYDAIRNYVTQKPYSKDKFKLYFQNPQFMGGWDKDKETDY | |
| | RATILRYGSKYYLAIMDKKYAKCLQKIDKDDVNGNYEKINYKLLPGPNKMLPKVFFSKKWMAYYN | |
| | PSEDIQKIYKNGTFKKGDMFNLNDCHKLIDFFKDSISRYPKWSNAYDENFSETEKYKDIAGFYRE | |
| | VEEQGYKVSFESASKKEVDKLVEEGKLYMFQIYNKDFSDKSHGTPNLHTMYFKLLFDENNHGQIR | |
| | LSGGAELFMRRASLKKEELVVHPANSPIANKNPDNPKKTTTLSYDVYKDKRFSEDQYELHIPIAI | |
| | NKCPKNIFKINTEVRVLLKHDDNPYVIGIARGERNLLYIVVVDGKGNIVEQYSLNEIINNENGIR | |
| | IKTDYHSLLDKKEKERFEARQNWTSIENIKELKAGYISQVVHKICELVEKYDAVIALEDLNSGFK | |
| | NSRVKVEKQVYQKFEKMLIDKLNYMVDKKSNPCATGGALKGYQITNKFESFKSMSTQNGFIFYIP | |
| | AWLTSKIDPSTGFVNLLKTKYTSIADSKKFISSFDRIMYVPEEDLFEFALDYKNFSRTDADYIKK | |
| | WKLYSYGNRIRIFRNPKKNNVFDWEEVCLTSAYKELFNKYGINYQQGDIRALLCEQSDKAFYSSF | |
| | MALMSLMLQMRNSITGRTDVDFLISPVKNSDGIFYDSRNYEAQENAILPKNADANGAYNIARKVL | |
| | WAIGQFKKAEDEKLDKVKIAISNKEWLEYAQTSVKKRPAATKKAGQAKKKKGSSGGSGGSGGSPK | |
| | KKRKV | |
| TBN04 ABE8e | MKRTADGSEFESPKKKRKVSEVEFSHEYWMRHALTLAKRARDEREVPVGAVLVLNNRVIGEGWNR | 388 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFGGSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbAA9 ABE8e | MKRTADGSEFESPKKKRKVSEVEFSHEYWMRHALTLAKRARDEREVPVGAVLVLNNRVIGEGWNR | 389 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDLGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLISKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbAA19 ABE8e | MKRTADGSEFESPKKKRKVSEVEFSHEYWMRHALTLAKRARDEREVPVGAVLVLNNRVIGEGWNR | 390 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDKGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbEF1s9 ABE8e | MKRTADGSEFESPKKKRKVSEVEFSHEYWMRHALTLAKRARDEREVPVGAVLVLNNRVIGEGWNR | 391 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDENFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbAA23 ABE8e | MKRTADGSEFESPKKKRKVSEVEFSHEYWMRHALTLAKRARDEREVPVGAVLVLNNRVIGEGWNR | 392 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFENSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVE | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbMS07 ABE8e | MKRTADGSEFESPKKKRKVSEVEFSHEYWMRHALTLAKRARDEREVPVGAVLVLNNRVIGEGWNR | 393 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFGNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbAA49 ABE8e | MKRTADGSEFESPKKKRKVSEVEFSHEYWMRHALTLAKRARDEREVPVGAVLVLNNRVIGEGWNR | 394 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEGQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbAC10 ABE8e | MKRTADGSEFESPKKKRKVSEVEFSHEYWMRHALTLAKRARDEREVPVGAVLVLNNRVIGEGWNR | 395 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbMS3n5 ABE8e | MKRTADGSEFESPKKKRKVSEVEFSHEYWMRHALTLAKRARDEREVPVGAVLVLNNRVIGEGWNR | 396 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNENGIRIKTDYHSLLDKKEKERFEARQN | |
| | WTSIENIKELKAGYISQVVHKICELVEKYDAVIALEDLNGGFGGSRGKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbTN37 ABE8e | MKRTADGSEFESPKKKRKVSEVEFSHEYWMRHALTLAKRARDEREVPVGAVLVLNNRVIGEGWNR | 397 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEGQVYKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbTN39 ABE8e | MKRTADGSEFESPKKKRKVSEVEFSHEYWMRHALTLAKRARDEREVPVGAVLVLNNRVIGEGWNR | 398 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGEGNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEKQVYKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbTN2 ABE8e | MKRTADGSEFESPKKKRKVSEVEFSHEYWMRHALTLAKRARDEREVPVGAVLVLNNRVIGEGWNR | 399 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVTQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM14 ABE8e | MKRTADGSEFESPKKKRKVSEVEFSHEYWMRHALTLAKRARDEREVPVGAVLVLNNRVIGEGWNR | 400 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGEGNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSGVKVEKQVYKKFEKMLIDKL | |
| | NYMVDKKSNPCATGGALKGYQITNKFESFKSMSTQNGFIFYIPAWLISKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM17 ABE8e | MKRTADGSEFESPKKKRKVSEVEFSHEYWMRHALTLAKRARDEREVPVGAVLVLNNRVIGEGWNR | 401 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSGVKVEKQVTQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM28 ABE8e | MKRTADGSEFESPKKKRKVSEVEFSHEYWMRHALTLAKRARDEREVPVGAVLVLNNRVIGEGWNR | 402 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIETILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKKVTQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM44 ABE8e | MKRTADGSEFESPKKKRKVSEVEFSHEYWMRHALTLAKRARDEREVPVGAVLVLNNRVIGEGWNR | 403 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSRVKVEKQVYQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM51 ABE8e | MKRTADGSEFESPKKKRKVSEVEFSHEYWMRHALTLAKRARDEREVPVGAVLVLNNRVIGEGWNR | 404 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVF | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGERNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFGGSRVKVEKQVFKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM64 ABE8e | MKRTADGSEFESPKKKRKVSEVEFSHEYWMRHALTLAKRARDEREVPVGAVLVLNNRVIGEGWNR | 405 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMENL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDRGEGNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSGVKVEKQVYKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM65 ABE8e | MKRTADGSEFESPKKKRKVSEVEFSHEYWMRHALTLAKRARDEREVPVGAVLVLNNRVIGEGWNR | 406 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFKNSGVKVEKQVTQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM67 ABE8e | MKRTADGSEFESPKKKRKVSEVEFSHEYWMRHALTLAKRARDEREVPVGAVLVLNNRVIGEGWNR | 407 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDFNFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIDMGDRNLLYIVVVDGKGNIVEQYSLNEIINNFNGIRIKTDYHSLLDKKEKERFEARQN | |
| | WTSIENIKELKAGYISQVVHKICELVEKYDAVIALEDLNSGFKNSRVKVEKKVTQKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |
| LbFM76 ABE8e | MKRTADGSEFESPKKKRKVSEVEFSHEYWMRHALTLAKRARDEREVPVGAVLVLNNRVIGEGWNR | 408 |
| | AIGLHDPTAHAEIMALRQGGLVMQNYRLIDATLYVTFEPCVMCAGAMIHSRIGRVVFGVRNSKRG | |
| | AAGSLMNVLNYPGMNHRVEITEGILADECAALLCDFYRMPRQVFNAQKKAQSSINSGGSSGGSSG | |
| | SETPGTSESATPESSGGSSGGSSKLEKFTNCYSLSKTLRFKAIPVGKTQENIDNKRLLVEDEKRA | |
| | EDYKGVKKLLDRYYLSFINDVLHSIKLKNLNNYISLFRKKTRTEKENKELENLEINLRKEIAKAF | |
| | KGNEGYKSLFKKDIIATILPEFLDDKDEIALVNSFNGFTTAFTGFFDNRENMFSEEAKSTSIAFR | |
| | CINENLTRYISNMDIFEKVDAIFDKHEVQEIKEKILNSDYDVEDFFEGEFFNFVLTQEGIDVYNA | |
| | IIGGFVTESGEKIKGLNEYINLYNQKTKQKLPKFKPLYKQVLSDRESLSFYGEGYTSDEEVLEVE | |
| | RNTLNKNSEIFSSIKKLEKLFKNFDEYSSAGIFVKNGPAISTISKDIFGEWNVIRDKWNAEYDDI | |
| | HLKKKAVVTEKYEDDRRKSFKKIGSFSLEQLQEYADADLSVVEKLKEIIIQKVDEIYKVYGSSEK | |
| | LFDADFVLEKSLKKNDAVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATILRYGSKYYLAIMDKKYAK | |
| | CLQKIDKDDVNGNYEKINYKLLPGPNKMLPKVFFSKKWMAYYNPSEDIQKIYKNGTFKKGDMFNL | |
| | NDCHKLIDFFKDSISRYPKWSNAYDENFSETEKYKDIAGFYREVEEQGYKVSFESASKKEVDKLV | |
| | EEGKLYMFQIYNKDFSDKSHGTPNLHTMYFKLLFDENNHGQIRLSGGAELFMRRASLKKEELVVH | |
| | PANSPIANKNPDNPKKTTTLSYDVYKDKRFSEDQYELHIPIAINKCPKNIFKINTEVRVLLKHDD | |
| | NPYVIGIARGERNLLYIVVVDGKGNIVEQYSLNEIINNENGIRIKTDYHSLLDKKEKERFEARQN | |
| | WTSIENIKELKAGYISQVVHKICELVEKYDAVIALEDLNSGFGGSRVKVEKQVFKKFEKMLIDKL | |
| | NYMVDKKSNPCATGGALKGYQITNKFESFKSMSTQNGFIFYIPAWLTSKIDPSTGFVNLLKTKYT | |
| | SIADSKKFISSFDRIMYVPEEDLFEFALDYKNFSRTDADYIKKWKLYSYGNRIRIFRNPKKNNVF | |
| | DWEEVCLTSAYKELFNKYGINYQQGDIRALLCEQSDKAFYSSFMALMSLMLQMRNSITGRTDVDF | |
| | LISPVKNSDGIFYDSRNYEAQENAILPKNADANGAYNIARKVLWAIGQFKKAEDEKLDKVKIAIS | |
| | NKEWLEYAQTSVKSGGSKRTADGSEFEPKKKRKV | |

*Protein Elements ordered from N-terminus to C-terminus

In some embodiments, a fusion protein comprises the amino acid sequence of any one of SEQ ID NO: 163-185. In some embodiments, a fusion protein comprises an amino acid sequence that includes any one or more mutation(s) (e.g., amino acid substitution(s)) described herein and has at least 80% (e.g., at least 80%, at least 85%, at least 90%, at least 95%, or at least 98%) identity to the amino acid sequence of any one of the fusion protein in Table 5 (e.g., SEQ ID NOs: 163-185).

### VIII. Cells

Aspects of the present disclosure relate to cells comprising any more of the variant Cas12a endonucleases described herein. Further, as described below, the variant Cas12a endonucleases described herein may be used to modify cells.

The cells may be eukaryotic cells or prokaryotic cells. Non-limiting examples of eukaryotic cells include animal cells, plant cells, and fungal cells. In some embodiments, the cells are mammalian cells. In some embodiments, the cells are human cells (e.g., human primary cells or human immortalized cells). In some embodiments, the cells are stem cells, such as adult stem cells or induced pluripotent stem cells (iPSCs). Non-human cells are also provided herein. For example, the cells may be selected from non-human primate cells, porcine cells, bovine cells, canine cells, feline cells, or rodent cells (e.g., rat or mouse cells).

Various human cell types are contemplated herein, including without limitation, immune cells (e.g., T cells (e.g., NKT cells, CD4+ T cells, CD8+ T cells, regulatory T cells, engineered T cells, e.g., CAR-T, TCR)), B cells, NK cells, tumor-infiltrating lymphocytes, etc.), neural cells, cardiovascular cells, epidermal cells, and metabolic cells. The cells may be cancerous or non-cancerous. In some embodiments, the cells are tumor cells. Other cell types are contemplated herein.

### IX. Methods of Use

Cas12a endonucleases provided herein have numerous uses, many of which are known in the art. The variant Cas12a endonucleases of the present disclosure maybe used, in some instances, to improve those uses. Several non-limiting examples of such uses include genome editing, bioengineering, diagnostics, and agricultural advancement.

### A. Genome Editing

In some embodiments, the variant Cas12a endonuclease are used for genome editing. Genome editing is a type of genetic engineering where a DNA is inserted, deleted or replaced in the genome of a living organism. Application of CRISPR-Cas systems as molecular tools for genome editing exploits their ability to produce a double strand break (DSB) at a specific genomic locus and depends entirely on the host cell DNA repair machinery to fix the lesion produced by these systems. The repair mechanisms can be either of the following processes: homology-directed repair (HDR) or non-homologous end joining (NHEJ). HDR utilizes a template DNA that is homologous to the break site (an unbroken sister chromatid or a homologous chromosome) to repair the DSB, whereas NHEJ is based on direct joining of broken ends of the DSB, making NHEJ the more error prone mechanism of the two. HDR can thus be used to supply exogenous template DNA to implement a user defined change in the host genome. NHEJ can be applied for gene disruption whereas HDR allows for the scope of introducing new genetic information or direct correction of the sequence at a specific locus.

At the center of CRISPR mediated genome engineering today is Cas9, with applications including, but not limited to, gene knockout and precise genome editing. Despite the rapid advances in genome editing by Cas9, it still presents challenges owing to the possibility of off-target effects and difficulty of delivering the ribonucleoprotein particle. Cas12a, owing to its substantial differences with Cas9, presents an alternate molecular genome editing tool. The use of Cas12a in genome editing for various cell types has been probed in several studies up to date. Comparative studies of gene repression by catalytically dead Cas9 from S. pyogenes (SpdCas9) and catalytically dead Cas12a from Eubacterium eligens (EedCas12a) revealed that the latter displays a higher gene repression in the template strand of the target DNA than SpdCas9. It was also shown that the pre-crRNA processing activity of Cas12a makes it an attractive candidate for multiplex gene regulation, which is cumbersome when attempted with Cas9. This auto-processing of its own crRNA has been used to modify multiple genetic elements simultaneously generating constitutive, conditional, inducible, orthogonal and multiplexed genome engineering of endogenous targets using multiple CRISPR RNAs delivered on a single plasmid.

The viability of this approach has been further established by other studies, in which multiplex gene regulation by Cas12a was successfully observed in bacteria, plants, as well as in mammalian cells. Cas12a can also serve as a solution in cell types where use of Cas9 is toxic, such as in some industrial strains of Streptomyces.

Targeted mutagenesis in plants can also be achieved through co-expression of Cas12a and its cognate crRNA in vivo, as was shown in rice. Additionally, it was also shown that the mutagenesis was more efficient through the use of pre-crRNAs with full-length direct repeat sequences than with mature crRNAs. Efficient mutagenesis through delivery of the pre-assembled ribonucleoprotein (RNP) particle was also observed in soybean and wild tobacco. The RNP was assembled from recombinantly expressed Cas12a and in vitro transcribed or chemically synthesized crRNAs.

Successful gene editing of mammalian cells using Cas12a include correction of mutations causing Duchenne muscular dystrophy (DMD) in patient derived induced pluripotent stem cells (iPSCs) and in mdx mice, a popular model for studying DMD. Dystrophin expression was reinstated in iPSCs after Cas12a-mediated gene editing, while in the mdx mice, corrections in the pathophysiological ballmarks of muscular dystrophy were observed. Delivery of the adenovirus vector with an AsCas12a expression cassette yielded successful mutations in primary human hepatocytes from humanized mice with chimeric liver. Cas12a-mediated genome editing was also used to engineer rat models that mimic human atherosclerosis and this system may have potential applications in understanding early stage atherosclerosis.

See Paul, B. & Montoya, G. et al. Biomedical Journal 2020; 43 (1): 8-17, incorporated herein in its entirety.

### B. Bioengineering

In some embodiments, the variant Cas12a endonuclease are used for bioengineering. Currently, a vast effort is ongoing to redesign all these tools for biomedical and biotechnological applications. However, recent studies have envisioned the possibility of using CRISPR-Cas nucleases in bioengineering of smart materials, for example hydrogels. These water-filled polymers are encapsulated by DNA. Cas12a has been used to specifically degrade the DNA scaffold of DNA hydrogels, thus opening the possibility that this smart cutter can be turned into a programmable device to deliver the cargo of DNA encaged hydrogels in a determined location at a certain time. The cleavage properties of Cas12a make it an ideal candidate to promote controlled delivery of the cargo. See Paul, B. & Montoya, G. et al. Biomedical Journal 2020; 43 (1): 8-17, incorporated herein in its entirety.

### C. Nucleic Acid Detection and Quantification (e.g., Diagnostics)

In some embodiments, the variant Cas12a endonucleases are used for detecting and/or quantifying nucleic acids. For example, the variants may be used as in vitro diagnostic tools for pathogenic (e.g., bacterial or viral) nucleic acids, or for identification of biomarkers indicative of disease, such as cancer (e.g., for identification and quantification of single CpG methylation sites-see, for example, van Dongen, J E et al. Biosensors and Bioelectronics 2021; 194 (15): 113624).

In some embodiments, the variant Cas12a endonucleases are used with the Specific Enhancer for Detection of PCR-amplified Nucleic Acids (SENA) method, which combines the transcleavage activity of Cas12a with the sensitivity offered by real-time PCR. See, e.g., Huang W, et al. EBioMedicine 2020; 61:103036.

In some embodiments, the variant Cas12a endonucleases are used with the DNA Endonuclease-Targeted CRISPR Trans Reporter (DETECTR) technology, which performs simultaneous reverse transcription and isothermal amplification using loop-mediated amplification (RT-LAMP) for RNA extracted from a biological sample. See, e.g., Broughton J P, et al. Nature Biotechnology 2020; 38:870-874.

In some embodiments, the variant Cas12a endonucleases are used with the one-hour low-cost multipurpose highly efficient system (HOLMES) technology. See, e.g., Li, L. et al. ACS Synth Biol. 2019; 8 (10): 2228-2237.

Other nucleic acid detection methods are contemplated herein.

### D. Agriculture

In some embodiments, the variant Cas12a endonuclease are used for applications relating to agricultural advancement. Cas12a editing has been widely utilized in many crops including rice, wheat, maize, soybean, cotton, tomato, citrus, tobacco, and the model plant Arabidopsis. At present, three Cas12a genome editing systems AsCas12a, FnCas12a, and LbCas12a have been demonstrated in plants with varied efficiency.

Rice is one of the most well-studied crops due to its agricultural importance, small genome size, ease of transformation and available genetic resources making it an ideal flagship genome for the grasses. These factors have also made it an ideal testing ground for developing genome editing technologies. Codon optimized FnCas12a binary vectors were utilized for targeted mutagenesis in rice (OsDL, OsALS, OsNCED1, OsAO1) and tobacco (NtPDS and NtSTF1) with average targeted mutation frequencies of 47.2% and 28.2%, respectively. Utilizing the LbCas12a nuclease two endogenous rice genes OsPDS and OsBEL were targeted with mutation frequencies of 21.4 and 41.2%, respectively. An independent study that targeted the disruption of OsPDS by LbCas12a resulted in a similarly high editing frequency of 32.3%. It was also demonstrated that pre-crRNAs were more efficient in generating mutants than mature crRNAs in rice. However, the opposite was observed in HEK293T cells. In addition to these proof-of-concept experiments, LbCas12a was also used to create loss-of-function alleles of OsEPFL9 which regulates stomatal density. These lines increased water use efficiency eight-fold in T2 generation plants. See, Bandyopadhyay, A. et al. Front. Plant Sci. 2020.

### Additional Embodiments

Additional embodiments are described in the following numbered paragraphs:

1. An engineered variant Cas12a endonuclease comprising a polypeptide sequence comprising a mutation at an amino acid position corresponding to position E95, E125, V245, N260, Y277, R747, H759, 1765, F810, N813, T814, 1831, T870, G902, K960, S982, K984, or T988 with reference to amino acid position numbering of LbCas12a ND2006.

2. The engineered variant Cas12a endonuclease of paragraph 1, wherein the variant Cas12a endonuclease exhibits hyperactivity, low single indiscriminate strand deoxyribonuclease (DNase) activity, target nickase activity (or a preference for cleaving one strand over the other of a dsDNA), or protospacer adjacent motif (PAM) nickase activity.

3. The engineered variant Cas12a endonuclease of paragraph 1 or 2, wherein the mutation is an amino acid substitution.

4. The engineered variant Cas12a endonuclease of paragraph 3, wherein the amino acid substitution is an amino acid having an equivalent charge, polarity, and/or chemical class.

5. The engineered variant Cas12a endonuclease of any one of paragraphs 1-4, wherein (a) the polypeptide sequence comprises a mutation at an amino acid position corresponding to position E95, E125, V245, Y277, R747, H759, 1765, F810, or T814 with reference to amino acid position numbering of LbCas12a ND2006 and (b) the variant Cas12a endonuclease exhibits hyperactivity.

6. The engineered variant Cas12a endonuclease of paragraph 5, wherein the polypeptide sequence comprises a mutation at an amino acid position corresponding to position E95 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

7. The engineered variant Cas12a endonuclease of paragraph 6, wherein the mutation is a substitution of a polar, positively charged, and/or basic amino acid at position E95 with reference to amino acid position numbering of LbCas12a ND2006, preferably E95R or E95H, more preferably E95R.

8. The engineered variant Cas12a endonuclease of paragraph 6, wherein the mutation is a substitution of a polar, uncharged, and/or aromatic amino acid at position E95 with reference to amino acid position numbering of LbCas12a ND2006, preferably E95Y.

9. The engineered variant Cas12a endonuclease of paragraph 5, wherein the polypeptide sequence comprises a mutation at an amino acid position corresponding to position E125 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

10. The engineered variant Cas12a endonuclease of paragraph 9, wherein the mutation is a substitution of a nonpolar, uncharged, and/or aliphatic amino acid at position E125 with reference to amino acid position numbering of LbCas12a ND2006, preferably E125A, E125G, E125I, E125L, E125P, E125V, more preferably E125A.

14. The engineered variant Cas12a endonuclease of paragraph 5, wherein the polypeptide sequence comprises a mutation at an amino acid position corresponding to position V245 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

15. The engineered variant Cas12a endonuclease of paragraph 14, wherein the mutation is a substitution of a nonpolar, uncharged, and/or aliphatic amino acid at position V245 with reference to amino acid position numbering of LbCas12a ND2006, preferably V245I, V245A, V245G, V245L, or V245P, more preferably V245I.

16. The engineered variant Cas12a endonuclease of paragraph 14, wherein the mutation is a substitution of a polar, uncharged, and/or aromatic amino acid at position V245 with reference to amino acid position numbering of LbCas12a ND2006, preferably V245Y.

21. The engineered variant Cas12a endonuclease of paragraph 5, wherein the polypeptide sequence comprises a mutation at an amino acid position corresponding to position Y277 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

22. The engineered variant Cas12a endonuclease of paragraph 21, wherein the mutation is a substitution of a polar, uncharged, and/or hydroxyl amino acid at position Y277 with reference to amino acid position numbering of LbCas12a ND2006, preferably Y277S or Y277T, more preferably Y277S.

29. The engineered variant Cas12a endonuclease of paragraph 5, wherein the polypeptide sequence comprises a mutation at an amino acid position corresponding to position R747 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

30. The engineered variant Cas12a endonuclease of paragraph 29, wherein the mutation is a substitution of a polar, uncharged, and/or aromatic amino acid at position R747 with reference to amino acid position numbering of LbCas12a ND2006, preferably R747Y.

31. The engineered variant Cas12a endonuclease of paragraph 5, wherein the polypeptide sequence comprises a mutation at an amino acid position corresponding to position H759 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

32. The engineered variant Cas12a endonuclease of paragraph 31, wherein the mutation is a substitution of a nonpolar, uncharged, and/or aliphatic amino acid at position H759 with reference to amino acid position numbering of LbCas12a ND2006, preferably H759V, H759A, H759G, H7591, H759L, or H759P, more preferably H759V.

33. The engineered variant Cas12a endonuclease of paragraph 31, wherein the mutation is a substitution of a polar, negatively charged, and/or acidic amino acid at position H759 with reference to amino acid position numbering of LbCas12a ND2006, preferably H759D.

34. The engineered variant Cas12a endonuclease of paragraph 5, wherein the polypeptide sequence comprises a mutation at an amino acid position corresponding to position 1765 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

35. The engineered variant Cas12a endonuclease of paragraph 34, wherein the mutation is a substitution of a polar, uncharged, and/or aromatic amino acid at position 1765 with reference to amino acid position numbering of LbCas12a ND2006, preferably I765Y.

36. The engineered variant Cas12a endonuclease of paragraph 5, wherein the polypeptide sequence comprises a mutation at an amino acid position corresponding to position F810 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

37. The engineered variant Cas12a endonuclease of paragraph 36, wherein the mutation is a substitution of a nonpolar, uncharged, and/or aromatic amino acid at position F810 with reference to amino acid position numbering of LbCas12a ND2006, preferably F810W.

38. The engineered variant Cas12a endonuclease of paragraph 36, wherein the mutation is a substitution of a polar, charged, and/or acidic amino acid at position F810 with reference to amino acid position numbering of LbCas12a ND2006, preferably F810Q.

39. The engineered variant Cas12a endonuclease of paragraph 5, wherein the polypeptide sequence comprises a mutation at an amino acid position corresponding to position T814 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

40. The engineered variant Cas12a endonuclease of paragraph 39, wherein the mutation is a substitution of a nonpolar, uncharged, and/or aromatic amino acid at position T814 with reference to amino acid position numbering of LbCas12a ND2006, preferably T814E.

41. The engineered variant Cas12a endonuclease of paragraph 39, wherein the mutation is a substitution of a polar, positively charged, and/or basic amino acid at position T814 with reference to amino acid position numbering of LbCas12a ND2006, preferably T814R, T814H, or T814K, more preferably T814R.

42. The engineered variant Cas12a endonuclease of any one of paragraphs 1-4, wherein (a) the polypeptide sequence comprises a mutation at an amino acid position corresponding to position N813, 1831, T870, G902, S982, K984, or T988 with reference to amino acid position numbering of LbCas12a ND2006 and (b) the variant Cas12a endonuclease exhibits low indiscriminate single strand deoxyribonuclease (ssDNase) activity.

43. The engineered variant Cas12a endonuclease of paragraph 42, wherein the polypeptide sequence comprises a mutation at an amino acid position corresponding to position N813 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

44. The engineered variant Cas12a endonuclease of paragraph 43, wherein the mutation is a substitution of a nonpolar, uncharged, and/or aromatic amino acid at position N813 with reference to amino acid position numbering of LbCas12a ND2006, preferably N813W.

45. The engineered variant Cas12a endonuclease of paragraph 43, wherein the mutation is a substitution of a polar, positively charged, and/or basic amino acid at position N813 with reference to amino acid position numbering of LbCas12a ND2006, preferably N813R, N813H, or N813K, more preferably N813R or N813H.

46. The engineered variant Cas12a endonuclease of paragraph 42, wherein the polypeptide sequence comprises a mutation at an amino acid position corresponding to position I831 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

47. The engineered variant Cas12a endonuclease of paragraph 46, wherein the mutation is a substitution of a nonpolar, uncharged, and/or aliphatic amino acid at position I831 with reference to amino acid position numbering of LbCas12a ND2006, preferably I831A, I831G, I831L, I831P, or I831V, more preferably I831A.

48. The engineered variant Cas12a endonuclease of paragraph 46, wherein the mutation is a substitution of a polar, uncharged, and/or aromatic amino acid at position I831 with reference to amino acid position numbering of LbCas12a ND2006, preferably I831Y.

49. The engineered variant Cas12a endonuclease of paragraph 42, wherein the polypeptide sequence comprises a mutation at an amino acid position corresponding to position T870 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

50. The engineered variant Cas12a endonuclease of paragraph 47, wherein the mutation is a substitution of a polar, uncharged, and/or aromatic amino acid at position T870 with reference to amino acid position numbering of LbCas12a ND2006, preferably T870Y.

51. The engineered variant Cas12a endonuclease of paragraph 47, wherein the mutation is a substitution of a nonpolar, uncharged, and/or aromatic amino acid at position T870 with reference to amino acid position numbering of LbCas12a ND2006, preferably T870F or T870W, more preferably T870F.

52. The engineered variant Cas12a endonuclease of paragraph 42, wherein the polypeptide sequence comprises a mutation at an amino acid position corresponding to position G902 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

53. The engineered variant Cas12a endonuclease of paragraph 52, wherein the mutation is a substitution of a polar, positively charged, and/or basic amino acid at position G902 with reference to amino acid position numbering of LbCas12a ND2006, preferably G902R, G902H, or G902K, more preferably G902R.

54. The engineered variant Cas12a endonuclease of paragraph 52, wherein the mutation is a substitution of a nonpolar, uncharged, and/or aromatic amino acid at position G902 with reference to amino acid position numbering of LbCas12a ND2006, preferably G902W or G902F, more preferably G902W.

55. The engineered variant Cas12a endonuclease of paragraph 42, wherein the polypeptide sequence comprises a mutation at an amino acid position corresponding to position S982 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

56. The engineered variant Cas12a endonuclease of paragraph 55, wherein the mutation is a substitution of a nonpolar, uncharged, and/or aromatic amino acid at position S982 with reference to amino acid position numbering of LbCas12a ND2006, preferably S982F or S982W, more preferably S982W.

57. The engineered variant Cas12a endonuclease of paragraph 42, wherein the polypeptide sequence comprises a mutation at an amino acid position corresponding to position K984 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

58. The engineered variant Cas12a endonuclease of paragraph 57, wherein the mutation is a substitution of a nonpolar, uncharged, and/or aromatic amino acid at position K984 with reference to amino acid position numbering of LbCas12a ND2006, preferably K984F or K984W, more preferably K984F.

59. The engineered variant Cas12a endonuclease of paragraph 57, wherein the mutation is a substitution of a polar, positively charged, and/or basic amino acid at position K984 with reference to amino acid position numbering of LbCas12a ND2006, preferably K984R, K984H, or K984K, more preferably K984R.

60. The engineered variant Cas12a endonuclease of paragraph 42, wherein the polypeptide sequence comprises a mutation at an amino acid position corresponding to position T988 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

61. The engineered variant Cas12a endonuclease of paragraph 60, wherein the mutation is a substitution of a nonpolar, uncharged, and/or aromatic amino acid at position T988 with reference to amino acid position numbering of LbCas12a ND2006, preferably T988F or T988W, more preferably T988F.

62. The engineered variant Cas12a endonuclease of any one of paragraphs 1-4, wherein (a) the polypeptide sequence comprises a mutation at an amino acid position corresponding to position N260 or G902 with reference to amino acid position numbering of LbCas12a ND2006 and (b) the variant Cas12a endonuclease exhibits target nickase activity (or a preference for cleaving one strand over the other of a dsDNA).

63. The engineered variant Cas12a endonuclease of paragraph 62, wherein the polypeptide sequence comprises a mutation at an amino acid position corresponding to position N260 with reference to amino acid position numbering of LbCas12a, optionally wherein the polypeptide sequence has at least 90% identity to a wild-type reference Cas12a endonuclease, optionally to a wild-type reference Cas12a endonuclease of Table 1.

64. The engineered variant Cas12a endonuclease of paragraph 63, wherein the mutation is a substitution of a polar, positively charged, and/or basic amino acid at position N260 with reference to amino acid position numbering of LbCas12a ND2006, preferably N260R, N260H, or N260K, more preferably N260R.

65. The engineered variant Cas12a endonuclease of paragraph 62, wherein the polypeptide sequence comprises a mutation at an amino acid position corresponding to position G902 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

66. The engineered variant Cas12a endonuclease of paragraph 65, wherein the mutation is a substitution of a nonpolar, uncharged, and/or aromatic amino acid at position G902 with reference to amino acid position numbering of LbCas12a ND2006, preferably G902W or G902F, more preferably G902W.

67. The engineered variant Cas12a endonuclease of any one of paragraphs 1-4, wherein (a) polypeptide sequence comprises a mutation at an amino acid position corresponding to position K960 with reference to amino acid position numbering of LbCas12a ND2006 and (b) the variant Cas12a endonuclease exhibits PAM nickase activity (or a preference for cleaving one strand over the other of a dsDNA).

68. The engineered variant Cas12a endonuclease of paragraph 67, wherein the mutation is a substitution of a polar, negatively charged, and/or amide amino acid amino acid at position K960 with reference to amino acid position numbering of LbCas12a ND2006, preferably K960E, optionally wherein the polypeptide sequence has at least 90% identity to a wild-type reference Cas12a endonuclease, optionally to a wild-type reference Cas12a endonuclease of Table 1.

69. The engineered variant Cas12a endonuclease of any one of the preceding paragraphs comprising an amino acid sequence having at least 85%, at least 90%, or least 95%, but less than 100% identity with the amino acid sequence of a wild-type Cas12a endonuclease selected from Acidaminococcus sp., Lachnospiraceae sp., and Francisella sp.

70. The engineered variant Cas12a endonuclease of any one of the preceding paragraphs further comprising no more than 1, 2, 3, 4, 5, 6, 7, 8, 9, or 10 additional amino acid substitutions relative to a wild-type reference Cas12a endonuclease.

71. An engineered variant Cas12a endonuclease comprising a polypeptide sequence comprising the amino acid sequence of any one of SEQ ID NOs: 48-119.

72. A polynucleotide encoding the variant Cas12a endonuclease of any one of the preceding paragraphs.

73. A cell comprising (a) the variant Cas12a endonuclease of any one of the preceding paragraphs or the polynucleotide of paragraph 72 and (b) a guide RNA or a polynucleotide encoding a guide RNA.

74. A method comprising introducing into a cell (a) the variant Cas12a endonuclease of any one of the preceding paragraphs or the polynucleotide of paragraph 72 and optionally (b) a guide RNA or a polynucleotide encoding a guide RNA.

75. Use of the variant Cas12a endonuclease of any one of the preceding paragraphs for cleaving a nucleic acid.

76. A method for introducing a double strand break in a target nucleic acid, comprising introducing into a cell comprising a target nucleic acid (a) the variant Cas12a endonuclease of any one of paragraphs 5-41 and (b) a guide RNA; and incubating the cell to produce a double strand break in the target nucleic acid.

77. A method for introducing a double strand break in a target nucleic acid, comprising introducing into a cell comprising a target nucleic acid (a) the variant Cas12a endonuclease of any one of paragraphs 42-61 and (b) a guide RNA; and incubating the cell to produce a double strand break in the target nucleic acid.

78. The method of paragraph 77, wherein off-target single strand nucleic acid cleavage in the cell is reduced relative to off-target single strand nucleic acid cleavage in a control cell comprising a wild-type Cas12a endonuclease and a guide RNA.

79. A method for introducing a single strand break in a target nucleic acid, comprising introducing into a cell comprising a target nucleic acid (a) the variant Cas12a endonuclease of any one of paragraphs 62-68 and (b) a guide RNA; and incubating the cell to produce a single strand break in the target nucleic acid.

### Further Embodiments

Further embodiments are described in the following numbered paragraphs:

1. An engineered variant Cas12a endonuclease comprising a polypeptide sequence comprising a mutation at an amino acid position corresponding to position R833, E835, R836, S929, F931, K932, N933, S934, R935, V936, K937, V938, K940, Q941, Y943, Q944, F983, or M986 with reference to amino acid position numbering of LbCas12a ND2006.

2. The engineered variant Cas12a endonuclease of paragraph 1, wherein the engineered variant Cas12a endonuclease exhibits hyperactivity, low indiscriminate single strand deoxyribonuclease (DNase) activity, or target nickase activity (or a preference for cleaving one strand over the other of a dsDNA).

3. The engineered variant Cas12a endonuclease of paragraph 1 or 2, wherein the mutation is an amino acid substitution.

4. The engineered variant Cas12a endonuclease of paragraph 3, wherein the amino acid substitution is an amino acid having an equivalent charge, polarity, and/or chemical class.

5. The engineered variant Cas12a endonuclease of any one of paragraphs 1-4, wherein (a) the polypeptide sequence comprises a mutation at an amino acid position corresponding to position K932, N933, or V936, with reference to amino acid position numbering of LbCas12a ND2006 and (b) the engineered variant Cas12a endonuclease exhibits hyperactivity.

6. The engineered variant Cas12a endonuclease of paragraph 5, wherein the polypeptide sequence comprises a mutation at an amino acid position corresponding to position K932 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

7. The engineered variant Cas12a endonuclease of paragraph 6, wherein the mutation is a substitution of a nonpolar, uncharged, and/or aliphatic amino acid at position K932 with reference to amino acid position numbering of LbCas12a ND2006, preferably K932I, K932L, K932V, K932A, or K932P, more preferably K932I, K932L, or K932V.

8. The engineered variant Cas12a endonuclease of paragraph 5, wherein the polypeptide sequence comprises a mutation at an amino acid position corresponding to position N933 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

9. The engineered variant Cas12a endonuclease of paragraph 8, wherein the mutation is a substitution of a nonpolar, uncharged, and/or aliphatic amino acid at position N933 with reference to amino acid position numbering of LbCas12a, preferably N933L, N933A, N933G, N933I, or N933P, more preferably N933L.

10. The engineered variant Cas12a endonuclease of paragraph 5, wherein the polypeptide sequence comprises a mutation at an amino acid position corresponding to position V936 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

11. The engineered variant Cas12a endonuclease of paragraph 10, wherein the mutation is a substitution of a nonpolar, uncharged, and/or sulfuric amino acid at position V936 with reference to amino acid position numbering of LbCas12a, preferably V936M or V936C, more preferably V936M.

12. The engineered variant Cas12a endonuclease of any one of paragraphs 1-4, wherein (a) the polypeptide sequence comprises a mutation at an amino acid position corresponding to position S929, K932, N933, S934, V936, K937, Q944, F983, or M986 with reference to amino acid position numbering of LbCas12a ND2006 and (b) the variant Cas12a endonuclease exhibits low indiscriminate single strand deoxyribonuclease (ssDNase) activity.

13. The engineered variant Cas12a endonuclease of paragraph 12, wherein the polypeptide sequence comprises a mutation at an amino acid position corresponding to position S929 with reference to amino acid position numbering of LbCas12a, optionally wherein the polypeptide sequence has at least 90% identity to a wild-type reference Cas12a endonuclease, optionally to a wild-type reference Cas12a endonuclease of Table 1.

14. The engineered variant Cas12a endonuclease of paragraph 13, wherein the mutation is a substitution of a nonpolar, uncharged, and/or aliphatic amino acid at position S929 with reference to amino acid position numbering of LbCas12a, preferably S929L, S929A, S929I, S929P, or S929V, more preferably S929L.

15. The engineered variant Cas12a endonuclease of paragraph 12, wherein the polypeptide sequence comprises a mutation at an amino acid position corresponding to position K932 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

16. The engineered variant Cas12a endonuclease of paragraph 15, wherein the mutation is a substitution of a polar, positively charged, and/or basic amino acid at position K932 with reference to amino acid position numbering of LbCas12a, preferably K932R.

17. The engineered variant Cas12a endonuclease of paragraph 15, wherein the mutation is a substitution of a polar, uncharged, and/or hydroxyl amino acid at position K932 with reference to amino acid position numbering of LbCas12a, preferably K932T.

18. The engineered variant Cas12a endonuclease of paragraph 15, wherein the mutation is a substitution of a nonpolar, uncharged, and/or aromatic amino acid at position K932 with reference to amino acid position numbering of LbCas12a, preferably K932F or K932W.

19. The engineered variant Cas12a endonuclease of paragraph 15, wherein the mutation is a substitution of a polar, uncharged, and/or aromatic amino acid at position K932 with reference to amino acid position numbering of LbCas12a, preferably K932Y.

20. The engineered variant Cas12a endonuclease of paragraph 12, wherein the polypeptide sequence comprises a mutation at an amino acid position corresponding to position N933 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

21. The engineered variant Cas12a endonuclease of paragraph 20, wherein the mutation is a substitution of a polar, negatively charged, and/or amide amino acid at position N933 with reference to amino acid position numbering of LbCas12a, preferably N933E.

22. The engineered variant Cas12a endonuclease of paragraph 20, wherein the mutation is a substitution of a nonpolar, uncharged, and/or aliphatic amino acid at position N933 with reference to amino acid position numbering of LbCas12a, preferably N933V, N933A, N933G, N933I, or N933P, more preferably N933V.

23. The engineered variant Cas12a endonuclease of paragraph 12, wherein the polypeptide sequence comprises a mutation at an amino acid position corresponding to position S934 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

24. The engineered variant Cas12a endonuclease of paragraph 23, wherein the mutation is a substitution of a polar, uncharged, and/or acidic amino acid at position S934 with reference to amino acid position numbering of LbCas12a, preferably S934Q.

25. The engineered variant Cas12a endonuclease of paragraph 12, wherein the polypeptide sequence comprises a mutation at an amino acid position corresponding to position V936 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

26. The engineered variant Cas12a endonuclease of paragraph 25, wherein the mutation is a substitution of a polar, negatively charged, and/or amide amino acid at position V936 with reference to amino acid position numbering of LbCas12a, preferably V936E.

27. The engineered variant Cas12a endonuclease of paragraph 25, wherein the mutation is a substitution of a polar, positively charged, and/or basic amino acid at position V936 with reference to amino acid position numbering of LbCas12a, preferably V936K, V936R, or V936H, more preferably V936K.

28. The engineered variant Cas12a endonuclease of paragraph 12, wherein the polypeptide sequence comprises a mutation at an amino acid position corresponding to position K937 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

29. The engineered variant Cas12a endonuclease of paragraph 28, wherein the mutation is a substitution of a polar, uncharged, and/or aromatic amino acid at position K937 with reference to amino acid position numbering of LbCas12a, preferably K937Y.

30. The engineered variant Cas12a endonuclease of paragraph 12, wherein the polypeptide sequence comprises a mutation at an amino acid position corresponding to position Q944 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

31. The engineered variant Cas12a endonuclease of paragraph 30, wherein the mutation is a substitution of a polar, negatively charged, and/or acidic amino acid at position Q944 with reference to amino acid position numbering of LbCas12a, preferably Q944D.

32. The engineered variant Cas12a endonuclease of paragraph 30, wherein the mutation is a substitution of a nonpolar, uncharged, and/or sulfur amino acid at position Q944 with reference to amino acid position numbering of LbCas12a, preferably Q944M or Q944C, more preferably Q944M.

33. The engineered variant Cas12a endonuclease of paragraph 30, wherein the mutation is a substitution of a nonpolar, uncharged, and/or aliphatic amino acid at position Q944 with reference to amino acid position numbering of LbCas12a, preferably Q944G, Q944I, Q944A, Q944L, Q944P, or Q944V, more preferably Q944G.

34. The engineered variant Cas12a endonuclease of paragraph 12, wherein the polypeptide sequence comprises a mutation at an amino acid position corresponding to position F983 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

35. The engineered variant Cas12a endonuclease of paragraph 34, wherein the mutation is a substitution of a nonpolar, uncharged, and/or aliphatic amino acid at position F983 with reference to amino acid position numbering of LbCas12a, preferably F983L, F983A, F983G, F9831, F983P, or F983V, more preferably F983L.

36. The engineered variant Cas12a endonuclease of paragraph 12, wherein the polypeptide sequence comprises a mutation at an amino acid position corresponding to position M986 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

37. The engineered variant Cas12a endonuclease of paragraph 36, wherein the mutation is a substitution of a nonpolar, uncharged, and/or aromatic amino acid at position M986 with reference to amino acid position numbering of LbCas12a, preferably M986F or M986W, more preferably M986F.

38. The engineered variant Cas12a endonuclease of paragraph 36, wherein the mutation is a substitution of a polar, uncharged, and/or hydroxyl amino acid at position M986 with reference to amino acid position numbering of LbCas12a, preferably M986S or M986T, more preferably M986S.

39. The engineered variant Cas12a endonuclease of any one of paragraphs 1-4, wherein (a) the polypeptide sequence comprises a mutation at an amino acid position corresponding to position R833, E835, R836, F931, K932, R935, V936, V938, K940, Q941, Y943, Q944, or M986 with reference to amino acid position numbering of LbCas12a ND2006 and (b) the variant Cas12a endonuclease exhibits target nickase activity (or a preference for cleaving one strand over the other of a dsDNA).

40. The engineered variant Cas12a endonuclease of paragraph 39, wherein the polypeptide sequence comprises a mutation at an amino acid position corresponding to position R833 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

41. The engineered variant Cas12a endonuclease of paragraph 40, wherein the mutation is a substitution of a polar, positively charged, and/or basic amino acid at position R833 with reference to amino acid position numbering of LbCas12a, preferably R833K or R833H, more preferably R833K optionally wherein the engineered variant Cas12a endonuclease also exhibits low indiscriminate ssDNase activity.

42. The engineered variant Cas12a endonuclease of paragraph 40 wherein the mutation is a substitution of a nonpolar, uncharged, and/or sulfuric amino acid at position R833 with reference to amino acid position numbering of LbCas12a, preferably R833M or R833C, more preferably R833M, optionally wherein the engineered variant Cas12a endonuclease also exhibits low indiscriminate ssDNase activity.

43. The engineered variant Cas12a endonuclease of paragraph 40, wherein the mutation is a substitution of a nonpolar, uncharged, and/or aliphatic amino acid at position R833 with reference to amino acid position numbering of LbCas12a, preferably R833L, R833A, R833I, R833P, or R833V, more preferably R833L, optionally wherein the engineered variant Cas12a endonuclease also exhibits low indiscriminate ssDNase activity.

44. The engineered variant Cas12a endonuclease of paragraph 39, wherein the polypeptide sequence comprises a mutation at an amino acid position corresponding to position E835 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

45. The engineered variant Cas12a endonuclease of paragraph 44, wherein the mutation is a substitution of a polar, negatively charged, and/or acidic amino acid at position E835 with reference to amino acid position numbering of LbCas12a, preferably E835D, optionally wherein the engineered variant Cas12a endonuclease exhibits hypoactivity.

46. The engineered variant Cas12a endonuclease of paragraph 44, wherein the mutation is a substitution of a nonpolar, uncharged, and/or aliphatic amino acid at position E835 with reference to amino acid position numbering of LbCas12a, preferably E835G, E835A, E835I, E835L, E835P, or E835V, more preferably E835G or E835A.

47. The engineered variant Cas12a endonuclease of paragraph 39, wherein the polypeptide sequence comprises a mutation at an amino acid position corresponding to position R836 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

48. The engineered variant Cas12a endonuclease of paragraph 47, wherein the mutation is a substitution of a nonpolar, uncharged, and/or aliphatic amino acid at position R836 with reference to amino acid position numbering of LbCas12a, preferably R836G, R836A, R836I, R836L, R836P, or R836V, more preferably R836G or R836A.

49. The engineered variant Cas12a endonuclease of paragraph 39, wherein the polypeptide sequence comprises a mutation at an amino acid position corresponding to position F931 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

50. The engineered variant Cas12a endonuclease of paragraph 49, wherein the mutation is a substitution of a polar, positively charged, and/or basic amino acid at position F931 with reference to amino acid position numbering of LbCas12a, preferably F931H or F931K, more preferably F931H, optionally wherein the engineered variant Cas12a endonuclease exhibits hypoactivity.

51. The engineered variant Cas12a endonuclease of paragraph 49, wherein the mutation is a substitution of a nonpolar, uncharged, and/or aliphatic amino acid at position F931 with reference to amino acid position numbering of LbCas12a, preferably F931L, F931A, F931G, F931I, F931P, or F931V, more preferably F931L.

52. The engineered variant Cas12a endonuclease of paragraph 39, wherein the polypeptide sequence comprises a mutation at an amino acid position corresponding to position K932 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

53. The engineered variant Cas12a endonuclease of paragraph 52, wherein the mutation is a substitution of a nonpolar, uncharged, and/or aliphatic amino acid at position K932 with reference to amino acid position numbering of LbCas12a, preferably K932G or K932P, more preferably K932G, optionally wherein the engineered variant Cas12a endonuclease exhibits hypoactivity.

54. The engineered variant Cas12a endonuclease of paragraph 52, wherein the mutation is a substitution of a polar, negatively charged, and/or amide amino acid at position K932 with reference to amino acid position numbering of LbCas12a, preferably K932E, optionally wherein the engineered variant Cas12a endonuclease exhibits hypoactivity.

55. The engineered variant Cas12a endonuclease of paragraph 52, wherein the mutation is a substitution of a polar, positively charged, and/or basic amino acid at position K932 with reference to amino acid position numbering of LbCas12a, preferably K932H or K932R, more preferably K932H.

56. The engineered variant Cas12a endonuclease of paragraph 52, wherein the mutation is a substitution of a nonpolar, uncharged, and/or sulfur amino acid at position K932 with reference to amino acid position numbering of LbCas12a, preferably K932M or K932C, more preferably K932M.

57. The engineered variant Cas12a endonuclease of paragraph 52, wherein the mutation is a substitution of a polar, uncharged, and/or amide amino acid at position K932 with reference to amino acid position numbering of LbCas12a, preferably K932N.

58. The engineered variant Cas12a endonuclease of paragraph 52, wherein the mutation is a substitution of a polar, uncharged, and/or acidic amino acid at position K932 with reference to amino acid position numbering of LbCas12a, preferably K932Q.

59. The engineered variant Cas12a endonuclease of paragraph 52, wherein the mutation is a substitution of a polar, uncharged, and/or hydroxyl amino acid at position K932 with reference to amino acid position numbering of LbCas12a, preferably K932S.

60. The engineered variant Cas12a endonuclease of paragraph 39, wherein the polypeptide sequence comprises a mutation at an amino acid position corresponding to position R935 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

61. The engineered variant Cas12a endonuclease of paragraph 60, wherein the mutation is a substitution of a nonpolar, uncharged, and/or aliphatic amino acid at position R935 with reference to amino acid position numbering of LbCas12a, preferably R935L, R935G, R935I, or R935P, more preferably R935L, R935G, or R935I, optionally wherein the engineered variant Cas12a endonuclease having the R935I substitution exhibits hypoactivity.

62. The engineered variant Cas12a endonuclease of paragraph 60, wherein the mutation is a substitution of a polar, positively charged, and/or basic amino acid at position R935 with reference to amino acid position numbering of LbCas12a, preferably R935H or R935K.

63. The engineered variant Cas12a endonuclease of paragraph 60, wherein the mutation is a substitution of a nonpolar, uncharged, and/or aromatic amino acid at position R935 with reference to amino acid position numbering of LbCas12a, preferably R935F or R935W, optionally wherein the engineered variant Cas12a endonuclease having the R935W substitution exhibits hypoactivity.

64. The engineered variant Cas12a endonuclease of paragraph 60, wherein the mutation is a substitution of a nonpolar, uncharged, and/or sulfur amino acid at position R935 with reference to amino acid position numbering of LbCas12a, preferably R935M or R935C, more preferably R935M.

65. The engineered variant Cas12a endonuclease of paragraph 60, wherein the mutation is a substitution of a polar, uncharged, and/or amide amino acid at position R935 with reference to amino acid position numbering of LbCas12a, preferably R935N.

66. The engineered variant Cas12a endonuclease of paragraph 60, wherein the mutation is a substitution of a polar, uncharged, and/or hydroxyl amino acid at position R935 with reference to amino acid position numbering of LbCas12a, preferably R935S or R935T.

67. The engineered variant Cas12a endonuclease of paragraph 39, wherein the polypeptide sequence comprises a mutation at an amino acid position corresponding to position V936 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

68. The engineered variant Cas12a endonuclease of paragraph 67, wherein the mutation is a substitution of a nonpolar, uncharged, and/or aliphatic amino acid at position V936 with reference to amino acid position numbering of LbCas12a, preferably V936G, V936I, V936L, or V936P, more preferably V936G, optionally wherein the engineered variant Cas12a endonuclease also exhibits low indiscriminate ssDNase activity.

69. The engineered variant Cas12a endonuclease of paragraph 39, wherein the polypeptide sequence comprises a mutation at an amino acid position corresponding to position V938 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

70. The engineered variant Cas12a endonuclease of paragraph 69, wherein the mutation is a substitution of a polar, negatively charged, and/or amide amino acid at position V938 with reference to amino acid position numbering of LbCas12a, preferably V938E, optionally wherein the engineered variant Cas12a endonuclease also exhibits low indiscriminate ssDNase activity.

71. The engineered variant Cas12a endonuclease of paragraph 39, wherein the polypeptide sequence comprises a mutation at an amino acid position corresponding to position K940 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

72. The engineered variant Cas12a endonuclease of paragraph 71, wherein the mutation is a substitution of a nonpolar, uncharged, and/or aliphatic amino acid at position K940 with reference to amino acid position numbering of LbCas12a, preferably K940G, K940A, K940I, K940L, K940P, or K940V, more preferably K940G.

73. The engineered variant Cas12a endonuclease of paragraph 39, wherein the polypeptide sequence comprises a mutation at an amino acid position corresponding to position Q941 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

74. The engineered variant Cas12a endonuclease of paragraph 73, wherein the mutation is a substitution of a polar, positively charged, and/or basic amino acid at position Q941 with reference to amino acid position numbering of LbCas12a, preferably Q941K, Q941R, or Q941H.

75. The engineered variant Cas12a endonuclease of paragraph 73, wherein the mutation is a substitution of a polar, uncharged, and/or aromatic amino acid at position Q941 with reference to amino acid position numbering of LbCas12a, preferably Q941Y.

76. The engineered variant Cas12a endonuclease of paragraph 39, wherein the polypeptide sequence comprises a mutation at an amino acid position corresponding to position Y943 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

77. The engineered variant Cas12a endonuclease of paragraph 76, wherein the mutation is a substitution of a polar, uncharged, and/or hydroxyl amino acid at position Y943 with reference to amino acid position numbering of LbCas12a, preferably Y943T or Y943S, more preferably Y943T.

78. The engineered variant Cas12a endonuclease of paragraph 76, wherein the mutation is a substitution of a nonpolar, uncharged, and/or aromatic amino acid at position Y943 with reference to amino acid position numbering of LbCas12a, preferably Y943F or Y943W, more preferably Y943F.

79. The engineered variant Cas12a endonuclease of paragraph 39, wherein the polypeptide sequence comprises a mutation at an amino acid position corresponding to position Q944 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

80. The engineered variant Cas12a endonuclease of paragraph 79, wherein the mutation is a substitution of a polar, positively charged, and/or basic amino acid at position Q944 with reference to amino acid position numbering of LbCas12a, preferably Q944K, Q944R, or Q944H, more preferably Q944K.

81. The engineered variant Cas12a endonuclease of paragraph 79, wherein the mutation is a substitution of a polar, negatively charged, and/or amide amino acid at position Q944 with reference to amino acid position numbering of LbCas12a, preferably Q944E.

82. The engineered variant Cas12a endonuclease of paragraph 39, wherein the polypeptide sequence comprises a mutation at an amino acid position corresponding to position M986 with reference to amino acid position numbering of LbCas12a ND2006, optionally wherein the polypeptide sequence has at least 90% identity to a wild-type reference Cas12a endonuclease, optionally to a wild-type reference Cas12a endonuclease of Table 1.

83. The engineered variant Cas12a endonuclease of paragraph 82, wherein the mutation is a substitution of a nonpolar, uncharged, and/or aliphatic amino acid at position M986 with reference to amino acid position numbering of LbCas12a, preferably M986G, M986A, M986I, M986P, or M986V, more preferably M986G, optionally wherein the engineered variant Cas12a endonuclease also exhibits low indiscriminate ssDNase activity.

84. An engineered variant Cas12a endonuclease comprising a polypeptide sequence comprising amino acid substitutions corresponding to the following amino acid substitutions: R833L, S929L, K932M, Q944F, and E947M, with reference to amino acid position numbering of LbCas12a ND20006, preferably wherein the engineered variant Cas12a endonuclease exhibits low indiscriminate single strand DNase activity, optionally wherein the engineered variant Cas12a endonuclease also exhibits hypoactivity.

85. An engineered variant Cas12a endonuclease comprising a polypeptide sequence comprising amino acid substitutions corresponding to the following amino acid substitutions: N933L and Q944M, with reference to amino acid position numbering of LbCas12a ND20006, preferably wherein the engineered variant Cas12a endonuclease exhibits low indiscriminate single strand DNase activity, optionally wherein the engineered variant Cas12a endonuclease also exhibits hypoactivity.

86. An engineered variant Cas12a endonuclease comprising a polypeptide sequence comprising amino acid substitutions corresponding to the following amino acid substitutions: R833K and E947D, with reference to amino acid position numbering of LbCas12a ND20006, preferably wherein the engineered variant Cas12a endonuclease exhibits target nickase activity (or a preference for cleaving one strand over the other of a dsDNA).

87. An engineered variant Cas12a endonuclease comprising a polypeptide sequence comprising amino acid substitutions corresponding to the following amino acid substitutions: E835G and E880G, with reference to amino acid position numbering of LbCas12a ND20006, preferably wherein the engineered variant Cas12a endonuclease exhibits target nickase activity (or a preference for cleaving one strand over the other of a dsDNA).

88. An engineered variant Cas12a endonuclease comprising a polypeptide sequence comprising amino acid substitutions corresponding to the following amino acid substitutions: S929G, K932G, and N933G, with reference to amino acid position numbering of LbCas12a ND20006, preferably wherein the engineered variant Cas12a endonuclease exhibits target nickase activity (or a preference for cleaving one strand over the other of a dsDNA).

89. An engineered variant Cas12a endonuclease comprising a polypeptide sequence comprising amino acid substitutions corresponding to the following amino acid substitutions: S929G, K932G, N933G, and V936G, with reference to amino acid position numbering of LbCas12a ND20006, preferably wherein the engineered variant Cas12a endonuclease exhibits target nickase activity (or a preference for cleaving one strand over the other of a dsDNA).

90. An engineered variant Cas12a endonuclease comprising a polypeptide sequence comprising amino acid substitutions corresponding to the following amino acid substitutions: S929G, K932G, N933G, and V936F, with reference to amino acid position numbering of LbCas12a ND20006, preferably wherein the engineered variant Cas12a endonuclease exhibits target nickase activity (or a preference for cleaving one strand over the other of a dsDNA).

91. An engineered variant Cas12a endonuclease comprising a polypeptide sequence comprising amino acid substitutions corresponding to the following amino acid substitutions: S929G, V936G, F983G, and M986G, with reference to amino acid position numbering of LbCas12a ND20006, preferably wherein the engineered variant Cas12a endonuclease exhibits target nickase activity (or a preference for cleaving one strand over the other of a dsDNA).

92. An engineered variant Cas12a endonuclease comprising a polypeptide sequence comprising amino acid substitutions corresponding to the following amino acid substitutions: G930A and F931L, with reference to amino acid position numbering of LbCas12a ND20006, preferably wherein the engineered variant Cas12a endonuclease exhibits target nickase activity (or a preference for cleaving one strand over the other of a dsDNA) and low indiscriminate single strand DNase activity.

93. An engineered variant Cas12a endonuclease comprising a polypeptide sequence comprising amino acid substitutions corresponding to the following amino acid substitutions: G930A, F931L, and S934Q with reference to amino acid position numbering of LbCas12a, preferably wherein the engineered variant Cas12a endonuclease exhibits target nickase activity (or a preference for cleaving one strand over the other of a dsDNA).

94. An engineered variant Cas12a endonuclease comprising a polypeptide sequence comprising amino acid substitutions corresponding to the following amino acid substitutions: K932G and N933G, with reference to amino acid position numbering of LbCas12a ND20006, preferably wherein the engineered variant Cas12a endonuclease exhibits target nickase activity (or a preference for cleaving one strand over the other of a dsDNA).

95. An engineered variant Cas12a endonuclease comprising a polypeptide sequence comprising amino acid substitutions corresponding to the following amino acid substitutions: K932G, N933G, and V936F, with reference to amino acid position numbering of LbCas12a ND20006, preferably wherein the engineered variant Cas12a endonuclease exhibits target nickase activity (or a preference for cleaving one strand over the other of a dsDNA).

96. An engineered variant Cas12a endonuclease comprising a polypeptide sequence comprising amino acid substitutions corresponding to the following amino acid substitutions: V936G, F983G, and M986G, with reference to amino acid position numbering of LbCas12a ND20006, preferably wherein the engineered variant Cas12a endonuclease exhibits target nickase activity (or a preference for cleaving one strand over the other of a dsDNA).

97. An engineered variant Cas12a endonuclease comprising a polypeptide sequence comprising amino acid substitutions corresponding to the following amino acid substitutions: F983G and M986G, with reference to amino acid position numbering of LbCas12a ND20006, preferably wherein the engineered variant Cas12a endonuclease exhibits target nickase activity (or a preference for cleaving one strand over the other of a dsDNA) and low indiscriminate single strand DNase activity.

98. An engineered variant Cas12a endonuclease comprising a polypeptide sequence comprising a Lid-hub domain, wherein the polypeptide comprises a mutation at an amino acid position in the Lid-hub domain or in the vicinity of the Lid-Hub domain.

99. The engineered variant Cas12a endonuclease of paragraph 98, wherein the mutation is a substitution at a position corresponding to the following amino acid positions: R833, E835, E880, R836, S929, G930, F931, K932, N933, S934, R935, V936, K937, V938, K940, Q941, Y943, Q944, E947, F983, or M986, with reference to amino acid position numbering of LbCas12a ND20006.

100. The engineered variant Cas12a endonuclease of paragraph 98 or 99, wherein the engineered variant Cas12a endonuclease exhibits hyperactivity.

101. The engineered variant Cas12a endonuclease of paragraph 100, wherein the mutation is a substitution corresponding to any one of the following amino acid substitutions: K932I, K932L, K932V, N933L, or V936M.

102. The engineered variant Cas12a endonuclease of paragraph 98 or 99, wherein the engineered variant Cas12a endonuclease exhibits low indiscriminate single strand deoxyribonuclease (DNase) activity.

103. The engineered variant Cas12a endonuclease of paragraph 102, wherein the mutation is a substitution corresponding to any one of the following amino acid substitutions: S929L, K932F, K932R, K932T, K932Y, K932W, N933E, N933V, S934Q, V936E, V936K, K937Y, Q944D, Q944G, Q944M, F983L, M986F, or M986S.

104. The engineered variant Cas12a endonuclease of paragraph 98 or 99, wherein the engineered variant Cas12a endonuclease exhibits target nickase activity (or a preference for cleaving one strand over the other of a dsDNA).

105. The engineered variant Cas12a endonuclease of paragraph 104, wherein the mutation is a substitution corresponding to any one of the following amino acid substitutions: R833K, R833L, R833M, E835A, E835D, E835G, R836A, R836G, F931H, F931L, K932E, K932G, K932H, K932M, K932N, K932Q, K932S, R935F, R935G, R935H, R935I, R935K, R935L, R935M, R935N, R935S, R935T, R935W, V936G, V938E, K940G, Q941H, Q941K, Q941R, Q941Y, Y943F, Y943T, Q944E, Q944K, or M986G.

106. The engineered variant Cas12a endonuclease of any one of the preceding paragraphs comprising an amino acid sequence having at least 85%, at least 90%, or least 95%, but less than 100% identity with the amino acid sequence of a wild-type Cas12a endonuclease selected from Acidaminococcus sp., Lachnospiraceae sp., and Francisella sp.

107. The engineered variant Cas12a endonuclease of any one of the preceding paragraphs further comprising no more than 1, 2, 3, 4, 5, 6, 7, 8, 9, or 10 additional amino acid substitutions relative to a wild-type reference Cas12a endonuclease.

108. An engineered variant Cas12a endonuclease comprising a polypeptide sequence comprising the amino acid sequence of any one of SEQ ID NOs: 48-119.

109. A polynucleotide encoding the variant Cas12a endonuclease of any one of the preceding paragraphs.

110. A cell comprising (a) the variant Cas12a endonuclease of any one of the preceding paragraphs or the polynucleotide of paragraph 109 and (b) a guide RNA or a polynucleotide encoding a guide RNA.

111. A method comprising introducing into a cell (a) the variant Cas12a endonuclease of any one of the preceding paragraphs or the polynucleotide of paragraph 109 and optionally (b) a guide RNA or a polynucleotide encoding a guide RNA.

112. Use of the variant Cas12a endonuclease of any one of the preceding paragraphs for cleaving a nucleic acid.

113. A method for introducing a double strand break in a target nucleic acid, comprising introducing into a cell comprising a target nucleic acid (a) the variant Cas12a endonuclease of any one of paragraphs 5-11 and (b) a guide RNA; and incubating the cell to produce a double strand break in the target nucleic acid.

114. A method for introducing a double strand break in a target nucleic acid, comprising introducing into a cell comprising a target nucleic acid (a) the variant Cas12a endonuclease of any one of paragraphs 12-38, 84, or 85 and (b) a guide RNA; and incubating the cell to produce a double strand break in the target nucleic acid.

115. The method of paragraph 114, wherein off-target single strand nucleic acid cleavage in the cell is reduced relative to off-target single strand nucleic acid cleavage in a control cell comprising a wild-type Cas12a endonuclease and a guide RNA.

116. A method for introducing a single strand break in a target nucleic acid, comprising introducing into a cell comprising a target nucleic acid (a) the variant Cas12a endonuclease of any one of paragraphs 39-83 or 86-97 and (b) a guide RNA; and incubating the cell to produce a single strand break in the target nucleic acid.

### Examples

### Example 1. Cas12a Endonuclease Variants

Purified Cas12a variants of Table 3 in complex with crRNA were tested against (1) dsDNA containing a quencher on one site of the cleavage site and a fluorophore on the other site of the cleavage site, on separate DNA strands, and (2) a combination dsDNA containing no quencher or fluorophore but together with a ssDNA containing both a quencher and fluorophore. The retained activity on dsDNA was confirmed by (1) where the quencher and fluorophore were separated upon cleavage, and the emission signal increases over time.

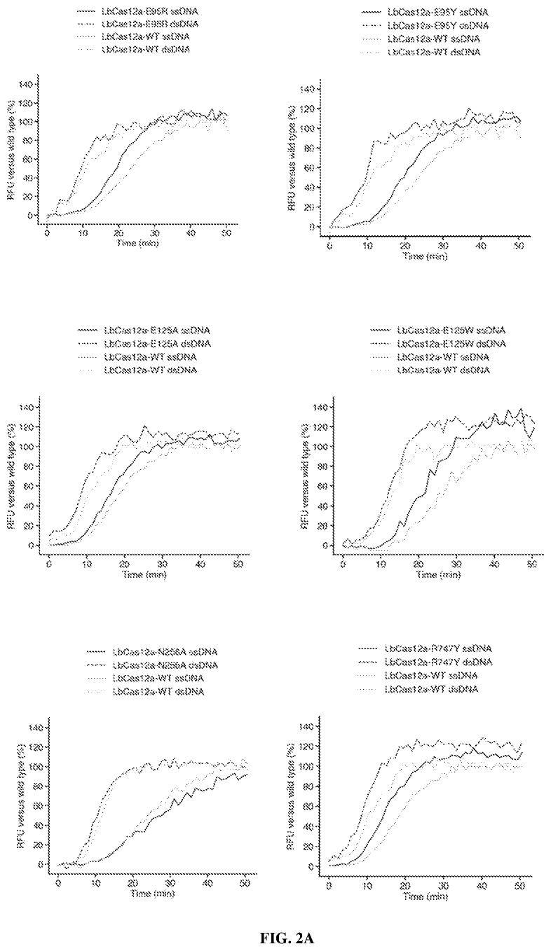

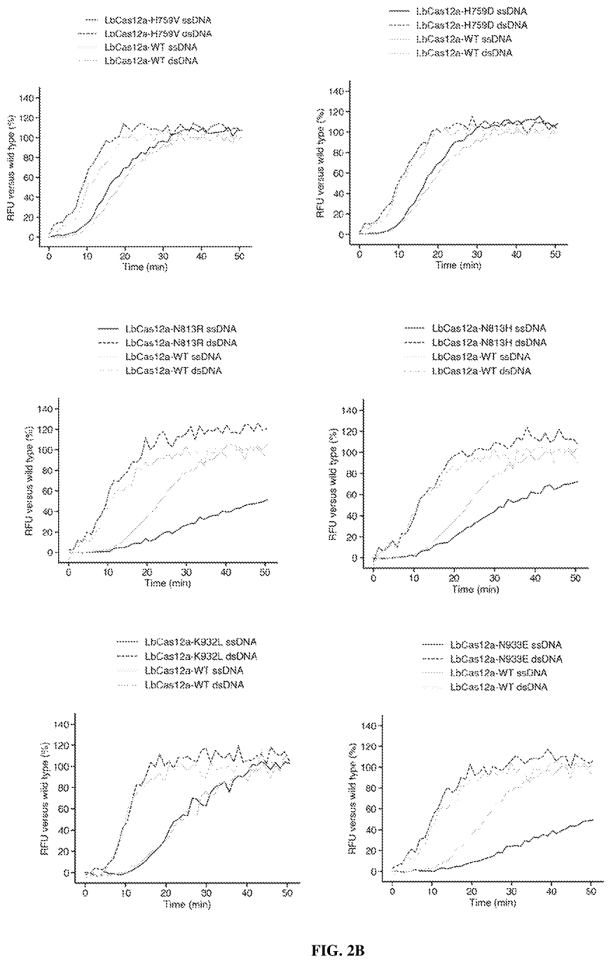

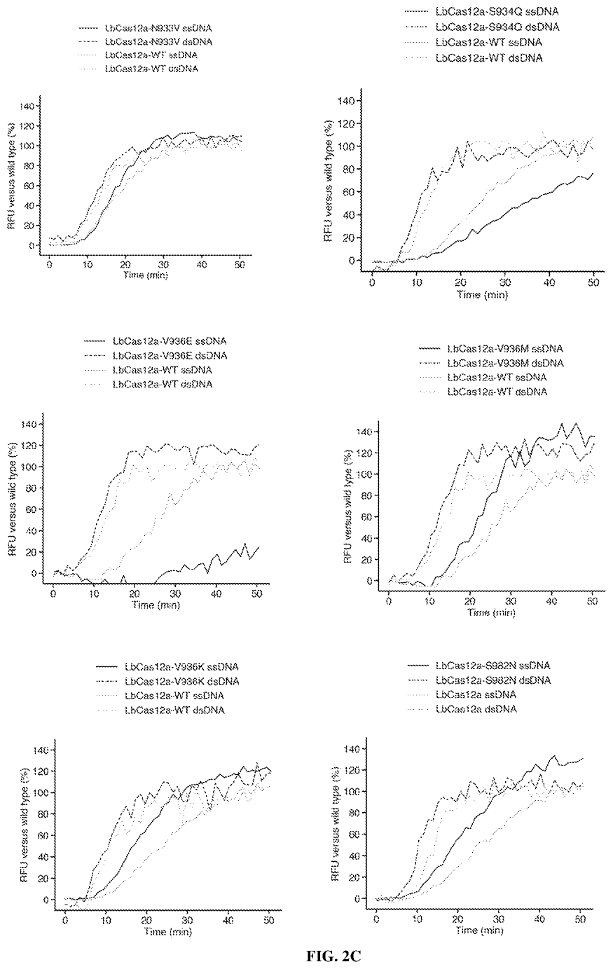

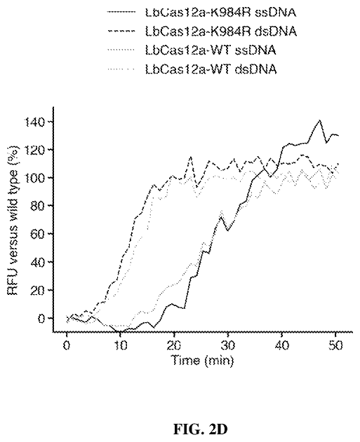

Hyperactive Cas12a endonuclease variants were identified as having a higher reaction speed or by initiating the reaction faster than the naturally-occurring (i.e., wildtype (WT)) Cas12a. Within the subset of Cas12a endonuclease variants that had hyperactivity, several variants were also identified as having low ssDNase activity. Low ssDNase activity is identified by a lowered ability to cleave the ssDNA and thereby separate the quencher and fluorophore when activated by specific dsDNA, thus by lacking an emission signal in comparable magnitude to that of the wildtype Cas12a in (2). See FIGS. 2A-2D and Table 6.

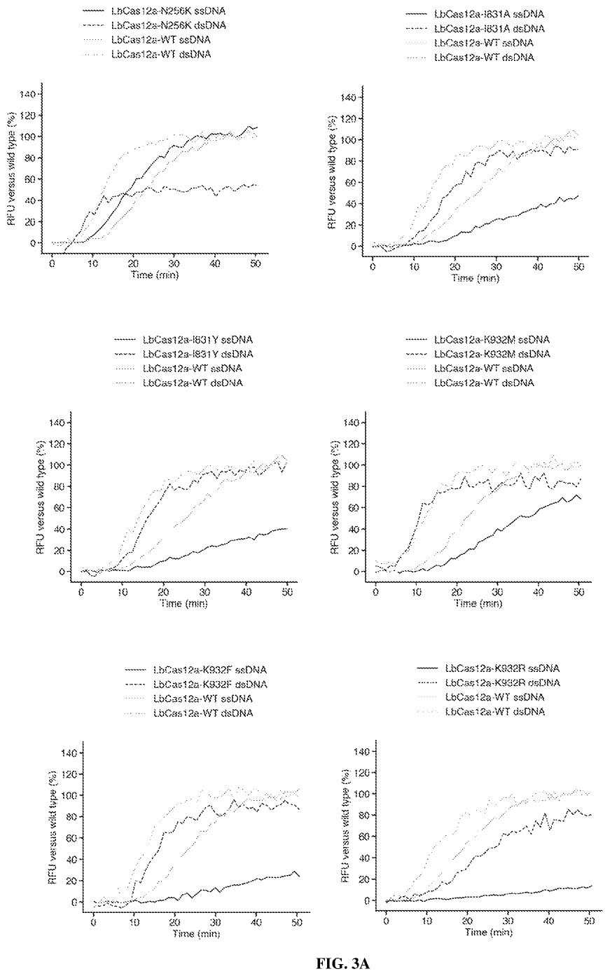

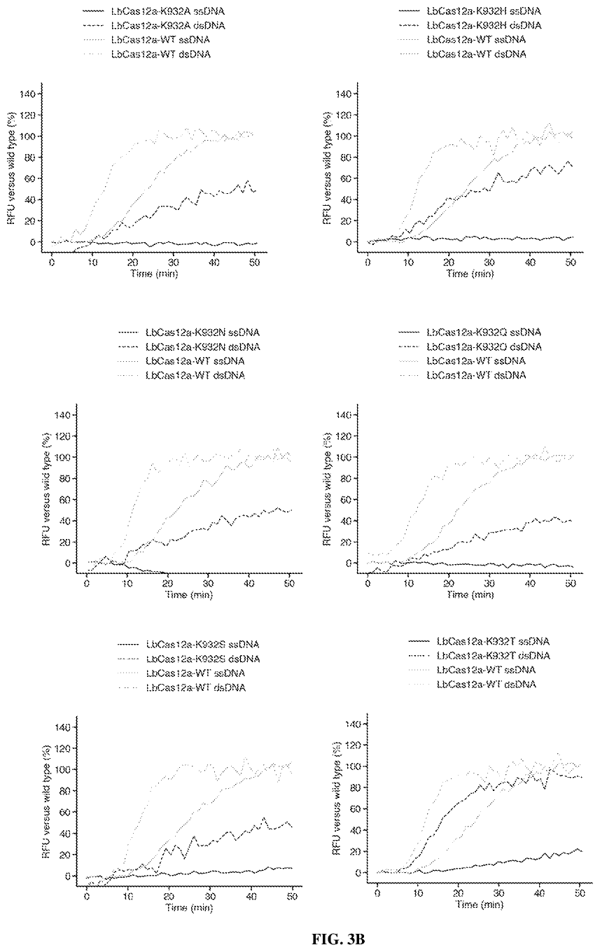

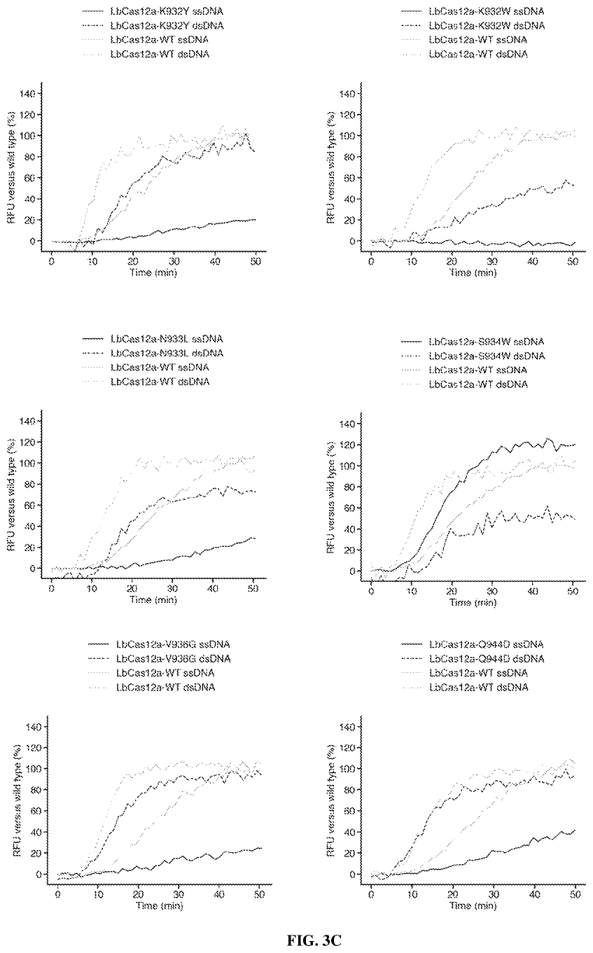

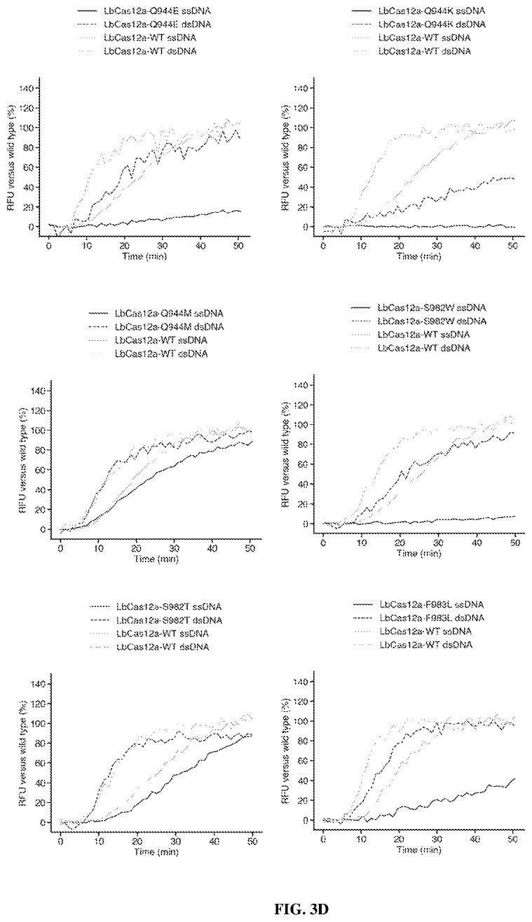

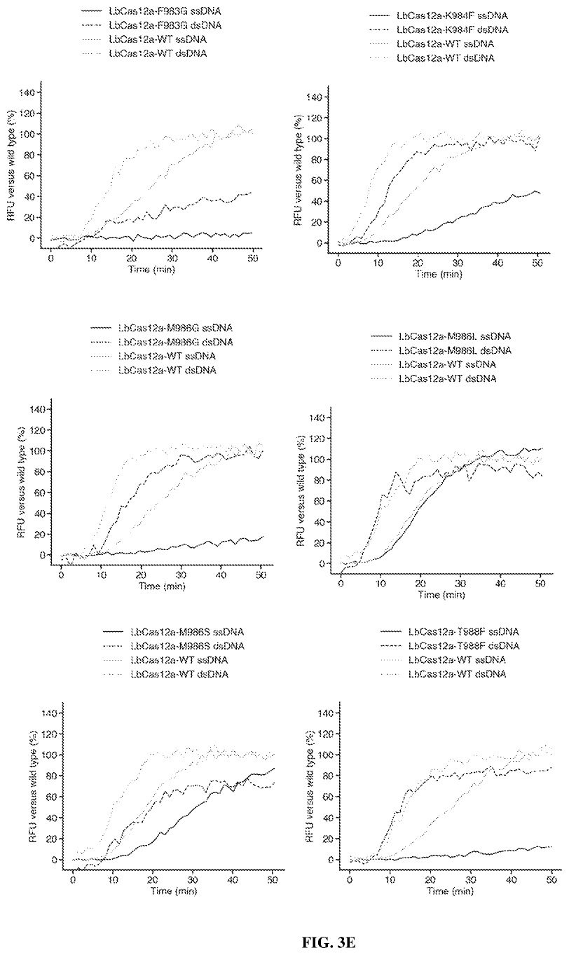

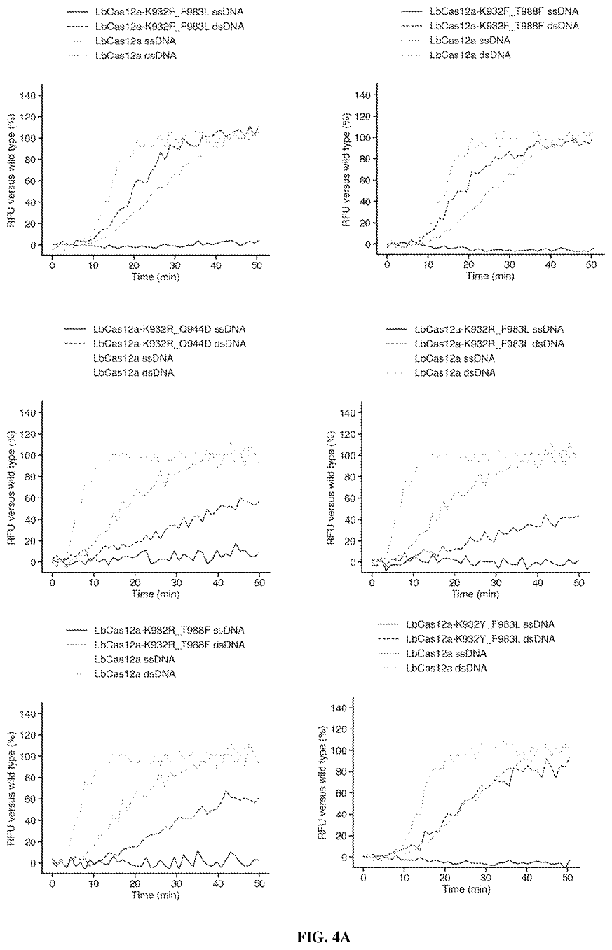

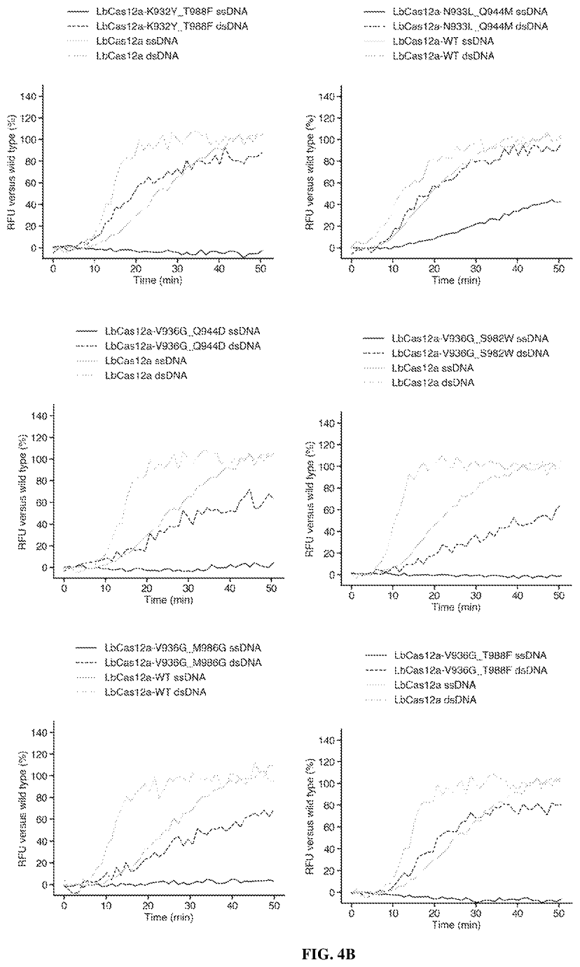

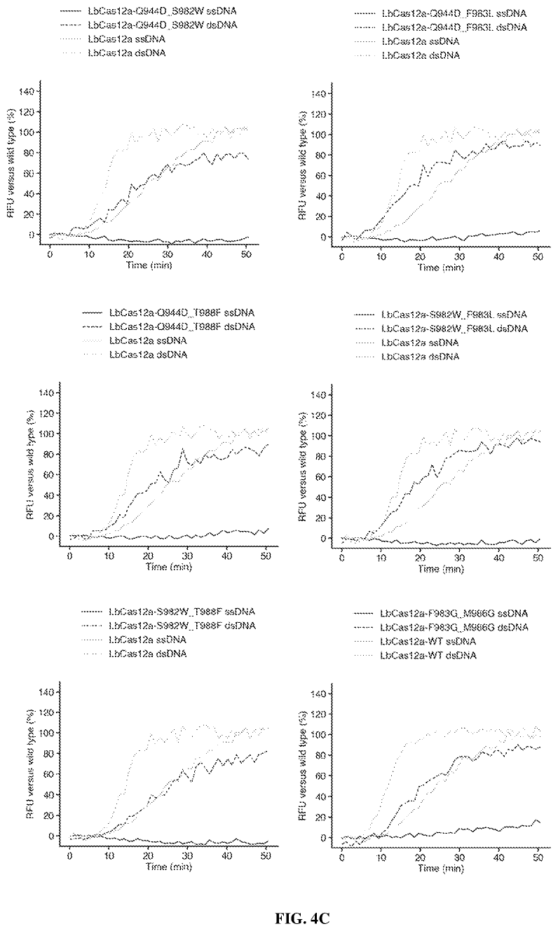

Hypoactive Cas12a endonuclease variants were identified as having a lower reaction speed or by initiating the reaction slower than the wildtype (WT) Cas12a. Within the subset of Cas12a endonuclease variants that had hypoactivity, several variants were also identified as having low ssDNase activity. Low ssDNase activity is identified by a lowered ability to cleave the ssDNA and thereby separate the quencher and fluorophore when activated by specific dsDNA, thus by lacking an emission signal in comparable magnitude to that of the wildtype Cas12a in (2). See FIGS. 3A-3E, 4A-4C, and Table 6.

Three Cas12a endonuclease variants were identified as having similar activity profile as wildtype Cas12a while demonstrating low ssDNase activity. See FIG. 5 and Table 6.

TABLE 6
Variant Cas12a Endonucleases of Example 1

| | | Reduced/Lowered |
|---|---|---|
| Variant | dsDNA Phenotype | Activity towards ssDNA? |
| E95R | Hyperactive | |
| E95Y | Hyperactive | |
| E125A | Hyperactive | |
| E125W | Hyperactive | |
| N256A | Hyperactive | |
| N256K | Hypoactive | |
| R747Y | Hyperactive | |
| H759V | Hyperactive | |
| H759D | Hyperactive | |
| N813R | Hyperactive | Yes |
| N813W | Wildtype | Yes |
| N813H | Hyperactive | Yes |
| I831A | Hypoactive | Yes |
| I831Y | Hypoactive | Yes |
| K932L | Hyperactive | |
| K932I | Wildtype | |
| K932V | Wildtype | |
| K932M | Hypoactive | Yes |
| K932F | Hypoactive | Yes |
| K932R | Hypoactive | Yes |
| K932A | Hypoactive | Yes |
| K932H | Hypoactive | Yes |
| K932N | Hypoactive | Yes |
| K932Q | Hypoactive | Yes |
| K932S | Hypoactive | Yes |
| K932T | Hypoactive | Yes |
| K932Y | Hypoactive | Yes |
| K932W | Hypoactive | Yes |
| N933E | Hyperactive | Yes |
| N933V | Hyperactive | |
| N933L | Hypoactive | Yes |
| S934Q | Hyperactive | Yes |
| S934K | Wildtype | Yes |
| S934W | Hypoactive | |
| V936E | Hyperactive | Yes |
| V936M | Hyperactive | |
| V936K | Hyperactive | |
| V936G | Hypoactive | Yes |
| Q944D | Hypoactive | Yes |
| Q944E | Hypoactive | Yes |
| Q944K | Hypoactive | Yes |
| Q944M | Hypoactive | |
| S982W | Hypoactive | Yes |
| S982T | Hypoactive | |
| S982A | Wildtype | |
| S982N | Hyperactive | |
| F983L | Hypoactive | Yes |
| F983G | Hypoactive | Yes |
| K984R | Hyperactive | |
| K984F | Hypoactive | Yes |
| M986G | Hypoactive | Yes |
| M986F | Wildtype | Yes |
| M986L | Hypoactive | |
| M986S | Hypoactive | |
| T988F | Hypoactive | Yes |
| K932F, F983L | Hypoactive | Yes |
| K932F, T988F | Hypoactive | Yes |
| K932R, Q944D | Hypoactive | Yes |
| K932R, F983L | Hypoactive | Yes |
| K932R, T988F | Hypoactive | Yes |
| K932Y, F983L | Hypoactive | Yes |
| K932Y, T988F | Hypoactive | Yes |
| N933L, Q944M | Hypoactive | Yes |
| V936G, Q944D | Hypoactive | Yes |
| V936G, S982W | Hypoactive | Yes |
| V936G, M986G | Hypoactive | Yes |
| V936G, T988F | Hypoactive | Yes |
| Q944D, S982W | Hypoactive | Yes |
| Q944D, F983L | Hypoactive | Yes |
| Q944D, T988F | Hypoactive | Yes |
| S982W, F983L | Hypoactive | Yes |
| S982W, T988F | Hypoactive | Yes |
| F983G, M986G | Hypoactive | Yes |

### Example 2. Enzyme-to-Enzyme Linker Assessment

The activity of different C-to-T base editors (fusion proteins comprising a Cas12a variant and cytidine deaminase) containing linkers of varying sizes and sequences between different domains was tested in U2OS cells. U2OS cells were transfected using a plasmid expressing the base editor together with a plasmid expressing a gRNA (AGCCTCAC₈C₉C₁₀CTC₁₃TAGCCCT (SEQ ID NO: 186)). Transfected cells were sorted after 3 days and re-cultured for another 3 days after which the cells were lysed and genomic DNA was extracted. Genomic region around the gRNA was PCR amplified and editing was analyzed by next generation sequencing. In these experiments, LbCas12a was either catalytic inactive (inactive Cas12a comprising a D832A mutation) LbCas12a or a LbCas12a variant (TBN04).

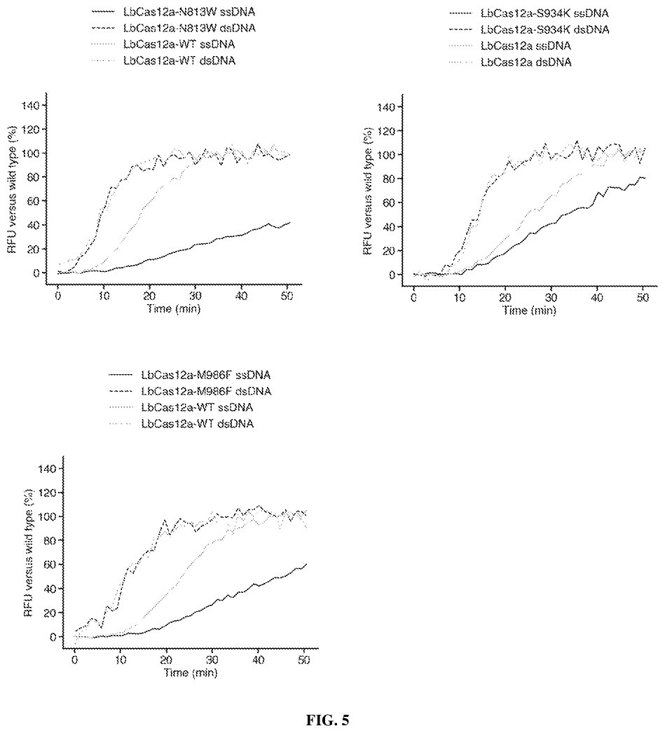

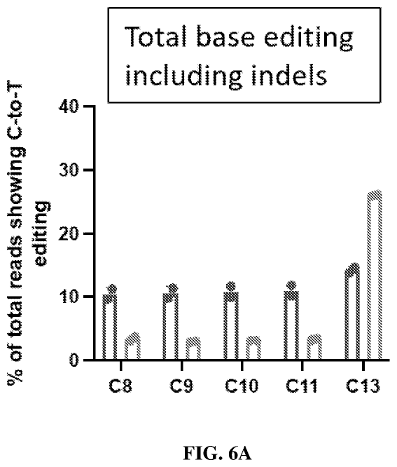

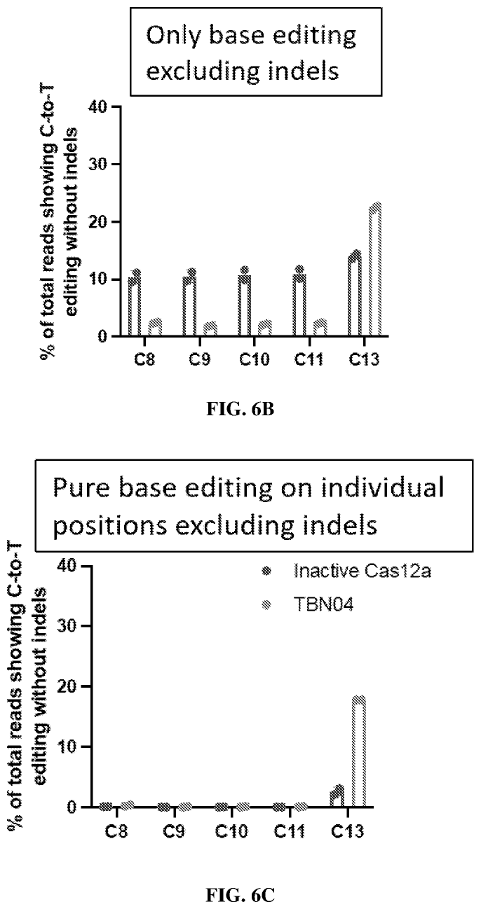

LbBEv2 (comprising Linker4 between rAPOBEC1 and LbCas12a and Linker5 between LbCas12a and UGI) showed efficient base editing (FIG. 6A-6C). FIG. 6A: % of total reads showing base editing at individual positions. This includes the reads that show base editing without indels plus the reads that harbor base editing as well as the indels. FIG. 6B: % of total reads showing only base editing at individual positions. This only includes the reads that show base editing but no indels. FIG. 6C: % of total reads showing base editing at only one specific position. This excludes all the reads with base editing at multiple positions as well as the reads harboring indels. Inactive Cas12a was used as the control.

The improvement in the base editing efficiency with LbBEv2 was more significant when using a base editor containing LbCas12a variant (TBN04). These data suggest that inclusion of a longer linker between the Cas12a variant and the base editing enzyme in the fusion proteins described herein is useful in improving base editing efficiency (likely by decreasing undesired interactions, e.g., steric interactions, between the Cas12a variant and the base editing enzyme).

Structure of LbBEv2 is as follows:

Linker3-rAPOBEC1-Linker4-LbCas12a-NP NLS-Linker5-UGI-Linker2-SV40 NLS; wherein Linker3 sequence is the amino acid sequence of GS, Linker4 sequence is the amino acid sequence of SGGSSGGSSGSETPGTSESATPESSGGSSGGS (SEQ ID NO: 204) and Linker5 sequence is the amino acid sequence of GSSGGSGGSGGS (SEQ ID NO: 207).

### Example 3. Nuclear Localization Signal Assessment

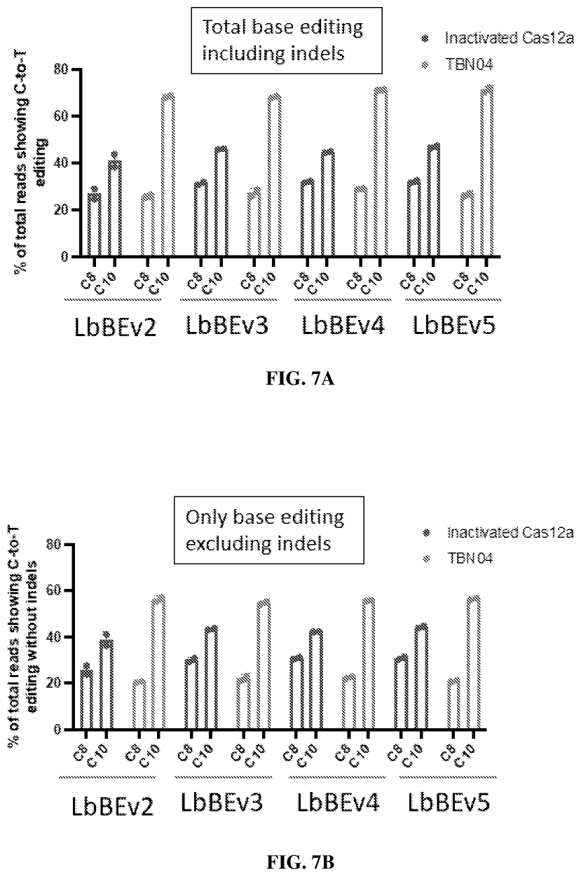

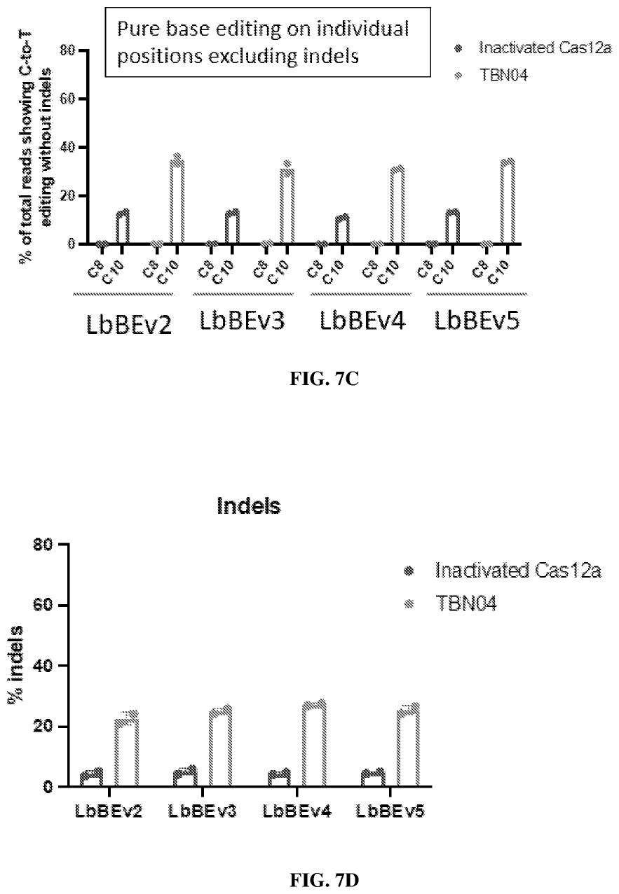

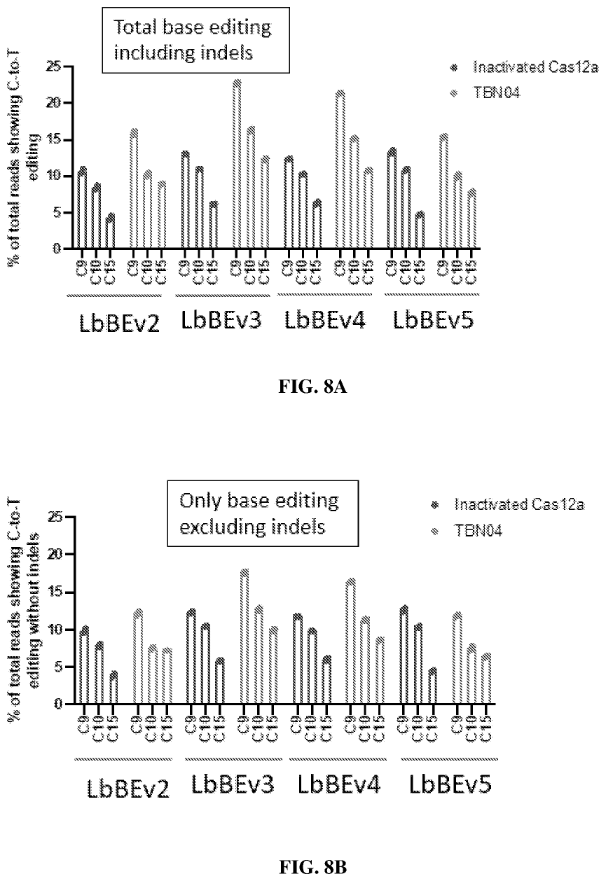

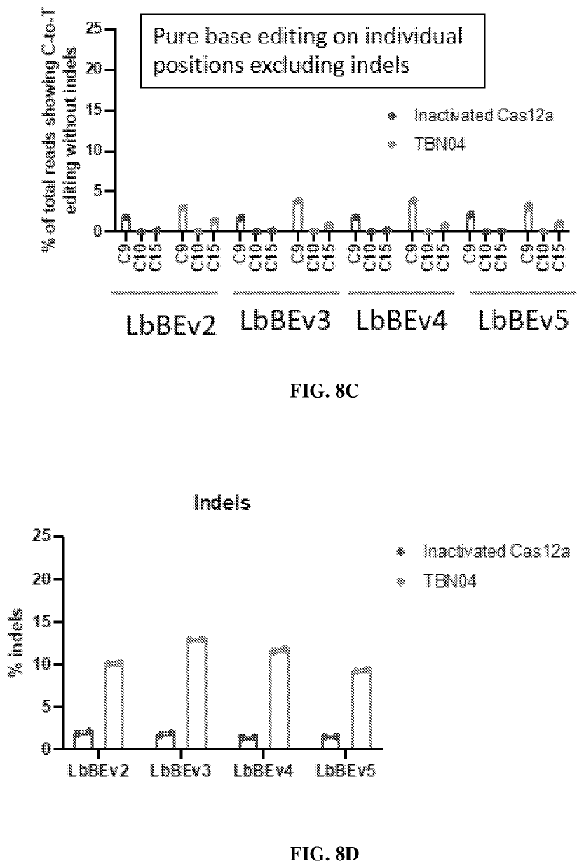

The activity of C-to-T base editors (fusion proteins comprising a LbCas12a Cas12a variant (TBN04) and cytidine deaminase) with different NLSs at the N-terminus (LbVEv3, LbBEv4 and LbBEv5) was tested in U2OS cells and compared with LbBEv2, which lacks an NLS at the N-terminus. U2OS cells were transfected using a plasmid expressing the base editor together with a plasmid expressing the gRNA. Transfected cells were sorted after 3 days and re-cultured for another 3 days after which the cells were lysed, and genomic DNA was extracted. Genomic region around the gRNA was PCR amplified and editing was analyzed by next generation sequencing. Two different gRNAs were tested-TTCTCCCC₈TC₁₀TGCTGGATAC (SEQ ID NO:187) (FIGS. 7A-7D) and CTGATGGTC₉C₁₀ATGTC₁₅TGTTA (SEQ ID NO: 191) (FIGS. 8A-8D).

FIG. 7A: % of total reads showing base editing at individual positions. This includes the reads that show base editing without indels plus the reads that harbor base editing as well as the indels. FIG. 7B: % of total reads showing only base editing at individual positions. This only includes the reads that show base editing but no indels. FIG. 7C: % of total reads showing base editing at only one specific position. This excludes all the reads with base editing at multiple positions as well as the reads harboring indels. FIG. 7D: % of total reads showing indels. Inactive Cas12a was used as the control.

In these experiments, LbCas12a can be catalytic inactive (inactive LbCas12a comprising a D832A mutation) or a variant LbCas12a (TBN04). Relative to LbBEv2 (i.e., lacking an NLS at the N-terminus), C-to-T base editors with N-terminus NLS (LbBEv3, LbBEv4 and LbBEv5) showed improved base editing efficiency in a gRNA-specific manner (FIG. 8A-8D). FIG. 8A: % of total reads showing base editing at individual positions. This includes the reads that show base editing without indels plus the reads that harbor base editing as well as the indels. FIG. 8B: % of total reads showing only base editing at individual positions. This only includes the reads that show base editing but no indels. FIG. 8C: % of total reads showing base editing at only one specific position. This excludes all the reads with base editing at multiple positions as well as the reads harboring indels. FIG. 8D: % of total reads showing indels. Inactive Cas12a was used as the control.

These data demonstrate that inclusion of an NLS located at or near the N-terminal of the fusion proteins described herein is useful in improving base editing efficiency (likely by increasing the amount of fusion protein located in the nucleus).

Structure of LbBEv3 is as follows:

SV40 NLS-Linker3-rAPOBEC1-Linker4-LbCas12a-NP NLS-Linker5-UGI-Linker2-SV40 NLS

Structure of LbBEv4 is as follows:

NP NLS-Linker3-rAPOBEC1-Linker4-LbCas12a-NP NLS-Linker5-UGI-Linker2-SV40 NLS

Structure of LbBEv5 is as follows:

Linker3-BP NLS-Linker3-rAPOBEC1-Linker4-LbCas12a-NP NLS-Linker5-UGI-Linker2-SV40 NLS

### Example 4. Efficiency and Specificity of C-to-T Base Editors

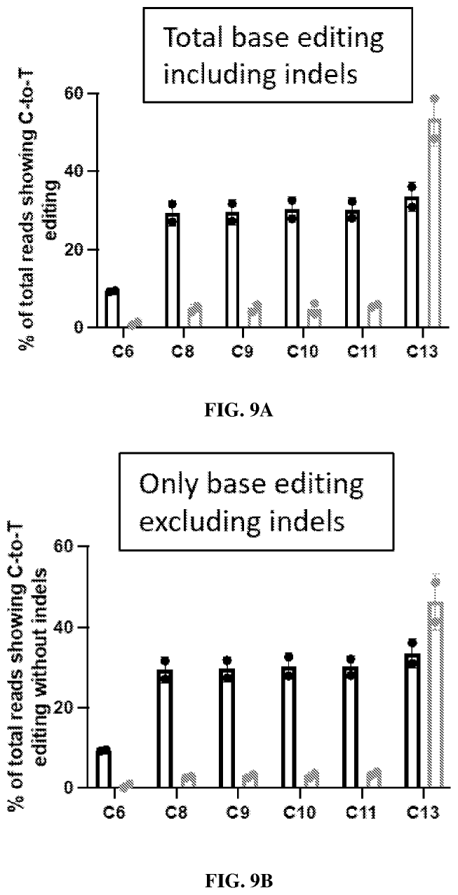

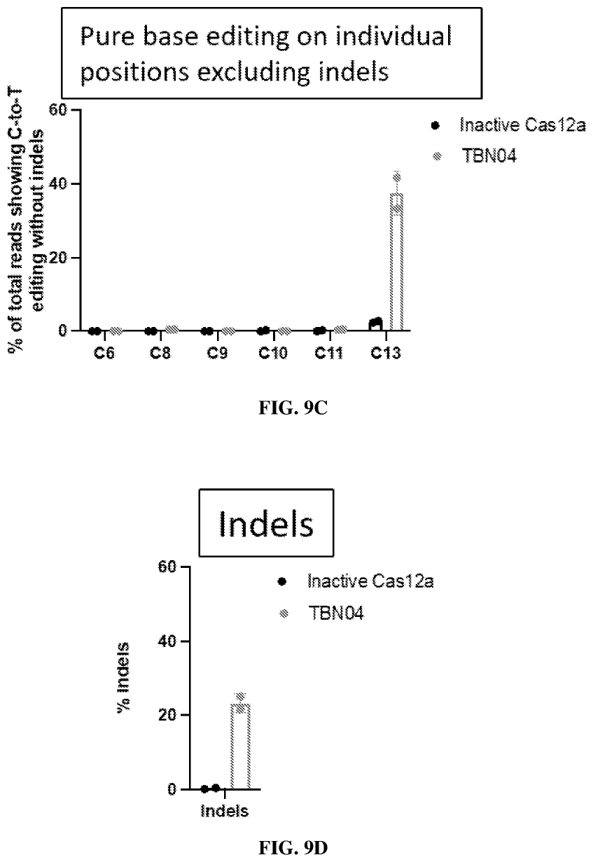

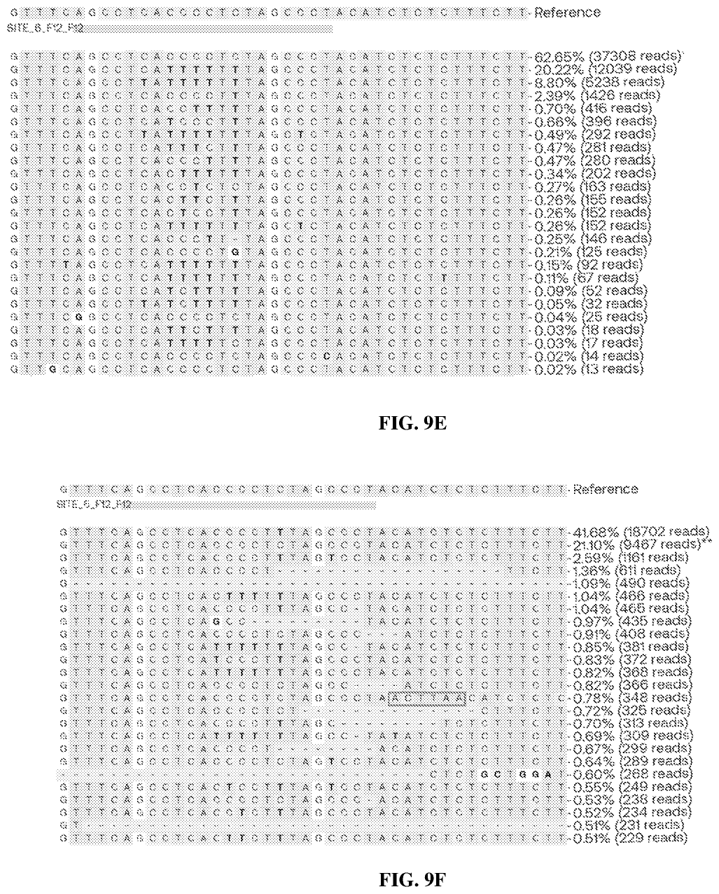

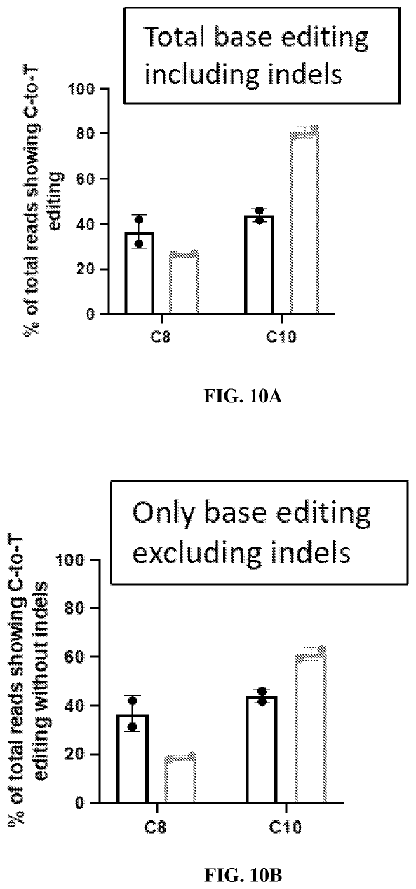

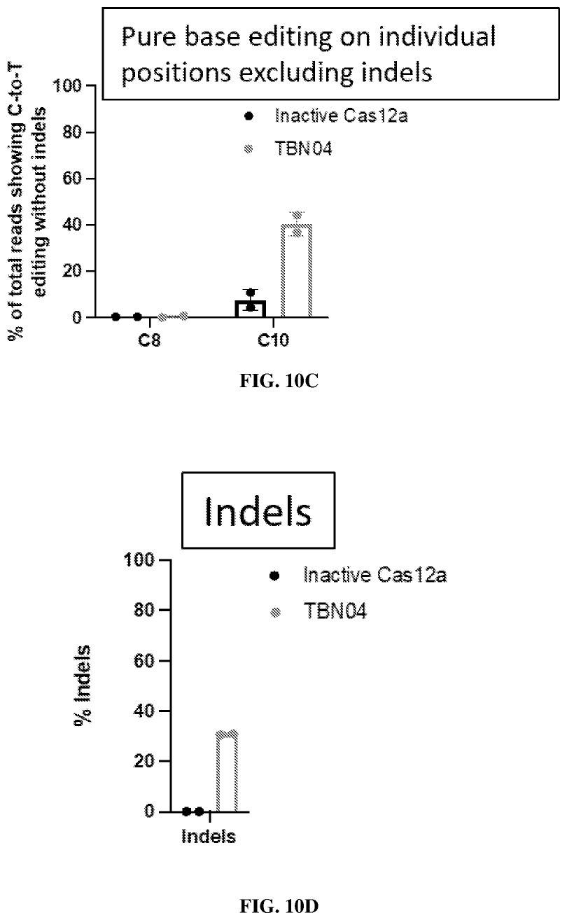

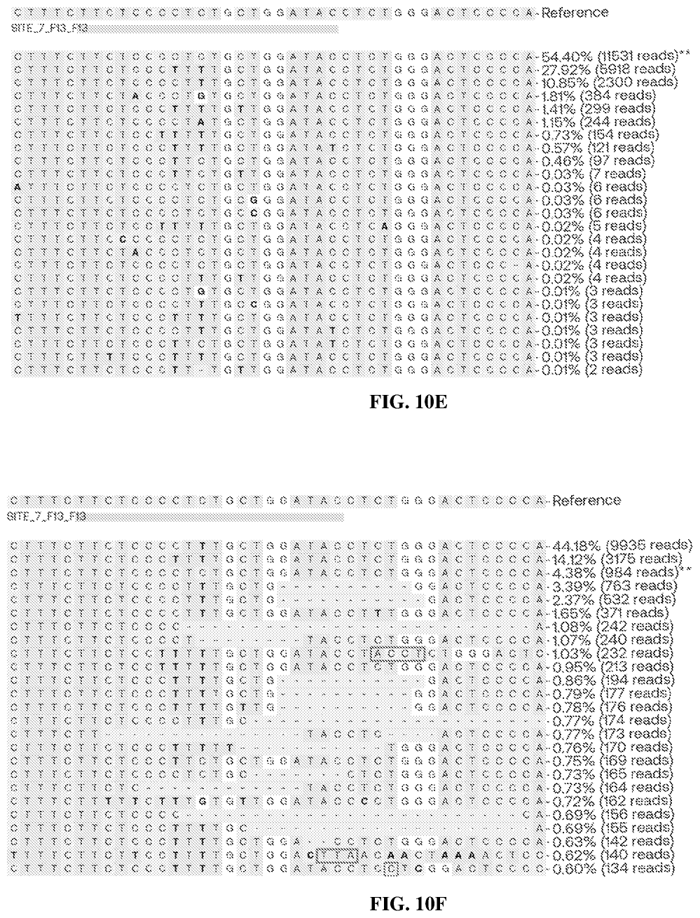

The base editing efficiency and specificity of C-to-T base editors (fusion proteins comprising a Cas12a variant and cytidine deaminase) was determined when comprising either inactive LbCas12a (Cas12a comprising a D832A mutation) or TBN04 (LbCas12a variant). U2OS cells were transfected using a plasmid expressing the base editor together with a plasmid expressing the gRNA. Transfected cells were sorted after 3 days and re-cultured for another 3 days after which the cells were lysed and genomic DNA was extracted. Genomic region around the gRNA was PCR amplified and editing was analyzed by next generation sequencing. Two different gRNAs were tested-AGCCTC₆AC₈C₉C₁₀C₁₁TC₁₃TAGCCCT (SEQ ID NO: 192) (FIG. 9A-9F) and TTCTCCCC₉TC₁₀TGCTGGATAC (SEQ ID NO: 187) (FIG. 10A-10F).

C-to-T base editor containing the TBN04 shows overall higher base editing efficiency than base editors containing inactive Cas12a. Moreover, TBN04 base editor shows remarkable base selectivity where the majority of edited alleles has editing only at one specific position whereas C-to-T base editor containing inactive LbCas12a lacks base selectivity and edits multiple Cs in the editing window.

For example, for gRNA AGCCTC₆AC₈C₉C₁₀C₁₁TC₁₃TAGCCCT (SEQ ID NO: 192), base editor containing TBN04 shows editing primarily at C₁₃ whereas base editor with inactive LbCas12a edits all the Cs between position 6 and 13 with comparable efficiency (FIG. 9A-9F).

FIGS. 9A-9D provide graphs of data comparing percent (%) of total reads having a C-to-T nucleotide edit at genomic positions corresponding to positions C6, C8, C9, C10, C11, and C13 of the gRNA using the LbBEv5 base editor. FIG. 9A: % of total reads showing base editing at individual positions. This includes the reads that show base editing without indels plus the reads that harbor base editing as well as the indels. FIG. 9B: % of total reads showing only base editing at individual positions. This only includes the reads that show base editing but no indels. FIG. 9C: % of total reads showing base editing at only one specific position. This excludes all the reads with base editing at multiple positions as well as the reads harboring indels. FIG. 9D: % of total reads showing indels. FIGS. 9E-9F show allele frequency tables around the gRNA. FIG. 9E: frequency of alleles harboring different edits by LbBEv5 containing inactive LbCas12a and gRNA AGCCTC₆AC₈C₉C₁₀C₁₁TC₁₃TAGCCCT (SEQ ID NO: 192) in U2OS cells. FIG. 9F: frequency of alleles harboring different edits by LbBEv5 containing TBN04 (LbCas12a) and gRNA AGCCTC₆AC₈C₉C₁₀C₁₁TC₁₃TAGCCCT (SEQ ID NO: 192) in U2OS cells.

Likewise, for gRNA TTCTCCCC₈TC₁₀TGCTGGATAC (SEQ ID NO: 187), base editor containing TBN04 shows editing primarily at C₁₀ whereas base editor with inactive LbCas12a edits both C₈ and C₁₀ with comparable efficiency (FIG. 10A-10F).

FIGS. 10A-10D provide graphs of data comparing percent (%) of total reads having a C-to-T nucleotide edit at genomic positions corresponding to positions C8 and C10 of the gRNA using the LbBEv5 base editor. FIG. 10A: % of total reads showing base editing at individual positions. This includes the reads that show base editing without indels plus the reads that harbor base editing as well as the indels. FIG. 10B: % of total reads showing only base editing at individual positions. This only includes the reads that show base editing but no indels. FIG. 10C: % of total reads showing base editing at only one specific position. This excludes all the reads with base editing at multiple positions as well as the reads harboring indels. FIG. 10D: % of total reads showing indels. FIGS. 10E-10F show allele frequency tables around the gRNA. FIG. 10E: frequency of alleles harboring different edits by LbBEv5 containing inactive LbCas12a and gRNA TTCTCCCC₈TC₁₀TGCTGGATAC (SEQ ID NO: 187) in U2OS cells. FIG. 10F: frequency of alleles harboring different edits by LbBEv5 containing TBN04 (LbCas12a) and gRNA TTCTCCCC₈TC₁₀TGCTGGATAC (SEQ ID NO: 187) in U2OS cells.

### Example 5. Efficiency and Specificity of A-to-G Base Editors

The base editing efficiency and specificity of A-to-G base editor (fusion protein comprising a Cas12a variant and adenosine deaminase) containing inactive LbCas12a (Cas12a comprising a D832A mutation) or TBN04 (LbCas12a variant) was determined. U2OS cells were transfected using a plasmid expressing the base editor together with a plasmid expressing the gRNA. Transfected cells were sorted after 3 days and re-cultured for another 3 days after which the cells were lysed and genomic DNA was extracted. Genomic region around the gRNA was PCR amplified and editing was analyzed by next generation sequencing.

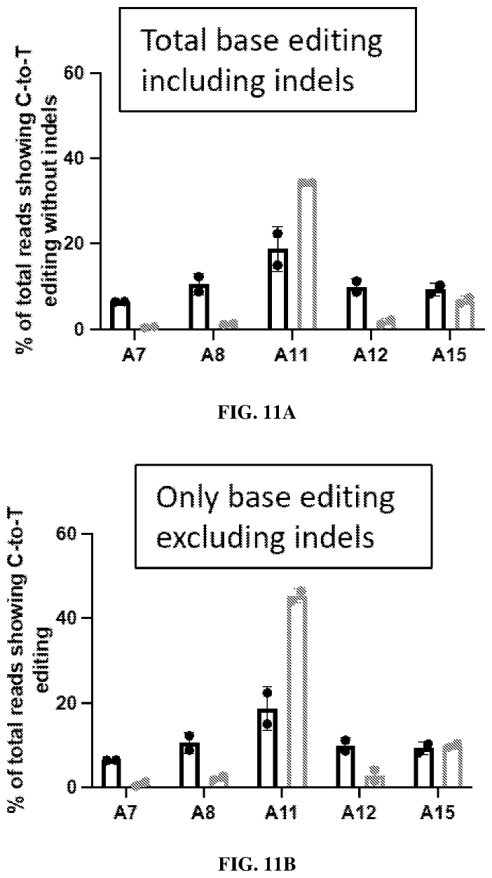

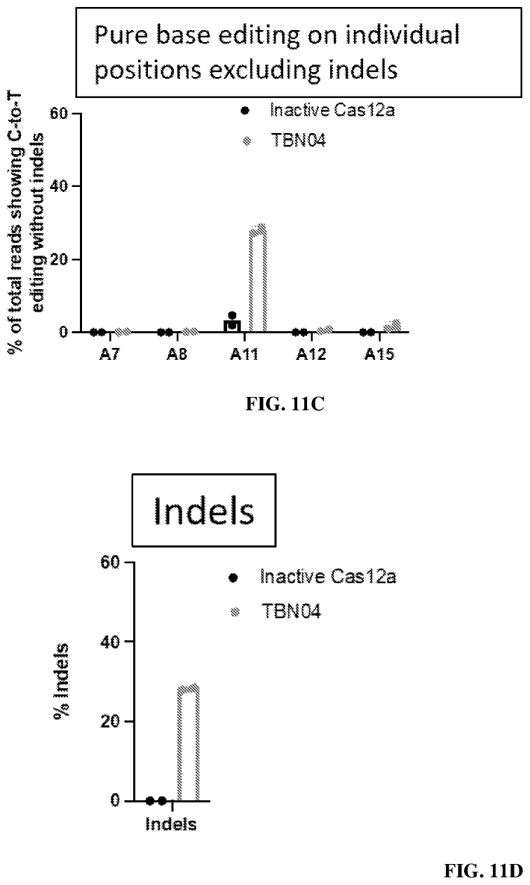

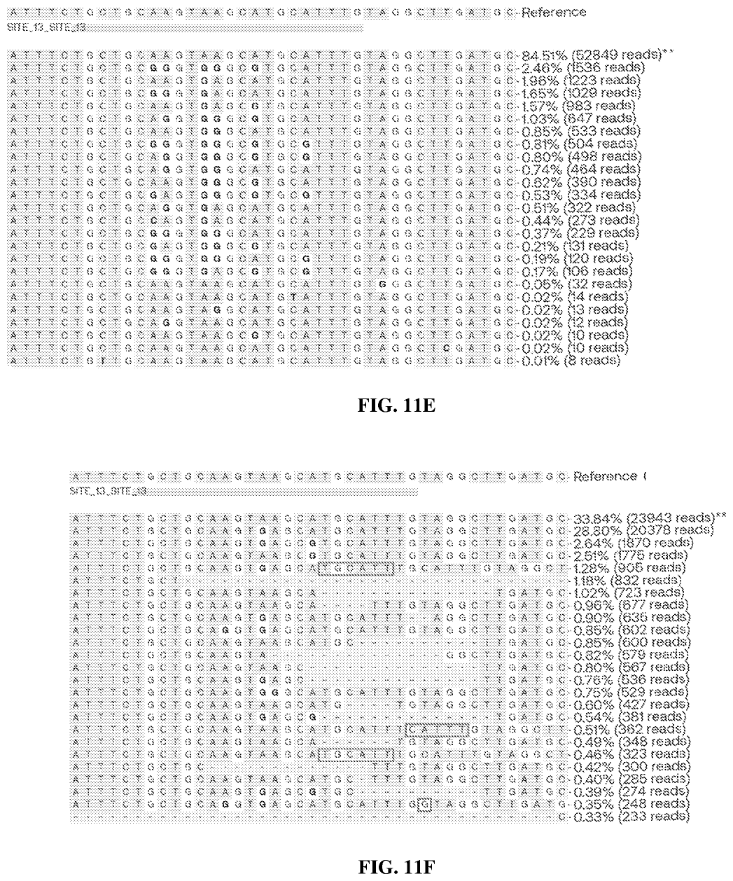

The A-to-G base editor containing the TBN04 shows overall higher base editing efficiency than base editors containing inactive Cas12a. Moreover, TBN04 base editor shows significant base selectivity where the majority of edited alleles has editing only at one specific position. This is in contrast to the A-to-G base editor containing inactive LbCas12a which lacked base selectivity and edited multiple Aₛ in the editing window. For example, for gRNA TGCTGCA₇A₈GTA₁₁A₁₂GCA₁₅TGCATTTG (SEQ ID NO: 188), base editor containing TBN04 shows editing primarily at A11 whereas base editor with inactive LbCas12a edits all the As between position 7 and 15 with comparable efficiency (FIG. 11A-11F).

FIGS. 11A-11D provide graphs of data comparing percent (%) of total reads having a C-to-T nucleotide edit at genomic positions corresponding to positions A7, A8, A11, A12, and A15 of the gRNA using the LbABE8e base editor. FIG. 11A: % of total reads showing A-to-G base editing at individual positions. This includes the reads that show base editing without indels plus the reads that harbor base editing as well as the indels. FIG. 11B: % of total reads showing only base editing at individual positions. This only includes the reads that show base editing but no indels. FIG. 11C: % of total reads showing base editing at only one specific position. This excludes all the reads with base editing at multiple positions as well as the reads harboring indels. FIG. 11D: % of total reads showing indels. FIGS. 11E-11F show allele frequency tables around the gRNA. FIG. 11E: frequency of alleles harboring different edits by LbABE8e containing inactive LbCas12a and gRNA TGCTGCA₇A₈GTA₁₁A₁₂GCA₁₅TGCATTTG (SEQ ID NO: 188) in U2OS cells. FIG. 11F: frequency of alleles harboring different edits by LbABE8e containing TBN04 (LbCas12a) and gRNA TGCTGCA₇A₈GTA₁₁A₁₂GCA₁₅TGCATTTG (SEQ ID NO: 188) in U2OS cells.

The structure of LbABE8e is as follows:

BP NLS-TadA-Linker4-LbCas12a-Linker2-BP NLS; wherein LbCas12a can be inactive LbCas12a or TBN04 (LbCas12a variant).

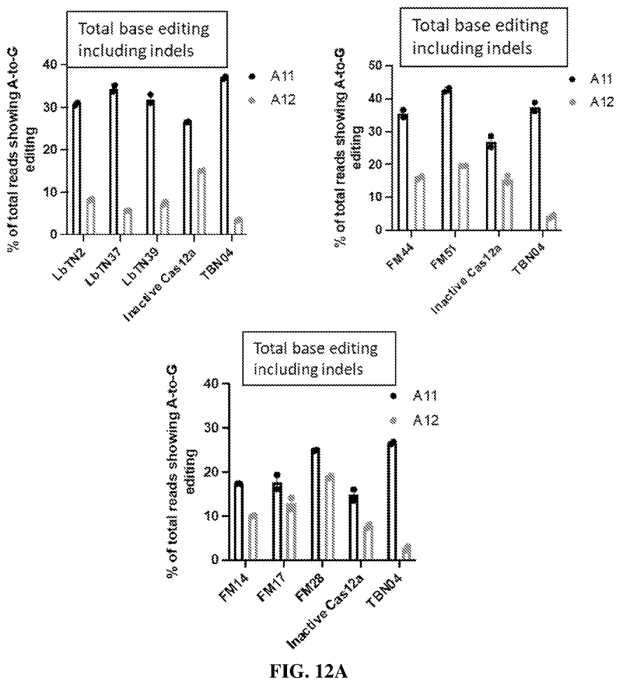

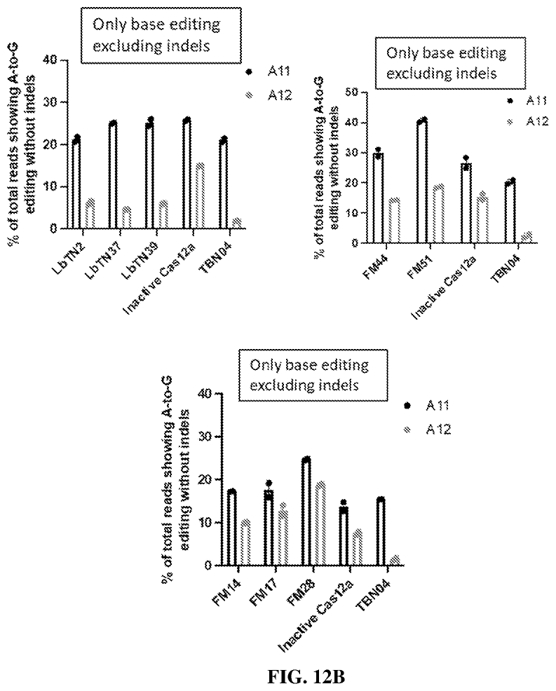

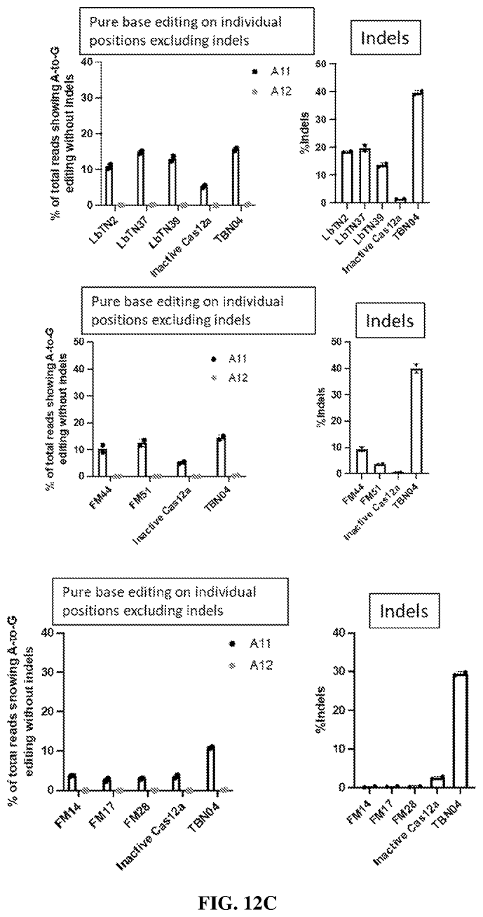

A selection of fusion proteins comprising a Cas12a variants as provided in Table 7 were assayed for their ability to produce indels and perform an A-to-G base conversion. The Cas12a variants were placed into the LbABE8e structural framework. The data in Table 7 and FIGS. 12A-12C demonstrate that the A-to-G base editors were selective for the A₁₁ in the gRNA TGCTGCA₇A₈GTA₁₁A₁₂GCA₁₅TGCATTTG (SEQ ID NO: 188). These data demonstrate that single and combinatorial mutations in Cas12a endonuclease provide higher base editing efficiency and higher base editing selectivity relative to inactive Cas12 and TBN04.

FIGS. 12A-12C provide graphs of data comparing percent (%) of total reads having a A-to-G nucleotide edit at genomic positions corresponding to positions A11 and A12 of the gRNA using the base editors. FIG. 12A: % of total reads showing A-to-G base editing at individual positions. This includes the reads that show base editing without indels plus the reads that harbor base editing as well as the indels. FIG. 12B: % of total reads showing only base editing at individual positions. This only includes the reads that show base editing but no indels. FIG. 12C: % of total reads showing base editing at only one specific position (left) and % of total reads showing indels (right). This excludes all the reads with base editing at multiple positions as well as the reads harboring indels.

TABLE 7
Cas12a variant for use in A-to-G base editors*

| | | | | | Unique base |
|---|---|---|---|---|---|
| | | | | Only base | editing at |
| | | | | editing | single |
| | | | Total base | without | position |
| | | Indels | editing | indels | without |
| | | (Normal- | (Normal- | (Normal- | indels |
| Variant | | ized to | ized to | ized to | (Normalized |
| name | Mutation | TBN04) | TBN04) | TBN04) | to TBN04) |
| Inactive | D832A | 2.9 | 65.6 | 109.2 | 28.2 |
| Cas12a | | | | | |
| TBN04 | K932G, | 100 | 100 | 100 | 100 |
| | N933G | | | | |
| LbAA9 | R833L | 101.7 | 60.1 | 35.2 | 34.7 |
| LbAA19 | R833K | 105.4 | 94 | 94.4 | 92.3 |
| LbEF1s9 | R833M | 111.6 | 69 | 40.5 | 38.7 |
| LbAA23 | K932E | 88.6 | 82.6 | 84.8 | 78.8 |
| LbMS07 | Q944K | 108.2 | 90.4 | 83.9 | 87.1 |
| LbAA49 | K940G | 130.4 | 86.8 | 62.5 | 58 |
| LbAC10 | Q944K | 189.5 | 119.4 | 98.2 | 84.5 |
| LbMS3n5 | K932G, | 106.3 | 92.5 | 90.8 | 87.7 |
| | N933G, | | | | |
| | V936G, | | | | |
| | S929G | | | | |
| LbTN37 | K940G, | 37.7 | 75.7 | 100.9 | 83.8 |
| | Q944K | | | | |
| LbTN39 | R836G, | 21.3 | 63.3 | 93.7 | 61.3 |
| | Q944K | | | | |
| LbTN2 | R833M, | 52.9 | 105.4 | 132.3 | 77.8 |
| | E835D, | | | | |
| | Y943T | | | | |
| LbFM14 | R836G, | 0.7 | 65.4 | 112 | 34.3 |
| | Q944K, | | | | |
| | R935G | | | | |
| LbFM17 | R833M, | 1.2 | 66.7 | 113.8 | 24.8 |
| | E835D, | | | | |
| | Y943T, | | | | |
| | R935G | | | | |
| LbFM28 | R833M, | 1 | 93.7 | 160.2 | 27.5 |
| | E835D, | | | | |
| | Y943T, | | | | |
| | Q941K | | | | |
| LbFM44 | R833M, | 23.2 | 94.5 | 146.8 | 71.7 |
| | E835D, | | | | |
| | E125A | | | | |
| LbFM51 | Y943F, | 9.3 | 113.8 | 199.9 | 86.7 |
| | Q944K, | | | | |
| | K932G, | | | | |
| | N933G, | | | | |
| | E125A | | | | |
| LbFM64 | R836G, | 0.6 | 145.8 | 262.0 | 85.4 |
| | Q944K, | | | | |
| | R935G, | | | | |
| | E125A | | | | |
| LbFM65 | R833M, | 0.4 | 131.2 | 235.8 | 43.1 |
| | E835D, | | | | |
| | Y943T, | | | | |
| | R935G, | | | | |
| | E125A | | | | |
| LbFM67 | R833M, | 0.4 | 134.5 | 241.4 | 42.9 |
| | E835D, | | | | |
| | Y943T, | | | | |
| | Q941K, | | | | |
| | E125A | | | | |
| LbFM76 | D832A, | 1.0 | 153.8 | 276.1 | 65.8 |
| | Y943F, | | | | |
| | Q944K, | | | | |
| | K932G, | | | | |
| | N933G, | | | | |
| | E125A | | | | |

*based on Base Editor LbABE8e SEQ ID NO: 177 and gRNA SEQ ID NO: 189 in HEK 293T cells. Editing efficiencies are corresponding to A-to-G base editing at position A11.

### Example 6. Efficiency and Specificity of A-to-C Base Editors

The N-methyl purine glycosylase (MPG) coding sequence was inserted at the N-terminus (LbABE8e_nMPG) or at the C-terminus (LbABE8e_cMPG) of the LbABE8e construct (A-to-G base editor). The TadA domain functions to convert adenine to inosine. In the absence of MPG, the inosine is then replaced by guanine during DNA replication and DNA repair. However, in the presence of MPG, inosine is instead removed by MPG as part of the base excision (BER) repair pathway to form an AP site, which is further processed to result in an A-to-C conversion.

The base editing efficiency and specificity of these A-to-C base editors (containing inactive LbCas12a or TBN04 (LbCas12a variant)) were determined. HEK 293T cells were transfected using a plasmid expressing the base editor together with a plasmid expressing the gRNA. Transfected cells were sorted after 3 days and re-cultured for another 3 days after which the cells were lysed and genomic DNA was extracted. Genomic region around the gRNA was PCR amplified and editing was analyzed by next generation sequencing.

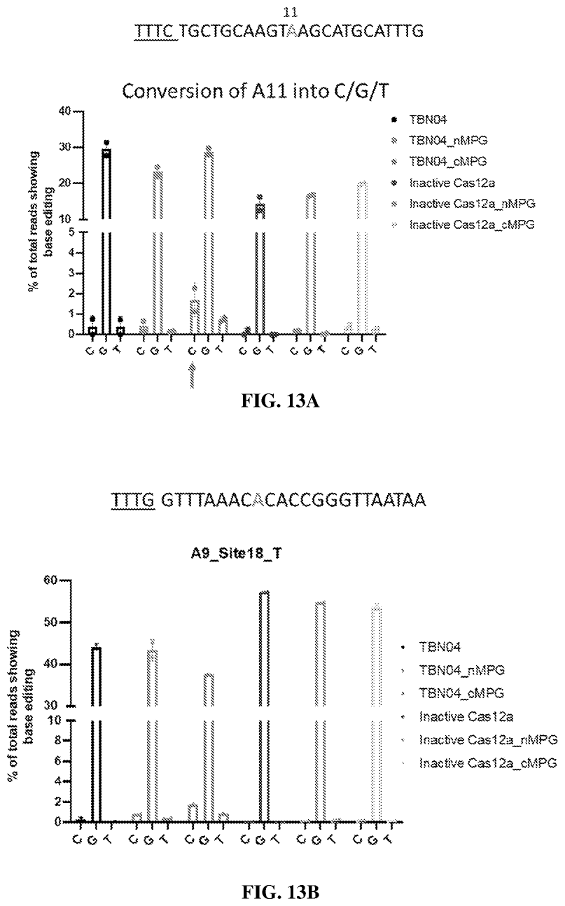

The A-to-C base editor containing the TBN04 and MPG (TBN04_nMPG and TBN04_cMPG) showed higher A-to-C base editing efficiency than base editors containing inactive Cas12a. The A-to-C base editors demonstrated high selectivity for specific sites. For gRNA TGCTGCA₇A₈GTA₁₁A₁₂GCA₁₅TGCATTTG (SEQ ID NO: 189), the base editors showed selective editing at A11 (FIG. 13A). For gRNA GTTTA₅A₆A₇CA₉CA₁₁CCGGGTTA₁₉A₂₀TA₂₂A₂₃ (SEQ ID NO: 190), the base editors showed selective editing at A9 (FIG. 13B).

FIG. 13A: % of total reads showing A-to-C, A-to-G, or A-to-T base editing at position A₁₁ of TGCTGCA₇A₈GTA₁₁A₁₂GCA₁₅TGCATTTG (SEQ ID NO: 189). FIG. 13B: % of total reads showing A-to-C, A-to-G, or A-to-T base editing at position A9 of GTTTA₅A₆A₇CA₉CA₁₁CCGGGTTA₁₉A₂₀TA₂₂A₂₃ (SEQ ID NO: 190).

### Construct Structures

The structure of LbABE8e nMPG is as follows:

MPG-Linker5-BP NLS-TadA-Linker4-LbCas12a-Linker2-BP NLS; wherein LbCas12a can be inactive LbCas12a or TBN04 (LbCas12a variant).

The structure of LbABE8e_cMPG is as follows:

BP NLS-TadA-Linker4-LbCas12a-Linker5-MPG-Linker2-BP NLS; wherein LbCas12a can be inactive LbCas12a or TBN04 (LbCas12a variant).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. An engineered variant Cas12a endonuclease comprising a polypeptide sequence comprising a mutation at an amino acid position corresponding to position E95, E125, N256, R747, H759, N813, K932, N933, S934, V936, S982, or K984 with reference to amino acid position numbering of LbCas12a ND2006, optionally wherein the endonuclease exhibits hyperactivity.

2. The engineered variant Cas12a endonuclease of claim 1, wherein the mutation is E95R, E95Y, E125A, E125W, N256A, R747Y, H759V, H759D, N813R, N813H, K932L, N933E, N933V, S934Q, V936E, V936M, V936K, S982N, or K984R.

3-15. (canceled)

16. An engineered variant Cas12a endonuclease comprising a polypeptide sequence comprising a mutation at an amino acid position corresponding to position N256, 1831, K932, N933, S934, V936, Q944, S982, F983, K984, M986, or T988 with reference to amino acid position numbering of LbCas12a ND2006, optionally wherein the endonuclease exhibits hypoactivity.

17. The engineered variant Cas12a endonuclease of claim 16, wherein the mutation is N256K, I831A, I831Y, K932A, K932F, K932H, K932M, K932N, K932Q, K932R, K932S, K932T, K932W, K932Y, N933L, S934W, V936G, Q944D, Q944E, Q944K, Q944M, S982T, S982W, F983G, F983L, K984F, M986G, M986L, M986S, or T988F.

18-24. (canceled)

25. The engineered variant Cas12a endonuclease of claim 16, comprising a mutation at an amino acid position corresponding to position Q944, optionally wherein the mutation is Q944D, Q944E, Q944K, or Q944M, further optionally wherein the polypeptide sequence has at least 80%, at least 85%, at least 90%, at least 95%, at least 98%, or 100% identity to the amino acid sequence of SEQ ID NO: 86, 87, 88, or 89.

26-30. (canceled)

31. The engineered variant Cas12a endonuclease of claim 16, wherein the polypeptide sequence comprises mutations selected from: K932F and F983L; K932F and T988F; K932R and Q944D; K932R and F983L; K932R and T988F; K932Y and F983L; K932Y and T988F; N933L and Q944M; V936G and Q944D; V936G and S982W; V936G and M986G; V936G and T988F; Q944D and S982W; Q944D and F983L; Q944D and T988F; S982W and F983L; S982W and T988F; or F983G and M986G.

32-33. (canceled)

34. The engineered variant Cas12a endonuclease of claim 31, comprising any of the following mutations;
(i) K932R and Q944D, optionally wherein the polypeptide sequence has at least 80%, at least 85%, at least 90%, at least 95%, at least 98%, or 100% identity to the amino acid sequence of SEQ ID NO: 104;
(ii) N933L and Q944M, optionally wherein the polypeptide sequence has at least 80%, at least 85%, at least 90%, at least 95%, at least 98%, or 100% identity to the amino acid sequence of SEQ ID NO: 109;
(iii) V936G and Q944D, optionally wherein the polypeptide sequence has at least 80%, at least 85%, at least 90%, at least 95%, at least 98%, or 100% identity to the amino acid sequence of SEQ ID NO: 110;
(iv) Q944D and S982W, optionally wherein the polypeptide sequence has at least 80%, at least 85%, at least 90%, at least 95%, at least 98%, or 100% identity to the amino acid sequence of SEQ ID NO: 114;
(v) Q944D and F983L, optionally wherein the polypeptide sequence has at least 80%, at least 85%, at least 90%, at least 95%, at least 98%, or 100% identity to the amino acid sequence of SEQ ID NO: 115; or
(vi) Q944D and T988F, optionally wherein the polypeptide sequence has at least 80%, at least 85%, at least 90%, at least 95%, at least 98%, or 100% identity to the amino acid sequence of SEQ ID NO: 116.

35-49. (canceled)

50. An engineered variant Cas12a endonuclease comprising a polypeptide sequence comprising a mutation at an amino acid position corresponding to position N813, 1831, K932, N933, S934, V936, Q944, S982, F983, K984, M986, or T988 with reference to amino acid position numbering of LbCas12a ND2006, wherein the endonuclease exhibits low indiscriminate ssDNase activity.

51. The engineered variant Cas12a endonuclease of claim 50, wherein the mutation is N813H, N813R, N813W, I831A, I831Y, K932A, K932F, K932H, K932M, K932N, K932Q, K932R, K932S, K932T, K932W, K932Y, N933E, N933L, S934K, S934Q, V936E, V936G, Q944D, Q944E, Q944K, S982W, F983G, F983L, K984F, M986F, M986G, or T988F.

52-58. (canceled)

59. The engineered variant Cas12a endonuclease of claim 50, comprising a mutation at an amino acid position corresponding to position Q944, optionally wherein the mutation is Q944D, Q944E, or Q944K, further optionally wherein the polypeptide sequence has at least 80%, at least 85%, at least 90%, at least 95%, at least 98%, or 100% identity to the amino acid sequence of SEQ ID NO: 86, 87, or 88.

60-64. (canceled)

65. The engineered variant Cas12a endonuclease of claim 50, wherein the mutations positions: are (i) N933L and Q944M or (ii) F983G and M986G.

66. The engineered variant Cas12a endonuclease of claim 65, comprising the mutations N933L and Q944M, optionally wherein the polypeptide sequence has at least 80%, at least 85%, at least 90%, at least 95%, at least 98%, or 100% identity to the amino acid sequence of SEQ ID NO: 109.

67-75. (canceled)

76. A fusion protein comprising an engineered variant Cas12a endonuclease of claim 110 and a base editing enzyme, optionally wherein the base editing enzyme comprises a deaminase, a guanine oxidase, or a guanine methyltransferase.

77-80. (canceled)

81. The fusion protein of claim 76, wherein the deaminase is a cytidine deaminase or an adenosine deaminase, optionally wherein the deaminase comprises a rAPOBEC1 polypeptide, an evoAPOBEC1 polypeptide, a hAPOBEC3A polypeptide, an evoCDA polypeptide, an evoFERNY polypeptide, or a TadA polypeptide.

82. (canceled)

83. The fusion protein of claim 81, further comprising:
(i) a uracil glycosylase inhibitor (UGI);
(ii) one or more nuclear localization signal (NLS), optionally selected from an SV40 NLS, a nucleoprotein (NP) NLS, and a bipartite (BP) NLS;
(iii) a uracil DNA glycosylase (UNG), optionally a human UNG (hUNG) or an Escherichia coli UNG (eUNG);
(iv) a N-methyl purine glycosylase (MPG), optionally wherein the MPG is positioned at or near the N-terminal or C-terminal ends of the fusion protein;
(v) one or more linker, optionally wherein the linker comprises the sequence of SGSETPGTSESATPES (SEQ ID NO: 203) or SGGSSGGSSGSETPGTSESATPESSGGSSGGS (SEQ ID NO: 204); and/or
(vi) a DNA binding domain (DBD), optionally wherein the DBD is a Rad51 DBD.

84-91. (canceled)

92. A polynucleotide encoding an engineered variant Cas12a endonuclease of claim 110.

93. A cell comprising (a) an engineered variant Cas12a endonuclease of claim 110 and (b)
a guide RNA (gRNA) or a polynucleotide encoding a gRNA, optionally wherein the cell is a human cell.

94-97. (canceled)

98. A method of gene editing comprising
(i) contacting a target nucleic acid sequence with the fusion protein of claim 76 and a guide RNA, wherein the target nucleic acid comprises a target nucleobase; and
(ii) modifying the target nucleobase.

99-105. (canceled)

106. The method of claim 98, wherein the method is performed in vitro, ex vivo, or in vivo.

107-109. (canceled)

110. An engineered variant Cas12a endonuclease comprising a polypeptide sequence comprising one or more mutations at amino acid positions corresponding to positions R833, E835, R836, F931, R935, K940, Q941, Y943, and/or Q944, with reference to amino acid position numbering of LbCas12a ND2006.

111. The engineered variant Cas12a endonuclease of claim 110, wherein the one or more mutations are selected from R833L, R833K, R833M, E835D, R836G, R935G, K940G, Q941K, Y943T, Y943F, and Q944K.

112. The engineered variant Cas12a endonuclease of claim 111, wherein the mutations are K940G and Q944K; R836G and Q944K; R833M, E835D, and Y943T; R836G, Q944K, and R935G; R833M, E835D, Y943T, and R935G; or R833M, E835D, Y943T, and Q941K.
